(19) United States
(12) Patent Application Publication
(10) Pub. No.: US 2025/0118393 A1
(43) Pub. Date: Apr. 10, 2025

(54) METHODS AND SYSTEMS FOR DATA DOWN-SAMPLING

(71) Applicant: Regeneron Pharmaceuticals, Inc., Tarrytown, NY (US)

(72) Inventors: Kaitlyn Gayvert, Tarrytown, NY (US); Sheldon McKay, Tarrytown, NY (US); Wei Keat Lim, Tarrytown, NY (US); Richard Copin, Tarrytown, NY (US); Gurinder S. Atwal, Tarrytown, NY (US)

(21) Appl. No.: 18/908,492

(22) Filed: Oct. 7, 2024

Related U.S. Application Data
(60) Provisional application No. 63/588,551, filed on Oct. 6, 2023.

(51) Int. Cl. G16B 50/50; G16B 30/00

(52) U.S. Cl. CPC G16B 50/50; G16B 30/00

(57) ABSTRACT

Methods and systems data down-sampling are disclosed. A method includes selecting data samples from a first set of data samples, each associated with a sampling date, that meet at least one quality control criterion to generate a second set of data samples, and down-sampling the second set of data samples into a plurality of subsets of data samples. Each subset of the plurality of subsets of data samples contains a data sample predetermined quantity and is associated with a different time period. Down-sampling includes generating, based on the sampling date, a sampling date distribution for the second set of data samples, and selecting, for each time period of the plurality of sequential time periods, based on the sampling date distribution, data samples from the second set of data samples until each subset of the plurality of subsets of data samples contains the predetermined quantity of data samples.

### CROSS-REFERENCE TO RELATED PATENT APPLICATION

This application claims priority to U.S. Provisional Application No. 63/588,551, filed on Oct. 6, 2023, the entirety of which is incorporated by reference herein.

### BACKGROUND

With the advent of big data and the increase in the volume of digitally stored information, datasets comprising tens or hundreds of millions of records have become commonplace. The manipulation and analysis of these extensive datasets pose significant computational challenges due to their size and complexity. Traditional processing techniques often prove inefficient and costly in terms of required time and computational resources, creating bottlenecks that hinder timely data analysis and interpretation. Furthermore, as the size of datasets grows, the likelihood of redundant or irrelevant data inclusion also increases, which may compromise the accuracy and reliability of any analytical insights derived from the data.

In light of these challenges, there is a critical need for innovative down-sampling methods that effectively reduce the size of large datasets while maintaining the integrity and representativeness of the original data. Such novel techniques should be capable of intelligently selecting subsets of data that accurately reflect the characteristics and variability inherent in the full dataset, thereby enabling more efficient and accurate computational processes. The introduction of effective down-sampling techniques would not only expedite data processing tasks but would also facilitate more robust and reliable analytical outcomes, fostering better decision-making and strategy formulation in various fields, including but not limited to, finance, healthcare, and information technology.

### BRIEF SUMMARY

Methods and systems for down-sampling data are disclosed. An example method may include selecting data samples from a first set of data samples that meet at least one quality control (QC) criterion to generate a second set of data samples. Each of the first set of data samples is associated with a sampling date. The method may further include down-sampling the second set of data samples into a plurality of subsets of data samples. Each subset of the plurality of subsets of data samples contains a predetermined quantity of data samples, and each subset of the plurality of subsets of data samples is associated with a different time period of a plurality of sequential time periods. Down-sampling the second set of data samples into the plurality of subsets of data samples may include generating, based on the sampling date associated with each data sample of the second set of data samples, a sampling date distribution for the second set of data samples, and selecting, for each time period of the plurality of sequential time periods, based on the sampling date distribution, data samples from the second set of data samples until each subset of the plurality of subsets of data samples contains the predetermined quantity of data samples.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows an example method to down-sample data in accordance with embodiments of the disclosure.

FIG. 2A-2E shows SARS-CoV-2 diversity across geographic regions and phylogenetic lineages. FIG. 2A shows cumulative count of SARS-CoV-2 genome sequences in GISAID by collection date and continent of sample origin as of Nov. 1, 2022. FIG. 2B shows distribution of high frequency variants in the spike protein sequence. The RBD and NTD domains are labeled, respectively. For a number of residues with high deviation from wild type sequence (e.g. 417, 452, 484, 501, 614), the most common mutation is indicated. FIG. 2C shows distribution of haplotypes of the 6 previous and current variants of concern (Alpha, Beta, Gamma, Delta, Omicron, Omicron BA.4/5), mapped onto the phylogeny. FIG. 2D shows SARS-CoV-2 maximum-likelihood phylogeny of high-diversity 1000-genome subset. The tree is outgroup rooted with the related bat CoV genome RATG13. The root branch length is truncated to emphasize relationships amongst SARS-CoV-2 genomes. The geographic origin of each of the sequences is denoted by branch shading. FIG. 2E shows distribution of mutations under convergent evolution (L452R, N501Y), mapped onto the phylogeny.

FIG. 3 shows a gene-level summary of the HFV and sites under in accordance with embodiments of the disclosure.

FIG. 4 shows a protein-level summary of the HFV and sites under selection in accordance with embodiments of the disclosure.

FIGS. 5A-5D show amino acid sites under natural selection in SARS-CoV-2 genome in accordance with embodiments of the disclosure. FIG. 5A shows gene length normalized counts (counts per kb) of sites under significant purifying selection (lighter-shaded bars) and diversifying selection (darker-shaded bars). FIG. 5B shows a scatterplot of percent of sites under purifying selection vs percent of sites under diversifying selection in each protein. While overall the genome was under strong purifying selection, a substantial number of sites were identified as under diversifying selection in certain regions (egg. ORF3a, ORF10, N). FIG. 5C shows a percentage of sites under purifying (top panel) and diversifying (bottom panel) selection per gene across the three defining time periods of the pandemic: (1) the pre-vaccine era, (2) the post-vaccine era, and (3) the emergence of omicron lineages. FIG. 5D shows a level of evidence (1—probability of selection) (black lines and points) and global frequency of mutations (gray lines and points) at selected sites under diversifying selection in spike across the three defining time periods of the pandemic: (1) the pre-vaccine era, (2) the post-vaccine era, and (3) the emergence of omicron lineages. alpha: synonymous mutation rate; beta: non-synonymous mutation rate; Prob[alpha>=beta]: 1—probability of diversifying selection at the site.

FIGS. 6A-6C show amino acid sites under natural selection in SARS-CoV-2 genome in accordance with embodiments of the disclosure. FIG. 6A shows a gene length normalized counts (counts per kb) of sites under significant purifying selection (lighter-shaded bars) and diversifying selection (darker-shaded bars). FIG. 6B shows a scatterplot of percent of sites under purifying selection vs percent of sites under diversifying selection in each protein. While overall the genome was under strong purifying selection, a substantial number of sites were identified as under diversifying selection in certain regions (egg. ORF3a, ORF10, N). FIG. 6C shows a percentage of sites under purifying (top panel) and diversifying (bottom panel) selection per gene across the three defining time periods of the pandemic: (1) the pre-vaccine era, (2) the post-vaccine era, and (3) the emergence of omicron lineages.

FIG. 7 shows amino acid sites under natural selection in SARS-CoV-2 genome, in accordance with embodiments of the disclosure, with level of evidence (1—probability of selection) (black lines and points) and global frequency of mutations (gray lines and points) at selected sites under diversifying selection in spike across the three defining time periods of the pandemic: (1) the pre-vaccine era, (2) the post-vaccine era, and (3) the emergence of omicron lineages. alpha: synonymous mutation rate; beta: non-synonymous mutation rate; Prob[alpha>=beta]: 1—probability of diversifying selection at the site.

FIG. 8 shows the probability of selection for the top 90 spike sites that have undergone diversifying selection, in accordance with embodiments of the disclosure, grouped by whether they were under selection at the Sep. 30, 2022 analysis date (top) or no longer under selection (bottom).

FIGS. 9A-9E show an example impact of selective forces on SARS-CoV-2 infectivity and antibody neutralization, in accordance with embodiments of the disclosure. FIG. 9A shows that for each possible amino acid substitution within the RBD, the effect of the mutation on RBD expression (x-axis) and ACE2 binding (y-axis). Each substitution is colored according to whether its associated site is under diversifying (black), purifying (medium gray), or neutral (light gray) selection. FIG. 9B during the pre-vaccine and current post-Omicron eras. FIG. 9C shows sites detected to be under purifying selection (medium gray) or diversifying selection (black) within the RBD of the S protein. Overlayed are the sites associated with class 1, class 2, class 3, and class 4 antibody epitopes. FIG. 9D shows deep mutational scan site total escape from convalescent plasma values for sites under diversifying (black), purifying (medium gray), or neutral (light gray) selection. FIG. 9E shows a boxplot of the incident rates (cases per 100,000) for each sample in the pre-vaccination era (collected in 2020), grouped by whether that sample contained a mutation that reduced neutralization (G446V or mutations at sites L452, E484, or K417), increased ACE2 binding without any impact on neutralization (T478K or mutations at sites N501 or S477), or had no effect on neutralization and binding (A520S or mutations at site A522).

FIG. 10A shows a boxplot of the number of possible amino acid substitutions that decrease ACE2 binding for each position in the RBD, grouped by whether the site is under diversifying (black), purifying (medium gray), or neutral (light gray) selection, in accordance with embodiments of the disclosure.

FIG. 10B shows, for each possible amino acid substitution within the RBD, scatterplot of the effect on ACE2 binding, effect on RBD expression, and average escape from convalescent antibodies, in accordance with embodiments of the disclosure.

FIG. 10C shows, for each possible amino acid substitution within the RBD, scatterplot of the effect on ACE2 binding, effect on RBD expression, and average escape from convalescent antibodies, in accordance with embodiments of the disclosure.

FIG. 11 shows deep mutational scan site total escape values from convalescent plasma, antibodies from Moderna vaccinated patients, and therapeutic antibodies for sites under diversifying (black), purifying (medium gray), or neutral (light gray) selection, in accordance with embodiments of the disclosure.

FIG. 12A shows a number of isolates from the pre-vaccination era (collected on or before Dec. 31, 2020) with any combinations of RBD mutations which confer increased ACE2 binding (L452X, E484X, K417X, G446V, N501X, S477X, T478K), escape from convalescent antibodies (L452X, E484X, K417X, G446V), loss of ACE2 binding (loss) (K417X), or neither (no change) (A520S, A522X), in accordance with embodiments of the disclosure.

FIG. 12B shows, for each US state, correlation of the CDC seroprevalence estimates from Jan. 24, 2021 with the incident rate (cases per 100,000) corresponding to three weeks prior (Dec. 26, 2020), in accordance with embodiments of the disclosure.

FIGS. 13A-13F shows selective pressures within candidate variable T cell epitopes, in accordance with embodiments of the disclosure. FIGS. 13A and 13B show a distribution of the number of sequence high frequency variants (found in 10,000+ isolates) observed in (FIG. 13A) putative memory CD8+ T cell epitope sequences from recovered COVID patients, and FIG. 13B epitopes from RBD and non-RBD regions. (C-D) Number of putative memory CD8+ T cell epitope sequences from recovered COVID patients that contain sites under (FIG. 13C) diversifying and (FIG. 13D) purifying selection per gene.

FIG. 13E shows a distribution of the number of sites under selection in epitopes, broken down by diversifying and negative selection in spike peptides. FIG. 13F shows a distribution of the number of sites under purifying selection in peptides (blue) and genome-wide (grey) 15-mers.

FIG. 14 shows percent of epitopes with high frequency variants (HFV, found in 1000+ isolates), broken down by gene, in accordance with embodiments of the disclosure.

FIG. 15 shows percent of epitopes with high frequency variants (HFV, found in 1000+ isolates), broken down by ORF1ab protein product, in accordance with embodiments of the disclosure.

FIGS. 16A and 16B show a distribution of the number of HFV observed in (FIG. 16A) putative memory CD8+ T cell epitope sequences, and (FIG. 16B) epitopes from RBD and non-RBD regions, in accordance with embodiments of the disclosure.

FIGS. 17A-17C show a number of putative memory CD8+ T cell epitope sequences that contain sites under (FIG. 17A) diversifying and (FIG. 17B) purifying selection per gene (FIG. 17C), in accordance with embodiments of the disclosure. Distribution of the number of sites under selection in epitopes, broken down by diversifying and negative selection in spike peptides (top panel) putative CD4 T cell epitopes from SARS-CoV-2 naïve individuals (middle panel) putative CD8 T cell epitopes from SARS-CoV-2 naïve individuals (bottom panel) putative CD8 T cell epitopes recovered COVID patients.

FIG. 18 shows a distribution of the number of sites under purifying and diversifying selection in spike, N, ORFa1, ORF1b, ORF3a, and ORF7a peptides and genome-wide 15-mers. Significance was assessed using a one-sided Wilcoxon rank-sum test, in accordance with embodiments of the disclosure.

FIG. 19A shows Pearson correlation coefficients of the log probabilities of purifying (upper right) and diversifying (lower left) selection across six different down-sampled replicates, in accordance with embodiments of the disclosure.

FIG. 19B shows a scatterplot of the log probabilities of purifying selection between two down-sampled replicates, in accordance with embodiments of the disclosure.

FIG. 19C shows a scatterplot of the log probabilities of diversifying selection between two down-sampled replicates, in accordance with embodiments of the disclosure.

FIG. 20 shows select amino acid sites under positive selection in the SARS-CoV-2 Spike RBD, in accordance with embodiments of the disclosure.

FIG. 21 shows level of evidence (1—probability of selection) (black lines and points) and global frequency of mutations (red lines and points) at selected sites under diversifying selection in spike across the three defining time periods of the pandemic: (1) the pre-vaccine era, (2) the post-vaccine era, and (3) the emergence of omicron lineages. alpha: synonymous mutation rate; beta: non-synonymous mutation rate; Prob[alpha>=beta]: 1—probability of diversifying selection at the site, in accordance with embodiments of the disclosure.

FIG. 22 shows BA.2.86 and EG.5.1 associated mutations and evidence of positive selection in November 2022, in accordance with embodiments of the disclosure.

FIG. 23 shows level of evidence (1—probability of selection) (black lines and points) and global frequency of mutations (gray lines and points) at selected sites under diversifying selection in spike across the three defining time periods of the pandemic: (1) the pre-vaccine era, (2) the post-vaccine era, and (3) the emergence of omicron lineages. alpha: synonymous mutation rate; beta: non-synonymous mutation rate; Prob[alpha>=beta]: 1—probability of diversifying selection at the site, in accordance with embodiments of the disclosure.

FIG. 24 shows an example operational environment, in accordance with embodiments of the disclosure.

FIG. 25 shows an example method to down-sample data, in accordance with embodiments of the disclosure.

### DETAILED DESCRIPTION

The disclosed methods and systems may be understood more readily by reference to the following detailed description of particular embodiments and the Example included therein and to the Figures and their previous and following description.

It is understood that the disclosed methods and systems are not limited to the particular methodology, protocols, and reagents described as these may vary. It is also to be understood that the terminology used herein is for the purpose of describing particular embodiments only, and is not intended to limit the scope of the present invention which will be limited only by the appended claims.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of the peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### Definitions

It must be noted that as used herein and in the appended claims, the singular forms “a,” “can,” and “the” include plural reference unless the context clearly dictates otherwise. Thus, for example, reference to “a peptide” includes a plurality of such peptides, reference to “the peptide” is a reference to one or more peptide and equivalents thereof known to those skilled in the art, and so forth.

The word “or” as used herein means any one member of a particular list and also includes any combination of members of that list.

As used herein, the term “amino acid sequence” refers to a list of abbreviations, letters, characters or words representing amino acid residues. The amino acid abbreviations used herein are conventional one letter codes for the amino acids and are expressed as follows: A, alanine; C, cysteine; D aspartic acid; E, glutamic acid; F, phenylalanine; G, glycine; H histidine; I isoleucine; K, lysine; L, leucine; M, methionine; N, asparagine; P, proline; Q, glutamine; R, arginine; S, serine; T, threonine; V, valine; W, tryptophan; and Y, tyrosine.

As used herein, “align,” alignment,” and “aligning” in the context of nucleic acids refers to arranging sequences of DNA or RNA to identify regions of similarity. Similarity may be related to the nucleotide sequence, structural, functional, and/or evolutionary relationships between the sequences. Alignment of DNA sequences involves alignment of genomic DNA of one sequence to genomic DNA of at least one other sequence. Such alignment may exclude non-genomic DNA, such as a molecular barcode, padding bases, and the like. For example, genomic DNA of a sequence read may be aligned to genomic DNA of a reference DNA sequence, excluding any molecular tag or adapter sequence that may be attached to the sequence read.

As used herein, recitation that nucleotides “correspond to” nucleotides in a sequence refers to nucleotides identified upon alignment with the sequence to maximize identity using an alignment algorithm, such as the GAP algorithm.

As used herein, “sequence identity,” “sequence homology,” or “identity” refers to the number of identical or similar nucleotide bases in an alignment between two or more polynucleotide sequences. In one non-limiting example, “at least 90% identical to” refers to percent identities from 90 to 100% relative to the reference polynucleotide. Identity at a level of 90% or more is indicative of the fact that, assuming for exemplification purposes a test and reference polynucleotide length of 100 nucleotides are compared, no more than 10% (i.e., 10 out of 100) of nucleotides in the test polynucleotide differs from that of the reference polynucleotide. Such differences can be represented as point mutations randomly distributed over the entire length of a nucleotide sequence or they can be clustered in one or more locations of varying length up to the maximum allowable, e.g., 10/100 nucleotide difference (approximately 90% identity). Differences are defined as nucleic acid substitutions, insertions or deletions.

Sequence identity can be determined by sequence alignment of nucleic acid sequences to identify regions of similarity or identity. For purposes herein, sequence identity is generally determined by alignment to identify identical bases. The alignment can be local or global. Matches, mismatches and gaps can be identified between compared sequences. Gaps are null nucleotides inserted between the bases of aligned sequences so that identical or similar characters are aligned. Generally, there can be internal and terminal gaps. Sequence identity can be determined by taking into account gaps as the number of identical bases/length of the shortest sequence×100. When using gap penalties, sequence identity can be determined with no penalty for end gaps (e.g., terminal gaps are not penalized). Alternatively, sequence identity can be determined without taking into account gaps as the number of identical positions/length of the total aligned sequence×100.

As used herein, a “global alignment” is an alignment that aligns two sequences from beginning to end, aligning each base in each sequence only once. An alignment is produced regardless of whether or not there is similarity or identity between the sequences. For example, 50% sequence identity based on “global alignment” means that in an alignment of the full sequence of two compared sequences each of 100 nucleotides in length, 50% of the bases are the same. It is understood that global alignment also can be used in determining sequence identity even when the length of the aligned sequences is not the same. The differences in the terminal ends of the sequences will be taken into account in determining sequence identity, unless the “no penalty for end gaps” is selected. Generally, a global alignment is used on sequences that share significant similarity over most of their length. Exemplary algorithms for performing global alignment include the Needleman-Wunsch algorithm (Needleman et al. J. Mol. Biol. 48: 443 (1970). Exemplary programs for performing global alignment are publicly available and include the Global Sequence Alignment Tool available at the National Center for Biotechnology Information (NCBI) website (ncbi.nlm.nih.gov/), and the program available at deepc2.psi.iastate.edu/aat/align/align.html.

As used herein, a “local alignment” is an alignment that aligns two sequences, but only aligns those portions of the sequences that share similarity or identity. Hence, a local alignment determines if sub-segments of one sequence are present in another sequence. If there is no similarity, no alignment will be returned. Local alignment algorithms include BLAST or Smith-Waterman algorithm (Adv. Appl. Math. 2: 482 (1981)). For example, 50% sequence identity based on “local alignment” means that in an alignment of the full sequence of two compared sequences of any length, a region of similarity or identity of 100 nucleotides in length has 50% of the bases that are the same in the region of similarity or identity.

As used herein, “gene” refers to any segment of DNA associated with a biological function. Thus, genes include coding sequences and optionally, the regulatory sequences required for their expression. Genes also optionally include non-expressed DNA segments that, for example, form recognition sequences for other proteins.

The term “DNA (deoxyribonucleic acid)” refers to a chain of nucleotides comprising deoxyribonucleosides that each comprise one of four nucleobases, namely, adenine (A), thymine (T), cytosine (C), and guanine (G). The term “RNA (ribonucleic acid)” refers to a chain of nucleotides comprising four types of ribonucleosides that each comprise one of four nucleobases, namely; A, uracil (U), G, and C. Certain pairs of nucleotides specifically bind to one another in a complementary fashion (called complementary base pairing). In DNA, adenine (A) pairs with thymine (T) and cytosine (C) pairs with guanine (G). In RNA, adenine (A) pairs with uracil (U) and cytosine (C) pairs with guanine (G). When a first nucleic acid strand binds to a second nucleic acid strand made up of nucleotides that are complementary to those in the first strand, the two strands bind to form a double strand. A “polynucleotide,” “nucleic acid,” “nucleic acid molecule,” or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Oligonucleotides often range in size from a few monomeric units, e.g. 3-4, to hundreds of monomeric units. Whenever a polynucleotide is represented by a sequence of letters, such as “ATGCCTG,” it will be understood that the nucleotides are in 5′->3′ order from left to right and that “A” denotes adenosine, “C” denotes cytosine, “G” denotes guanosine, and “T” denotes thymidine, unless otherwise noted. The letters A, C, G, and T may be used to refer to the bases themselves, to nucleosides, or to nucleotides comprising the bases, as is standard in the art.

As used herein, “nucleic acid sequencing data,” “nucleic acid sequencing information,” “nucleic acid sequence,” “nucleotide sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine or uracil) in a molecule (e.g., a whole genome, whole transcriptome, exome, oligonucleotide, polynucleotide, or fragment) of a nucleic acid such as DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, and electronic signature-based systems.

A nucleic acid sequence may be eukaryotic in origin. In this regard, eukaryotic genes are comprised of “exons” and “introns.” The term “exon,” as used herein, refers to a nucleic acid sequence present in a gene which is represented in the mature form of an RNA molecule after excision of introns during transcription. Exons are translated into protein. The term “intron,” as used herein, refers to a nucleic acid sequence present in a given gene which is not translated into protein and is generally found between exons. During transcription, introns are removed from precursor messenger RNA (pre-mRNA), and exons are joined via RNA splicing. Thus, in an embodiment, a nucleic acid sequence may comprise one or more exons and introns. The term “transcription,” as used herein, is the process of creating an equivalent RNA copy of a sequence of DNA, and involves the steps of initiation, elongation, termination, and RNA processing (which includes splicing) (see, e.g., Griffiths et al., eds., Modern Genetic Analysis: Integrating Genes and Genomes, 2nd ed., W.H. Freeman and Co., New York (2002)).

As used herein, the terms “sequencing” or “sequencer” refer to any of a number of technologies used to determine the sequence of a biomolecule, e.g., a nucleic acid such as DNA or RNA. Exemplary sequencing methods include, but are not limited to, targeted sequencing, single molecule real-time sequencing, exon sequencing, electron microscopy-based sequencing, panel sequencing, transistor-mediated sequencing, direct sequencing, random shotgun sequencing, Sanger dideoxy termination sequencing, whole-genome sequencing, sequencing by hybridization, pyrosequencing, duplex sequencing, cycle sequencing, single-base extension sequencing, solid-phase sequencing, high-throughput sequencing, massively parallel signature sequencing, emulsion PCR, co-amplification at lower denaturation temperature-PCR (COLD-PCR), multiplex PCR, sequencing by reversible dye terminator, paired-end sequencing, near-term sequencing, exonuclease sequencing, sequencing by ligation, short-read sequencing, single-molecule sequencing, sequencing-by-synthesis, real-time sequencing, reverse-terminator sequencing, nanopore sequencing, 454 sequencing, Solexa Genome Analyzer sequencing, SOLiD™ sequencing, MS-PET sequencing, and a combination thereof. In some embodiments, sequencing can be performed by a gene analyzer such as, for example, gene analyzers commercially available from Illumina or Applied Biosystems.

As used herein, “sequence fragment” refers to a piece of a nucleic acid molecule that can vary in length and can carry the sequence information (or sequence data) of the nucleic acid molecule. The sequence information can be derived from sequencing reads obtained from sequencing the sequence fragments.

As used herein, “read,” “sequence read,” or “sequencing read” refers to the sequence of base pairs corresponding to all or a part of a sequence fragment.

“Optional” or “optionally” means that the subsequently described event, circumstance, or material may or may not occur or be present, and that the description includes instances where the event, circumstance, or material occurs or is present and instances where it does not occur or is not present.

“Peptide” as used herein refers to any peptide, oligopeptide, polypeptide, gene product, expression product, or protein. A peptide is comprised of consecutive amino acids. The term “peptide” encompasses naturally occurring or synthetic molecules.

As used herein, “sample” is meant to mean an animal; a tissue or organ from an animal; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

The term “viral DNA” as used herein refers to a sequence of DNA that is found in virus particles.

The term “genome,” as used herein, generally refers to the complete set of genetic information in the form of one or more nucleic acid sequences, including text or in silico representations thereof. A genome may include either DNA or RNA, depending upon its organism of origin. Most organisms have DNA genomes while some viruses have RNA genomes. As used herein, the term “genome” need not comprise the complete set of genetic information. The term may also refer to at least a majority portion of a genome such as at least 50% to 100% of an entire genome or any whole or fractional percentage therebetween.

The term “viral genome” as used herein refers to the totality of the DNA that is found in virus particles, and that contains all the elements necessary for virus replication. The genome is replicated and transmitted to the virus progeny at each cycle of virus replication.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents forms part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### DESCRIPTION

Comprehensively manipulating and analyzing large data sets can be computationally infeasible, especially for extremely large datasets and/or extremely complex datasets. For example, datasets with tens or hundreds of millions of records have become commonplace. The manipulation and analysis of these extensive datasets pose significant computational challenges due to their size and complexity. This disclosure describes methods and systems that use down-sampling techniques for time-series data samples to reduce a number of samples to be manipulated and analyzed, while still maintaining a representative cross-section of high quality data samples, to form a computationally feasible data set. By reducing a number of data samples, manipulation and analysis may be more efficiently and timely completed to avoid bottlenecks that exist in traditional data analysis methods. The methods described herein may also improve the quality of the analyzed data samples by eliminating data samples that fail to meet a quality metric. The described methods are capable of intelligently selecting subsets of data that accurately reflect the characteristics and variability inherent in the full dataset, thereby enabling more efficient and accurate computational processes. The introduction of effective down-sampling techniques would not only expedite data processing tasks but would also facilitate more robust and reliable analytical outcomes, fostering better decision-making and strategy formulation in various fields, including but not limited to, finance, healthcare, and information technology.

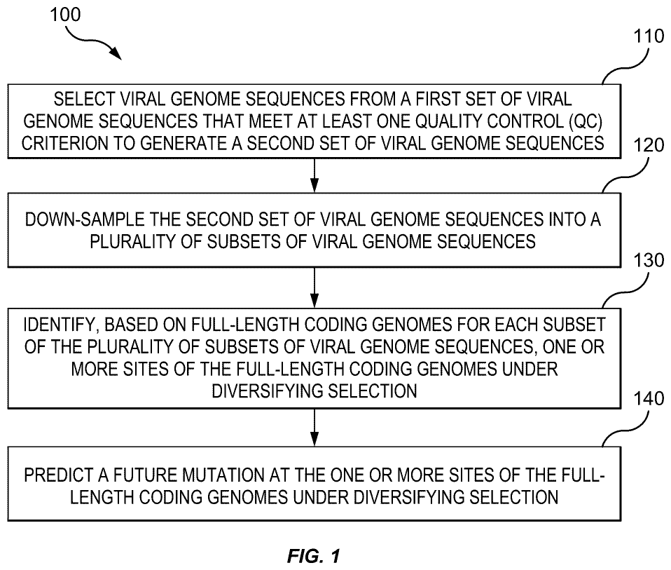

FIG. 1 shows an example method 100 to down-sample data in accordance with embodiments of the disclosure. The method 100 may be performed via a computing device configured to execute instructions encoded with the steps of the method 100.

The method 100 includes selecting viral genome sequences from a first set of viral genome sequences that meet at least one quality control (QC) criterion to generate a second set of viral genome sequences, at 110. Each of the first set of viral genome sequences is associated with a sampling date. The at least one QC criterion may include, for example, one or more of: requiring complete accession metadata, allowing less than five percent ambiguous DNA nucleotides or translated amino sequences, preventing sequences with runs of six or more consecutive ambiguous nucleotides or amino sequences, requiring the presence of all canonical gene-based open reading frames with full-length Basic Local Alignment Search Tool (BLAST) hits against a reference genome, or excluding sequences with open reading frames truncated by premature stop codons, with the exception of ORF8.

In some examples, the method 100 includes receiving the first set of viral genome sequences of a same viral species. The first set of viral genome sequences may be received as part of performing a sequencing process and/or may be received by downloading the viral genome sequences from an electronic data source. Each viral genome sequence may be associated with a sampling date. For example, each viral genome sequence may be associated with patient-related metadata that can be used to determine a time of infection and a geographical origin of virus isolates. The viral species may be, for example, SARS-CoV-2 or any other virus. The first set of viral genome sequences may comprise millions, tens of millions, hundreds of millions of genome sequences, and the like. Detailed analysis (e.g., phylogenetic analysis) of a data set on the order of millions of genome samples is computationally challenging due to the combinatorial complexity of evaluating relationships between all samples. To obtain a computationally feasible data set, a down-sampling method is disclosed to select a subset of representative viral genomes. The disclosed down-sampling method may comprise one or more stages.

The method 100 may further include down-sampling the second set of viral genome sequences into a plurality of subsets of viral genome sequences, at 120. Each subset of the plurality of subsets of viral genome sequences contains a predetermined quantity of viral genome sequences, and each subset of the plurality of subsets of viral genome sequences is associated with a different timepoint of a plurality of timepoints. Down-sampling the second set of viral genome sequences to contain the predetermined quantity of viral genome sequences may include generating, based on the sampling date associated with each viral genome sequence of the second set of viral genome sequences, a sampling date distribution for the second set of viral genome sequences, and selecting, for each timepoint of the plurality of timepoints, based on the sampling date distribution, viral genome sequences from the second set of viral genome sequences until each subset of the plurality of subsets of viral genome sequences contains the predetermined quantity of viral genome sequences.

The predetermined quantity may include any predetermined quantity of viral genome sequences required to achieve a desired size of each subset of the plurality of subsets of viral genome sequences. The desired size may be, for example, at least ten thousand.

In some examples, selecting the viral genome sequences from the second set of viral genome sequences may include, for any timepoint of the plurality of timepoints with more than the predetermined quantity of viral genome sequences available, randomly selecting viral genome sequences from the second set of viral genome sequences until the subset of the plurality of subsets of viral genome sequences associated with the timepoint contains the predetermined quantity of viral genome sequences. In addition, for any timepoint with less than the predetermined quantity of viral genome sequences available, the selection may include retaining all viral genome sequences for the subset of the plurality of subsets of viral genome sequences associated with the timepoint. This selection of viral genome sequences may ensure that each subset has a same quantity if more than the quantity are available, and for timepoints that have fewer than the quantity, all of the available samples are included to ensure each subset is as close as possible to perform the statistical analysis and follow-on prediction of a future mutation.

In some examples, the method 100 may further include selecting, for each timepoint of the plurality of timepoints, a representative viral genome sequence from the second set of viral genome sequences of each lineage of a plurality of lineages for each subset of the plurality of subsets of viral genome sequences. The plurality of lineages may include, for example, a plurality of Phylogenetic Assignment of Named Global Outbreak (Pango) viral lineages. Making sure each viral lineage is included may better capture past history and currently emerging lineages and improve accuracy in a follow-on prediction as compared with implementations that do not include each viral lineage.

The plurality of timepoints may represent an end of a month, three-month quarter, a 6-month half year, a year, and the like. Down-sampling the second set of viral genome sequences into the plurality of subsets of viral genome sequences may include generating, based on the sampling dates associated with each viral genome sequence of the second set of viral genome sequences, a sampling date distribution for the second set of viral genome sequences. The method 100 may further include selecting, for each timepoint of the plurality of timepoints, based on the sampling date distribution, viral genome sequences from the second set of viral genome sequences until each subset contains the second predetermined quantity of viral genome sequences. Generating the sampling date distribution may include fitting a quantity of viral genome sequences from the second set of viral genome sequences to a log curve or an exponential curve. Fitting the quantity of viral genome sequences from the second set of viral genome sequences to the log curve or to the exponential curve may include generating the sampling date distribution by fitting the quantity of viral genome sequences from the second set of viral genome sequences for each month to the log curve or the exponential curve. In other examples, generating the sampling date distribution may include fitting a quantity of viral genome sequences from the second set of viral genome sequences to a predetermined statistical curve or trend. Fitting the quantity of viral genome sequences to an exponential curve, log curve, or other statistical curve or trend may provide a more representative selection of samples in cases where the quantity of available viral genome sequences for each time point are not consistent (e.g., many more available viral genome sequences for a first time point than are available for a later time point).

The subsets of viral genome sequences may be configured for selection analysis. The selection analysis is facilitated by the computationally feasible number of viral genome sequences within each subset of viral genome sequences. By reducing a number of data samples, manipulation and analysis may be more efficiently and timely and computationally feasibly completed as compared with using all available viral genome sequences. Because amino acids are encoded by triplet codon sequences in the DNA, which are organized into open reading frames (e.g., viral genes), the DNA sequences have to be aligned with a codon aware aligner that maintains the codon reading frame. Thus, in an embodiment, the method 100 may include, performing selection analysis. To perform selection analysis, the method 100 may include, identifying, based on full-length coding genomes for each subset of the plurality of subsets of viral genome sequences, one or more sites of the full-length coding genomes under diversifying selection, at 130. In some examples, the method 100 may further include, within each subset of viral genome sequences, aligning coding sequences of each full-length gene-based open reading frame. Aligning coding sequences of each full-length gene-based open reading frame may include applying a codon-aware multiple sequence aligner. In some examples, the method 100 may include, for each subset of viral genome sequences, assembling the aligned open reading frame sequences into the full-length coding genome.

The method 100 may further include predicting a future mutation at the one or more sites of the full-length coding genomes under diversifying selection, at 140. The method 100 may further include developing a therapeutic based on the one or more future mutations.

Performing selection analysis may include generating a phylogenetic tree using one or more of the full-length coding genomes. Performing selection analysis may include natural selection analysis. A selection test may be performed using, for example, a Fast Unconstrained Bayesian AppRoximation (FUBAR) method from the hypothesis testing using phylogenies (HyPhy) Suite (https://stevenweaver.github.io/hyphy-site). The analysis may be conducted for one or more genes (e.g., each gene). The alignment and corresponding whole-genome phylogenies described above may be inputted into FUBAR to infer nonsynonymous (dN) and synonymous substitution (dS) rates at a per-site basis and test whether dN was significantly different from dS. Sites with probability values>0.9, for example, may be considered to be under non-neutral selection. This may repeated at the timepoints to understand the changes over time. For the purposes of interpretation, the timepoints may binned into one or more pandemic phases, such as pre-vaccine, post-vaccine, and/or post-novel lineage periods.

The severe acute respiratory syndrome coronavirus 2 (SARS-CoV-2), the etiologic agent of the coronavirus disease 2019 (COVID-19) global pandemic, has prompted an extensive effort to understand its adaptation mechanisms. Scientists and medical professionals worldwide have sequenced the SARS-CoV-2 genome from patient isolates, and their findings have been rapidly disseminated through curated data repositories such as the global initiative on sharing all influenza data (GISAID, https://www.gisaid.org). This unprecedented level of data sharing has provided a unique dataset critical to determine transmission patterns and identify variants that may be associated with virulence and disease severity.

The biological forces driving the conservation and diversity of SARS-COV-2 genome are currently not well understood. In the past, understanding these forces has been critical to inform treatment development against other life-threatening infections. While mutations emerge randomly in the genome, the accumulation or loss of variants can be driven by changes within the host environment. For example, vaccination or emergence of a new variant can lead to sequence diversity or conservation of circulating viral genomes. During infection, pressure to escape recognition by antigen-targeting immune factors such as antibodies and T lymphocytes can lead to diversifying selection of viral antigens, while emergence of a dominant lineage in the viral population can reduce the diversity of the viral quasispecies and increase genome conservation.

Characterizing which regions in the genome are conserved or diverse with an emphasis on antigenic targets has critical implications for immune surveillance, drug development and resistance. To date, both B cell and T cell immune responses have been reported to play critical roles in controlling SARS-CoV-2 infections. B cells produce both anti-SARS-CoV-2 neutralizing and non-neutralizing antibodies, which comprise the polyclonal responses. Many of the most potently neutralizing anti-SARS-CoV-2 antibodies target the receptor-binding domain (RBD) of the viral spike protein, inhibiting the binding to the ACE2 cell host receptor. Antigen-specific T cells recognize short peptide epitopes generated by proteolysis of pathogen proteins, bound to HLA molecules on the surface of antigen-presenting cells. SARS-CoV-2 reactive memory CD4⁺ and CD8⁺ T cells have also been reported in unexposed individuals, suggesting that pre-existing cross-reactive T cell could drive disease outcomes in infected patients.

The disclosed methods and systems can be used to reduce a dataset to nevertheless assess and predict the evolution of a virus (e.g., SARS-CoV-2) and the future of a pandemic. In an embodiment, the disclosed methods and systems can generate a comprehensive dynamic map of sites under selection and conservation across a genome (e.g., a viral genome).

Amino acid variants spreading in the community at high frequency can be identified. An evolutionary relationship of SARS-CoV-2 isolates can be established and population genetic analyses can be employed to determine the selective forces driving genetic diversity in a virus at protein and/or amino acid levels. A time series dataset (e.g., quarterly) can be generated to determine how evolution of a viral genome has changed across defined time periods of a pandemic to explore the impact of broad infection, vaccination, and/or emergence of different lineages on viral evolution. For example, time periods of a pandemic may include a pre-vaccine era, a post-vaccine era, and the emergence of one or more lineages. In an embodiment, the disclosed methods and systems may conduct a comprehensive genetic analysis of known virus B and T cell epitopes to determine whether all viral antigens represent potential target of immune escape.

### EXAMPLES

### A. Example 1: Evolutionary Trajectory of SARS-CoV-2 Genome Shifts During Widespread Vaccination and Emergence of Omicron Variant

In this example, 13,128,166 SARS-CoV-2 genome sequences collected worldwide from the time of the first reported sequence in late 2019 to October 2022 were leveraged to assess and predict the evolution of the virus and the future of the pandemic. First, amino acid variants spreading in the community at high frequency were highlighted. The evolutionary relationship of SARS-CoV-2 isolates was established and population genetic analyses employed to determine the selective forces driving genetic diversity in SARS-CoV-2 at both protein and amino acid levels. To explore the impact of broad infection, vaccination and emergence of Omicron lineages on SARS-CoV-2 evolution, a quarterly time series dataset was generated that revealed how evolution of the SARS-CoV-2 genome has changed across three defining time periods of the pandemic, namely, the pre-vaccine era, the post-vaccine era and the emergence of omicron lineages. Finally, a comprehensive genetic analysis of the currently known SARS-CoV-2 B and T cell epitopes was presented to determine whether all SARS-CoV-2 antigens represent potential target of immune escape.

### 1. Results

i. Comprehensive Analysis of Sequence Variation in SARS-CoV-2.

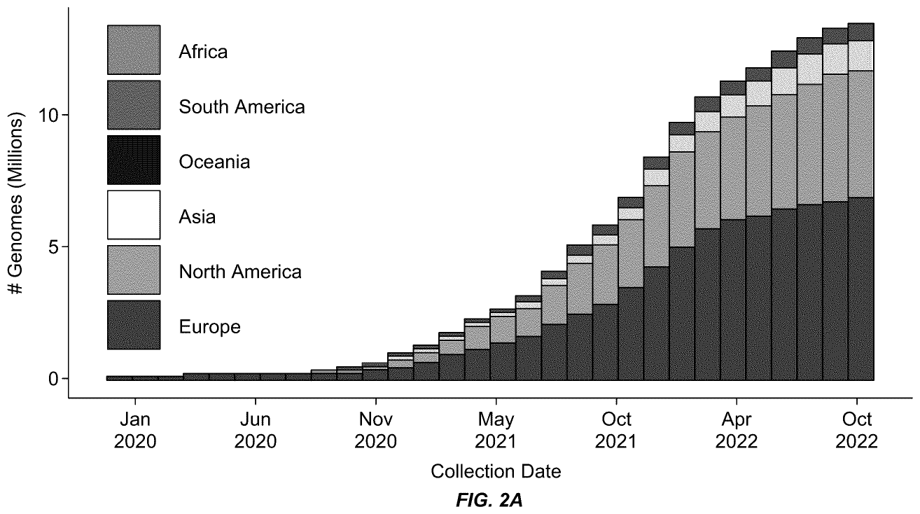

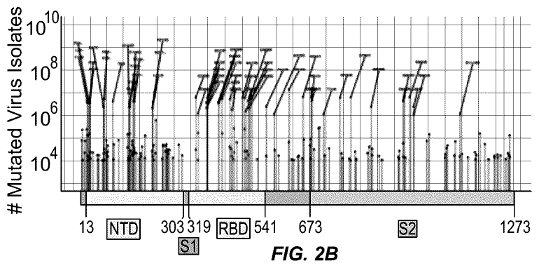

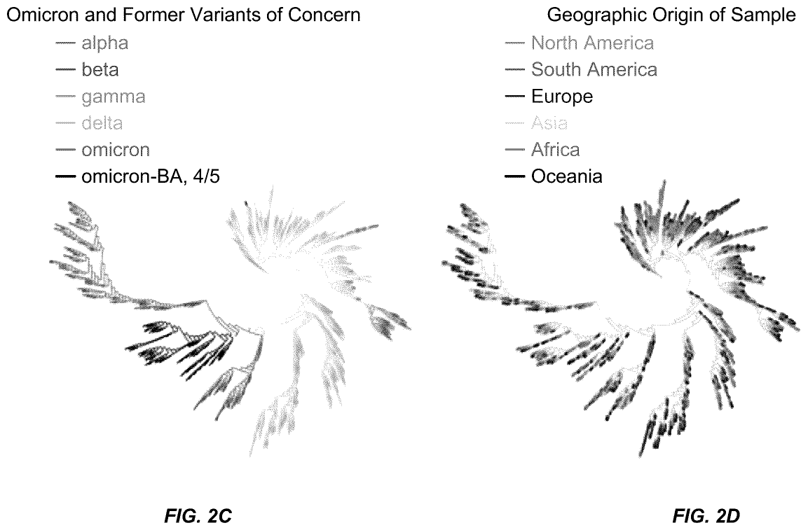

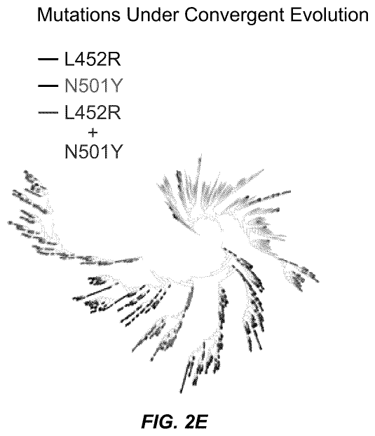

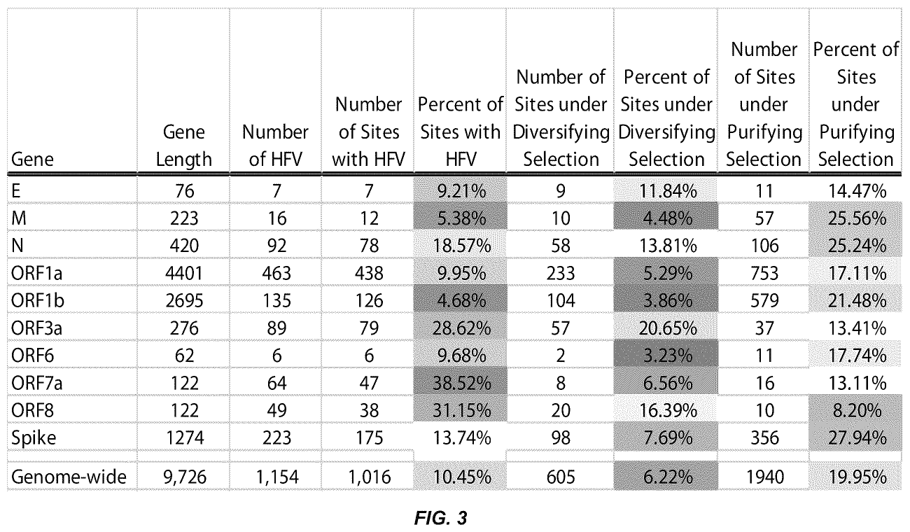

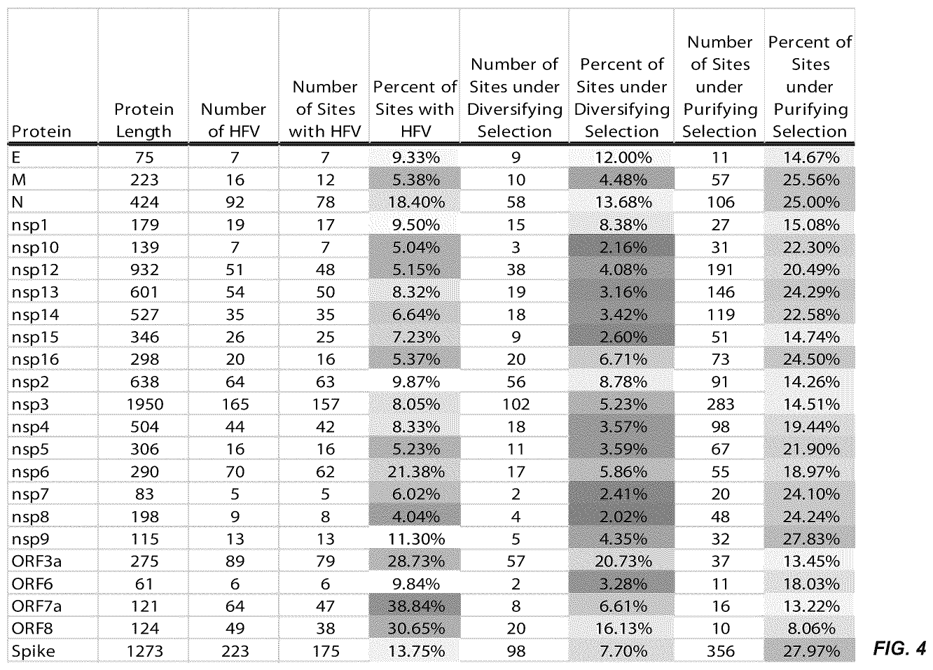

Since the first SARS-CoV-2 genome sequence was reported in early January 2020, there have been over thirteen million sequences deposited to GISAID (www.gisaid.org/). Each genome sequence is associated with comprehensive patient-related metadata that can be used to determine the time of infection and the geographical origin of the virus isolates (FIG. 2A). The identity of all coding sequences retrieved from 13,128,166 curated genomes was compared. It was found that 26% of all variants were identified in only one given isolate (singleton). However, there were 1,154 high frequency variants (HFV) that were shared across at least 10,000 isolates occurring at 10% of total sites genome-wide (1,016 out of a total 9,726 sites) (FIG. 3). A disproportionate number of HFV were found in ORF7a (39%; 47 out of a total 122 sites within the gene), ORF8 (31%; 38/122 sites), and ORF3a (29%; 79/276 sites). FIG. 3 shows gene-level summaries of the HFV and sites under selection. FIG. 4 shows protein-level summaries of the HFV and sites under selection.

The spike protein exhibited enrichment of sequence diversity (14%; 175/1,274 sites) compared to the entire genome sequence. HFV identified within the spike protein were found to be accumulating within the N-terminal domain (NTD) (24%; 71/291 sites) (FIG. 2B), while the RBD had a similar number of HFV (13%; 28/223 sites) when compared to the full spike protein and more HFV compared to the genome as a whole.

ii. SARS-CoV-2 Diversity Across Phylogenetic Lineages.

Detailed phylogenetic analysis of a data set on the order of millions of genome samples is computationally challenging due to the combinatorial complexity of evaluating relationships between all samples. To obtain a computationally feasible data set, a down-sampling method was used to select a subset of 10,000 representative genomes (see Methods). Genome-based phylogenetic analysis resolved relationships among samples and the evolution of the virus in relation to geographic distribution. Many lineages contained SARS-CoV-2 isolates from several continents, indicating limited or no apparent restrictions between lineages and local populations (FIG. 2C-D). Comparison of the diversity of individual genomes by lineage allowed identification of mutations following a pattern of convergent evolution (FIG. 2E). These mutations included N501Y and L452R, which were identified independently in genomes from at least 6 and 7 major lineages, respectively (Table 1). This indicates emergence of both mutations occurred as a result of similar selective forces in independent individuals rather than linear transmission. Since phylogeny-defining mutations can be critical to understand virus biology, diversifying and purifying selection and functional studies of how variants may impact SARS-CoV-2 biology was further examined.

TABLE 1
RBD Sites Under Convergent Evolution

| | | | | | Reduced | Reduced |
|---|---|---|---|---|---|---|
| | | | Increased | Decreased | neutralization | neutralization |
| | | Associated | ACE2 | ACE2 | by | by |
| | | major | binding | binding | convalescent | therapeutic |
| Effect | HFV | variants | affinity | affinity | sera | antibodies |
| Increased | L452* | delta, | X | | X | |
| binding | | epsilon, | | | | |
| affinity and | | iota, | | | | |
| reduced | | kappa, | | | | |
| neutralization | | lambda, | | | | |
| | | delta, | | | | |
| | | omicron | | | | |
| Increased | N501Y | alpha, | X | | | |
| binding | | beta, | | | | |
| affinity | | gamma, | | | | |
| | | theta, | | | | |
| | | mu, | | | | |
| | | omicron | | | | |
| | T478K | delta, | X | | | |
| | | omicron | | | | |
| Reduced | E484* | beta, | | | X | X |
| neutralization | | gamma, | | | | |
| | | zeta, | | | | |
| | | eta, | | | | |
| | | kappa, | | | | |
| | | iota, | | | | |
| | | theta, | | | | |
| | | mu, | | | | |
| | | omicron | | | | |
| | K417* | beta, | | X | X | X |
| | | gamma, | | | | |
| | | delta, | | | | |
| | | omicron | | | | |
| | R346* | mu, | | | | X |
| | | omicron | | | | |

iii. Selective Landscape of the SARS-CoV-2 Genome.

Current Bayesian estimates of the SARS-CoV-2 genome mutation rate are approximately 7×10⁻⁴ nucleotide substitutions per site per year, which is low compared to other human coronaviruses and RNA viruses indicating that purifying selection substantially shapes the SARS-CoV-2 genome. To characterize the selective forces driving SARS-CoV-2 gene sequence diversity, sites evolving under natural selection were identified by applying a Bayesian codon model, FUBAR. Codon substitution models are used to identify sites that are evolving significantly faster (diversifying selection) or slower (purifying selection) than expected. Quickly spreading mutations under positive selection, especially those that arise independently across multiple lineages via convergent evolution, can be identified through these approaches by their elevated nonsynonymous (dN) versus synonymous (dS) substitution rates. Similarly, sites where the reference alleles are conserved and mutations fall exclusively on shallow branches of the phylogeny are typically under purifying selection, as evidenced by low dN/dS ratios (<<1), and are more likely to negatively impact the fitness of the virus.

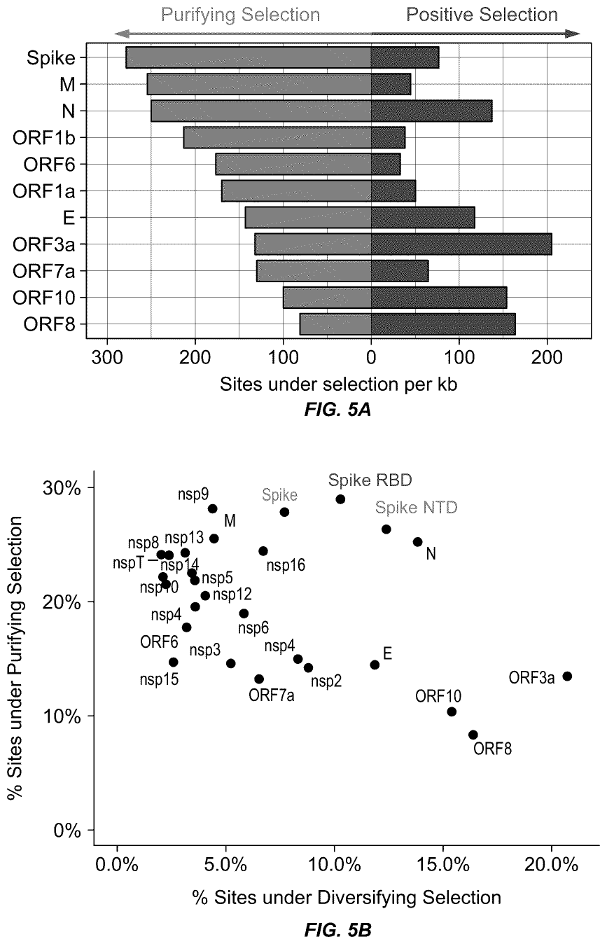

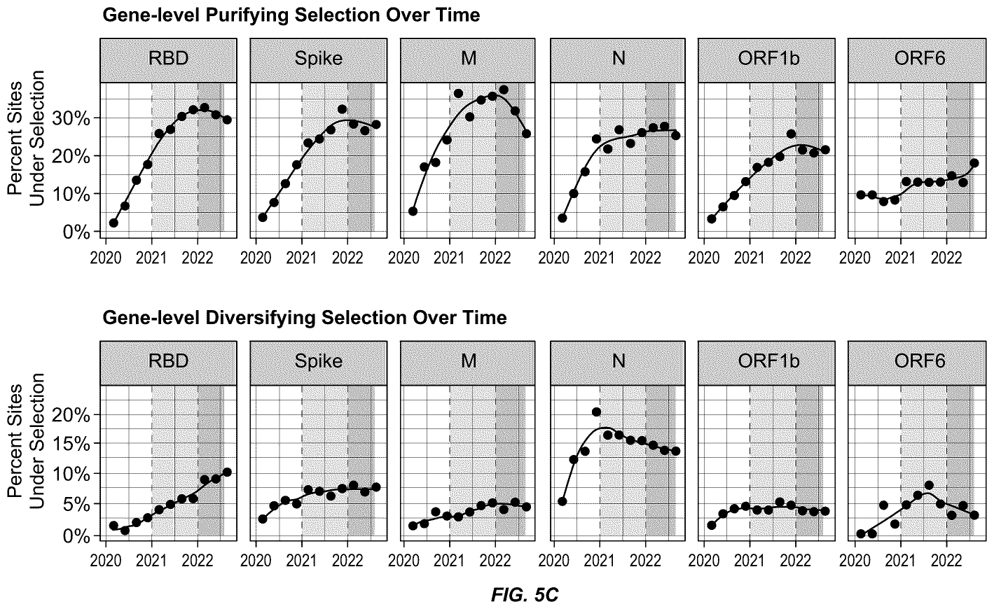

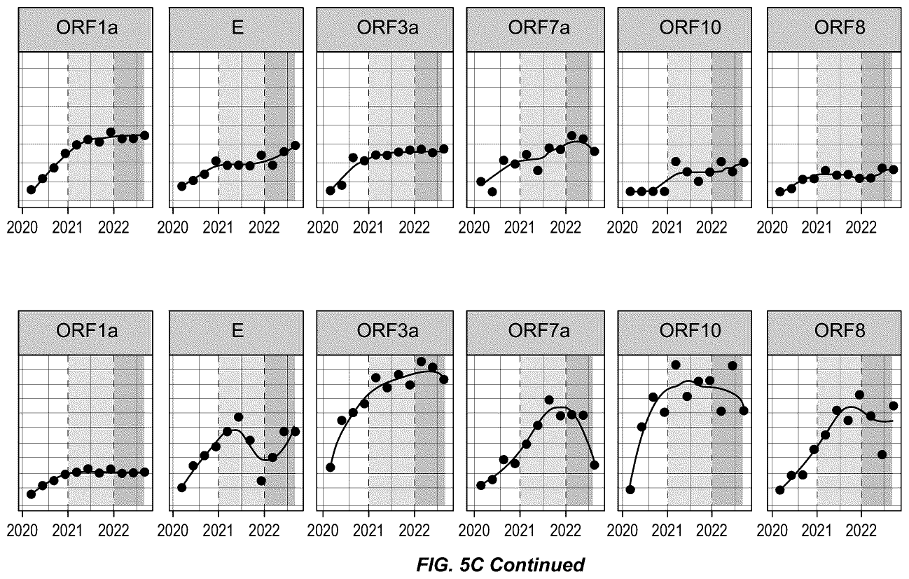

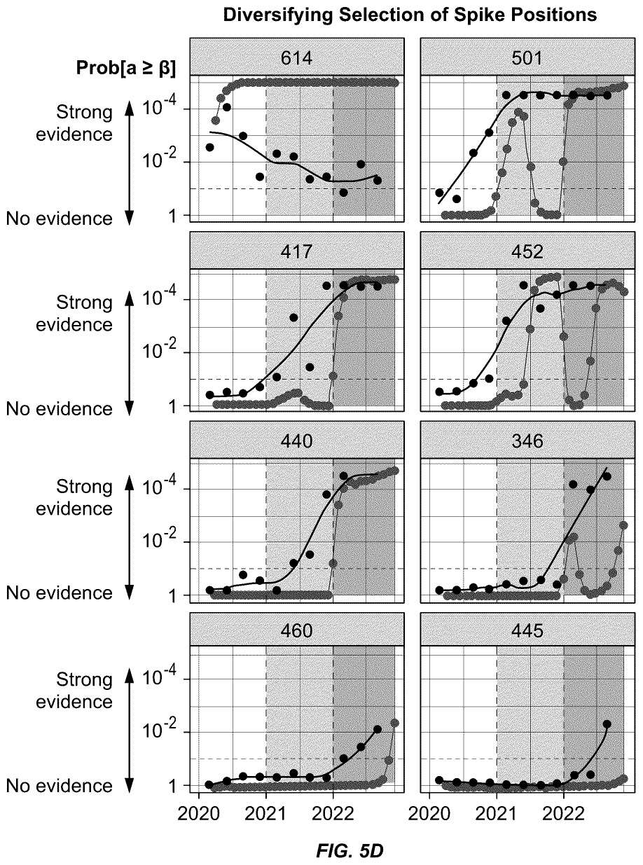

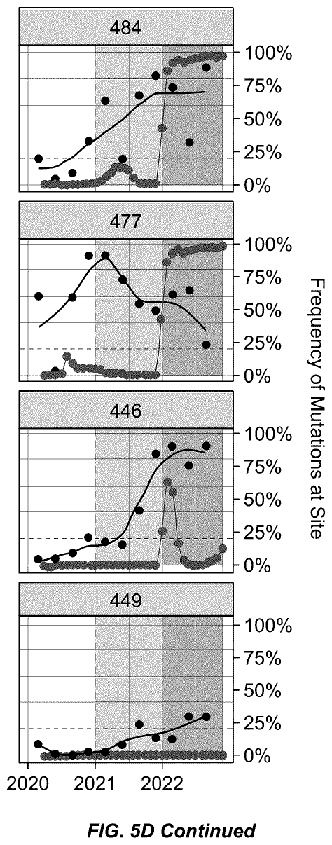

Overall, a gene-based measure of selection confirmed strong purifying selection acting on the SARS-CoV-2 genome but also identified a substantial number of sites under diversifying selection in several genes (FIG. 5A). Amongst structural and accessory protein-encoding genes, N, E, ORF3a, ORF8, and ORF10 genes showed an elevated number of sites under diversifying selection, indicating that these genes were under distinct selective forces when compared to the rest of the genome. Early stop codons are amongst the most frequent HFV in ORF3a (Q57H) and ORF8 (Q27*) and ORF10 is no longer treated as a protein-coding gene. Interestingly, most of these genes have been implicated in the suppression of the innate immune response, particularly in the suppression of type one interferon signaling (ORF8), blocking autophagy (ORF3a), and downregulation of antigen presentation (ORF8).

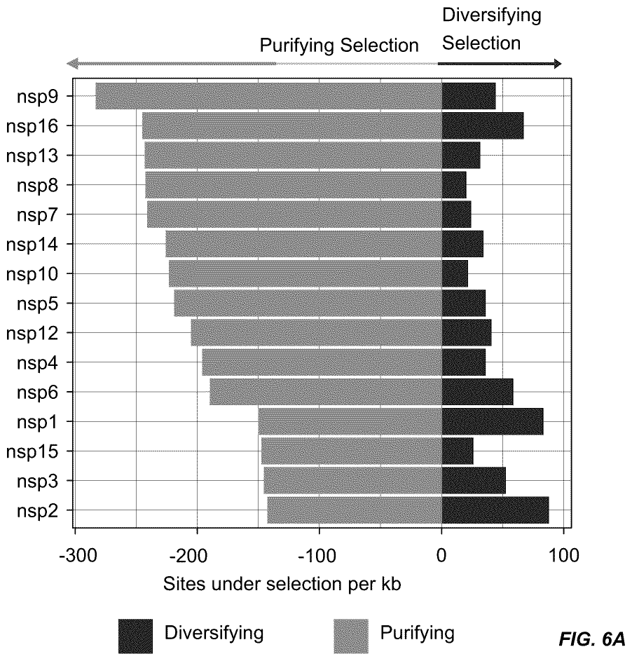

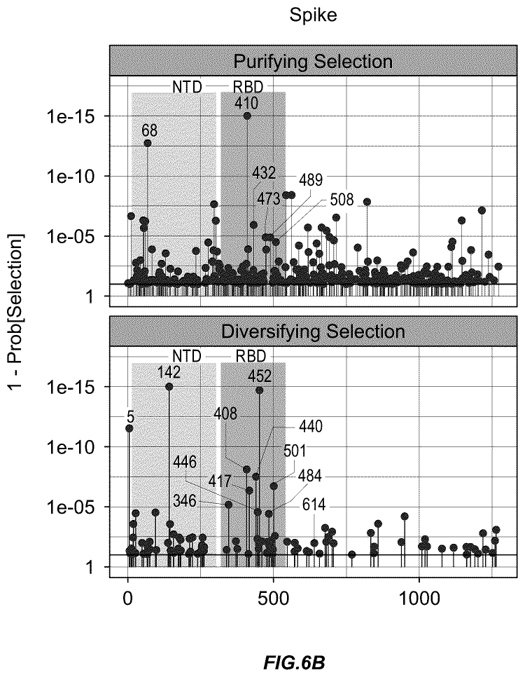

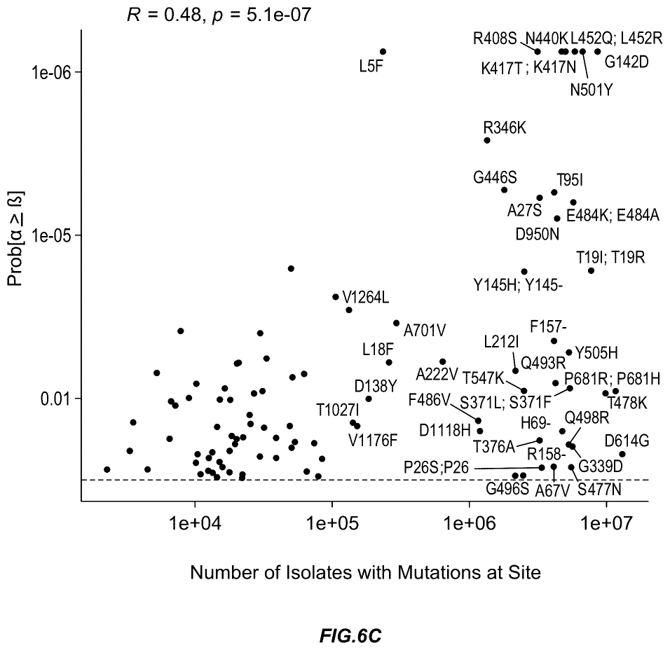

When compared to other proteins within the SARS-CoV-2 genome, the spike protein and its RBD region were under some of the highest levels of purifying selection observed, along with the M, Nsp7-ORF1a, and Nsp9-ORF1a proteins (FIG. 5B, FIG. 6A). While similar levels of HFV were present in the Spike protein and RBD, the RBD was under elevated diversifying selection (10%; 23/223 sites), including the seven of the most frequently mutated sites (339, 417, 452, 477, 478, 484, 501) (FIG. 6B-C), which may be consistent with the pressure imposed by the immune system on this domain. This indicated that despite having a globally conserved genome, evolutionary analysis at the single amino acid level revealed sites with significant diversity. A comprehensive list of all sites under selection is provided as a supplemental table (Table 2 and Table 3).

TABLE 2
Comprehensive list of sites under diversifying selection

| Gene | Protein | Site | alpha | beta | Prob[alpha < beta] |
|---|---|---|---|---|---|
| Spike | Spike | 142 | 0.387 | 50.000 | 1.000 |
| Spike | Spike | 452 | 0.619 | 15.520 | 1.000 |
| Spike | Spike | 5 | 0.355 | 15.524 | 1.000 |
| ORF3a | ORF3a | 155 | 0.163 | 15.511 | 1.000 |
| ORF1a | nsp6 | 3606 | 0.353 | 50.000 | 1.000 |
| ORF1a | nsp6 | 3829 | 0.860 | 15.518 | 1.000 |
| ORF1a | nsp5 | 3353 | 1.034 | 50.000 | 1.000 |
| ORF1a | nsp2 | 519 | 0.343 | 15.524 | 1.000 |
| ORF1b | nsp12 | 685 | 0.440 | 29.297 | 1.000 |
| N | N | 202 | 0.683 | 15.519 | 1.000 |
| N | N | 135 | 0.631 | 7.235 | 1.000 |
| N | N | 33 | 0.376 | 15.515 | 1.000 |
| M | M | 3 | 2.872 | 29.865 | 1.000 |
| Spike | Spike | 408 | 0.640 | 15.518 | 1.000 |
| ORF3a | ORF3a | 223 | 0.373 | 7.125 | 1.000 |
| ORF1b | nsp16 | 2557 | 1.002 | 29.471 | 1.000 |
| N | N | 379 | 0.236 | 7.125 | 1.000 |
| Spike | Spike | 440 | 1.606 | 29.359 | 1.000 |
| ORF1b | nsp12 | 662 | 0.430 | 16.763 | 1.000 |
| N | N | 205 | 0.479 | 7.124 | 1.000 |
| Spike | Spike | 501 | 0.457 | 15.517 | 1.000 |
| Spike | Spike | 417 | 0.476 | 15.528 | 1.000 |
| ORF8 | ORF8 | 65 | 0.578 | 7.125 | 1.000 |
| ORF3a | ORF3a | 151 | 0.216 | 6.897 | 1.000 |
| ORF1b | nsp16 | 2610 | 0.595 | 7.183 | 1.000 |
| Spike | Spike | 346 | 0.789 | 15.519 | 1.000 |
| M | M | 63 | 0.385 | 7.126 | 1.000 |
| ORF10 | ORF10 | 24 | 0.228 | 5.059 | 1.000 |
| N | N | 18 | 0.188 | 2.875 | 1.000 |
| ORF1a | nsp6 | 3718 | 0.397 | 8.798 | 1.000 |
| Spike | Spike | 446 | 0.400 | 7.204 | 1.000 |
| Spike | Spike | 95 | 0.721 | 7.126 | 1.000 |
| ORF1a | nsp3 | 1640 | 0.423 | 7.121 | 1.000 |
| ORF1a | nsp2 | 207 | 0.269 | 6.986 | 1.000 |
| ORF1b | nsp16 | 2613 | 7.047 | 28.604 | 1.000 |
| Spike | Spike | 27 | 0.480 | 7.125 | 1.000 |
| Spike | Spike | 484 | 0.559 | 15.519 | 1.000 |
| ORF10 | ORF10 | 31 | 0.254 | 4.662 | 1.000 |
| ORF1b | nsp14 | 1550 | 0.324 | 7.058 | 1.000 |
| Spike | Spike | 950 | 1.004 | 18.794 | 1.000 |
| ORF3a | ORF3a | 54 | 0.286 | 2.841 | 1.000 |
| ORF3a | ORF3a | 110 | 0.286 | 2.841 | 1.000 |
| E | E | 62 | 0.406 | 7.476 | 1.000 |
| N | N | 90 | 0.188 | 2.791 | 1.000 |
| ORF1a | nsp4 | 3201 | 0.479 | 7.137 | 1.000 |
| N | N | 204 | 0.497 | 7.124 | 1.000 |
| ORF1a | nsp2 | 445 | 0.386 | 6.874 | 1.000 |
| N | N | 49 | 0.188 | 2.796 | 1.000 |
| N | N | 24 | 0.188 | 2.796 | 1.000 |
| ORF1a | nsp6 | 3729 | 0.525 | 15.403 | 1.000 |
| N | N | 377 | 1.295 | 7.124 | 1.000 |
| ORF1a | nsp3 | 998 | 0.444 | 7.916 | 1.000 |
| ORF1a | nsp4 | 3143 | 0.507 | 7.140 | 1.000 |
| ORF1b | nsp13 | 1504 | 0.463 | 7.101 | 1.000 |
| ORF1a | nsp6 | 3758 | 0.433 | 7.123 | 1.000 |
| Spike | Spike | 859 | 0.534 | 7.157 | 1.000 |
| ORF3a | ORF3a | 108 | 0.283 | 2.724 | 1.000 |
| ORF1a | nsp6 | 3777 | 0.459 | 9.853 | 1.000 |
| N | N | 211 | 0.189 | 2.756 | 1.000 |
| Spike | Spike | 19 | 0.554 | 7.125 | 1.000 |
| Spike | Spike | 145 | 2.719 | 7.284 | 1.000 |
| N | N | 152 | 0.413 | 2.874 | 1.000 |
| ORF1a | nsp3 | 1168 | 0.393 | 6.794 | 1.000 |
| ORF7a | ORF7a | 14 | 0.288 | 2.800 | 1.000 |
| ORF1b | nsp12 | 314 | 0.438 | 7.340 | 1.000 |
| ORF1b | nsp12 | 17 | 0.438 | 7.293 | 1.000 |
| ORF3a | ORF3a | 77 | 0.281 | 2.680 | 1.000 |
| ORF1b | nsp12 | 638 | 0.576 | 7.459 | 1.000 |
| ORF1b | nsp13 | 1315 | 0.489 | 7.089 | 1.000 |
| N | N | 215 | 0.428 | 2.819 | 0.999 |
| ORF1a | nsp1 | 110 | 0.312 | 6.135 | 0.999 |
| ORF1a | nsp6 | 3750 | 0.703 | 7.135 | 0.999 |
| ORF1a | nsp8 | 4090 | 0.458 | 7.661 | 0.999 |
| Spike | Spike | 677 | 0.511 | 9.015 | 0.999 |
| ORF1b | nsp16 | 2523 | 0.293 | 3.271 | 0.999 |
| ORF1a | nsp3 | 971 | 0.658 | 7.025 | 0.999 |
| ORF1a | nsp10 | 4355 | 0.459 | 7.174 | 0.999 |
| ORF3a | ORF3a | 172 | 0.528 | 3.008 | 0.999 |
| ORF1a | nsp3 | 1822 | 0.695 | 7.106 | 0.999 |
| N | N | 119 | 0.192 | 2.675 | 0.999 |
| ORF3a | ORF3a | 53 | 0.531 | 2.812 | 0.999 |
| ORF1a | nsp2 | 728 | 0.676 | 3.771 | 0.999 |
| ORF1a | nsp1 | 124 | 0.326 | 3.750 | 0.999 |
| Spike | Spike | 1264 | 1.461 | 7.125 | 0.999 |
| ORF1a | nsp3 | 2529 | 0.503 | 7.095 | 0.999 |
| ORF3a | ORF3a | 224 | 0.530 | 2.800 | 0.999 |
| ORF1b | nsp12 | 512 | 0.466 | 7.543 | 0.999 |
| N | N | 193 | 0.256 | 2.778 | 0.999 |
| ORF3a | ORF3a | 41 | 0.690 | 2.814 | 0.999 |
| M | M | 82 | 1.366 | 10.787 | 0.999 |
| ORF1a | nsp3 | 944 | 0.513 | 7.125 | 0.999 |
| ORF8 | ORF8 | 115 | 0.312 | 2.744 | 0.999 |
| Spike | Spike | 701 | 0.538 | 7.102 | 0.999 |
| E | E | 55 | 0.411 | 6.144 | 0.999 |
| ORF1a | nsp2 | 590 | 0.444 | 2.831 | 0.999 |
| N | N | 67 | 0.196 | 2.542 | 0.999 |
| ORF1a | nsp9 | 4164 | 0.462 | 7.024 | 0.999 |
| ORF3a | ORF3a | 18 | 0.270 | 2.371 | 0.999 |
| N | N | 32 | 0.287 | 2.976 | 0.999 |
| Spike | Spike | 834 | 0.392 | 4.681 | 0.999 |
| Spike | Spike | 1219 | 0.306 | 2.914 | 0.998 |
| ORF1a | nsp1 | 87 | 0.445 | 6.915 | 0.998 |
| ORF1b | nsp12 | 813 | 1.294 | 18.412 | 0.998 |
| ORF1b | nsp16 | 2633 | 0.583 | 7.108 | 0.998 |
| Spike | Spike | 157 | 0.582 | 2.953 | 0.998 |
| ORF10 | ORF10 | 37 | 0.757 | 2.817 | 0.998 |
| ORF7a | ORF7a | 34 | 0.290 | 2.610 | 0.998 |
| ORF1a | nsp6 | 3580 | 0.297 | 2.826 | 0.998 |
| ORF1a | nsp2 | 656 | 0.516 | 2.871 | 0.998 |
| ORF1a | nsp4 | 2994 | 0.513 | 6.828 | 0.998 |
| ORF1a | nsp2 | 403 | 0.518 | 2.811 | 0.997 |
| ORF1a | nsp3 | 1921 | 0.544 | 7.124 | 0.997 |
| N | N | 362 | 0.940 | 7.125 | 0.997 |
| ORF1a | nsp10 | 4304 | 0.467 | 6.870 | 0.997 |
| ORF1a | nsp1 | 81 | 0.381 | 2.894 | 0.997 |
| ORF1b | nsp13 | 1219 | 0.907 | 7.081 | 0.997 |
| Spike | Spike | 505 | 0.581 | 2.818 | 0.997 |
| ORF3a | ORF3a | 15 | 0.295 | 2.215 | 0.997 |
| ORF1a | nsp5 | 3523 | 2.628 | 7.116 | 0.997 |
| ORF3a | ORF3a | 100 | 0.687 | 2.753 | 0.997 |
| N | N | 413 | 0.471 | 2.764 | 0.997 |
| ORF1b | nsp16 | 2635 | 0.509 | 6.198 | 0.997 |
| N | N | 13 | 2.800 | 7.124 | 0.997 |
| ORF1a | nsp6 | 3615 | 0.382 | 5.945 | 0.997 |
| ORF1a | nsp3 | 1786 | 0.451 | 5.490 | 0.997 |
| Spike | Spike | 688 | 0.433 | 5.653 | 0.997 |
| ORF1a | nsp3 | 2648 | 0.509 | 6.906 | 0.997 |
| ORF1b | nsp13 | 1383 | 0.612 | 7.125 | 0.997 |
| Spike | Spike | 222 | 0.455 | 2.835 | 0.996 |
| N | N | 151 | 0.748 | 5.384 | 0.996 |
| Spike | Spike | 18 | 0.456 | 2.840 | 0.996 |
| ORF1a | nsp5 | 3284 | 0.480 | 6.416 | 0.996 |
| Spike | Spike | 176 | 0.456 | 2.811 | 0.996 |
| Spike | Spike | 257 | 0.456 | 2.808 | 0.996 |
| ORF8 | ORF8 | 67 | 0.563 | 2.831 | 0.996 |
| ORF1a | nsp4 | 3209 | 1.024 | 7.043 | 0.996 |
| ORF1a | nsp3 | 1049 | 0.466 | 2.836 | 0.996 |
| ORF1b | nsp12 | 814 | 0.575 | 6.857 | 0.996 |
| ORF1b | nsp12 | 164 | 0.324 | 2.781 | 0.996 |
| ORF1b | nsp12 | 735 | 0.608 | 10.405 | 0.996 |
| ORF1a | nsp3 | 1113 | 0.811 | 3.274 | 0.996 |
| ORF1b | nsp12 | 730 | 0.609 | 9.880 | 0.996 |
| ORF1a | nsp3 | 1125 | 0.463 | 2.947 | 0.996 |
| Spike | Spike | 212 | 1.777 | 7.099 | 0.995 |
| M | M | 17 | 0.404 | 2.791 | 0.995 |
| ORF1b | nsp16 | 2431 | 0.571 | 6.473 | 0.995 |
| ORF1b | nsp13 | 1220 | 0.356 | 2.720 | 0.995 |
| ORF1a | nsp3 | 1754 | 0.464 | 4.756 | 0.995 |
| Spike | Spike | 445 | 0.806 | 6.755 | 0.995 |
| ORF1a | nsp1 | 131 | 0.347 | 2.670 | 0.995 |
| Spike | Spike | 1020 | 0.999 | 7.054 | 0.995 |
| N | N | 144 | 0.523 | 2.880 | 0.995 |
| ORF1b | nsp13 | 1184 | 0.719 | 8.844 | 0.995 |
| ORF1a | nsp4 | 3090 | 0.526 | 6.433 | 0.995 |
| Spike | Spike | 181 | 1.428 | 7.098 | 0.994 |
| E | E | 21 | 0.429 | 3.333 | 0.994 |
| ORF1b | nsp12 | 176 | 0.787 | 7.267 | 0.994 |
| M | M | 19 | 8.000 | 35.031 | 0.994 |
| ORF3a | ORF3a | 229 | 0.780 | 2.783 | 0.994 |
| ORF1b | nsp13 | 1129 | 1.086 | 7.705 | 0.994 |
| ORF1a | nsp3 | 1298 | 0.461 | 6.571 | 0.994 |
| Spike | Spike | 493 | 0.659 | 6.977 | 0.993 |
| ORF1a | nsp2 | 400 | 0.348 | 2.641 | 0.993 |
| Spike | Spike | 1260 | 0.348 | 2.724 | 0.993 |
| ORF1a | nsp3 | 1655 | 0.516 | 6.229 | 0.993 |
| ORF1a | nsp3 | 1763 | 0.516 | 6.226 | 0.993 |
| ORF1a | nsp2 | 591 | 0.886 | 6.502 | 0.993 |
| ORF1a | nsp3 | 1119 | 0.594 | 2.711 | 0.993 |
| ORF1b | nsp13 | 1264 | 1.148 | 12.637 | 0.993 |
| ORF1a | nsp4 | 3027 | 0.534 | 6.233 | 0.993 |
| N | N | 326 | 0.422 | 2.491 | 0.992 |
| Spike | Spike | 371 | 0.831 | 3.302 | 0.992 |
| ORF1a | nsp3 | 1001 | 0.466 | 2.722 | 0.992 |
| Spike | Spike | 460 | 0.516 | 5.845 | 0.992 |
| E | E | 9 | 0.582 | 7.152 | 0.992 |
| ORF1b | nsp13 | 1427 | 1.494 | 8.027 | 0.992 |
| ORF1b | nsp12 | 469 | 0.618 | 8.060 | 0.992 |
| ORF3a | ORF3a | 104 | 0.665 | 2.673 | 0.992 |
| ORF1b | nsp13 | 1092 | 1.101 | 7.098 | 0.992 |
| Spike | Spike | 547 | 0.569 | 6.321 | 0.992 |
| Spike | Spike | 681 | 5.156 | 15.393 | 0.992 |
| Spike | Spike | 75 | 0.449 | 2.614 | 0.992 |
| ORF1a | nsp5 | 3371 | 0.779 | 7.033 | 0.992 |
| ORF1a | nsp3 | 1862 | 0.497 | 2.837 | 0.991 |
| ORF1a | nsp2 | 184 | 0.487 | 2.579 | 0.991 |
| Spike | Spike | 478 | 0.736 | 7.233 | 0.991 |
| Spike | Spike | 939 | 0.545 | 5.727 | 0.991 |
| ORF1b | nsp16 | 2548 | 0.472 | 4.130 | 0.991 |
| ORF3a | ORF3a | 25 | 0.231 | 1.233 | 0.991 |
| E | E | 73 | 0.430 | 2.927 | 0.991 |
| ORF1a | nsp3 | 1840 | 0.716 | 6.333 | 0.991 |
| ORF3a | ORF3a | 14 | 0.241 | 1.487 | 0.991 |
| ORF1b | nsp13 | 1291 | 0.783 | 7.127 | 0.991 |
| ORF8 | ORF8 | 51 | 0.557 | 2.677 | 0.990 |
| ORF1b | nsp12 | 249 | 0.335 | 3.297 | 0.990 |
| ORF1a | nsp6 | 3676 | 0.923 | 11.487 | 0.990 |
| ORF1a | nsp3 | 1155 | 0.464 | 2.708 | 0.990 |
| ORF1a | nsp3 | 913 | 0.516 | 3.743 | 0.990 |
| Spike | Spike | 573 | 0.505 | 2.849 | 0.990 |
| Spike | Spike | 138 | 0.518 | 3.005 | 0.990 |
| Spike | Spike | 640 | 0.506 | 2.783 | 0.990 |
| ORF1b | nsp14 | 1643 | 1.185 | 7.113 | 0.990 |
| Spike | Spike | 49 | 0.519 | 2.818 | 0.990 |
| ORF3a | ORF3a | 107 | 0.603 | 2.654 | 0.990 |
| ORF8 | ORF8 | 122 | 4.271 | 47.940 | 0.989 |
| ORF1a | nsp4 | 2900 | 0.772 | 6.969 | 0.989 |
| ORF1a | nsp6 | 3677 | 0.695 | 8.408 | 0.989 |
| ORF1b | nsp14 | 1918 | 0.591 | 5.736 | 0.989 |
| ORF1a | nsp2 | 804 | 0.464 | 2.632 | 0.989 |
| Spike | Spike | 706 | 0.505 | 2.767 | 0.989 |
| ORF1a | nsp1 | 135 | 0.438 | 2.729 | 0.989 |
| ORF1b | nsp13 | 1511 | 0.787 | 7.079 | 0.989 |
| ORF3a | ORF3a | 165 | 0.229 | 1.145 | 0.988 |
| ORF1a | nsp2 | 690 | 0.738 | 2.856 | 0.988 |
| ORF8 | ORF8 | 11 | 0.431 | 2.653 | 0.988 |
| ORF1a | nsp6 | 3620 | 0.363 | 2.827 | 0.988 |
| Spike | Spike | 450 | 0.534 | 4.948 | 0.988 |
| ORF1a | nsp3 | 1543 | 0.609 | 7.178 | 0.988 |
| ORF1a | nsp2 | 550 | 0.884 | 2.843 | 0.988 |
| ORF8 | ORF8 | 54 | 0.429 | 2.629 | 0.987 |
| ORF1a | nsp2 | 609 | 0.739 | 2.810 | 0.987 |
| ORF1a | nsp3 | 2274 | 0.458 | 5.688 | 0.987 |
| ORF1a | nsp6 | 3644 | 0.365 | 2.776 | 0.987 |
| ORF1a | nsp2 | 498 | 0.619 | 3.527 | 0.987 |
| ORF3a | ORF3a | 59 | 0.229 | 1.146 | 0.987 |
| ORF1a | nsp3 | 1000 | 0.464 | 2.586 | 0.986 |
| ORF1a | nsp2 | 337 | 0.354 | 2.404 | 0.986 |
| ORF1b | nsp12 | 248 | 0.454 | 3.743 | 0.986 |
| ORF8 | ORF8 | 121 | 2.874 | 7.151 | 0.986 |
| N | N | 391 | 0.426 | 2.354 | 0.986 |
| M | M | 125 | 1.105 | 6.883 | 0.986 |
| ORF1a | nsp5 | 3351 | 0.616 | 6.108 | 0.986 |
| ORF1b | nsp15 | 2070 | 0.620 | 6.875 | 0.986 |
| ORF1a | nsp3 | 1803 | 0.496 | 2.671 | 0.985 |
| ORF1a | nsp4 | 2857 | 2.770 | 7.100 | 0.985 |
| ORF1a | nsp6 | 3646 | 0.367 | 2.741 | 0.985 |
| ORF1a | nsp2 | 575 | 0.557 | 3.073 | 0.985 |
| ORF1a | nsp3 | 1895 | 0.887 | 7.131 | 0.985 |
| ORF7a | ORF7a | 104 | 0.297 | 1.755 | 0.984 |
| Spike | Spike | 255 | 0.715 | 4.028 | 0.984 |
| ORF8 | ORF8 | 52 | 0.485 | 2.784 | 0.984 |
| ORF1a | nsp1 | 166 | 0.504 | 2.453 | 0.983 |
| ORF3a | ORF3a | 213 | 0.525 | 2.923 | 0.983 |
| ORF1a | nsp7 | 3915 | 0.370 | 2.673 | 0.983 |
| ORF1b | nsp16 | 2612 | 0.502 | 3.362 | 0.983 |
| ORF3a | ORF3a | 30 | 0.536 | 1.971 | 0.983 |
| N | N | 155 | 0.223 | 1.354 | 0.982 |
| N | N | 217 | 0.224 | 1.353 | 0.982 |
| ORF1a | nsp6 | 3705 | 0.371 | 2.647 | 0.982 |
| ORF1a | nsp3 | 995 | 0.532 | 2.884 | 0.982 |
| ORF1a | nsp3 | 1013 | 0.532 | 2.884 | 0.982 |
| ORF1a | nsp3 | 1881 | 0.737 | 5.559 | 0.982 |
| Spike | Spike | 486 | 0.561 | 2.816 | 0.981 |
| ORF3a | ORF3a | 134 | 0.228 | 1.050 | 0.980 |
| Spike | Spike | 1009 | 0.559 | 3.960 | 0.980 |
| Spike | Spike | 1027 | 0.560 | 3.957 | 0.980 |
| ORF3a | ORF3a | 175 | 0.545 | 2.904 | 0.979 |
| Spike | Spike | 845 | 1.674 | 7.128 | 0.979 |
| ORF1b | nsp16 | 2432 | 1.201 | 8.612 | 0.979 |
| ORF1a | nsp3 | 953 | 0.498 | 2.687 | 0.979 |
| ORF1a | nsp3 | 935 | 0.498 | 2.686 | 0.979 |
| ORF3a | ORF3a | 185 | 0.520 | 2.646 | 0.978 |
| ORF1a | nsp5 | 3504 | 0.504 | 2.848 | 0.978 |
| Spike | Spike | 1162 | 1.871 | 13.501 | 0.978 |
| ORF1a | nsp2 | 561 | 0.496 | 2.335 | 0.978 |
| Spike | Spike | 1176 | 0.563 | 3.658 | 0.978 |
| Spike | Spike | 254 | 0.434 | 2.179 | 0.978 |
| ORF1b | nsp12 | 192 | 0.479 | 3.197 | 0.978 |
| N | N | 398 | 0.439 | 2.786 | 0.977 |
| N | N | 166 | 0.439 | 2.793 | 0.977 |
| Spike | Spike | 262 | 0.717 | 2.799 | 0.977 |
| ORF3a | ORF3a | 22 | 0.584 | 2.324 | 0.977 |
| ORF3a | ORF3a | 50 | 0.228 | 1.028 | 0.977 |
| N | N | 22 | 0.293 | 1.721 | 0.977 |
| ORF1b | nsp16 | 2576 | 0.524 | 3.567 | 0.976 |
| E | E | 71 | 0.733 | 4.226 | 0.976 |
| ORF3a | ORF3a | 24 | 0.229 | 1.065 | 0.976 |
| ORF6 | ORF6 | 5 | 0.366 | 2.095 | 0.976 |
| ORF1b | nsp16 | 2537 | 1.187 | 7.157 | 0.975 |
| ORF1a | nsp3 | 2511 | 1.380 | 10.138 | 0.975 |
| Spike | Spike | 1118 | 0.701 | 3.582 | 0.975 |
| Spike | Spike | 69 | 0.594 | 2.832 | 0.975 |
| ORF1b | nsp12 | 520 | 1.611 | 13.499 | 0.974 |
| ORF1b | nsp12 | 88 | 0.463 | 2.730 | 0.974 |
| ORF1a | nsp3 | 1158 | 0.737 | 2.783 | 0.974 |
| ORF1a | nsp3 | 1733 | 0.520 | 2.847 | 0.974 |
| ORF1b | nsp14 | 1567 | 0.527 | 3.402 | 0.974 |
| ORF3a | ORF3a | 27 | 0.785 | 2.831 | 0.973 |
| ORF1a | nsp2 | 681 | 0.738 | 2.651 | 0.973 |
| ORF1a | nsp2 | 642 | 0.738 | 2.651 | 0.973 |
| ORF3a | ORF3a | 89 | 0.532 | 2.568 | 0.973 |
| ORF3a | ORF3a | 67 | 0.504 | 2.424 | 0.973 |
| ORF1a | nsp2 | 560 | 0.738 | 2.650 | 0.973 |
| ORF1b | nsp15 | 2256 | 0.835 | 5.720 | 0.973 |
| ORF1a | nsp3 | 856 | 0.534 | 2.593 | 0.972 |
| ORF1a | nsp3 | 989 | 0.736 | 2.817 | 0.972 |
| ORF1b | nsp12 | 99 | 0.463 | 2.823 | 0.972 |
| ORF1a | nsp2 | 350 | 0.365 | 1.977 | 0.972 |
| ORF1a | nsp3 | 2084 | 0.464 | 3.632 | 0.972 |
| N | N | 125 | 0.227 | 1.091 | 0.971 |
| ORF1b | nsp16 | 2425 | 0.610 | 4.820 | 0.971 |
| Spike | Spike | 583 | 0.608 | 2.990 | 0.971 |
| ORF1b | nsp14 | 1570 | 0.533 | 3.053 | 0.971 |
| M | M | 209 | 0.652 | 3.010 | 0.971 |
| ORF3a | ORF3a | 52 | 0.455 | 1.464 | 0.971 |
| ORF1a | nsp9 | 4159 | 0.851 | 7.170 | 0.970 |
| ORF1b | nsp16 | 2531 | 0.458 | 2.547 | 0.970 |
| ORF1a | nsp8 | 4065 | 0.851 | 7.170 | 0.970 |
| Spike | Spike | 12 | 0.714 | 2.689 | 0.970 |
| ORF1a | nsp8 | 4016 | 0.569 | 3.651 | 0.970 |
| ORF1b | nsp12 | 275 | 0.344 | 2.550 | 0.970 |
| ORF1b | nsp14 | 1884 | 0.604 | 3.181 | 0.969 |
| Spike | Spike | 1078 | 1.003 | 6.200 | 0.969 |
| ORF1a | nsp3 | 940 | 0.534 | 2.616 | 0.969 |
| Spike | Spike | 449 | 0.575 | 2.656 | 0.969 |
| ORF1a | nsp3 | 1160 | 1.020 | 6.252 | 0.969 |
| Spike | Spike | 173 | 0.472 | 2.208 | 0.968 |
| ORF1b | nsp12 | 440 | 0.471 | 3.001 | 0.968 |
| ORF1b | nsp12 | 414 | 0.471 | 3.000 | 0.968 |
| ORF3a | ORF3a | 251 | 0.228 | 0.964 | 0.968 |
| ORF1a | nsp3 | 1682 | 0.654 | 4.664 | 0.968 |
| ORF1a | nsp3 | 951 | 0.737 | 2.725 | 0.968 |
| ORF1a | nsp4 | 2980 | 1.909 | 7.620 | 0.968 |
| Spike | Spike | 376 | 0.713 | 2.648 | 0.967 |
| N | N | 207 | 0.228 | 0.993 | 0.966 |
| ORF8 | ORF8 | 26 | 0.299 | 1.319 | 0.966 |
| ORF3a | ORF3a | 97 | 0.228 | 0.947 | 0.966 |
| ORF1a | nsp2 | 552 | 0.481 | 2.026 | 0.966 |
| ORF1a | nsp3 | 842 | 0.753 | 2.751 | 0.966 |
| Spike | Spike | 1191 | 0.680 | 4.784 | 0.966 |
| ORF1a | nsp9 | 4220 | 0.589 | 3.075 | 0.966 |
| ORF1b | nsp12 | 220 | 0.494 | 2.803 | 0.965 |
| Spike | Spike | 76 | 0.713 | 2.618 | 0.964 |
| ORF1a | nsp3 | 2166 | 0.484 | 3.286 | 0.964 |
| ORF1b | nsp13 | 941 | 1.884 | 7.429 | 0.964 |
| ORF1a | nsp2 | 302 | 0.833 | 2.923 | 0.964 |
| ORF3a | ORF3a | 270 | 0.229 | 0.955 | 0.964 |
| Spike | Spike | 1228 | 0.517 | 2.925 | 0.964 |
| ORF1a | nsp3 | 1280 | 0.594 | 5.286 | 0.964 |
| N | N | 243 | 0.437 | 1.717 | 0.964 |
| ORF1b | nsp12 | 146 | 0.492 | 3.128 | 0.963 |
| ORF1a | nsp9 | 4249 | 0.856 | 6.846 | 0.963 |
| ORF1a | nsp3 | 999 | 0.626 | 2.719 | 0.963 |
| ORF8 | ORF8 | 68 | 0.598 | 2.862 | 0.963 |
| Spike | Spike | 498 | 0.610 | 2.847 | 0.963 |
| ORF7a | ORF7a | 73 | 0.429 | 1.356 | 0.963 |
| ORF3a | ORF3a | 31 | 0.450 | 1.330 | 0.962 |
| ORF1b | nsp14 | 1687 | 0.603 | 2.769 | 0.962 |
| ORF1a | nsp2 | 318 | 0.580 | 2.741 | 0.962 |
| ORF1a | nsp4 | 3255 | 3.012 | 7.622 | 0.962 |
| ORF1a | nsp2 | 224 | 0.836 | 2.830 | 0.962 |
| ORF1b | nsp12 | 82 | 1.091 | 7.636 | 0.962 |
| N | N | 236 | 0.444 | 1.715 | 0.962 |
| ORF1b | nsp13 | 1003 | 0.667 | 4.798 | 0.961 |
| N | N | 418 | 0.450 | 2.430 | 0.961 |
| Spike | Spike | 339 | 0.760 | 2.746 | 0.961 |
| ORF1a | nsp2 | 309 | 1.955 | 7.127 | 0.961 |
| ORF1a | nsp6 | 3618 | 0.442 | 2.570 | 0.961 |
| ORF1a | nsp3 | 959 | 0.457 | 2.043 | 0.960 |
| ORF1a | nsp5 | 3509 | 0.523 | 2.602 | 0.960 |
| Spike | Spike | 98 | 0.420 | 1.749 | 0.960 |
| N | N | 414 | 0.445 | 1.664 | 0.959 |
| ORF1a | nsp2 | 540 | 0.737 | 2.510 | 0.959 |
| ORF1a | nsp3 | 2193 | 0.490 | 2.780 | 0.959 |
| ORF1a | nsp3 | 2103 | 0.490 | 2.780 | 0.959 |
| N | N | 141 | 0.454 | 2.375 | 0.958 |
| ORF1a | nsp3 | 2106 | 0.490 | 2.729 | 0.958 |
| ORF1a | nsp3 | 1633 | 0.587 | 3.292 | 0.958 |
| ORF3a | ORF3a | 140 | 0.447 | 1.246 | 0.958 |
| ORF1a | nsp3 | 2622 | 0.692 | 5.582 | 0.957 |
| ORF1a | nsp3 | 1018 | 0.626 | 2.633 | 0.957 |
| ORF1a | nsp3 | 1720 | 0.587 | 3.279 | 0.957 |
| N | N | 63 | 1.439 | 6.584 | 0.957 |
| ORF1b | nsp14 | 1774 | 1.175 | 7.216 | 0.957 |
| N | N | 26 | 0.305 | 1.238 | 0.957 |
| ORF1a | nsp3 | 2062 | 0.490 | 2.663 | 0.956 |
| ORF1a | nsp9 | 4158 | 0.582 | 2.856 | 0.956 |
| ORF3a | ORF3a | 174 | 0.372 | 1.058 | 0.956 |
| ORF1a | nsp3 | 1887 | 0.798 | 3.321 | 0.956 |
| Spike | Spike | 182 | 0.614 | 2.577 | 0.956 |
| Spike | Spike | 147 | 0.616 | 2.587 | 0.956 |
| ORF1b | nsp15 | 2117 | 0.974 | 6.817 | 0.956 |
| ORF1a | nsp3 | 1102 | 0.496 | 2.239 | 0.956 |
| ORF1a | nsp3 | 1473 | 0.604 | 3.063 | 0.956 |
| ORF8 | ORF8 | 38 | 0.731 | 2.502 | 0.955 |
| ORF10 | ORF10 | 8 | 0.474 | 1.317 | 0.955 |
| ORF1a | nsp2 | 347 | 0.567 | 2.623 | 0.954 |
| ORF7a | ORF7a | 5 | 0.296 | 0.980 | 0.954 |
| N | N | 220 | 0.226 | 0.918 | 0.954 |
| Spike | Spike | 6 | 0.415 | 1.621 | 0.954 |
| ORF1a | nsp2 | 388 | 0.679 | 2.489 | 0.954 |
| ORF8 | ORF8 | 66 | 0.300 | 1.134 | 0.954 |
| ORF1a | nsp3 | 2265 | 0.489 | 2.668 | 0.953 |
| ORF1b | nsp12 | 647 | 0.471 | 2.526 | 0.953 |
| ORF1a | nsp3 | 1055 | 0.627 | 2.588 | 0.953 |
| ORF1a | nsp3 | 1597 | 0.608 | 2.876 | 0.952 |
| ORF1a | nsp3 | 1567 | 0.608 | 2.875 | 0.952 |
| ORF1b | nsp16 | 2513 | 0.514 | 2.495 | 0.952 |
| ORF1b | nsp16 | 2488 | 1.685 | 6.723 | 0.952 |
| Spike | Spike | 614 | 0.471 | 2.021 | 0.952 |
| ORF1b | nsp12 | 348 | 0.545 | 3.773 | 0.952 |
| Spike | Spike | 261 | 0.707 | 2.424 | 0.951 |
| ORF1b | nsp13 | 1095 | 0.624 | 2.772 | 0.951 |
| ORF1a | nsp3 | 2146 | 0.489 | 2.477 | 0.951 |
| ORF1a | nsp3 | 1716 | 0.512 | 2.018 | 0.950 |
| ORF1a | nsp3 | 2589 | 0.967 | 7.021 | 0.950 |
| ORF1a | nsp1 | 99 | 0.371 | 1.507 | 0.950 |
| ORF1a | nsp1 | 29 | 0.371 | 1.507 | 0.950 |
| ORF1a | nsp3 | 1021 | 0.832 | 3.905 | 0.950 |
| ORF3a | ORF3a | 90 | 0.228 | 0.867 | 0.950 |
| ORF3a | ORF3a | 95 | 0.679 | 2.029 | 0.950 |
| N | N | 189 | 0.226 | 0.879 | 0.950 |
| ORF1a | nsp3 | 2510 | 0.946 | 8.154 | 0.949 |
| N | N | 265 | 0.225 | 0.947 | 0.949 |
| Spike | Spike | 572 | 0.820 | 2.915 | 0.948 |
| ORF6 | ORF6 | 6 | 1.001 | 2.831 | 0.948 |
| N | N | 46 | 0.445 | 1.394 | 0.948 |
| ORF1a | nsp1 | 144 | 0.665 | 2.325 | 0.948 |
| ORF1a | nsp4 | 2846 | 1.052 | 5.680 | 0.948 |
| ORF1b | nsp16 | 2514 | 0.470 | 2.321 | 0.946 |
| Spike | Spike | 250 | 0.706 | 2.390 | 0.946 |
| Spike | Spike | 180 | 0.606 | 2.401 | 0.946 |
| ORF1a | nsp3 | 2061 | 0.489 | 2.427 | 0.946 |
| ORF1a | nsp7 | 3922 | 0.467 | 2.960 | 0.946 |
| ORF3a | ORF3a | 240 | 0.954 | 2.811 | 0.945 |
| N | N | 208 | 0.947 | 2.817 | 0.945 |
| N | N | 195 | 0.557 | 2.723 | 0.945 |
| ORF3a | ORF3a | 99 | 0.956 | 2.817 | 0.945 |
| M | M | 7 | 0.387 | 1.673 | 0.945 |
| Spike | Spike | 215 | 0.833 | 2.962 | 0.945 |
| ORF1a | nsp2 | 727 | 0.920 | 2.756 | 0.945 |
| ORF3a | ORF3a | 190 | 0.229 | 0.858 | 0.944 |
| ORF1a | nsp3 | 872 | 0.205 | 0.809 | 0.944 |
| ORF7a | ORF7a | 96 | 0.294 | 0.918 | 0.942 |
| ORF1b | nsp14 | 1555 | 0.956 | 5.578 | 0.942 |
| ORF1a | nsp2 | 372 | 0.590 | 2.463 | 0.942 |
| ORF1a | nsp3 | 2154 | 0.490 | 2.336 | 0.942 |
| ORF1b | nsp15 | 2163 | 1.262 | 6.596 | 0.941 |
| ORF1b | nsp15 | 2178 | 0.657 | 3.889 | 0.941 |
| ORF1a | nsp2 | 446 | 0.459 | 1.588 | 0.941 |
| ORF1a | nsp2 | 442 | 0.735 | 2.386 | 0.941 |
| ORF1a | nsp1 | 82 | 0.372 | 1.547 | 0.940 |
| ORF8 | ORF8 | 72 | 0.662 | 2.456 | 0.940 |
| ORF1b | nsp14 | 1681 | 2.111 | 7.102 | 0.940 |
| N | N | 72 | 0.225 | 0.804 | 0.940 |
| Spike | Spike | 622 | 0.815 | 2.711 | 0.940 |
| ORF1a | nsp3 | 903 | 0.958 | 4.213 | 0.939 |
| ORF1a | nsp3 | 2207 | 0.489 | 2.388 | 0.939 |
| ORF1b | nsp14 | 1621 | 0.592 | 2.420 | 0.939 |
| ORF3a | ORF3a | 225 | 0.446 | 1.142 | 0.939 |
| ORF1b | nsp16 | 2560 | 0.925 | 4.111 | 0.939 |
| ORF1a | nsp3 | 2242 | 0.895 | 5.983 | 0.938 |
| Spike | Spike | 14 | 0.773 | 2.558 | 0.938 |
| ORF1a | nsp4 | 3158 | 0.594 | 2.996 | 0.938 |
| ORF1a | nsp3 | 1760 | 0.731 | 2.769 | 0.938 |
| ORF1a | nsp3 | 2739 | 0.636 | 2.661 | 0.938 |
| ORF3a | ORF3a | 129 | 0.229 | 0.812 | 0.937 |
| ORF1b | nsp12 | 252 | 0.477 | 2.526 | 0.937 |
| ORF1a | nsp3 | 1207 | 0.597 | 2.437 | 0.937 |
| ORF1b | nsp13 | 1223 | 0.531 | 2.249 | 0.936 |
| ORF1a | nsp1 | 100 | 0.403 | 1.532 | 0.935 |
| ORF1b | nsp15 | 2132 | 0.670 | 3.533 | 0.935 |
| N | N | 325 | 0.467 | 1.980 | 0.934 |
| N | N | 334 | 0.467 | 1.978 | 0.934 |
| N | N | 401 | 0.744 | 2.393 | 0.934 |
| N | N | 147 | 0.220 | 0.811 | 0.933 |
| ORF1a | nsp6 | 3832 | 0.476 | 2.307 | 0.933 |
| ORF1a | nsp1 | 75 | 0.924 | 3.160 | 0.933 |
| ORF1a | nsp2 | 558 | 0.453 | 1.458 | 0.933 |
| ORF8 | ORF8 | 76 | 0.163 | 0.661 | 0.932 |
| ORF1a | nsp2 | 516 | 0.926 | 2.682 | 0.932 |
| ORF3a | ORF3a | 101 | 0.607 | 1.520 | 0.932 |
| ORF1a | nsp4 | 2866 | 0.598 | 2.453 | 0.931 |
| Spike | Spike | 158 | 0.946 | 3.931 | 0.931 |
| ORF1a | nsp3 | 1500 | 0.688 | 3.470 | 0.931 |
| ORF1b | nsp12 | 188 | 0.735 | 4.094 | 0.931 |
| Spike | Spike | 1252 | 0.297 | 1.008 | 0.930 |
| ORF1a | nsp3 | 2488 | 0.630 | 2.541 | 0.930 |
| Spike | Spike | 477 | 3.113 | 7.146 | 0.930 |
| ORF1a | nsp2 | 352 | 0.677 | 2.827 | 0.929 |
| E | E | 61 | 0.740 | 2.529 | 0.929 |
| Spike | Spike | 26 | 0.871 | 2.815 | 0.929 |
| ORF1a | nsp4 | 2909 | 1.990 | 7.198 | 0.929 |
| N | N | 55 | 0.225 | 0.803 | 0.929 |
| N | N | 27 | 0.959 | 4.994 | 0.929 |
| ORF1b | nsp12 | 464 | 0.470 | 2.163 | 0.929 |
| ORF1a | nsp3 | 891 | 0.630 | 2.319 | 0.929 |
| ORF1a | nsp4 | 2800 | 0.629 | 2.552 | 0.928 |
| ORF1a | nsp2 | 268 | 0.792 | 2.608 | 0.928 |
| ORF1b | nsp12 | 797 | 0.470 | 2.148 | 0.928 |
| ORF1b | nsp14 | 1598 | 0.880 | 3.024 | 0.927 |
| ORF1a | nsp2 | 340 | 0.370 | 1.196 | 0.927 |
| ORF8 | ORF8 | 69 | 0.421 | 1.336 | 0.927 |
| ORF3a | ORF3a | 58 | 0.392 | 1.015 | 0.927 |
| ORF3a | ORF3a | 57 | 0.858 | 2.935 | 0.927 |
| ORF1b | nsp16 | 2565 | 0.480 | 2.081 | 0.927 |
| ORF1a | nsp4 | 3162 | 0.666 | 3.247 | 0.926 |
| Spike | Spike | 8 | 0.325 | 0.999 | 0.926 |
| Spike | Spike | 844 | 0.609 | 2.361 | 0.925 |
| ORF1b | nsp12 | 145 | 0.505 | 2.422 | 0.924 |
| N | N | 366 | 1.579 | 6.518 | 0.924 |
| ORF1a | nsp3 | 2165 | 0.571 | 2.549 | 0.923 |
| ORF1a | nsp2 | 300 | 0.395 | 1.276 | 0.923 |
| ORF8 | ORF8 | 32 | 0.419 | 1.327 | 0.923 |
| Spike | Spike | 1202 | 1.740 | 7.636 | 0.923 |
| ORF1a | nsp2 | 222 | 0.395 | 1.271 | 0.923 |
| ORF1b | nsp15 | 2313 | 0.693 | 2.832 | 0.922 |
| ORF1b | nsp15 | 2213 | 0.690 | 2.829 | 0.922 |
| ORF1a | nsp3 | 1952 | 0.728 | 2.509 | 0.922 |
| ORF1b | nsp14 | 2040 | 0.685 | 2.817 | 0.922 |
| ORF1a | nsp2 | 592 | 0.733 | 2.171 | 0.922 |
| ORF1a | nsp3 | 2618 | 0.624 | 2.418 | 0.922 |
| ORF1a | nsp3 | 1307 | 0.629 | 2.447 | 0.921 |
| Spike | Spike | 658 | 0.720 | 2.253 | 0.921 |
| ORF1a | nsp1 | 112 | 1.099 | 3.225 | 0.921 |
| N | N | 267 | 0.477 | 1.685 | 0.920 |
| M | M | 69 | 0.950 | 2.915 | 0.919 |
| Spike | Spike | 54 | 0.981 | 2.743 | 0.919 |
| ORF10 | ORF10 | 15 | 0.255 | 0.826 | 0.919 |
| ORF1a | nsp2 | 365 | 0.367 | 1.180 | 0.919 |
| ORF8 | ORF8 | 93 | 0.291 | 0.847 | 0.919 |
| Spike | Spike | 240 | 0.400 | 1.186 | 0.918 |
| ORF8 | ORF8 | 77 | 0.292 | 0.871 | 0.918 |
| ORF1a | nsp3 | 2054 | 0.566 | 2.610 | 0.918 |
| ORF1a | nsp3 | 2065 | 0.566 | 2.606 | 0.918 |
| ORF1a | nsp3 | 2034 | 0.632 | 3.409 | 0.917 |
| ORF1a | nsp2 | 474 | 0.250 | 0.789 | 0.917 |
| ORF10 | ORF10 | 23 | 0.229 | 0.727 | 0.917 |
| ORF1b | nsp14 | 1936 | 0.579 | 2.042 | 0.917 |
| ORF3a | ORF3a | 122 | 0.528 | 1.626 | 0.917 |
| N | N | 212 | 0.869 | 2.415 | 0.917 |
| ORF1a | nsp3 | 1520 | 0.581 | 2.109 | 0.916 |
| ORF1a | nsp2 | 362 | 0.672 | 2.797 | 0.916 |
| ORF1b | nsp13 | 1273 | 2.033 | 7.142 | 0.916 |
| ORF1a | nsp3 | 1532 | 0.697 | 2.905 | 0.916 |
| ORF1a | nsp2 | 314 | 0.673 | 2.786 | 0.915 |
| ORF8 | ORF8 | 41 | 0.184 | 0.639 | 0.915 |
| ORF1a | nsp2 | 614 | 0.443 | 1.285 | 0.915 |
| Spike | Spike | 414 | 0.853 | 3.441 | 0.915 |
| ORF1a | nsp8 | 4129 | 0.888 | 4.826 | 0.914 |
| ORF1a | nsp3 | 1828 | 0.819 | 2.557 | 0.914 |
| ORF1a | nsp10 | 4364 | 0.888 | 4.823 | 0.914 |
| ORF1b | nsp13 | 1078 | 0.584 | 2.119 | 0.914 |
| ORF1a | nsp2 | 450 | 0.733 | 2.102 | 0.914 |
| ORF1a | nsp3 | 993 | 0.704 | 2.168 | 0.914 |
| Spike | Spike | 210 | 0.445 | 1.352 | 0.914 |
| ORF1a | nsp2 | 371 | 0.601 | 2.064 | 0.914 |
| ORF1b | nsp15 | 2306 | 0.660 | 2.481 | 0.913 |
| ORF1b | nsp13 | 1242 | 0.715 | 3.313 | 0.913 |
| ORF1a | nsp3 | 1538 | 0.582 | 2.123 | 0.913 |
| ORF1a | nsp2 | 676 | 0.825 | 2.562 | 0.913 |
| ORF1b | nsp12 | 909 | 0.577 | 2.015 | 0.913 |
| ORF1a | nsp3 | 859 | 0.894 | 2.449 | 0.913 |
| ORF1a | nsp3 | 2162 | 0.246 | 1.032 | 0.912 |
| ORF3a | ORF3a | 217 | 0.229 | 0.755 | 0.912 |
| ORF3a | ORF3a | 42 | 0.965 | 2.566 | 0.912 |
| ORF1b | nsp12 | 98 | 0.633 | 2.485 | 0.912 |
| ORF1a | nsp2 | 232 | 0.640 | 1.897 | 0.912 |
| N | N | 139 | 0.467 | 1.008 | 0.912 |
| N | N | 393 | 0.224 | 0.779 | 0.912 |
| Spike | Spike | 67 | 0.978 | 2.815 | 0.912 |
| ORF1a | nsp3 | 2200 | 0.644 | 3.100 | 0.911 |
| ORF1b | nsp12 | 370 | 0.485 | 1.978 | 0.911 |
| Spike | Spike | 496 | 0.451 | 1.516 | 0.911 |
| ORF1a | nsp2 | 226 | 0.552 | 1.881 | 0.910 |
| ORF7a | ORF7a | 94 | 0.692 | 2.327 | 0.910 |
| Spike | Spike | 13 | 0.461 | 1.303 | 0.909 |
| ORF1a | nsp2 | 415 | 0.448 | 1.399 | 0.909 |
| ORF1a | nsp2 | 661 | 0.448 | 1.399 | 0.909 |
| ORF1a | nsp3 | 2405 | 0.654 | 3.187 | 0.908 |
| ORF1b | nsp14 | 1869 | 1.303 | 5.401 | 0.908 |
| ORF1a | nsp3 | 1093 | 0.449 | 1.436 | 0.908 |
| ORF1a | nsp3 | 1291 | 0.578 | 2.036 | 0.908 |
| ORF1a | nsp3 | 885 | 0.630 | 2.052 | 0.907 |
| Spike | Spike | 1163 | 2.287 | 7.090 | 0.907 |
| ORF3a | ORF3a | 21 | 0.903 | 2.806 | 0.907 |
| ORF7a | ORF7a | 82 | 1.136 | 2.813 | 0.907 |
| ORF1a | nsp3 | 1995 | 0.267 | 1.140 | 0.907 |
| ORF1b | nsp16 | 2685 | 0.487 | 1.947 | 0.907 |
| ORF1b | nsp12 | 132 | 0.842 | 3.076 | 0.906 |
| ORF1a | nsp3 | 1151 | 0.993 | 2.789 | 0.906 |
| N | N | 25 | 0.449 | 1.094 | 0.906 |
| ORF1b | nsp14 | 1649 | 0.579 | 2.046 | 0.906 |
| ORF1a | nsp5 | 3546 | 0.494 | 1.965 | 0.906 |
| ORF1b | nsp14 | 1914 | 0.631 | 2.480 | 0.906 |
| ORF1b | nsp12 | 861 | 0.581 | 2.097 | 0.906 |
| Spike | Spike | 769 | 1.170 | 4.695 | 0.906 |
| ORF1a | nsp4 | 2825 | 0.568 | 1.986 | 0.905 |
| E | E | 49 | 0.415 | 1.557 | 0.905 |
| ORF3a | ORF3a | 182 | 0.285 | 0.832 | 0.905 |
| ORF1b | nsp12 | 7 | 0.843 | 2.955 | 0.904 |
| ORF1a | nsp4 | 3076 | 0.593 | 2.064 | 0.904 |
| ORF1a | nsp2 | 335 | 0.397 | 1.302 | 0.904 |
| ORF1a | nsp1 | 28 | 0.597 | 1.992 | 0.903 |
| ORF1a | nsp5 | 3287 | 0.813 | 2.955 | 0.903 |
| ORF1a | nsp3 | 2532 | 0.614 | 2.166 | 0.903 |
| ORF3a | ORF3a | 49 | 0.505 | 1.061 | 0.902 |
| M | M | 23 | 0.578 | 1.914 | 0.902 |
| E | E | 58 | 0.425 | 1.572 | 0.902 |
| ORF1b | nsp14 | 1895 | 1.041 | 2.884 | 0.901 |
| ORF1a | nsp5 | 3547 | 0.515 | 1.997 | 0.901 |
| ORF1a | nsp5 | 3454 | 0.812 | 2.873 | 0.901 |
| ORF1a | nsp2 | 431 | 0.847 | 2.388 | 0.901 |
| ORF3a | ORF3a | 220 | 0.529 | 1.216 | 0.900 |
| ORF3a | ORF3a | 72 | 0.444 | 1.059 | 0.900 |

TABLE 3
Comprehensive list of sites under purifying selection

| Gene | Protein | Site | alpha | beta | Prob[alpha > beta] |
|---|---|---|---|---|---|
| Spike | Spike | 410 | 15.523 | 0.216 | 1.000 |
| ORF1b | nsp13 | 1140 | 29.716 | 0.343 | 1.000 |
| ORF1a | nsp1 | 84 | 29.272 | 0.631 | 1.000 |
| N | N | 110 | 7.187 | 0.022 | 1.000 |
| N | N | 274 | 7.187 | 0.022 | 1.000 |
| M | M | 53 | 29.361 | 0.201 | 1.000 |
| ORF1b | nsp16 | 2596 | 29.009 | 0.523 | 1.000 |
| ORF10 | ORF10 | 19 | 13.791 | 0.356 | 1.000 |
| N | N | 333 | 7.142 | 0.050 | 1.000 |
| Spike | Spike | 68 | 29.059 | 2.770 | 1.000 |
| ORF1a | nsp3 | 2638 | 28.228 | 0.529 | 1.000 |
| M | M | 93 | 15.246 | 0.166 | 1.000 |
| ORF1a | nsp3 | 924 | 15.156 | 0.151 | 1.000 |
| ORF1a | nsp2 | 549 | 11.652 | 0.675 | 1.000 |
| ORF1a | nsp2 | 186 | 7.328 | 0.201 | 1.000 |
| ORF1b | nsp12 | 757 | 15.484 | 0.280 | 1.000 |
| N | N | 346 | 6.234 | 0.021 | 1.000 |
| M | M | 112 | 10.162 | 0.151 | 1.000 |
| N | N | 309 | 7.124 | 0.062 | 1.000 |
| ORF1a | nsp8 | 4058 | 8.217 | 0.187 | 1.000 |
| ORF1b | nsp13 | 1361 | 9.941 | 0.220 | 1.000 |
| ORF1a | nsp6 | 3839 | 46.643 | 2.157 | 1.000 |
| ORF1a | nsp3 | 2376 | 12.151 | 0.135 | 1.000 |
| ORF1b | nsp15 | 2196 | 38.884 | 0.391 | 1.000 |
| ORF3a | ORF3a | 8 | 6.234 | 0.099 | 1.000 |
| ORF1a | nsp5 | 3368 | 7.372 | 0.193 | 1.000 |
| E | E | 4 | 14.723 | 0.261 | 1.000 |
| ORF1b | nsp12 | 831 | 14.985 | 0.262 | 1.000 |
| ORF1b | nsp14 | 1763 | 14.174 | 0.222 | 1.000 |
| Spike | Spike | 562 | 7.076 | 0.083 | 1.000 |
| Spike | Spike | 543 | 7.074 | 0.083 | 1.000 |
| ORF1a | nsp4 | 3048 | 29.761 | 0.314 | 1.000 |
| ORF1b | nsp13 | 1279 | 13.937 | 0.514 | 1.000 |
| ORF1a | nsp3 | 1330 | 12.639 | 0.221 | 1.000 |
| N | N | 86 | 6.723 | 0.050 | 1.000 |
| N | N | 360 | 6.719 | 0.050 | 1.000 |
| ORF1b | nsp14 | 2019 | 14.035 | 0.515 | 1.000 |
| N | N | 315 | 3.181 | 0.021 | 1.000 |
| N | N | 53 | 3.180 | 0.021 | 1.000 |
| ORF1a | nsp4 | 3053 | 24.939 | 0.571 | 1.000 |
| M | M | 179 | 9.288 | 0.201 | 1.000 |
| Spike | Spike | 821 | 15.462 | 0.357 | 1.000 |
| ORF1a | nsp2 | 188 | 7.129 | 0.246 | 1.000 |
| ORF1b | nsp13 | 931 | 12.368 | 0.246 | 1.000 |
| N | N | 318 | 6.948 | 0.270 | 1.000 |
| M | M | 120 | 7.140 | 0.152 | 1.000 |
| Spike | Spike | 296 | 3.233 | 0.049 | 1.000 |
| ORF1a | nsp3 | 2421 | 7.229 | 0.289 | 1.000 |
| ORF1a | nsp3 | 1438 | 16.402 | 0.777 | 1.000 |
| N | N | 291 | 6.655 | 0.056 | 1.000 |
| ORF1a | nsp4 | 3055 | 15.502 | 0.762 | 1.000 |
| ORF1b | nsp12 | 591 | 15.720 | 0.409 | 1.000 |
| N | N | 268 | 5.402 | 0.050 | 1.000 |
| ORF1a | nsp3 | 1773 | 26.574 | 0.699 | 1.000 |
| M | M | 172 | 13.532 | 0.175 | 1.000 |
| Spike | Spike | 1215 | 13.928 | 0.539 | 1.000 |
| ORF1a | nsp9 | 4252 | 15.481 | 0.560 | 1.000 |
| ORF1a | nsp3 | 1356 | 13.679 | 0.327 | 1.000 |
| ORF1b | nsp12 | 419 | 7.464 | 0.203 | 1.000 |
| ORF1a | nsp6 | 3853 | 7.142 | 0.245 | 1.000 |
| ORF1b | nsp13 | 1403 | 13.501 | 0.547 | 1.000 |
| ORF1b | nsp12 | 66 | 11.961 | 0.306 | 1.000 |
| ORF1b | nsp12 | 378 | 15.532 | 0.373 | 1.000 |
| M | M | 143 | 8.146 | 0.175 | 1.000 |
| ORF1a | nsp6 | 3585 | 7.130 | 0.387 | 1.000 |
| ORF1a | nsp4 | 3100 | 7.135 | 0.149 | 1.000 |
| M | M | 135 | 22.059 | 0.288 | 1.000 |
| N | N | 302 | 6.167 | 0.062 | 1.000 |
| Spike | Spike | 11 | 3.020 | 0.057 | 1.000 |
| E | E | 6 | 9.021 | 0.204 | 1.000 |
| ORF1a | nsp5 | 3377 | 15.042 | 0.554 | 1.000 |
| ORF1a | nsp6 | 3603 | 7.102 | 0.216 | 1.000 |
| Spike | Spike | 715 | 5.137 | 0.082 | 1.000 |
| ORF3a | ORF3a | 43 | 7.124 | 0.831 | 1.000 |
| ORF1a | nsp2 | 491 | 8.030 | 0.525 | 1.000 |
| ORF1b | nsp13 | 1433 | 7.158 | 0.230 | 1.000 |
| M | M | 132 | 7.058 | 0.126 | 1.000 |
| M | M | 124 | 7.143 | 0.401 | 1.000 |
| ORF1b | nsp12 | 486 | 14.549 | 0.579 | 1.000 |
| ORF1b | nsp13 | 1418 | 9.090 | 0.242 | 1.000 |
| ORF1b | nsp15 | 2265 | 15.213 | 0.366 | 1.000 |
| M | M | 41 | 13.751 | 0.264 | 1.000 |
| ORF1a | nsp3 | 1337 | 9.816 | 0.319 | 1.000 |
| ORF1b | nsp16 | 2609 | 11.266 | 0.226 | 1.000 |
| Spike | Spike | 53 | 4.765 | 0.100 | 1.000 |
| Spike | Spike | 1146 | 27.784 | 2.941 | 1.000 |
| Spike | Spike | 302 | 4.371 | 0.090 | 1.000 |
| N | N | 392 | 6.942 | 0.513 | 1.000 |
| ORF1b | nsp16 | 2404 | 9.147 | 0.434 | 1.000 |
| ORF3a | ORF3a | 207 | 4.154 | 0.387 | 1.000 |
| Spike | Spike | 60 | 2.844 | 0.056 | 1.000 |
| N | N | 337 | 2.983 | 0.044 | 1.000 |
| ORF1a | nsp5 | 3535 | 7.175 | 0.253 | 1.000 |
| ORF1a | nsp6 | 3689 | 14.370 | 0.682 | 1.000 |
| ORF1b | nsp12 | 5 | 23.300 | 0.319 | 1.000 |
| ORF1a | nsp6 | 3769 | 7.058 | 0.216 | 1.000 |
| N | N | 298 | 3.182 | 0.050 | 1.000 |
| ORF1a | nsp6 | 3831 | 5.807 | 0.153 | 1.000 |
| ORF1b | nsp14 | 1730 | 7.127 | 0.292 | 1.000 |
| ORF1b | nsp16 | 2452 | 7.870 | 0.259 | 1.000 |
| ORF1b | nsp14 | 1759 | 9.924 | 0.338 | 1.000 |
| ORF1b | nsp16 | 2459 | 6.564 | 0.154 | 1.000 |
| ORF1b | nsp13 | 1345 | 7.063 | 0.198 | 1.000 |
| ORF1b | nsp16 | 2552 | 7.858 | 0.217 | 1.000 |
| ORF1b | nsp15 | 2172 | 9.430 | 0.561 | 1.000 |
| ORF1a | nsp3 | 1907 | 6.715 | 0.195 | 1.000 |
| Spike | Spike | 432 | 5.698 | 0.098 | 1.000 |
| ORF1b | nsp12 | 491 | 38.436 | 0.434 | 1.000 |
| ORF1a | nsp2 | 341 | 6.206 | 0.161 | 1.000 |
| ORF1a | nsp3 | 2470 | 10.166 | 0.262 | 1.000 |
| N | N | 19 | 13.238 | 0.785 | 1.000 |
| ORF1a | nsp3 | 1868 | 7.119 | 0.299 | 1.000 |
| ORF1a | nsp7 | 3920 | 6.501 | 0.183 | 1.000 |
| ORF1a | nsp2 | 740 | 6.730 | 0.292 | 1.000 |
| ORF1a | nsp9 | 4168 | 12.289 | 0.261 | 1.000 |
| ORF1a | nsp5 | 3568 | 7.006 | 0.183 | 1.000 |
| N | N | 35 | 12.820 | 2.284 | 1.000 |
| ORF1b | nsp14 | 1531 | 7.033 | 0.203 | 1.000 |
| Spike | Spike | 665 | 2.924 | 0.075 | 1.000 |
| Spike | Spike | 618 | 14.893 | 0.616 | 1.000 |
| M | M | 117 | 8.299 | 0.246 | 1.000 |
| ORF6 | ORF6 | 61 | 34.253 | 15.518 | 1.000 |
| Spike | Spike | 55 | 2.838 | 0.057 | 1.000 |
| M | M | 121 | 7.711 | 0.244 | 1.000 |
| N | N | 30 | 7.196 | 0.598 | 1.000 |
| ORF1a | nsp3 | 1426 | 7.111 | 0.239 | 1.000 |
| ORF1b | nsp12 | 446 | 8.696 | 0.287 | 1.000 |
| ORF1a | nsp3 | 1643 | 7.102 | 0.280 | 1.000 |
| ORF1b | nsp15 | 2135 | 8.878 | 0.281 | 1.000 |
| ORF1a | nsp3 | 1415 | 14.067 | 0.840 | 1.000 |
| ORF1a | nsp3 | 839 | 6.940 | 0.202 | 1.000 |
| ORF1b | nsp12 | 75 | 14.184 | 0.376 | 1.000 |
| ORF1a | nsp3 | 1873 | 7.108 | 0.299 | 1.000 |
| Spike | Spike | 682 | 4.933 | 0.101 | 1.000 |
| ORF1a | nsp4 | 2986 | 7.057 | 0.142 | 1.000 |
| ORF1a | nsp4 | 3179 | 8.126 | 0.157 | 1.000 |
| ORF1a | nsp4 | 3234 | 6.131 | 0.152 | 1.000 |
| ORF1a | nsp6 | 3796 | 7.025 | 0.429 | 1.000 |
| ORF3a | ORF3a | 7 | 6.524 | 0.387 | 1.000 |
| M | M | 161 | 6.997 | 0.190 | 1.000 |
| ORF1a | nsp4 | 3198 | 7.156 | 0.196 | 1.000 |
| M | M | 100 | 7.045 | 0.393 | 1.000 |
| ORF1a | nsp10 | 4361 | 10.469 | 0.243 | 1.000 |
| ORF1a | nsp4 | 3180 | 7.122 | 0.195 | 1.000 |
| ORF1b | nsp14 | 1969 | 6.997 | 0.250 | 1.000 |
| ORF1a | nsp2 | 454 | 7.123 | 0.897 | 1.000 |
| ORF3a | ORF3a | 212 | 2.925 | 0.178 | 1.000 |
| ORF1a | nsp4 | 3153 | 7.056 | 0.151 | 1.000 |
| N | N | 312 | 6.096 | 0.041 | 1.000 |
| M | M | 203 | 7.137 | 0.242 | 1.000 |
| ORF1b | nsp16 | 2412 | 7.090 | 0.250 | 1.000 |
| ORF1a | nsp4 | 2895 | 7.133 | 0.490 | 1.000 |
| ORF1b | nsp16 | 2530 | 7.348 | 0.527 | 1.000 |
| ORF1a | nsp3 | 1627 | 7.045 | 0.280 | 1.000 |
| ORF1a | nsp3 | 2445 | 7.098 | 0.314 | 1.000 |
| ORF1a | nsp3 | 1345 | 7.350 | 0.233 | 1.000 |
| ORF1a | nsp3 | 1673 | 7.314 | 0.379 | 1.000 |
| ORF1a | nsp5 | 3414 | 7.130 | 0.292 | 1.000 |
| M | M | 56 | 7.133 | 0.511 | 1.000 |
| Spike | Spike | 692 | 7.108 | 0.571 | 1.000 |
| ORF1a | nsp3 | 1144 | 6.989 | 0.251 | 1.000 |
| ORF1a | nsp4 | 3238 | 4.230 | 0.128 | 1.000 |
| ORF1a | nsp4 | 2955 | 8.203 | 0.185 | 1.000 |
| M | M | 114 | 8.751 | 0.128 | 1.000 |
| Spike | Spike | 473 | 6.904 | 0.136 | 1.000 |
| Spike | Spike | 489 | 5.718 | 0.126 | 1.000 |
| ORF1b | nsp13 | 942 | 7.070 | 0.188 | 1.000 |
| ORF1a | nsp5 | 3321 | 6.984 | 0.388 | 1.000 |
| ORF1a | nsp3 | 1118 | 5.826 | 0.223 | 1.000 |
| ORF1a | nsp10 | 4266 | 19.566 | 0.522 | 1.000 |
| ORF1a | nsp4 | 2884 | 6.956 | 0.153 | 1.000 |
| N | N | 54 | 4.867 | 0.099 | 1.000 |
| ORF1a | nsp8 | 4045 | 3.982 | 0.111 | 1.000 |
| ORF1b | nsp15 | 2227 | 6.994 | 0.242 | 1.000 |
| ORF3a | ORF3a | 71 | 2.994 | 0.201 | 1.000 |
| M | M | 160 | 7.141 | 0.493 | 1.000 |
| M | M | 35 | 6.985 | 0.218 | 1.000 |
| ORF7a | ORF7a | 31 | 4.420 | 0.398 | 1.000 |
| ORF1a | nsp4 | 3007 | 7.116 | 0.215 | 1.000 |
| ORF1b | nsp14 | 1617 | 8.553 | 0.348 | 1.000 |
| M | M | 138 | 4.985 | 0.140 | 1.000 |
| ORF1b | nsp13 | 1036 | 7.185 | 0.256 | 1.000 |
| ORF1b | nsp14 | 1673 | 6.120 | 0.234 | 1.000 |
| Spike | Spike | 707 | 4.757 | 0.120 | 1.000 |
| ORF1a | nsp6 | 3753 | 15.820 | 2.103 | 1.000 |
| ORF1a | nsp1 | 140 | 7.112 | 0.506 | 1.000 |
| ORF1a | nsp2 | 735 | 6.496 | 0.603 | 1.000 |
| ORF1b | nsp12 | 882 | 11.050 | 0.592 | 1.000 |
| Spike | Spike | 1114 | 7.186 | 0.570 | 1.000 |
| ORF1b | nsp14 | 1907 | 7.145 | 0.362 | 1.000 |
| Spike | Spike | 508 | 3.862 | 0.115 | 1.000 |
| ORF1a | nsp6 | 3813 | 6.956 | 0.289 | 1.000 |
| ORF1a | nsp6 | 3629 | 4.874 | 0.210 | 1.000 |
| ORF3a | ORF3a | 130 | 2.804 | 0.271 | 1.000 |
| ORF1a | nsp5 | 3508 | 7.082 | 0.279 | 1.000 |
| Spike | Spike | 276 | 2.844 | 0.094 | 1.000 |
| ORF1a | nsp4 | 3197 | 7.518 | 0.177 | 1.000 |
| Spike | Spike | 648 | 2.816 | 0.095 | 1.000 |
| N | N | 192 | 7.095 | 0.532 | 1.000 |
| ORF1a | nsp8 | 3965 | 8.121 | 0.297 | 1.000 |
| ORF1a | nsp9 | 4205 | 7.584 | 0.679 | 1.000 |
| ORF1b | nsp13 | 965 | 6.897 | 0.184 | 1.000 |
| ORF1b | nsp12 | 875 | 7.071 | 0.218 | 1.000 |
| ORF6 | ORF6 | 4 | 2.887 | 0.519 | 1.000 |
| ORF1a | nsp5 | 3291 | 7.082 | 0.291 | 1.000 |
| N | N | 111 | 2.772 | 0.052 | 1.000 |
| ORF1a | nsp3 | 1925 | 5.981 | 0.434 | 1.000 |
| ORF1a | nsp8 | 4070 | 6.871 | 0.253 | 1.000 |
| ORF1a | nsp2 | 387 | 2.961 | 0.184 | 1.000 |
| Spike | Spike | 587 | 5.010 | 0.104 | 1.000 |
| ORF7a | ORF7a | 40 | 3.917 | 0.522 | 1.000 |
| ORF8 | ORF8 | 36 | 7.125 | 1.683 | 1.000 |
| M | M | 207 | 7.443 | 0.557 | 1.000 |
| ORF1b | nsp14 | 1760 | 7.127 | 0.779 | 1.000 |
| ORF1b | nsp14 | 1627 | 6.661 | 0.292 | 1.000 |
| ORF1b | nsp13 | 1163 | 7.053 | 0.223 | 1.000 |
| ORF1b | nsp13 | 1150 | 7.041 | 0.222 | 1.000 |
| ORF1a | nsp5 | 3397 | 3.309 | 0.135 | 1.000 |
| ORF1a | nsp2 | 811 | 8.647 | 0.553 | 1.000 |
| ORF1a | nsp3 | 1652 | 3.036 | 0.212 | 1.000 |
| ORF1a | nsp9 | 4185 | 6.925 | 0.248 | 1.000 |
| Spike | Spike | 1110 | 9.067 | 0.325 | 1.000 |
| ORF1b | nsp14 | 1901 | 7.124 | 0.842 | 1.000 |
| ORF1a | nsp3 | 2338 | 2.847 | 0.227 | 1.000 |
| Spike | Spike | 789 | 2.882 | 0.109 | 1.000 |
| ORF1a | nsp3 | 1439 | 7.105 | 0.359 | 1.000 |
| ORF1a | nsp2 | 266 | 2.792 | 0.121 | 1.000 |
| ORF6 | ORF6 | 16 | 3.340 | 0.317 | 1.000 |
| ORF1a | nsp5 | 3297 | 7.147 | 0.647 | 1.000 |
| ORF1b | nsp13 | 1060 | 6.987 | 0.191 | 1.000 |
| ORF1a | nsp7 | 3899 | 4.978 | 0.436 | 1.000 |
| ORF1a | nsp10 | 4358 | 12.245 | 0.372 | 1.000 |
| ORF1b | nsp13 | 954 | 6.985 | 0.218 | 1.000 |
| ORF1a | nsp4 | 2907 | 7.036 | 0.216 | 1.000 |
| N | N | 157 | 2.818 | 0.295 | 1.000 |
| ORF1a | nsp2 | 565 | 2.875 | 0.420 | 1.000 |
| ORF1a | nsp3 | 927 | 10.661 | 0.610 | 1.000 |
| ORF1a | nsp3 | 1707 | 6.877 | 0.486 | 1.000 |
| M | M | 102 | 6.085 | 0.212 | 1.000 |
| Spike | Spike | 413 | 2.892 | 0.097 | 1.000 |
| ORF1a | nsp8 | 4015 | 17.971 | 0.445 | 1.000 |
| Spike | Spike | 83 | 2.843 | 0.491 | 1.000 |
| ORF1b | nsp12 | 830 | 7.472 | 0.229 | 1.000 |
| ORF1a | nsp3 | 2675 | 2.874 | 0.181 | 1.000 |
| ORF1a | nsp10 | 4284 | 6.496 | 0.243 | 1.000 |
| Spike | Spike | 475 | 2.946 | 0.320 | 1.000 |
| ORF3a | ORF3a | 36 | 2.759 | 0.245 | 1.000 |
| ORF1a | nsp7 | 3897 | 8.584 | 0.938 | 1.000 |
| ORF1a | nsp5 | 3479 | 7.124 | 0.647 | 1.000 |
| ORF1a | nsp2 | 190 | 3.638 | 0.552 | 1.000 |
| Spike | Spike | 1178 | 6.940 | 0.374 | 1.000 |
| ORF1b | nsp14 | 1804 | 6.987 | 0.362 | 1.000 |
| ORF1a | nsp6 | 3636 | 6.531 | 0.583 | 1.000 |
| ORF1b | nsp12 | 590 | 6.374 | 0.213 | 1.000 |
| ORF8 | ORF8 | 120 | 15.517 | 7.122 | 1.000 |
| Spike | Spike | 294 | 2.693 | 0.064 | 1.000 |
| ORF1a | nsp10 | 4312 | 7.881 | 0.433 | 1.000 |
| ORF1b | nsp12 | 619 | 7.093 | 0.318 | 1.000 |
| ORF6 | ORF6 | 32 | 6.651 | 0.691 | 1.000 |
| Spike | Spike | 234 | 2.763 | 0.090 | 1.000 |
| ORF1a | nsp6 | 3834 | 3.602 | 0.225 | 1.000 |
| ORF1a | nsp9 | 4172 | 7.133 | 0.587 | 1.000 |
| ORF1b | nsp12 | 692 | 7.112 | 0.221 | 1.000 |
| ORF1b | nsp12 | 585 | 4.583 | 0.150 | 1.000 |
| ORF1b | nsp13 | 952 | 6.344 | 0.185 | 1.000 |
| ORF1a | nsp1 | 97 | 2.825 | 0.184 | 1.000 |
| ORF1a | nsp1 | 136 | 2.825 | 0.184 | 1.000 |
| ORF1a | nsp4 | 2985 | 8.076 | 0.227 | 1.000 |
| N | N | 341 | 6.933 | 0.621 | 1.000 |
| Spike | Spike | 306 | 2.974 | 0.508 | 1.000 |
| ORF1a | nsp3 | 2560 | 6.951 | 0.361 | 1.000 |
| M | M | 164 | 5.360 | 0.205 | 1.000 |
| ORF1a | nsp3 | 1960 | 3.382 | 0.449 | 1.000 |
| Spike | Spike | 620 | 2.717 | 0.089 | 1.000 |
| ORF1a | nsp6 | 3785 | 3.137 | 0.199 | 1.000 |
| N | N | 292 | 2.972 | 0.609 | 1.000 |
| ORF3a | ORF3a | 20 | 3.051 | 0.580 | 1.000 |
| Spike | Spike | 979 | 11.160 | 0.636 | 1.000 |
| ORF1b | nsp12 | 368 | 6.931 | 0.424 | 1.000 |
| ORF1a | nsp9 | 4186 | 2.884 | 0.106 | 1.000 |
| ORF1b | nsp12 | 470 | 6.808 | 0.263 | 1.000 |
| ORF1a | nsp3 | 1523 | 6.874 | 0.299 | 1.000 |
| ORF1a | nsp7 | 3867 | 3.131 | 0.205 | 1.000 |
| ORF1a | nsp3 | 1700 | 5.859 | 0.298 | 1.000 |
| ORF1b | nsp13 | 987 | 7.113 | 0.564 | 1.000 |
| ORF1b | nsp14 | 1906 | 5.674 | 0.295 | 1.000 |
| ORF1b | nsp12 | 635 | 13.840 | 1.044 | 1.000 |
| ORF7a | ORF7a | 52 | 2.791 | 0.286 | 1.000 |
| Spike | Spike | 130 | 2.501 | 0.057 | 1.000 |
| ORF1a | nsp2 | 615 | 6.456 | 0.604 | 1.000 |
| Spike | Spike | 657 | 2.806 | 0.119 | 1.000 |
| ORF1a | nsp6 | 3803 | 5.596 | 0.286 | 1.000 |
| ORF1b | nsp13 | 1461 | 6.127 | 0.284 | 1.000 |
| ORF1a | nsp8 | 4073 | 3.058 | 0.125 | 1.000 |
| ORF1b | nsp14 | 1668 | 7.882 | 0.385 | 1.000 |
| ORF1a | nsp3 | 1749 | 7.109 | 0.811 | 1.000 |
| E | E | 23 | 4.642 | 0.243 | 1.000 |
| ORF1b | nsp12 | 704 | 7.008 | 0.316 | 1.000 |
| ORF1b | nsp12 | 23 | 6.638 | 0.387 | 1.000 |
| Spike | Spike | 1238 | 6.780 | 0.621 | 1.000 |
| ORF1a | nsp5 | 3394 | 7.768 | 0.266 | 1.000 |
| ORF1a | nsp3 | 1120 | 6.733 | 0.142 | 1.000 |
| ORF1b | nsp16 | 2493 | 3.052 | 0.108 | 1.000 |
| ORF1b | nsp14 | 1696 | 9.710 | 0.714 | 1.000 |
| ORF7a | ORF7a | 15 | 3.075 | 0.319 | 1.000 |
| ORF1b | nsp12 | 749 | 3.479 | 0.117 | 1.000 |
| ORF1b | nsp12 | 829 | 7.132 | 0.575 | 1.000 |
| M | M | 129 | 4.189 | 0.200 | 1.000 |
| ORF1b | nsp16 | 2418 | 4.594 | 0.184 | 1.000 |
| ORF1a | nsp3 | 1658 | 4.932 | 0.297 | 1.000 |
| ORF1a | nsp5 | 3502 | 6.170 | 0.235 | 1.000 |
| ORF1a | nsp2 | 440 | 8.548 | 0.738 | 1.000 |
| ORF1a | nsp2 | 748 | 7.000 | 0.996 | 1.000 |
| ORF1a | nsp3 | 1675 | 6.086 | 0.298 | 1.000 |
| ORF1b | nsp13 | 947 | 6.421 | 0.424 | 0.999 |
| ORF1a | nsp3 | 2676 | 2.877 | 0.232 | 0.999 |
| ORF1a | nsp4 | 2975 | 7.860 | 0.506 | 0.999 |
| ORF1a | nsp6 | 3690 | 5.382 | 0.481 | 0.999 |
| ORF3a | ORF3a | 13 | 7.461 | 0.839 | 0.999 |
| M | M | 202 | 3.351 | 0.166 | 0.999 |
| ORF1b | nsp13 | 1119 | 12.537 | 0.305 | 0.999 |
| ORF1a | nsp7 | 3928 | 6.963 | 0.589 | 0.999 |
| M | M | 190 | 4.008 | 0.190 | 0.999 |
| ORF1b | nsp12 | 336 | 11.932 | 0.349 | 0.999 |
| ORF1a | nsp2 | 416 | 2.761 | 0.262 | 0.999 |
| ORF1b | nsp12 | 824 | 7.616 | 0.258 | 0.999 |
| ORF1a | nsp3 | 1744 | 6.855 | 0.722 | 0.999 |
| ORF3a | ORF3a | 113 | 2.825 | 0.616 | 0.999 |
| ORF3a | ORF3a | 193 | 41.155 | 0.461 | 0.999 |
| ORF1b | nsp12 | 543 | 7.452 | 0.302 | 0.999 |
| ORF1a | nsp5 | 3338 | 7.332 | 2.725 | 0.999 |
| ORF1b | nsp14 | 1740 | 3.821 | 0.296 | 0.999 |
| ORF1a | nsp9 | 4195 | 12.570 | 0.344 | 0.999 |
| ORF1a | nsp1 | 88 | 2.946 | 0.266 | 0.999 |
| N | N | 158 | 2.187 | 0.052 | 0.999 |
| ORF1b | nsp12 | 781 | 6.820 | 0.312 | 0.999 |
| ORF1b | nsp13 | 925 | 5.871 | 0.181 | 0.999 |
| ORF6 | ORF6 | 20 | 7.704 | 1.173 | 0.999 |
| ORF1b | nsp13 | 1235 | 2.976 | 0.245 | 0.999 |
| ORF1b | nsp15 | 2387 | 4.187 | 0.178 | 0.999 |
| ORF1a | nsp6 | 3770 | 3.670 | 0.486 | 0.999 |
| ORF1a | nsp6 | 3828 | 6.830 | 0.579 | 0.999 |
| ORF1b | nsp14 | 1633 | 6.258 | 0.363 | 0.999 |
| ORF1b | nsp16 | 2427 | 7.015 | 0.232 | 0.999 |
| ORF1a | nsp3 | 1425 | 6.348 | 0.314 | 0.999 |
| N | N | 78 | 2.672 | 0.098 | 0.999 |
| ORF1a | nsp2 | 435 | 4.777 | 0.503 | 0.999 |
| ORF1a | nsp3 | 1441 | 7.035 | 0.631 | 0.999 |
| ORF1a | nsp3 | 2334 | 2.847 | 0.443 | 0.999 |
| ORF1a | nsp3 | 2584 | 13.103 | 2.587 | 0.999 |
| ORF1b | nsp15 | 2237 | 9.214 | 1.344 | 0.999 |
| ORF1a | nsp3 | 1263 | 5.312 | 0.216 | 0.999 |
| ORF1b | nsp13 | 1444 | 10.393 | 0.522 | 0.999 |
| N | N | 327 | 7.119 | 2.330 | 0.999 |
| ORF7a | ORF7a | 69 | 2.832 | 0.585 | 0.999 |
| M | M | 28 | 6.625 | 0.689 | 0.999 |
| Spike | Spike | 43 | 2.191 | 0.066 | 0.999 |
| ORF1a | nsp5 | 3426 | 2.744 | 0.148 | 0.999 |
| ORF10 | ORF10 | 25 | 2.911 | 0.331 | 0.999 |
| Spike | Spike | 856 | 14.592 | 1.695 | 0.999 |
| ORF1a | nsp5 | 3424 | 3.334 | 0.198 | 0.999 |
| ORF1b | nsp16 | 2585 | 12.645 | 1.073 | 0.999 |
| ORF3a | ORF3a | 84 | 2.506 | 0.200 | 0.999 |
| N | N | 159 | 2.394 | 0.070 | 0.999 |
| ORF1a | nsp3 | 2172 | 2.812 | 0.443 | 0.999 |
| ORF1b | nsp12 | 766 | 5.699 | 0.241 | 0.999 |
| Spike | Spike | 1148 | 2.679 | 0.150 | 0.999 |
| ORF1a | nsp3 | 2557 | 6.068 | 0.502 | 0.999 |
| ORF1b | nsp12 | 282 | 6.465 | 0.386 | 0.999 |
| ORF1a | nsp3 | 2433 | 2.698 | 0.199 | 0.999 |
| ORF3a | ORF3a | 133 | 1.840 | 0.111 | 0.999 |
| ORF1a | nsp2 | 402 | 6.426 | 0.853 | 0.999 |
| N | N | 367 | 6.778 | 0.727 | 0.999 |
| ORF1b | nsp14 | 2029 | 3.083 | 0.146 | 0.999 |
| ORF1a | nsp6 | 3719 | 5.548 | 0.286 | 0.999 |
| M | M | 175 | 6.351 | 0.584 | 0.999 |
| ORF1b | nsp12 | 408 | 17.334 | 0.378 | 0.999 |
| Spike | Spike | 960 | 5.149 | 0.320 | 0.999 |
| ORF1a | nsp5 | 3472 | 3.021 | 0.194 | 0.999 |
| Spike | Spike | 824 | 5.127 | 0.320 | 0.999 |
| Spike | Spike | 290 | 2.188 | 0.071 | 0.999 |
| Spike | Spike | 518 | 3.284 | 0.379 | 0.999 |
| ORF1a | nsp10 | 4328 | 3.030 | 0.151 | 0.999 |
| ORF8 | ORF8 | 17 | 6.313 | 0.969 | 0.999 |
| ORF1a | nsp4 | 3259 | 6.234 | 0.166 | 0.999 |
| ORF1a | nsp1 | 106 | 2.834 | 0.521 | 0.999 |
| ORF1a | nsp8 | 3950 | 5.828 | 0.711 | 0.999 |
| ORF1a | nsp9 | 4163 | 2.944 | 0.153 | 0.999 |
| ORF1a | nsp1 | 126 | 2.788 | 0.258 | 0.999 |
| ORF1a | nsp2 | 189 | 2.788 | 0.258 | 0.999 |
| ORF1a | nsp2 | 570 | 2.795 | 0.322 | 0.999 |
| M | M | 106 | 4.188 | 0.150 | 0.999 |
| ORF1a | nsp2 | 729 | 2.794 | 0.322 | 0.999 |
| Spike | Spike | 569 | 3.130 | 0.101 | 0.998 |
| ORF1a | nsp3 | 2226 | 2.820 | 0.300 | 0.998 |
| N | N | 116 | 2.513 | 0.085 | 0.998 |
| Spike | Spike | 636 | 3.624 | 0.118 | 0.998 |
| ORF1b | nsp14 | 1783 | 6.928 | 0.675 | 0.998 |
| ORF1a | nsp5 | 3450 | 4.881 | 0.250 | 0.998 |
| ORF1a | nsp4 | 2992 | 7.311 | 0.171 | 0.998 |
| ORF1a | nsp4 | 3044 | 7.292 | 0.170 | 0.998 |
| ORF1a | nsp3 | 1369 | 7.520 | 0.309 | 0.998 |
| ORF1b | nsp13 | 928 | 5.595 | 0.177 | 0.998 |
| Spike | Spike | 28 | 2.828 | 0.471 | 0.998 |
| ORF1b | nsp14 | 1850 | 3.815 | 0.521 | 0.998 |
| Spike | Spike | 312 | 2.695 | 0.381 | 0.998 |
| N | N | 257 | 3.867 | 0.093 | 0.998 |
| ORF1a | nsp5 | 3290 | 3.452 | 0.126 | 0.998 |
| Spike | Spike | 116 | 1.714 | 0.071 | 0.998 |
| M | M | 71 | 6.646 | 0.662 | 0.998 |
| ORF1a | nsp2 | 793 | 2.593 | 0.246 | 0.998 |
| Spike | Spike | 407 | 2.250 | 0.099 | 0.998 |
| ORF1b | nsp12 | 569 | 6.674 | 0.187 | 0.998 |
| ORF1a | nsp5 | 3273 | 3.555 | 0.133 | 0.998 |
| Spike | Spike | 709 | 3.631 | 0.135 | 0.998 |
| ORF1a | nsp3 | 2578 | 5.405 | 0.478 | 0.998 |
| ORF1a | nsp5 | 3439 | 3.974 | 0.238 | 0.998 |
| ORF1b | nsp12 | 558 | 2.831 | 0.145 | 0.998 |
| ORF1a | nsp4 | 2935 | 6.106 | 0.250 | 0.998 |
| ORF1a | nsp4 | 3092 | 2.537 | 0.185 | 0.998 |
| ORF1a | nsp2 | 447 | 4.830 | 0.588 | 0.998 |
| N | N | 353 | 1.844 | 0.090 | 0.998 |
| ORF1a | nsp4 | 3081 | 6.097 | 0.252 | 0.998 |
| ORF1a | nsp3 | 2523 | 7.064 | 0.857 | 0.998 |
| ORF1a | nsp3 | 1797 | 2.739 | 0.275 | 0.998 |
| ORF1b | nsp16 | 2558 | 3.127 | 0.150 | 0.998 |
| ORF1b | nsp14 | 2011 | 5.128 | 0.216 | 0.997 |
| ORF1a | nsp6 | 3755 | 2.813 | 0.436 | 0.997 |
| ORF1a | nsp3 | 1174 | 2.791 | 0.569 | 0.997 |
| ORF1b | nsp12 | 691 | 6.478 | 0.554 | 0.997 |
| ORF1a | nsp4 | 2940 | 11.039 | 0.310 | 0.997 |
| Spike | Spike | 937 | 4.885 | 0.202 | 0.997 |
| ORF1a | nsp3 | 1578 | 6.316 | 0.370 | 0.997 |
| Spike | Spike | 691 | 3.228 | 0.604 | 0.997 |
| ORF6 | ORF6 | 31 | 2.990 | 0.656 | 0.997 |
| ORF1b | nsp12 | 237 | 5.022 | 0.563 | 0.997 |
| ORF1a | nsp3 | 1025 | 4.397 | 0.646 | 0.997 |
| ORF1b | nsp13 | 1483 | 8.548 | 0.325 | 0.997 |
| ORF1a | nsp3 | 1544 | 2.587 | 0.228 | 0.997 |
| ORF1a | nsp3 | 2206 | 2.468 | 0.238 | 0.997 |
| ORF1a | nsp3 | 2490 | 5.938 | 0.568 | 0.997 |
| ORF1a | nsp5 | 3271 | 2.880 | 0.451 | 0.997 |
| Spike | Spike | 952 | 2.528 | 0.177 | 0.997 |
| ORF1a | nsp4 | 3098 | 4.310 | 0.290 | 0.997 |
| ORF1b | nsp14 | 1930 | 4.430 | 0.363 | 0.997 |
| ORF1a | nsp2 | 717 | 2.665 | 0.310 | 0.997 |
| ORF1a | nsp3 | 1702 | 5.889 | 0.232 | 0.997 |
| ORF1b | nsp13 | 1314 | 3.862 | 0.284 | 0.997 |
| N | N | 225 | 2.457 | 0.116 | 0.997 |
| ORF1a | nsp3 | 1849 | 7.128 | 1.913 | 0.997 |
| M | M | 5 | 3.290 | 0.222 | 0.997 |
| ORF1b | nsp12 | 603 | 3.274 | 0.121 | 0.997 |
| Spike | Spike | 316 | 1.985 | 0.068 | 0.997 |
| ORF1b | nsp15 | 2139 | 4.755 | 0.244 | 0.997 |
| N | N | 329 | 2.493 | 0.116 | 0.997 |
| ORF1b | nsp12 | 519 | 2.640 | 0.129 | 0.997 |
| N | N | 203 | 16.216 | 2.945 | 0.997 |
| M | M | 150 | 3.121 | 0.151 | 0.997 |
| ORF1b | nsp12 | 427 | 26.622 | 1.248 | 0.997 |
| ORF7a | ORF7a | 79 | 2.748 | 0.624 | 0.997 |
| Spike | Spike | 1026 | 2.951 | 0.176 | 0.997 |
| ORF1a | nsp4 | 3089 | 3.950 | 0.526 | 0.997 |
| Spike | Spike | 395 | 1.303 | 0.073 | 0.997 |
| ORF1b | nsp14 | 1667 | 8.447 | 0.379 | 0.997 |
| Spike | Spike | 1272 | 3.530 | 0.278 | 0.996 |
| ORF1a | nsp9 | 4247 | 3.616 | 0.158 | 0.996 |
| ORF1a | nsp1 | 160 | 4.362 | 0.574 | 0.996 |
| ORF1b | nsp16 | 2479 | 4.710 | 0.152 | 0.996 |
| ORF1b | nsp16 | 2683 | 2.803 | 0.142 | 0.996 |
| Spike | Spike | 1100 | 4.923 | 0.227 | 0.996 |
| Spike | Spike | 1062 | 2.343 | 0.148 | 0.996 |
| ORF1a | nsp2 | 742 | 2.954 | 0.312 | 0.996 |
| ORF1b | nsp12 | 51 | 6.761 | 0.902 | 0.996 |
| ORF1b | nsp12 | 251 | 6.757 | 0.901 | 0.996 |
| ORF1b | nsp16 | 2598 | 4.676 | 0.153 | 0.996 |
| ORF1b | nsp16 | 2597 | 4.674 | 0.153 | 0.996 |
| ORF1b | nsp12 | 332 | 7.802 | 0.305 | 0.996 |
| M | M | 184 | 3.037 | 0.123 | 0.996 |
| ORF1b | nsp14 | 1787 | 5.233 | 0.593 | 0.996 |
| ORF10 | ORF10 | 32 | 6.203 | 0.377 | 0.996 |
| Spike | Spike | 733 | 2.971 | 0.105 | 0.996 |
| ORF1a | nsp2 | 815 | 1.873 | 0.199 | 0.996 |
| ORF1a | nsp2 | 304 | 2.647 | 0.190 | 0.996 |
| Spike | Spike | 541 | 1.717 | 0.089 | 0.996 |
| Spike | Spike | 663 | 2.224 | 0.115 | 0.996 |
| ORF1a | nsp2 | 757 | 3.832 | 0.784 | 0.996 |
| ORF1a | nsp6 | 3700 | 2.780 | 0.202 | 0.996 |
| ORF1a | nsp8 | 3945 | 2.246 | 0.161 | 0.996 |
| ORF1a | nsp3 | 1423 | 3.709 | 0.340 | 0.996 |
| ORF1a | nsp7 | 3926 | 2.654 | 0.263 | 0.996 |
| ORF1b | nsp12 | 729 | 8.111 | 1.132 | 0.996 |
| ORF1b | nsp15 | 2124 | 6.804 | 0.333 | 0.996 |
| ORF1a | nsp7 | 3903 | 4.243 | 0.249 | 0.996 |
| M | M | 113 | 4.269 | 0.225 | 0.996 |
| ORF1a | nsp4 | 2962 | 4.747 | 0.213 | 0.996 |
| N | N | 174 | 5.315 | 0.148 | 0.996 |
| Spike | Spike | 267 | 1.409 | 0.072 | 0.995 |
| N | N | 404 | 1.019 | 0.077 | 0.995 |
| ORF1b | nsp12 | 285 | 2.985 | 0.299 | 0.995 |
| ORF1a | nsp4 | 3257 | 2.519 | 0.173 | 0.995 |
| Spike | Spike | 345 | 1.302 | 0.082 | 0.995 |
| ORF1b | nsp12 | 625 | 2.590 | 0.144 | 0.995 |
| ORF3a | ORF3a | 65 | 2.827 | 1.058 | 0.995 |
| ORF1b | nsp12 | 372 | 2.660 | 0.246 | 0.995 |
| ORF1a | nsp3 | 2623 | 6.093 | 0.411 | 0.995 |
| ORF1a | nsp2 | 809 | 4.532 | 0.192 | 0.995 |
| ORF1a | nsp3 | 1157 | 2.645 | 0.396 | 0.995 |
| ORF1a | nsp3 | 2679 | 11.544 | 0.434 | 0.995 |
| ORF1b | nsp16 | 2582 | 4.727 | 0.176 | 0.995 |
| ORF1b | nsp12 | 903 | 4.210 | 0.184 | 0.995 |
| ORF1a | nsp4 | 2998 | 11.596 | 0.737 | 0.995 |
| ORF1a | nsp3 | 1342 | 5.586 | 0.235 | 0.995 |
| Spike | Spike | 940 | 7.143 | 2.707 | 0.995 |
| ORF1a | nsp3 | 1565 | 4.971 | 0.539 | 0.995 |
| ORF1a | nsp7 | 3940 | 4.874 | 0.757 | 0.995 |
| ORF3a | ORF3a | 148 | 1.073 | 0.110 | 0.995 |
| ORF1b | nsp12 | 488 | 4.714 | 0.262 | 0.995 |
| Spike | Spike | 146 | 3.010 | 0.816 | 0.995 |
| ORF1b | nsp12 | 159 | 3.439 | 0.343 | 0.995 |
| E | E | 60 | 3.629 | 0.199 | 0.995 |
| Spike | Spike | 313 | 2.475 | 0.115 | 0.994 |
| ORF1a | nsp3 | 1903 | 2.714 | 0.342 | 0.994 |
| ORF3a | ORF3a | 253 | 2.797 | 1.032 | 0.994 |
| ORF1a | nsp8 | 4077 | 4.118 | 0.214 | 0.994 |
| ORF1a | nsp10 | 4283 | 4.114 | 0.214 | 0.994 |
| ORF1a | nsp3 | 1922 | 2.805 | 0.367 | 0.994 |
| ORF1a | nsp3 | 1919 | 6.049 | 0.382 | 0.994 |
| ORF1b | nsp16 | 2547 | 2.522 | 0.117 | 0.994 |
| ORF1a | nsp2 | 786 | 2.764 | 0.568 | 0.994 |
| Spike | Spike | 585 | 2.009 | 0.088 | 0.994 |
| ORF1a | nsp2 | 410 | 2.601 | 0.250 | 0.994 |
| ORF1a | nsp3 | 1273 | 6.726 | 0.690 | 0.994 |
| ORF1a | nsp9 | 4235 | 4.607 | 0.272 | 0.994 |
| ORF1b | nsp12 | 549 | 3.285 | 0.157 | 0.994 |
| ORF1a | nsp3 | 1203 | 7.064 | 1.264 | 0.994 |
| ORF1b | nsp13 | 1157 | 5.482 | 0.139 | 0.994 |
| N | N | 314 | 0.713 | 0.049 | 0.994 |
| ORF1a | nsp6 | 3774 | 4.252 | 0.284 | 0.994 |
| ORF1b | nsp16 | 2540 | 5.003 | 0.203 | 0.994 |
| ORF8 | ORF8 | 80 | 5.026 | 0.509 | 0.994 |
| ORF1b | nsp12 | 345 | 11.721 | 0.930 | 0.994 |
| ORF1a | nsp3 | 1489 | 5.425 | 0.323 | 0.994 |
| ORF1b | nsp14 | 1983 | 8.544 | 0.588 | 0.994 |
| ORF1b | nsp12 | 670 | 6.923 | 0.283 | 0.994 |
| ORF1a | nsp5 | 3329 | 2.253 | 0.144 | 0.994 |
| ORF1b | nsp14 | 1934 | 4.692 | 0.416 | 0.994 |
| Spike | Spike | 606 | 2.215 | 0.134 | 0.994 |
| ORF1a | nsp10 | 4325 | 3.344 | 0.142 | 0.994 |
| ORF1a | nsp3 | 1935 | 4.223 | 0.707 | 0.994 |
| N | N | 128 | 7.126 | 2.833 | 0.994 |
| Spike | Spike | 578 | 2.687 | 0.118 | 0.994 |
| ORF1a | nsp3 | 1139 | 3.687 | 0.346 | 0.994 |
| ORF1a | nsp7 | 3935 | 4.817 | 0.606 | 0.994 |
| ORF1b | nsp12 | 28 | 5.108 | 0.643 | 0.994 |
| ORF1a | nsp5 | 3314 | 2.739 | 0.238 | 0.993 |
| ORF1a | nsp2 | 616 | 2.832 | 0.779 | 0.993 |
| ORF1b | nsp13 | 1320 | 5.449 | 0.717 | 0.993 |
| Spike | Spike | 878 | 2.920 | 0.224 | 0.993 |
| Spike | Spike | 815 | 2.838 | 0.174 | 0.993 |
| ORF1a | nsp6 | 3849 | 2.248 | 0.198 | 0.993 |
| ORF1b | nsp15 | 2317 | 11.385 | 0.392 | 0.993 |
| ORF1b | nsp13 | 1422 | 1.932 | 0.175 | 0.993 |
| Spike | Spike | 32 | 2.796 | 0.756 | 0.993 |
| ORF1a | nsp2 | 791 | 1.872 | 0.193 | 0.993 |
| Spike | Spike | 431 | 1.745 | 0.119 | 0.993 |
| ORF1a | nsp3 | 2302 | 1.896 | 0.142 | 0.993 |
| ORF3a | ORF3a | 112 | 2.828 | 1.047 | 0.993 |
| ORF1b | nsp13 | 1005 | 9.649 | 0.576 | 0.993 |
| ORF1a | nsp4 | 2852 | 4.950 | 0.136 | 0.993 |
| ORF1a | nsp8 | 4063 | 2.686 | 0.120 | 0.993 |
| ORF1a | nsp2 | 441 | 6.487 | 0.629 | 0.993 |
| Spike | Spike | 389 | 1.833 | 0.081 | 0.993 |
| N | N | 228 | 2.854 | 0.601 | 0.993 |
| ORF1a | nsp3 | 833 | 2.474 | 0.267 | 0.993 |
| ORF1b | nsp14 | 1559 | 2.978 | 0.257 | 0.993 |
| ORF1a | nsp3 | 1393 | 7.329 | 1.149 | 0.992 |
| ORF1b | nsp12 | 394 | 2.993 | 0.331 | 0.992 |
| M | M | 66 | 3.025 | 0.166 | 0.992 |
| ORF7a | ORF7a | 56 | 2.383 | 0.414 | 0.992 |
| Spike | Spike | 795 | 5.292 | 0.159 | 0.992 |
| ORF1b | nsp13 | 986 | 3.090 | 0.136 | 0.992 |
| ORF1b | nsp14 | 2015 | 2.764 | 0.191 | 0.992 |
| ORF1a | nsp2 | 810 | 2.813 | 0.637 | 0.992 |
| ORF1b | nsp13 | 1179 | 6.891 | 0.971 | 0.992 |
| ORF1a | nsp4 | 2829 | 2.818 | 0.340 | 0.992 |
| Spike | Spike | 503 | 2.002 | 0.102 | 0.992 |
| ORF1a | nsp3 | 1796 | 3.760 | 0.296 | 0.992 |
| ORF1b | nsp12 | 826 | 5.514 | 0.165 | 0.992 |
| ORF1b | nsp13 | 933 | 5.506 | 0.164 | 0.992 |
| ORF1a | nsp8 | 4010 | 2.358 | 0.157 | 0.992 |
| ORF1a | nsp3 | 1581 | 7.652 | 1.594 | 0.992 |
| ORF1a | nsp5 | 3355 | 3.914 | 0.250 | 0.992 |
| ORF7a | ORF7a | 75 | 2.572 | 0.390 | 0.991 |
| ORF1a | nsp3 | 1680 | 2.479 | 0.243 | 0.991 |
| Spike | Spike | 58 | 1.002 | 0.082 | 0.991 |
| ORF1b | nsp12 | 818 | 5.573 | 0.496 | 0.991 |
| ORF1a | nsp3 | 2693 | 2.827 | 0.355 | 0.991 |
| ORF1a | nsp9 | 4173 | 3.223 | 0.233 | 0.991 |
| Spike | Spike | 172 | 1.384 | 0.075 | 0.991 |
| Spike | Spike | 1018 | 2.211 | 0.190 | 0.991 |
| Spike | Spike | 507 | 2.108 | 0.088 | 0.991 |
| ORF1a | nsp3 | 2633 | 3.338 | 0.228 | 0.991 |
| N | N | 50 | 3.467 | 0.878 | 0.991 |
| ORF1a | nsp10 | 4264 | 4.554 | 0.163 | 0.991 |
| ORF1a | nsp3 | 2678 | 6.751 | 0.858 | 0.991 |
| ORF1a | nsp3 | 2749 | 11.526 | 0.529 | 0.991 |
| ORF1b | nsp14 | 1946 | 4.998 | 0.402 | 0.991 |
| ORF1b | nsp13 | 1328 | 2.827 | 0.272 | 0.991 |
| ORF1a | nsp4 | 3171 | 2.155 | 0.222 | 0.990 |
| ORF1b | nsp12 | 410 | 2.280 | 0.145 | 0.990 |
| ORF1a | nsp3 | 1750 | 5.878 | 0.885 | 0.990 |
| ORF1b | nsp12 | 828 | 2.378 | 0.191 | 0.990 |
| ORF1a | nsp10 | 4253 | 10.454 | 0.429 | 0.990 |
| ORF7a | ORF7a | 116 | 4.844 | 1.083 | 0.990 |
| ORF3a | ORF3a | 272 | 1.386 | 0.165 | 0.990 |
| ORF1a | nsp5 | 3405 | 3.915 | 0.262 | 0.990 |
| ORF1a | nsp3 | 1811 | 2.118 | 0.148 | 0.990 |
| ORF1a | nsp3 | 1961 | 2.113 | 0.148 | 0.990 |
| ORF1a | nsp3 | 2238 | 4.303 | 0.627 | 0.990 |
| ORF1a | nsp5 | 3407 | 3.953 | 0.153 | 0.990 |
| ORF1a | nsp5 | 3402 | 3.948 | 0.153 | 0.990 |
| Spike | Spike | 848 | 2.351 | 0.219 | 0.990 |
| ORF1a | nsp3 | 1387 | 10.694 | 0.741 | 0.990 |
| ORF1b | nsp12 | 477 | 2.575 | 0.191 | 0.990 |
| ORF1b | nsp12 | 202 | 3.789 | 0.335 | 0.990 |
| ORF1a | nsp3 | 1437 | 7.680 | 0.737 | 0.990 |
| Spike | Spike | 109 | 1.381 | 0.080 | 0.990 |
| ORF1a | nsp10 | 4330 | 2.397 | 0.176 | 0.990 |
| Spike | Spike | 397 | 1.246 | 0.062 | 0.990 |
| ORF7a | ORF7a | 101 | 1.051 | 0.105 | 0.990 |
| ORF1a | nsp3 | 2351 | 1.809 | 0.217 | 0.990 |
| ORF1b | nsp12 | 403 | 4.137 | 0.644 | 0.990 |
| ORF1b | nsp15 | 2146 | 3.579 | 0.194 | 0.989 |
| ORF1a | nsp6 | 3856 | 1.749 | 0.159 | 0.989 |
| Spike | Spike | 321 | 2.722 | 0.146 | 0.989 |
| ORF1b | nsp12 | 217 | 7.424 | 2.500 | 0.989 |
| ORF7a | ORF7a | 86 | 1.264 | 0.207 | 0.989 |
| ORF1b | nsp12 | 732 | 2.917 | 0.146 | 0.989 |
| ORF1b | nsp12 | 462 | 2.917 | 0.146 | 0.989 |
| Spike | Spike | 1181 | 12.238 | 0.747 | 0.989 |
| ORF1a | nsp6 | 3728 | 5.601 | 0.711 | 0.989 |
| ORF1b | nsp13 | 1015 | 3.496 | 0.212 | 0.989 |
| ORF1a | nsp2 | 223 | 2.819 | 0.649 | 0.989 |
| ORF1b | nsp16 | 2529 | 2.492 | 0.160 | 0.989 |
| ORF1a | nsp3 | 2385 | 5.205 | 0.677 | 0.989 |
| ORF8 | ORF8 | 62 | 7.125 | 2.863 | 0.989 |
| ORF1a | nsp4 | 3132 | 2.225 | 0.250 | 0.989 |
| ORF1b | nsp12 | 835 | 5.505 | 0.192 | 0.989 |
| ORF1b | nsp12 | 210 | 2.275 | 0.235 | 0.989 |
| ORF1b | nsp13 | 1462 | 3.915 | 0.200 | 0.988 |
| M | M | 123 | 3.596 | 0.127 | 0.988 |
| ORF1b | nsp13 | 1300 | 3.900 | 0.200 | 0.988 |
| ORF1b | nsp13 | 1458 | 3.900 | 0.200 | 0.988 |
| ORF1a | nsp3 | 2516 | 3.336 | 0.256 | 0.988 |
| Spike | Spike | 666 | 3.129 | 0.352 | 0.988 |
| ORF1b | nsp12 | 489 | 2.673 | 0.196 | 0.988 |
| ORF1a | nsp8 | 4022 | 4.356 | 0.246 | 0.988 |
| ORF1b | nsp15 | 2272 | 2.156 | 0.156 | 0.988 |
| N | N | 177 | 5.855 | 0.643 | 0.988 |
| Spike | Spike | 668 | 2.116 | 0.102 | 0.988 |
| ORF1b | nsp13 | 1365 | 6.643 | 0.959 | 0.988 |
| Spike | Spike | 359 | 0.831 | 0.077 | 0.988 |
| ORF1a | nsp4 | 2906 | 5.216 | 0.664 | 0.988 |
| ORF1a | nsp8 | 4052 | 4.603 | 0.188 | 0.988 |
| ORF1a | nsp2 | 210 | 5.916 | 0.548 | 0.988 |
| Spike | Spike | 735 | 4.813 | 0.539 | 0.988 |
| ORF1b | nsp13 | 1034 | 5.545 | 0.199 | 0.988 |
| Spike | Spike | 1054 | 3.294 | 0.257 | 0.988 |
| ORF1a | nsp6 | 3730 | 3.888 | 0.847 | 0.988 |
| ORF1a | nsp4 | 2905 | 2.575 | 0.335 | 0.988 |
| ORF1b | nsp15 | 2169 | 8.926 | 0.627 | 0.988 |
| ORF1b | nsp14 | 1616 | 5.032 | 0.374 | 0.988 |
| ORF3a | ORF3a | 138 | 1.468 | 0.097 | 0.988 |
| ORF1a | nsp2 | 644 | 2.666 | 0.572 | 0.988 |
| ORF1a | nsp3 | 2686 | 4.122 | 0.514 | 0.988 |
| ORF1b | nsp14 | 1571 | 3.955 | 0.311 | 0.988 |
| ORF1b | nsp13 | 1324 | 2.450 | 0.334 | 0.988 |
| Spike | Spike | 110 | 1.865 | 0.121 | 0.987 |
| ORF1b | nsp12 | 297 | 2.275 | 0.243 | 0.987 |
| ORF1b | nsp12 | 755 | 3.258 | 0.177 | 0.987 |
| ORF1b | nsp14 | 2035 | 2.522 | 0.200 | 0.987 |
| ORF1b | nsp12 | 568 | 9.688 | 0.326 | 0.987 |
| ORF1b | nsp12 | 705 | 9.688 | 0.326 | 0.987 |
| ORF1a | nsp5 | 3361 | 3.977 | 0.175 | 0.987 |
| ORF1b | nsp14 | 1972 | 2.679 | 0.221 | 0.987 |
| Spike | Spike | 466 | 2.727 | 0.126 | 0.987 |
| ORF1a | nsp3 | 2489 | 2.553 | 0.330 | 0.987 |
| ORF1a | nsp3 | 2526 | 2.941 | 0.391 | 0.987 |
| ORF1a | nsp6 | 3679 | 2.984 | 0.628 | 0.987 |
| ORF3a | ORF3a | 210 | 2.846 | 0.968 | 0.987 |
| ORF1a | nsp5 | 3459 | 8.085 | 1.870 | 0.987 |
| ORF1a | nsp8 | 4019 | 4.729 | 0.278 | 0.986 |
| ORF1b | nsp15 | 2120 | 2.407 | 0.171 | 0.986 |
| ORF1a | nsp4 | 3088 | 4.843 | 0.188 | 0.986 |
| ORF6 | ORF6 | 51 | 8.531 | 1.006 | 0.986 |
| Spike | Spike | 816 | 2.841 | 0.177 | 0.986 |
| Spike | Spike | 1030 | 2.841 | 0.177 | 0.986 |
| ORF1a | nsp3 | 2145 | 2.293 | 0.217 | 0.986 |
| ORF1a | nsp3 | 2211 | 2.860 | 0.557 | 0.986 |
| ORF1a | nsp3 | 1485 | 3.797 | 0.527 | 0.986 |
| ORF8 | ORF8 | 105 | 2.000 | 0.200 | 0.986 |
| Spike | Spike | 361 | 1.191 | 0.072 | 0.986 |
| ORF1a | nsp4 | 3141 | 4.844 | 0.193 | 0.986 |
| ORF1a | nsp2 | 202 | 4.005 | 0.260 | 0.986 |
| ORF1a | nsp3 | 1847 | 2.805 | 0.713 | 0.986 |
| ORF1a | nsp3 | 1031 | 2.643 | 0.300 | 0.986 |
| ORF1a | nsp3 | 1651 | 3.761 | 0.366 | 0.986 |
| ORF1b | nsp12 | 633 | 2.916 | 0.170 | 0.986 |
| Spike | Spike | 590 | 1.773 | 0.101 | 0.986 |
| Spike | Spike | 760 | 1.773 | 0.101 | 0.986 |
| ORF1a | nsp3 | 2457 | 4.552 | 0.252 | 0.985 |
| ORF1b | nsp12 | 672 | 4.059 | 0.170 | 0.985 |
| ORF1a | nsp10 | 4299 | 2.953 | 0.186 | 0.985 |
| N | N | 149 | 1.005 | 0.121 | 0.985 |
| ORF1b | nsp14 | 1599 | 1.898 | 0.235 | 0.985 |
| ORF1a | nsp7 | 3906 | 4.296 | 0.322 | 0.985 |
| ORF1b | nsp13 | 1489 | 2.762 | 0.597 | 0.985 |
| ORF1a | nsp5 | 3378 | 2.597 | 0.216 | 0.985 |
| Spike | Spike | 603 | 3.744 | 0.431 | 0.985 |
| ORF1a | nsp9 | 4184 | 2.237 | 0.182 | 0.985 |
| ORF1b | nsp14 | 1966 | 3.110 | 0.337 | 0.985 |
| Spike | Spike | 107 | 0.923 | 0.066 | 0.985 |
| ORF1a | nsp3 | 1987 | 2.539 | 0.211 | 0.985 |
| ORF1a | nsp5 | 3306 | 2.677 | 0.527 | 0.985 |
| Spike | Spike | 849 | 1.667 | 0.159 | 0.985 |
| Spike | Spike | 1200 | 1.667 | 0.159 | 0.985 |
| ORF1a | nsp3 | 1570 | 3.797 | 0.541 | 0.984 |
| ORF1b | nsp14 | 1557 | 1.436 | 0.161 | 0.984 |
| ORF1a | nsp6 | 3743 | 2.337 | 0.301 | 0.984 |
| ORF1a | nsp5 | 3283 | 3.995 | 0.194 | 0.984 |
| ORF1b | nsp14 | 1543 | 2.729 | 0.257 | 0.984 |
| N | N | 403 | 0.572 | 0.047 | 0.984 |
| ORF1a | nsp4 | 2912 | 3.009 | 0.215 | 0.984 |
| Spike | Spike | 638 | 2.097 | 0.117 | 0.984 |
| ORF1a | nsp3 | 2436 | 2.141 | 0.255 | 0.984 |
| ORF1a | nsp2 | 545 | 3.409 | 0.405 | 0.984 |
| ORF3a | ORF3a | 228 | 0.905 | 0.122 | 0.984 |
| ORF1a | nsp4 | 3225 | 2.229 | 0.220 | 0.984 |
| Spike | Spike | 458 | 1.946 | 0.113 | 0.984 |
| ORF1a | nsp3 | 2646 | 4.559 | 0.267 | 0.984 |
| ORF1a | nsp3 | 1808 | 3.597 | 0.203 | 0.984 |
| ORF1a | nsp6 | 3703 | 4.328 | 0.543 | 0.984 |
| ORF1b | nsp16 | 2401 | 2.230 | 0.133 | 0.983 |
| ORF1b | nsp12 | 849 | 2.494 | 0.169 | 0.983 |
| Spike | Spike | 411 | 2.547 | 0.332 | 0.983 |
| ORF1b | nsp13 | 1237 | 2.008 | 0.204 | 0.983 |
| ORF1a | nsp2 | 473 | 5.763 | 1.071 | 0.983 |
| ORF1b | nsp15 | 2111 | 3.315 | 0.221 | 0.983 |
| ORF1b | nsp13 | 1217 | 1.905 | 0.249 | 0.983 |
| ORF1b | nsp14 | 2039 | 3.624 | 0.238 | 0.983 |
| ORF1b | nsp12 | 317 | 2.596 | 0.239 | 0.983 |
| M | M | 26 | 1.703 | 0.157 | 0.983 |
| ORF1b | nsp14 | 2037 | 11.466 | 0.496 | 0.983 |
| ORF1a | nsp1 | 16 | 3.440 | 0.950 | 0.983 |
| ORF1b | nsp13 | 1475 | 3.955 | 0.251 | 0.983 |
| Spike | Spike | 881 | 2.884 | 0.200 | 0.983 |
| ORF1b | nsp15 | 2206 | 4.323 | 0.210 | 0.983 |
| ORF1b | nsp15 | 2091 | 4.323 | 0.210 | 0.983 |
| ORF1a | nsp3 | 1456 | 3.262 | 0.619 | 0.983 |
| Spike | Spike | 1160 | 2.871 | 0.200 | 0.983 |
| Spike | Spike | 998 | 2.869 | 0.200 | 0.983 |
| ORF1a | nsp4 | 2787 | 2.829 | 0.702 | 0.983 |
| Spike | Spike | 38 | 1.270 | 0.132 | 0.982 |
| Spike | Spike | 342 | 1.187 | 0.082 | 0.982 |
| ORF1b | nsp15 | 2186 | 5.028 | 0.236 | 0.982 |
| ORF1b | nsp12 | 566 | 3.156 | 0.203 | 0.982 |
| ORF1a | nsp7 | 3895 | 1.850 | 0.212 | 0.982 |
| ORF1b | nsp14 | 2045 | 3.307 | 0.267 | 0.982 |
| ORF1b | nsp12 | 292 | 2.600 | 0.245 | 0.982 |
| ORF1a | nsp3 | 2475 | 2.406 | 0.306 | 0.982 |
| Spike | Spike | 808 | 4.665 | 0.595 | 0.982 |
| Spike | Spike | 921 | 5.675 | 0.317 | 0.982 |
| ORF1b | nsp14 | 2007 | 2.044 | 0.182 | 0.982 |
| N | N | 106 | 1.644 | 0.098 | 0.982 |
| N | N | 73 | 1.643 | 0.098 | 0.982 |
| ORF1b | nsp12 | 851 | 2.763 | 0.187 | 0.982 |
| ORF1a | nsp12 | 4396 | 8.924 | 0.697 | 0.982 |
| ORF1a | nsp3 | 2235 | 2.716 | 0.558 | 0.982 |
| Spike | Spike | 699 | 1.282 | 0.095 | 0.982 |
| Spike | Spike | 513 | 1.281 | 0.095 | 0.982 |
| ORF1b | nsp16 | 2647 | 3.158 | 0.151 | 0.982 |
| ORF1b | nsp14 | 1924 | 5.782 | 0.352 | 0.982 |
| ORF7a | ORF7a | 21 | 9.516 | 1.011 | 0.981 |
| ORF1b | nsp12 | 548 | 4.090 | 0.196 | 0.981 |
| ORF1b | nsp12 | 653 | 4.087 | 0.196 | 0.981 |
| ORF1a | nsp3 | 1368 | 2.488 | 0.225 | 0.981 |
| ORF1b | nsp13 | 1255 | 4.848 | 0.305 | 0.981 |
| ORF1b | nsp13 | 1313 | 4.848 | 0.305 | 0.981 |
| ORF1a | nsp2 | 782 | 1.817 | 0.161 | 0.981 |
| ORF1a | nsp8 | 4091 | 2.960 | 0.211 | 0.981 |
| ORF1a | nsp9 | 4206 | 2.960 | 0.211 | 0.981 |
| Spike | Spike | 114 | 0.762 | 0.088 | 0.981 |
| ORF1b | nsp13 | 1460 | 3.978 | 0.371 | 0.981 |
| N | N | 41 | 3.491 | 0.980 | 0.981 |
| ORF1a | nsp1 | 3 | 1.897 | 0.225 | 0.981 |
| ORF1a | nsp2 | 599 | 2.788 | 0.923 | 0.981 |
| ORF1a | nsp3 | 1264 | 3.689 | 0.296 | 0.981 |
| M | M | 181 | 1.993 | 0.183 | 0.981 |
| ORF1b | nsp14 | 1820 | 5.056 | 0.643 | 0.981 |
| ORF1a | nsp2 | 548 | 2.664 | 0.732 | 0.981 |
| E | E | 74 | 3.642 | 0.642 | 0.980 |
| ORF1a | nsp9 | 4156 | 1.903 | 0.180 | 0.980 |
| ORF1b | nsp16 | 2466 | 3.159 | 0.156 | 0.980 |
| ORF1b | nsp14 | 1761 | 3.376 | 0.320 | 0.980 |
| ORF1b | nsp14 | 1721 | 3.376 | 0.320 | 0.980 |
| ORF1a | nsp3 | 2386 | 1.375 | 0.212 | 0.980 |
| ORF1a | nsp3 | 2573 | 6.188 | 0.758 | 0.980 |
| Spike | Spike | 864 | 1.714 | 0.166 | 0.980 |
| ORF1a | nsp3 | 830 | 1.839 | 0.259 | 0.980 |
| ORF1a | nsp4 | 2830 | 6.589 | 0.295 | 0.980 |
| ORF1b | nsp13 | 1079 | 7.858 | 0.334 | 0.980 |
| ORF1a | nsp3 | 1722 | 3.593 | 0.229 | 0.980 |
| ORF1a | nsp2 | 448 | 2.081 | 0.316 | 0.980 |
| ORF1b | nsp13 | 1061 | 1.413 | 0.153 | 0.980 |
| ORF1a | nsp5 | 3373 | 4.104 | 0.309 | 0.979 |
| ORF1a | nsp2 | 424 | 2.637 | 0.486 | 0.979 |
| ORF1b | nsp12 | 474 | 2.244 | 0.207 | 0.979 |
| ORF1a | nsp3 | 1184 | 2.663 | 0.659 | 0.979 |
| ORF1b | nsp12 | 902 | 2.797 | 0.289 | 0.979 |
| ORF1b | nsp13 | 1100 | 2.797 | 0.289 | 0.979 |
| ORF1b | nsp13 | 979 | 4.002 | 0.255 | 0.979 |
| ORF1b | nsp13 | 1520 | 1.743 | 0.199 | 0.979 |
| Spike | Spike | 857 | 1.666 | 0.181 | 0.979 |
| N | N | 287 | 1.105 | 0.130 | 0.979 |
| N | N | 175 | 1.104 | 0.130 | 0.979 |
| ORF1a | nsp1 | 35 | 3.243 | 0.699 | 0.979 |
| ORF1a | nsp3 | 1864 | 2.425 | 0.237 | 0.979 |
| ORF1b | nsp14 | 1602 | 7.097 | 0.429 | 0.979 |
| ORF1a | nsp3 | 1076 | 3.465 | 0.612 | 0.979 |
| Spike | Spike | 31 | 0.920 | 0.080 | 0.979 |
| ORF1a | nsp3 | 2006 | 3.243 | 0.384 | 0.979 |
| ORF1b | nsp16 | 2526 | 3.155 | 0.164 | 0.979 |
| Spike | Spike | 297 | 1.233 | 0.093 | 0.978 |
| Spike | Spike | 877 | 1.848 | 0.219 | 0.978 |
| ORF1b | nsp16 | 2420 | 4.146 | 0.212 | 0.978 |
| M | M | 77 | 2.450 | 0.427 | 0.978 |
| Spike | Spike | 980 | 1.721 | 0.191 | 0.978 |
| ORF1a | nsp5 | 3296 | 5.381 | 0.670 | 0.978 |
| ORF3a | ORF3a | 273 | 2.404 | 0.598 | 0.978 |
| ORF1a | nsp4 | 3003 | 5.748 | 0.281 | 0.978 |
| Spike | Spike | 336 | 0.701 | 0.079 | 0.978 |
| Spike | Spike | 873 | 1.871 | 0.210 | 0.978 |
| ORF1b | nsp14 | 2042 | 2.763 | 0.260 | 0.978 |
| ORF1b | nsp13 | 1470 | 2.024 | 0.237 | 0.978 |
| ORF1b | nsp16 | 2646 | 4.148 | 0.216 | 0.978 |
| E | E | 75 | 2.213 | 0.286 | 0.978 |
| ORF1a | nsp3 | 1462 | 2.473 | 0.244 | 0.978 |
| Spike | Spike | 659 | 3.221 | 0.363 | 0.978 |
| ORF1b | nsp14 | 1705 | 2.623 | 0.584 | 0.978 |
| ORF1a | nsp3 | 1284 | 3.210 | 0.213 | 0.977 |
| ORF1b | nsp16 | 2483 | 3.302 | 0.177 | 0.977 |
| ORF1b | nsp12 | 521 | 2.913 | 0.215 | 0.977 |
| Spike | Spike | 117 | 1.003 | 0.127 | 0.977 |
| ORF1b | nsp13 | 1310 | 1.392 | 0.187 | 0.977 |
| ORF1a | nsp3 | 2447 | 3.497 | 0.654 | 0.977 |
| ORF1a | nsp6 | 3788 | 2.872 | 0.277 | 0.977 |
| Spike | Spike | 442 | 1.759 | 0.128 | 0.977 |
| Spike | Spike | 1065 | 3.637 | 0.704 | 0.977 |
| ORF1a | nsp2 | 180 | 2.223 | 0.246 | 0.977 |
| ORF1a | nsp5 | 3494 | 2.215 | 0.256 | 0.977 |
| Spike | Spike | 1008 | 1.722 | 0.179 | 0.977 |
| ORF1a | nsp3 | 1793 | 2.112 | 0.224 | 0.977 |
| ORF1a | nsp2 | 588 | 1.454 | 0.184 | 0.977 |
| ORF1b | nsp12 | 157 | 4.153 | 0.283 | 0.977 |
| ORF1b | nsp12 | 118 | 1.688 | 0.211 | 0.977 |
| ORF1a | nsp6 | 3717 | 1.270 | 0.163 | 0.977 |
| ORF1a | nsp7 | 3879 | 1.270 | 0.163 | 0.977 |
| Spike | Spike | 683 | 2.464 | 0.508 | 0.976 |
| ORF1a | nsp2 | 787 | 2.076 | 0.282 | 0.976 |
| ORF1b | nsp12 | 265 | 2.274 | 0.312 | 0.976 |
| ORF1a | nsp3 | 2479 | 5.172 | 0.352 | 0.976 |
| ORF1b | nsp14 | 1686 | 2.444 | 0.275 | 0.976 |
| Spike | Spike | 734 | 3.235 | 0.373 | 0.976 |
| ORF1a | nsp9 | 4192 | 5.951 | 0.316 | 0.976 |
| ORF1a | nsp9 | 4221 | 5.948 | 0.316 | 0.976 |
| Spike | Spike | 1075 | 1.371 | 0.152 | 0.976 |
| ORF1a | nsp10 | 4281 | 5.946 | 0.316 | 0.976 |
| ORF1a | nsp3 | 1985 | 1.908 | 0.242 | 0.976 |
| ORF1b | nsp12 | 383 | 1.686 | 0.214 | 0.976 |
| ORF1a | nsp10 | 4319 | 5.953 | 0.317 | 0.976 |
| ORF1a | nsp9 | 4208 | 5.953 | 0.317 | 0.976 |
| Spike | Spike | 206 | 1.068 | 0.093 | 0.976 |
| E | E | 65 | 2.343 | 0.345 | 0.976 |
| ORF1a | nsp3 | 1256 | 3.689 | 0.329 | 0.976 |
| Spike | Spike | 225 | 0.773 | 0.055 | 0.976 |
| ORF1a | nsp3 | 1788 | 7.248 | 2.769 | 0.976 |
| Spike | Spike | 674 | 1.757 | 0.132 | 0.976 |
| Spike | Spike | 612 | 1.755 | 0.132 | 0.976 |
| ORF1b | nsp16 | 2651 | 2.594 | 0.190 | 0.976 |
| ORF1b | nsp14 | 1588 | 4.183 | 0.639 | 0.976 |
| N | N | 112 | 0.715 | 0.099 | 0.975 |
| ORF1b | nsp12 | 643 | 1.752 | 0.172 | 0.975 |
| ORF1b | nsp16 | 2457 | 2.358 | 0.143 | 0.975 |
| ORF1a | nsp3 | 1156 | 2.770 | 0.606 | 0.975 |
| ORF1a | nsp3 | 2002 | 1.722 | 0.222 | 0.975 |
| ORF1b | nsp15 | 2187 | 3.653 | 0.282 | 0.975 |
| ORF1b | nsp15 | 2055 | 3.653 | 0.282 | 0.975 |
| ORF1b | nsp15 | 2133 | 3.653 | 0.282 | 0.975 |
| ORF1a | nsp3 | 879 | 1.177 | 0.246 | 0.975 |
| ORF1b | nsp16 | 2429 | 3.162 | 0.181 | 0.975 |
| Spike | Spike | 1242 | 1.875 | 0.216 | 0.975 |
| ORF1b | nsp16 | 2527 | 3.153 | 0.181 | 0.975 |
| ORF1a | nsp3 | 2643 | 3.376 | 0.363 | 0.974 |
| ORF1a | nsp3 | 2456 | 3.376 | 0.363 | 0.974 |
| ORF1b | nsp16 | 2471 | 2.358 | 0.145 | 0.974 |
| ORF1b | nsp16 | 2670 | 2.356 | 0.145 | 0.974 |
| ORF1b | nsp16 | 2605 | 3.038 | 0.177 | 0.974 |
| ORF1a | nsp3 | 2467 | 3.365 | 0.363 | 0.974 |
| ORF1b | nsp13 | 1004 | 2.330 | 0.160 | 0.974 |
| ORF6 | ORF6 | 57 | 4.136 | 0.667 | 0.974 |
| ORF1b | nsp12 | 819 | 1.989 | 0.227 | 0.974 |
| ORF1a | nsp3 | 1328 | 2.474 | 0.261 | 0.974 |
| ORF1a | nsp4 | 3245 | 2.511 | 0.227 | 0.974 |
| ORF1a | nsp3 | 1636 | 2.550 | 0.658 | 0.974 |
| ORF1b | nsp14 | 1615 | 1.688 | 0.261 | 0.974 |
| ORF1a | nsp8 | 4125 | 1.852 | 0.144 | 0.974 |
| ORF1b | nsp16 | 2634 | 5.156 | 0.506 | 0.974 |
| ORF1a | nsp3 | 886 | 4.091 | 0.492 | 0.974 |
| ORF1b | nsp13 | 969 | 3.997 | 0.285 | 0.974 |
| ORF1a | nsp10 | 4310 | 4.905 | 0.693 | 0.974 |
| ORF1a | nsp2 | 244 | 1.402 | 0.183 | 0.973 |
| N | N | 74 | 0.637 | 0.076 | 0.973 |
| ORF1a | nsp9 | 4233 | 4.692 | 0.281 | 0.973 |
| ORF1a | nsp2 | 421 | 1.084 | 0.196 | 0.973 |
| N | N | 223 | 0.698 | 0.109 | 0.973 |
| ORF1b | nsp15 | 2267 | 6.274 | 0.687 | 0.973 |
| N | N | 415 | 2.565 | 0.691 | 0.973 |
| ORF1b | nsp14 | 1986 | 3.401 | 0.368 | 0.973 |
| ORF1a | nsp8 | 3992 | 1.852 | 0.258 | 0.973 |
| ORF1a | nsp3 | 1243 | 3.567 | 0.668 | 0.973 |
| ORF1a | nsp8 | 4062 | 6.963 | 0.198 | 0.973 |
| ORF1a | nsp3 | 2090 | 2.657 | 0.469 | 0.973 |
| M | M | 119 | 1.568 | 0.165 | 0.973 |
| M | M | 205 | 4.846 | 0.258 | 0.972 |
| M | M | 180 | 4.846 | 0.258 | 0.972 |
| Spike | Spike | 1057 | 1.544 | 0.155 | 0.972 |
| ORF1a | nsp3 | 1800 | 3.988 | 0.413 | 0.972 |
| ORF1a | nsp3 | 1977 | 4.199 | 1.047 | 0.972 |
| ORF1a | nsp3 | 1770 | 2.546 | 0.604 | 0.972 |
| ORF1a | nsp3 | 2610 | 6.274 | 0.422 | 0.972 |
| ORF1a | nsp3 | 2478 | 6.270 | 0.422 | 0.972 |
| ORF1a | nsp2 | 711 | 2.649 | 0.574 | 0.972 |
| ORF1a | nsp3 | 2177 | 1.776 | 0.293 | 0.972 |
| ORF1a | nsp4 | 3200 | 1.791 | 0.173 | 0.972 |
| N | N | 286 | 0.501 | 0.040 | 0.972 |
| ORF1a | nsp2 | 231 | 1.063 | 0.180 | 0.972 |
| ORF1a | nsp9 | 4188 | 2.118 | 0.194 | 0.972 |
| ORF1a | nsp9 | 4169 | 2.118 | 0.194 | 0.972 |
| ORF1b | nsp12 | 49 | 6.030 | 0.400 | 0.971 |
| ORF1b | nsp15 | 2353 | 2.253 | 0.169 | 0.971 |
| ORF1b | nsp14 | 1915 | 9.084 | 0.947 | 0.971 |
| Spike | Spike | 669 | 1.272 | 0.120 | 0.971 |
| ORF1a | nsp3 | 2393 | 2.244 | 0.232 | 0.971 |
| Spike | Spike | 1051 | 1.225 | 0.154 | 0.971 |
| ORF1a | nsp6 | 3656 | 1.249 | 0.137 | 0.971 |
| ORF1b | nsp13 | 1165 | 5.818 | 0.401 | 0.971 |
| ORF1b | nsp12 | 451 | 2.150 | 0.218 | 0.971 |
| ORF1a | nsp9 | 4200 | 2.961 | 0.258 | 0.971 |
| ORF1a | nsp4 | 3144 | 3.292 | 0.311 | 0.970 |
| ORF1b | nsp12 | 525 | 2.914 | 0.247 | 0.970 |
| ORF1a | nsp3 | 1676 | 1.323 | 0.150 | 0.970 |
| ORF1a | nsp3 | 1679 | 9.500 | 2.899 | 0.970 |
| ORF1b | nsp16 | 2694 | 2.017 | 0.195 | 0.969 |
| ORF1b | nsp16 | 2486 | 2.032 | 0.175 | 0.969 |
| ORF1b | nsp15 | 2295 | 3.348 | 0.287 | 0.969 |
| ORF1b | nsp12 | 381 | 2.627 | 0.318 | 0.969 |
| Spike | Spike | 919 | 1.871 | 0.246 | 0.969 |
| Spike | Spike | 717 | 1.752 | 0.150 | 0.969 |
| ORF1a | nsp5 | 3517 | 1.660 | 0.150 | 0.969 |
| ORF1b | nsp12 | 674 | 4.154 | 0.263 | 0.969 |
| ORF1b | nsp12 | 212 | 2.624 | 0.318 | 0.969 |
| ORF1a | nsp4 | 2958 | 3.285 | 0.318 | 0.969 |
| ORF1a | nsp4 | 2824 | 3.285 | 0.318 | 0.969 |
| ORF1a | nsp5 | 3325 | 1.653 | 0.150 | 0.969 |
| Spike | Spike | 500 | 1.271 | 0.126 | 0.969 |
| ORF1a | nsp5 | 3386 | 1.650 | 0.150 | 0.969 |
| Spike | Spike | 57 | 0.635 | 0.056 | 0.969 |
| Spike | Spike | 337 | 0.635 | 0.056 | 0.969 |
| Spike | Spike | 295 | 0.635 | 0.056 | 0.969 |
| N | N | 104 | 0.550 | 0.067 | 0.969 |
| ORF1a | nsp3 | 2448 | 1.725 | 0.259 | 0.969 |
| Spike | Spike | 966 | 1.544 | 0.165 | 0.969 |
| Spike | Spike | 1001 | 1.544 | 0.165 | 0.969 |
| Spike | Spike | 948 | 1.543 | 0.165 | 0.969 |
| ORF1a | nsp3 | 2671 | 3.380 | 0.397 | 0.968 |
| ORF1a | nsp3 | 2736 | 3.376 | 0.397 | 0.968 |
| ORF1a | nsp4 | 2767 | 3.373 | 0.397 | 0.968 |
| ORF1a | nsp3 | 1385 | 3.200 | 0.257 | 0.968 |
| ORF1a | nsp3 | 1799 | 8.494 | 1.089 | 0.968 |
| Spike | Spike | 343 | 0.829 | 0.123 | 0.968 |
| ORF1b | nsp16 | 2536 | 2.336 | 0.165 | 0.968 |
| ORF1a | nsp3 | 1554 | 6.890 | 1.442 | 0.968 |
| ORF1b | nsp14 | 1630 | 1.554 | 0.186 | 0.968 |
| ORF1b | nsp16 | 2545 | 2.338 | 0.166 | 0.968 |
| Spike | Spike | 1149 | 2.115 | 0.218 | 0.968 |
| ORF1b | nsp14 | 2003 | 2.498 | 0.238 | 0.967 |
| ORF1a | nsp8 | 4135 | 1.837 | 0.162 | 0.967 |
| ORF1a | nsp2 | 492 | 4.458 | 0.595 | 0.967 |
| ORF1b | nsp13 | 1158 | 2.307 | 0.226 | 0.967 |
| M | M | 196 | 1.672 | 0.212 | 0.967 |
| Spike | Spike | 870 | 1.782 | 0.191 | 0.967 |
| Spike | Spike | 923 | 1.782 | 0.191 | 0.967 |
| Spike | Spike | 1172 | 1.780 | 0.191 | 0.967 |
| ORF1b | nsp16 | 2398 | 1.780 | 0.159 | 0.967 |
| ORF1a | nsp5 | 3300 | 1.727 | 0.228 | 0.967 |
| ORF1a | nsp4 | 3250 | 1.727 | 0.228 | 0.967 |
| ORF1a | nsp4 | 3226 | 1.727 | 0.228 | 0.967 |
| ORF1b | nsp13 | 1265 | 1.626 | 0.195 | 0.967 |
| ORF1b | nsp13 | 1367 | 1.626 | 0.195 | 0.967 |
| N | N | 75 | 1.514 | 0.212 | 0.967 |
| N | N | 153 | 1.512 | 0.212 | 0.967 |
| Spike | Spike | 903 | 1.543 | 0.172 | 0.966 |
| ORF1b | nsp12 | 189 | 2.631 | 0.332 | 0.966 |
| ORF1a | nsp5 | 3507 | 5.080 | 0.361 | 0.966 |
| ORF1a | nsp7 | 3908 | 1.709 | 0.182 | 0.966 |
| ORF1b | nsp13 | 1082 | 1.814 | 0.167 | 0.966 |
| ORF1b | nsp13 | 1384 | 1.293 | 0.167 | 0.966 |
| ORF1b | nsp14 | 1554 | 3.536 | 0.735 | 0.966 |
| ORF1a | nsp7 | 3892 | 4.726 | 0.949 | 0.966 |
| ORF1b | nsp13 | 1323 | 1.408 | 0.229 | 0.966 |
| ORF1b | nsp14 | 1650 | 5.042 | 0.729 | 0.966 |
| ORF1b | nsp14 | 1810 | 1.874 | 0.216 | 0.965 |
| ORF1a | nsp7 | 3891 | 1.707 | 0.184 | 0.965 |
| N | N | 109 | 0.693 | 0.097 | 0.965 |
| ORF1a | nsp6 | 3798 | 1.705 | 0.184 | 0.965 |
| ORF1b | nsp13 | 998 | 2.313 | 0.188 | 0.965 |
| ORF1a | nsp1 | 41 | 2.481 | 0.285 | 0.965 |
| Spike | Spike | 412 | 1.138 | 0.092 | 0.965 |
| Spike | Spike | 600 | 1.138 | 0.092 | 0.965 |
| Spike | Spike | 589 | 1.137 | 0.092 | 0.965 |
| Spike | Spike | 426 | 1.137 | 0.092 | 0.965 |
| Spike | Spike | 767 | 1.334 | 0.138 | 0.965 |
| ORF1a | nsp3 | 890 | 2.763 | 0.690 | 0.965 |
| ORF1a | nsp3 | 828 | 1.666 | 0.368 | 0.965 |
| Spike | Spike | 121 | 1.174 | 0.122 | 0.965 |
| ORF1a | nsp6 | 3706 | 1.687 | 0.185 | 0.965 |
| ORF1a | nsp3 | 2629 | 1.624 | 0.262 | 0.965 |
| ORF1a | nsp3 | 2052 | 2.406 | 0.470 | 0.965 |
| ORF1b | nsp13 | 1233 | 1.294 | 0.171 | 0.965 |
| ORF1b | nsp13 | 1409 | 1.293 | 0.171 | 0.965 |
| ORF1a | nsp3 | 2296 | 2.316 | 0.328 | 0.965 |
| ORF1b | nsp13 | 1258 | 1.297 | 0.171 | 0.964 |
| ORF1a | nsp4 | 3252 | 1.995 | 0.131 | 0.964 |
| ORF1a | nsp4 | 3253 | 1.995 | 0.131 | 0.964 |
| ORF1a | nsp5 | 3272 | 1.993 | 0.131 | 0.964 |
| ORF1a | nsp1 | 142 | 3.805 | 0.697 | 0.964 |
| N | N | 313 | 1.821 | 0.397 | 0.964 |
| ORF1b | nsp13 | 1221 | 1.469 | 0.245 | 0.964 |
| ORF1a | nsp3 | 1982 | 3.612 | 0.318 | 0.964 |
| ORF1a | nsp3 | 2487 | 1.887 | 0.212 | 0.964 |
| ORF1a | nsp3 | 2558 | 1.884 | 0.212 | 0.964 |
| ORF1b | nsp13 | 1020 | 2.310 | 0.194 | 0.963 |
| ORF1b | nsp12 | 823 | 2.625 | 0.148 | 0.963 |
| ORF1b | nsp12 | 435 | 4.212 | 0.293 | 0.963 |
| ORF1b | nsp12 | 859 | 2.621 | 0.148 | 0.963 |
| ORF1b | nsp13 | 995 | 2.308 | 0.194 | 0.963 |
| ORF1b | nsp14 | 1853 | 1.558 | 0.200 | 0.963 |
| ORF1b | nsp13 | 946 | 2.614 | 0.148 | 0.963 |
| ORF1b | nsp12 | 825 | 2.614 | 0.148 | 0.963 |
| Spike | Spike | 957 | 17.646 | 2.454 | 0.963 |
| ORF1a | nsp9 | 4209 | 1.713 | 0.216 | 0.963 |
| ORF1b | nsp14 | 1612 | 1.704 | 0.305 | 0.963 |
| M | M | 22 | 1.790 | 0.234 | 0.963 |
| ORF1b | nsp14 | 1595 | 5.292 | 0.830 | 0.963 |
| Spike | Spike | 924 | 2.180 | 0.463 | 0.963 |
| Spike | Spike | 118 | 0.634 | 0.064 | 0.963 |
| Spike | Spike | 270 | 0.634 | 0.064 | 0.963 |
| ORF1a | nsp3 | 1709 | 2.806 | 0.724 | 0.963 |
| ORF1b | nsp12 | 413 | 1.880 | 0.169 | 0.963 |
| ORF1a | nsp3 | 2721 | 1.951 | 0.268 | 0.962 |
| Spike | Spike | 1067 | 1.930 | 0.219 | 0.962 |
| Spike | Spike | 967 | 1.366 | 0.190 | 0.962 |
| ORF1b | nsp13 | 955 | 1.978 | 0.272 | 0.962 |
| ORF1b | nsp12 | 364 | 3.186 | 0.581 | 0.962 |
| ORF1b | nsp14 | 1978 | 1.561 | 0.205 | 0.962 |
| ORF1b | nsp14 | 1636 | 1.560 | 0.205 | 0.962 |
| ORF1b | nsp12 | 242 | 2.336 | 0.321 | 0.961 |
| ORF1a | nsp10 | 4372 | 1.392 | 0.204 | 0.961 |
| N | N | 181 | 3.273 | 0.221 | 0.961 |
| ORF1b | nsp14 | 1838 | 2.366 | 0.568 | 0.961 |
| Spike | Spike | 328 | 0.878 | 0.084 | 0.961 |
| ORF1a | nsp3 | 2599 | 4.758 | 0.493 | 0.961 |
| ORF8 | ORF8 | 43 | 0.714 | 0.115 | 0.961 |
| ORF1a | nsp8 | 3960 | 1.249 | 0.162 | 0.961 |
| ORF1a | nsp3 | 857 | 0.910 | 0.215 | 0.961 |
| Spike | Spike | 363 | 0.635 | 0.066 | 0.961 |
| ORF1a | nsp3 | 860 | 1.762 | 0.293 | 0.961 |
| ORF1a | nsp3 | 2301 | 2.317 | 0.343 | 0.961 |
| Spike | Spike | 559 | 0.980 | 0.092 | 0.961 |
| Spike | Spike | 464 | 0.979 | 0.092 | 0.961 |
| Spike | Spike | 782 | 0.978 | 0.092 | 0.961 |
| ORF1b | nsp14 | 1688 | 6.264 | 0.278 | 0.961 |
| Spike | Spike | 160 | 1.172 | 0.131 | 0.961 |
| Spike | Spike | 37 | 1.172 | 0.131 | 0.961 |
| Spike | Spike | 365 | 1.171 | 0.131 | 0.961 |
| ORF7a | ORF7a | 117 | 3.385 | 0.295 | 0.961 |
| Spike | Spike | 427 | 2.603 | 0.387 | 0.961 |
| Spike | Spike | 867 | 1.933 | 0.226 | 0.960 |
| N | N | 328 | 1.592 | 0.155 | 0.960 |
| ORF1a | nsp3 | 2312 | 3.889 | 0.584 | 0.960 |
| Spike | Spike | 971 | 1.542 | 0.189 | 0.960 |
| N | N | 316 | 1.586 | 0.155 | 0.960 |
| ORF1b | nsp13 | 1426 | 6.621 | 1.282 | 0.960 |
| ORF1a | nsp6 | 3742 | 1.249 | 0.164 | 0.960 |
| ORF1a | nsp4 | 3237 | 1.643 | 0.173 | 0.960 |
| ORF1b | nsp14 | 1645 | 2.817 | 0.462 | 0.960 |
| Spike | Spike | 770 | 1.445 | 0.116 | 0.960 |
| Spike | Spike | 598 | 1.444 | 0.116 | 0.960 |
| ORF1a | nsp2 | 488 | 2.654 | 0.649 | 0.960 |
| Spike | Spike | 1061 | 1.222 | 0.183 | 0.959 |
| Spike | Spike | 103 | 0.533 | 0.068 | 0.959 |
| ORF3a | ORF3a | 222 | 2.808 | 1.097 | 0.959 |
| ORF1a | nsp5 | 3304 | 1.773 | 0.184 | 0.959 |
| ORF1b | nsp13 | 1101 | 4.506 | 0.318 | 0.959 |
| ORF1a | nsp8 | 4068 | 1.388 | 0.210 | 0.959 |
| ORF1b | nsp12 | 108 | 6.091 | 0.521 | 0.959 |
| N | N | 66 | 0.716 | 0.191 | 0.959 |
| ORF1b | nsp14 | 1932 | 4.666 | 0.796 | 0.958 |
| ORF1b | nsp14 | 1845 | 2.064 | 0.317 | 0.958 |
| Spike | Spike | 391 | 0.574 | 0.078 | 0.958 |
| ORF1b | nsp13 | 1194 | 3.409 | 0.261 | 0.958 |
| Spike | Spike | 381 | 0.767 | 0.082 | 0.958 |
| ORF1b | nsp16 | 2567 | 2.261 | 0.192 | 0.958 |
| ORF1a | nsp2 | 654 | 0.891 | 0.195 | 0.958 |
| ORF1a | nsp2 | 230 | 0.857 | 0.179 | 0.958 |
| ORF1a | nsp6 | 3602 | 2.763 | 0.528 | 0.957 |
| Spike | Spike | 729 | 0.752 | 0.113 | 0.957 |
| ORF1a | nsp4 | 3096 | 2.353 | 0.167 | 0.957 |
| ORF1a | nsp4 | 3033 | 2.353 | 0.167 | 0.957 |
| ORF1a | nsp3 | 2502 | 2.162 | 0.258 | 0.957 |
| ORF1a | nsp3 | 2175 | 1.773 | 0.289 | 0.957 |
| ORF1a | nsp3 | 2113 | 1.773 | 0.289 | 0.957 |
| ORF1a | nsp2 | 539 | 2.873 | 0.584 | 0.957 |
| Spike | Spike | 695 | 0.860 | 0.146 | 0.957 |
| ORF1a | nsp2 | 606 | 1.453 | 0.244 | 0.957 |
| ORF1b | nsp15 | 2219 | 2.148 | 0.208 | 0.957 |
| ORF1b | nsp14 | 1991 | 2.294 | 0.191 | 0.957 |
| N | N | 288 | 0.873 | 0.169 | 0.956 |
| ORF1b | nsp14 | 1953 | 2.287 | 0.191 | 0.956 |
| ORF1a | nsp3 | 1046 | 1.282 | 0.249 | 0.956 |
| ORF1b | nsp16 | 2584 | 1.482 | 0.186 | 0.956 |
| Spike | Spike | 462 | 2.302 | 0.154 | 0.956 |
| E | E | 29 | 2.271 | 0.290 | 0.956 |
| M | M | 165 | 1.014 | 0.146 | 0.956 |
| ORF1a | nsp6 | 3572 | 1.819 | 0.196 | 0.956 |
| ORF1a | nsp9 | 4214 | 2.725 | 0.251 | 0.956 |
| ORF1a | nsp5 | 3496 | 1.816 | 0.196 | 0.956 |
| ORF1b | nsp12 | 863 | 3.984 | 0.488 | 0.956 |
| ORF1a | nsp3 | 1208 | 4.615 | 0.390 | 0.956 |
| ORF1b | nsp13 | 1120 | 3.406 | 0.270 | 0.956 |
| ORF1a | nsp3 | 1910 | 4.991 | 0.472 | 0.956 |
| ORF1b | nsp13 | 1236 | 1.302 | 0.195 | 0.956 |
| ORF1b | nsp13 | 1487 | 1.300 | 0.195 | 0.956 |
| ORF1b | nsp14 | 1803 | 1.888 | 0.250 | 0.955 |
| ORF1a | nsp3 | 1916 | 1.644 | 0.149 | 0.955 |
| ORF1a | nsp4 | 2851 | 1.724 | 0.326 | 0.955 |
| ORF1b | nsp14 | 1855 | 1.803 | 0.351 | 0.955 |
| ORF3a | ORF3a | 196 | 2.565 | 0.415 | 0.955 |
| Spike | Spike | 897 | 1.580 | 0.161 | 0.955 |
| ORF1b | nsp13 | 1234 | 1.645 | 0.234 | 0.955 |
| M | M | 54 | 1.041 | 0.177 | 0.955 |
| Spike | Spike | 238 | 0.746 | 0.085 | 0.955 |
| Spike | Spike | 377 | 0.746 | 0.085 | 0.955 |
| Spike | Spike | 392 | 0.746 | 0.085 | 0.955 |
| Spike | Spike | 106 | 0.746 | 0.085 | 0.955 |
| Spike | Spike | 86 | 0.746 | 0.085 | 0.955 |
| Spike | Spike | 338 | 0.746 | 0.085 | 0.955 |
| Spike | Spike | 329 | 0.746 | 0.085 | 0.955 |
| ORF1b | nsp14 | 1528 | 4.271 | 0.518 | 0.955 |
| Spike | Spike | 133 | 0.746 | 0.085 | 0.955 |
| Spike | Spike | 79 | 0.746 | 0.085 | 0.955 |
| M | M | 30 | 5.842 | 0.743 | 0.955 |
| ORF1b | nsp12 | 337 | 1.750 | 0.298 | 0.954 |
| ORF1b | nsp12 | 29 | 1.749 | 0.298 | 0.954 |
| ORF1b | nsp12 | 490 | 6.583 | 1.181 | 0.954 |
| ORF1b | nsp14 | 1830 | 4.915 | 0.773 | 0.954 |
| ORF1b | nsp13 | 1447 | 4.092 | 0.667 | 0.954 |
| ORF1b | nsp12 | 196 | 1.816 | 0.327 | 0.954 |
| Spike | Spike | 1147 | 5.927 | 0.870 | 0.954 |
| ORF1a | nsp3 | 864 | 0.788 | 0.184 | 0.954 |
| ORF1a | nsp3 | 2701 | 1.764 | 0.232 | 0.954 |
| ORF1b | nsp14 | 1665 | 2.553 | 0.460 | 0.954 |
| ORF3a | ORF3a | 168 | 0.621 | 0.121 | 0.954 |
| ORF10 | ORF10 | 26 | 1.007 | 0.251 | 0.953 |
| E | E | 25 | 3.537 | 0.289 | 0.953 |
| Spike | Spike | 662 | 0.978 | 0.104 | 0.953 |
| Spike | Spike | 738 | 0.978 | 0.104 | 0.953 |
| Spike | Spike | 525 | 0.977 | 0.104 | 0.953 |
| ORF1a | nsp2 | 411 | 0.939 | 0.211 | 0.953 |
| ORF1a | nsp2 | 741 | 0.939 | 0.211 | 0.953 |
| ORF1a | nsp7 | 3872 | 1.737 | 0.265 | 0.953 |
| ORF1a | nsp7 | 3894 | 1.737 | 0.265 | 0.953 |
| ORF1a | nsp4 | 2942 | 1.753 | 0.229 | 0.953 |
| ORF1a | nsp4 | 2999 | 1.749 | 0.229 | 0.953 |
| ORF1b | nsp13 | 1260 | 1.305 | 0.202 | 0.953 |
| ORF1b | nsp13 | 1366 | 1.304 | 0.202 | 0.953 |
| ORF1b | nsp13 | 1517 | 1.303 | 0.201 | 0.953 |
| Spike | Spike | 996 | 1.202 | 0.173 | 0.953 |
| N | N | 145 | 6.914 | 2.772 | 0.953 |
| ORF1b | nsp14 | 1708 | 1.264 | 0.249 | 0.953 |
| ORF1a | nsp3 | 2425 | 1.880 | 0.246 | 0.953 |
| Spike | Spike | 461 | 0.594 | 0.095 | 0.953 |
| N | N | 85 | 0.683 | 0.132 | 0.953 |
| ORF1a | nsp3 | 1038 | 3.972 | 0.643 | 0.953 |
| ORF1b | nsp13 | 1332 | 1.042 | 0.192 | 0.953 |
| ORF1a | nsp4 | 3232 | 1.765 | 0.201 | 0.953 |
| ORF1a | nsp4 | 3152 | 1.952 | 0.241 | 0.953 |
| E | E | 8 | 4.205 | 0.322 | 0.953 |
| ORF1b | nsp15 | 2118 | 2.005 | 0.157 | 0.953 |
| ORF1b | nsp12 | 743 | 1.860 | 0.195 | 0.952 |
| M | M | 204 | 2.974 | 0.487 | 0.952 |
| ORF1b | nsp15 | 2065 | 2.753 | 0.593 | 0.952 |
| N | N | 172 | 0.578 | 0.095 | 0.952 |
| ORF1b | nsp13 | 1375 | 1.171 | 0.187 | 0.952 |
| ORF1b | nsp13 | 1331 | 1.736 | 0.178 | 0.952 |
| ORF1a | nsp1 | 31 | 0.811 | 0.142 | 0.952 |
| ORF1a | nsp5 | 3322 | 1.739 | 0.200 | 0.952 |
| ORF1a | nsp3 | 1621 | 1.650 | 0.267 | 0.952 |
| Spike | Spike | 1173 | 1.938 | 0.253 | 0.952 |
| Spike | Spike | 563 | 2.304 | 0.163 | 0.952 |
| Spike | Spike | 762 | 2.304 | 0.163 | 0.952 |
| ORF1a | nsp9 | 4196 | 1.062 | 0.187 | 0.952 |
| ORF1a | nsp10 | 4269 | 1.062 | 0.187 | 0.952 |
| ORF1b | nsp15 | 2359 | 1.077 | 0.184 | 0.952 |
| ORF1a | nsp3 | 1097 | 0.982 | 0.187 | 0.952 |
| ORF1a | nsp2 | 802 | 0.983 | 0.187 | 0.952 |
| ORF1a | nsp6 | 3800 | 2.276 | 0.612 | 0.952 |
| ORF1b | nsp13 | 1113 | 1.974 | 0.306 | 0.952 |
| ORF1a | nsp8 | 4003 | 2.919 | 0.277 | 0.952 |
| ORF1b | nsp13 | 1152 | 2.596 | 0.176 | 0.952 |
| Spike | Spike | 20 | 6.908 | 2.776 | 0.951 |
| ORF1a | nsp8 | 4093 | 2.263 | 0.173 | 0.951 |
| ORF1a | nsp9 | 4143 | 2.919 | 0.278 | 0.951 |
| ORF1b | nsp13 | 1446 | 1.747 | 0.181 | 0.951 |
| ORF1a | nsp5 | 3365 | 2.515 | 0.258 | 0.951 |
| ORF1a | nsp4 | 3215 | 2.514 | 0.258 | 0.951 |
| ORF1b | nsp13 | 1459 | 1.743 | 0.181 | 0.951 |
| ORF1b | nsp13 | 1347 | 1.738 | 0.181 | 0.951 |
| ORF1b | nsp15 | 2298 | 1.756 | 0.199 | 0.951 |
| Spike | Spike | 334 | 0.701 | 0.123 | 0.951 |
| ORF1b | nsp13 | 1476 | 3.129 | 0.737 | 0.951 |
| ORF1a | nsp5 | 3530 | 1.984 | 0.161 | 0.951 |
| ORF1a | nsp1 | 89 | 1.644 | 0.214 | 0.951 |
| ORF1b | nsp12 | 46 | 1.204 | 0.218 | 0.951 |
| ORF1a | nsp3 | 966 | 2.140 | 0.529 | 0.951 |
| ORF1a | nsp3 | 1476 | 1.438 | 0.219 | 0.951 |
| ORF1b | nsp12 | 309 | 1.491 | 0.220 | 0.951 |
| ORF1a | nsp3 | 2044 | 2.258 | 0.561 | 0.951 |
| ORF1a | nsp3 | 2484 | 2.920 | 0.535 | 0.951 |
| ORF1b | nsp12 | 583 | 1.699 | 0.246 | 0.950 |
| ORF1a | nsp5 | 3277 | 2.512 | 0.261 | 0.950 |
| ORF1b | nsp12 | 167 | 2.206 | 0.516 | 0.950 |
| Spike | Spike | 652 | 0.752 | 0.124 | 0.950 |
| ORF1b | nsp12 | 387 | 1.666 | 0.239 | 0.950 |
| Spike | Spike | 1257 | 1.130 | 0.196 | 0.950 |
| Spike | Spike | 88 | 0.745 | 0.092 | 0.950 |
| Spike | Spike | 454 | 1.412 | 0.133 | 0.950 |
| Spike | Spike | 567 | 1.412 | 0.133 | 0.950 |
| N | N | 79 | 0.694 | 0.121 | 0.950 |
| Spike | Spike | 1097 | 1.582 | 0.173 | 0.950 |
| Spike | Spike | 810 | 1.580 | 0.173 | 0.950 |
| Spike | Spike | 611 | 0.751 | 0.094 | 0.950 |
| ORF1a | nsp4 | 2897 | 1.724 | 0.346 | 0.949 |
| ORF1a | nsp2 | 733 | 1.538 | 0.318 | 0.949 |
| ORF1a | nsp3 | 1379 | 1.434 | 0.222 | 0.949 |
| ORF1b | nsp14 | 1954 | 1.574 | 0.240 | 0.949 |
| ORF1a | nsp4 | 2983 | 1.722 | 0.346 | 0.949 |
| ORF1a | nsp3 | 2577 | 3.725 | 0.769 | 0.949 |
| Spike | Spike | 723 | 1.617 | 0.382 | 0.949 |
| ORF1a | nsp10 | 4307 | 5.194 | 0.505 | 0.949 |
| ORF1b | nsp13 | 1043 | 1.462 | 0.244 | 0.949 |
| ORF1b | nsp12 | 27 | 1.748 | 0.315 | 0.949 |
| Spike | Spike | 823 | 1.001 | 0.161 | 0.949 |
| ORF1b | nsp13 | 1438 | 1.655 | 0.254 | 0.949 |
| ORF1a | nsp1 | 58 | 3.909 | 0.579 | 0.949 |
| ORF1a | nsp2 | 518 | 2.495 | 0.328 | 0.949 |
| ORF1a | nsp2 | 794 | 2.495 | 0.328 | 0.949 |
| Spike | Spike | 40 | 0.575 | 0.089 | 0.949 |
| Spike | Spike | 556 | 2.423 | 0.610 | 0.949 |
| ORF1a | nsp3 | 2319 | 1.275 | 0.218 | 0.949 |
| Spike | Spike | 807 | 3.220 | 0.446 | 0.949 |
| ORF1a | nsp4 | 2839 | 2.550 | 0.677 | 0.949 |
| ORF1a | nsp10 | 4332 | 1.431 | 0.240 | 0.948 |
| ORF1a | nsp2 | 660 | 1.894 | 0.488 | 0.948 |
| ORF1a | nsp6 | 3830 | 1.138 | 0.139 | 0.948 |
| ORF1b | nsp14 | 1600 | 2.053 | 0.532 | 0.948 |
| ORF1a | nsp4 | 2881 | 1.942 | 0.254 | 0.948 |
| ORF1a | nsp4 | 2964 | 1.942 | 0.254 | 0.948 |
| ORF1a | nsp1 | 72 | 2.330 | 0.271 | 0.948 |
| ORF1a | nsp1 | 11 | 2.329 | 0.271 | 0.948 |
| ORF1a | nsp3 | 2453 | 1.879 | 0.261 | 0.948 |
| ORF1a | nsp3 | 2494 | 1.877 | 0.261 | 0.948 |
| ORF1b | nsp15 | 2375 | 2.235 | 0.242 | 0.948 |
| Spike | Spike | 45 | 0.533 | 0.081 | 0.948 |
| ORF1a | nsp3 | 2559 | 1.872 | 0.261 | 0.947 |
| ORF1a | nsp3 | 1459 | 1.364 | 0.258 | 0.947 |
| Spike | Spike | 956 | 1.582 | 0.179 | 0.947 |
| Spike | Spike | 893 | 1.582 | 0.179 | 0.947 |
| ORF1b | nsp12 | 496 | 2.002 | 0.162 | 0.947 |
| ORF1b | nsp12 | 668 | 2.002 | 0.162 | 0.947 |
| ORF1a | nsp6 | 3645 | 2.618 | 0.860 | 0.947 |
| Spike | Spike | 972 | 1.580 | 0.179 | 0.947 |
| Spike | Spike | 944 | 1.579 | 0.179 | 0.947 |
| Spike | Spike | 1204 | 3.682 | 0.513 | 0.947 |
| Spike | Spike | 602 | 1.131 | 0.124 | 0.947 |
| Spike | Spike | 1193 | 1.510 | 0.220 | 0.947 |
| ORF1b | nsp12 | 431 | 1.224 | 0.199 | 0.947 |
| ORF1b | nsp16 | 2447 | 1.603 | 0.237 | 0.947 |
| ORF1a | nsp2 | 187 | 1.671 | 0.288 | 0.947 |
| Spike | Spike | 991 | 1.205 | 0.187 | 0.947 |
| Spike | Spike | 951 | 1.205 | 0.187 | 0.947 |
| ORF1b | nsp15 | 2087 | 2.234 | 0.245 | 0.946 |
| ORF1b | nsp15 | 2352 | 2.234 | 0.245 | 0.946 |
| ORF1b | nsp14 | 1874 | 0.977 | 0.202 | 0.946 |
| ORF1a | nsp6 | 3586 | 4.311 | 0.472 | 0.946 |
| ORF1b | nsp12 | 104 | 1.666 | 0.249 | 0.946 |
| Spike | Spike | 843 | 3.212 | 0.577 | 0.946 |
| ORF1a | nsp2 | 514 | 3.152 | 0.566 | 0.946 |
| ORF7a | ORF7a | 87 | 2.117 | 0.651 | 0.946 |
| Spike | Spike | 1091 | 2.315 | 0.462 | 0.946 |
| ORF1a | nsp9 | 4150 | 3.748 | 0.353 | 0.946 |
| ORF1b | nsp15 | 2112 | 1.072 | 0.196 | 0.946 |
| ORF1a | nsp9 | 4237 | 1.504 | 0.228 | 0.946 |
| ORF1a | nsp9 | 4149 | 1.504 | 0.228 | 0.946 |
| ORF1b | nsp14 | 1545 | 4.263 | 0.568 | 0.945 |
| Spike | Spike | 274 | 0.634 | 0.086 | 0.945 |
| Spike | Spike | 236 | 0.634 | 0.086 | 0.945 |
| ORF1a | nsp3 | 1968 | 1.482 | 0.225 | 0.945 |
| Spike | Spike | 393 | 0.634 | 0.086 | 0.945 |
| Spike | Spike | 108 | 0.634 | 0.086 | 0.945 |
| ORF1b | nsp12 | 330 | 1.803 | 0.197 | 0.945 |
| Spike | Spike | 191 | 0.983 | 0.116 | 0.945 |
| Spike | Spike | 298 | 0.983 | 0.116 | 0.945 |
| ORF1b | nsp14 | 1574 | 1.045 | 0.208 | 0.945 |
| ORF1a | nsp3 | 2522 | 2.912 | 0.557 | 0.945 |
| ORF1a | nsp3 | 863 | 3.545 | 0.388 | 0.945 |
| ORF1a | nsp4 | 2956 | 0.950 | 0.211 | 0.945 |
| M | M | 183 | 1.007 | 0.166 | 0.945 |
| ORF1a | nsp4 | 3258 | 2.419 | 0.684 | 0.945 |
| ORF1a | nsp3 | 2252 | 1.276 | 0.227 | 0.945 |
| ORF1b | nsp12 | 367 | 1.493 | 0.236 | 0.945 |
| ORF1b | nsp12 | 390 | 1.493 | 0.236 | 0.945 |
| ORF1a | nsp5 | 3403 | 0.942 | 0.182 | 0.944 |
| ORF1a | nsp4 | 3241 | 0.942 | 0.182 | 0.944 |
| Spike | Spike | 637 | 0.751 | 0.101 | 0.944 |
| Spike | Spike | 514 | 0.751 | 0.101 | 0.944 |
| Spike | Spike | 591 | 0.751 | 0.101 | 0.944 |
| ORF1a | nsp1 | 162 | 1.399 | 0.291 | 0.944 |
| ORF1b | nsp13 | 1289 | 5.345 | 1.061 | 0.944 |
| ORF1a | nsp3 | 1674 | 1.539 | 0.235 | 0.944 |
| ORF1a | nsp3 | 1787 | 1.539 | 0.235 | 0.944 |
| ORF1b | nsp14 | 1544 | 2.167 | 0.478 | 0.943 |
| Spike | Spike | 1004 | 1.639 | 0.205 | 0.943 |
| Spike | Spike | 73 | 0.533 | 0.086 | 0.943 |
| ORF1b | nsp13 | 1330 | 1.751 | 0.203 | 0.943 |
| ORF1b | nsp15 | 2283 | 1.124 | 0.206 | 0.943 |
| ORF1b | nsp12 | 266 | 1.248 | 0.242 | 0.943 |
| ORF1b | nsp12 | 39 | 1.248 | 0.242 | 0.943 |
| ORF1b | nsp12 | 339 | 1.248 | 0.242 | 0.943 |
| Spike | Spike | 840 | 0.999 | 0.173 | 0.942 |
| ORF1b | nsp14 | 1971 | 1.306 | 0.283 | 0.942 |
| ORF1a | nsp2 | 504 | 2.503 | 0.350 | 0.942 |
| ORF1b | nsp14 | 1671 | 7.213 | 0.787 | 0.942 |
| ORF1b | nsp14 | 1679 | 7.213 | 0.787 | 0.942 |
| ORF1a | nsp8 | 3947 | 1.793 | 0.256 | 0.942 |
| ORF1a | nsp7 | 3918 | 2.151 | 0.499 | 0.942 |
| ORF1b | nsp12 | 654 | 1.642 | 0.208 | 0.942 |
| ORF1b | nsp13 | 1033 | 2.585 | 0.199 | 0.942 |
| Spike | Spike | 1121 | 1.204 | 0.160 | 0.942 |
| Spike | Spike | 970 | 1.204 | 0.160 | 0.942 |
| Spike | Spike | 1042 | 1.204 | 0.160 | 0.942 |
| ORF1b | nsp15 | 2129 | 1.561 | 0.264 | 0.941 |
| ORF1a | nsp10 | 4349 | 1.939 | 0.239 | 0.941 |
| ORF1a | nsp8 | 4013 | 1.939 | 0.239 | 0.941 |
| ORF1a | nsp10 | 4379 | 1.939 | 0.239 | 0.941 |
| ORF1b | nsp15 | 2302 | 1.597 | 0.287 | 0.941 |
| ORF1b | nsp12 | 384 | 1.490 | 0.244 | 0.941 |
| ORF1a | nsp6 | 3793 | 1.344 | 0.269 | 0.941 |
| ORF1a | nsp3 | 1077 | 3.184 | 0.741 | 0.941 |
| ORF1a | nsp3 | 846 | 1.778 | 0.362 | 0.941 |
| ORF1a | nsp6 | 3787 | 0.952 | 0.197 | 0.941 |
| ORF1a | nsp3 | 1646 | 1.276 | 0.291 | 0.941 |
| ORF1b | nsp12 | 116 | 4.405 | 0.537 | 0.941 |
| ORF1a | nsp4 | 3223 | 1.978 | 0.184 | 0.940 |
| ORF1a | nsp5 | 3464 | 1.977 | 0.184 | 0.940 |
| ORF1a | nsp3 | 2720 | 2.191 | 0.223 | 0.940 |
| ORF1a | nsp3 | 2658 | 2.191 | 0.223 | 0.940 |
| ORF1a | nsp8 | 4067 | 2.255 | 0.199 | 0.940 |
| ORF1a | nsp9 | 4183 | 2.255 | 0.199 | 0.940 |
| ORF1a | nsp8 | 4123 | 2.255 | 0.199 | 0.940 |
| ORF1a | nsp8 | 4023 | 2.254 | 0.199 | 0.940 |
| N | N | 126 | 2.581 | 0.838 | 0.940 |
| M | M | 191 | 1.567 | 0.152 | 0.940 |
| ORF1b | nsp16 | 2397 | 6.281 | 0.484 | 0.940 |
| N | N | 400 | 0.418 | 0.056 | 0.940 |
| ORF1b | nsp13 | 1373 | 2.565 | 0.723 | 0.940 |
| ORF1a | nsp4 | 2961 | 1.832 | 0.271 | 0.939 |
| ORF1a | nsp9 | 4171 | 1.424 | 0.261 | 0.939 |
| Spike | Spike | 1136 | 1.581 | 0.196 | 0.939 |
| ORF1a | nsp3 | 916 | 0.926 | 0.232 | 0.939 |
| ORF1a | nsp4 | 2939 | 1.551 | 0.329 | 0.939 |
| ORF1b | nsp14 | 1562 | 1.272 | 0.231 | 0.939 |
| ORF1a | nsp9 | 4229 | 1.418 | 0.262 | 0.939 |
| ORF3a | ORF3a | 121 | 0.818 | 0.121 | 0.939 |
| Spike | Spike | 575 | 0.751 | 0.109 | 0.939 |
| ORF1a | nsp2 | 736 | 1.039 | 0.158 | 0.939 |
| ORF1a | nsp2 | 370 | 0.811 | 0.165 | 0.939 |
| ORF1a | nsp4 | 3214 | 1.246 | 0.245 | 0.938 |
| Spike | Spike | 610 | 0.751 | 0.109 | 0.938 |
| Spike | Spike | 230 | 1.114 | 0.255 | 0.938 |
| Spike | Spike | 433 | 0.751 | 0.109 | 0.938 |
| ORF1b | nsp12 | 64 | 2.028 | 0.308 | 0.938 |
| ORF1b | nsp12 | 360 | 2.028 | 0.308 | 0.938 |
| ORF1b | nsp13 | 1412 | 1.499 | 0.327 | 0.938 |
| ORF1a | nsp2 | 765 | 3.204 | 0.636 | 0.938 |
| ORF1a | nsp2 | 594 | 2.406 | 0.574 | 0.938 |
| Spike | Spike | 358 | 0.551 | 0.096 | 0.938 |
| N | N | 305 | 0.881 | 0.121 | 0.938 |
| ORF1a | nsp2 | 225 | 3.358 | 0.752 | 0.938 |
| ORF1a | nsp10 | 4258 | 2.252 | 0.205 | 0.938 |
| ORF1a | nsp10 | 4300 | 2.251 | 0.205 | 0.938 |
| Spike | Spike | 1043 | 1.202 | 0.168 | 0.937 |
| Spike | Spike | 1119 | 1.360 | 0.252 | 0.937 |
| Spike | Spike | 1135 | 1.360 | 0.252 | 0.937 |
| ORF1b | nsp12 | 695 | 1.549 | 0.212 | 0.937 |
| ORF3a | ORF3a | 230 | 0.522 | 0.099 | 0.937 |
| M | M | 211 | 0.766 | 0.153 | 0.937 |
| ORF3a | ORF3a | 62 | 2.154 | 0.722 | 0.937 |
| ORF1b | nsp12 | 57 | 1.615 | 0.267 | 0.937 |
| ORF1a | nsp5 | 3305 | 2.838 | 0.512 | 0.937 |
| Spike | Spike | 1226 | 0.704 | 0.158 | 0.937 |
| ORF1b | nsp14 | 1791 | 2.334 | 0.247 | 0.937 |
| ORF1b | nsp14 | 1949 | 2.332 | 0.247 | 0.937 |
| ORF1a | nsp3 | 1772 | 1.198 | 0.290 | 0.937 |
| ORF1a | nsp2 | 744 | 1.852 | 0.304 | 0.937 |
| ORF1b | nsp14 | 1609 | 5.563 | 0.741 | 0.936 |
| ORF1a | nsp3 | 2687 | 1.714 | 0.364 | 0.936 |
| N | N | 282 | 2.798 | 1.055 | 0.936 |
| ORF1b | nsp12 | 571 | 1.175 | 0.219 | 0.936 |
| N | N | 304 | 0.473 | 0.072 | 0.936 |
| ORF1a | nsp3 | 2418 | 2.189 | 0.235 | 0.936 |
| Spike | Spike | 1066 | 2.320 | 0.497 | 0.936 |
| Spike | Spike | 386 | 0.980 | 0.131 | 0.936 |
| Spike | Spike | 41 | 0.980 | 0.131 | 0.936 |
| Spike | Spike | 202 | 0.980 | 0.131 | 0.936 |
| Spike | Spike | 129 | 0.980 | 0.131 | 0.936 |
| Spike | Spike | 310 | 0.981 | 0.131 | 0.936 |
| ORF1a | nsp3 | 897 | 1.149 | 0.279 | 0.936 |
| ORF1a | nsp3 | 1931 | 3.674 | 0.501 | 0.936 |
| ORF1b | nsp13 | 1070 | 1.255 | 0.257 | 0.935 |
| Spike | Spike | 571 | 0.659 | 0.131 | 0.935 |
| Spike | Spike | 586 | 0.659 | 0.131 | 0.935 |
| ORF1b | nsp13 | 1151 | 2.577 | 0.213 | 0.935 |
| ORF1b | nsp13 | 1154 | 2.575 | 0.213 | 0.935 |
| ORF1a | nsp3 | 1579 | 0.903 | 0.220 | 0.935 |
| N | N | 396 | 0.562 | 0.112 | 0.935 |
| N | N | 82 | 0.718 | 0.166 | 0.935 |
| N | N | 358 | 0.718 | 0.166 | 0.935 |
| M | M | 140 | 1.103 | 0.222 | 0.935 |
| ORF1b | nsp14 | 1756 | 5.501 | 0.711 | 0.935 |
| ORF1a | nsp3 | 1469 | 0.900 | 0.220 | 0.935 |
| Spike | Spike | 467 | 0.974 | 0.133 | 0.935 |
| Spike | Spike | 737 | 0.974 | 0.133 | 0.935 |
| ORF1a | nsp4 | 3218 | 1.749 | 0.246 | 0.934 |
| ORF1a | nsp4 | 3242 | 1.747 | 0.246 | 0.934 |
| ORF1a | nsp2 | 308 | 0.937 | 0.145 | 0.934 |
| ORF1b | nsp12 | 745 | 1.987 | 0.189 | 0.934 |
| ORF1b | nsp12 | 663 | 1.987 | 0.189 | 0.934 |
| ORF1b | nsp12 | 673 | 1.984 | 0.189 | 0.934 |
| ORF3a | ORF3a | 135 | 0.499 | 0.096 | 0.934 |
| ORF1b | nsp12 | 269 | 2.032 | 0.321 | 0.934 |
| Spike | Spike | 416 | 1.125 | 0.145 | 0.934 |
| Spike | Spike | 601 | 1.125 | 0.145 | 0.934 |
| ORF1a | nsp2 | 443 | 6.919 | 2.897 | 0.934 |
| ORF8 | ORF8 | 15 | 0.724 | 0.125 | 0.934 |
| ORF1a | nsp3 | 1434 | 2.447 | 0.494 | 0.934 |
| ORF1b | nsp12 | 107 | 2.330 | 0.493 | 0.934 |
| N | N | 42 | 0.555 | 0.112 | 0.933 |
| ORF1b | nsp15 | 2386 | 7.308 | 1.006 | 0.933 |
| ORF1a | nsp5 | 3362 | 0.999 | 0.141 | 0.933 |
| ORF1b | nsp14 | 1549 | 1.766 | 0.227 | 0.933 |
| ORF1b | nsp12 | 507 | 1.831 | 0.242 | 0.933 |
| ORF1b | nsp12 | 597 | 1.831 | 0.242 | 0.933 |
| ORF1b | nsp14 | 1952 | 2.342 | 0.257 | 0.933 |
| ORF1b | nsp12 | 719 | 1.830 | 0.242 | 0.933 |
| ORF1a | nsp2 | 777 | 1.885 | 0.366 | 0.933 |
| ORF1b | nsp12 | 515 | 11.090 | 0.921 | 0.933 |
| ORF1a | nsp8 | 4057 | 2.605 | 0.487 | 0.933 |
| ORF1b | nsp16 | 2504 | 1.706 | 0.173 | 0.933 |
| ORF1b | nsp16 | 2476 | 1.706 | 0.173 | 0.933 |
| ORF1b | nsp16 | 2442 | 1.706 | 0.173 | 0.933 |
| ORF1b | nsp13 | 973 | 1.070 | 0.223 | 0.933 |
| Spike | Spike | 519 | 2.607 | 0.514 | 0.932 |
| ORF1a | nsp6 | 3654 | 1.716 | 0.273 | 0.932 |
| ORF1a | nsp8 | 3954 | 1.716 | 0.273 | 0.932 |
| Spike | Spike | 113 | 2.208 | 0.550 | 0.932 |
| ORF1a | nsp6 | 3822 | 1.714 | 0.273 | 0.932 |
| ORF1b | nsp15 | 2225 | 1.997 | 0.200 | 0.932 |
| ORF1a | nsp3 | 2673 | 2.154 | 0.338 | 0.932 |
| Spike | Spike | 995 | 1.196 | 0.177 | 0.932 |
| Spike | Spike | 905 | 1.196 | 0.177 | 0.932 |
| ORF1b | nsp15 | 2275 | 1.129 | 0.197 | 0.932 |
| Spike | Spike | 423 | 0.973 | 0.137 | 0.932 |
| ORF1a | nsp3 | 2378 | 1.223 | 0.198 | 0.932 |
| ORF1b | nsp14 | 2002 | 2.230 | 0.286 | 0.932 |
| N | N | 293 | 1.242 | 0.150 | 0.932 |
| ORF1b | nsp14 | 1738 | 0.950 | 0.226 | 0.931 |
| Spike | Spike | 1080 | 0.921 | 0.181 | 0.931 |
| ORF1a | nsp8 | 3995 | 3.714 | 0.725 | 0.931 |
| ORF1a | nsp3 | 2341 | 0.830 | 0.151 | 0.931 |
| ORF1a | nsp3 | 2013 | 0.830 | 0.151 | 0.931 |
| ORF1b | nsp12 | 254 | 3.584 | 0.693 | 0.931 |
| Spike | Spike | 1187 | 3.237 | 0.642 | 0.931 |
| ORF1a | nsp4 | 3124 | 2.257 | 0.230 | 0.930 |
| ORF1b | nsp12 | 334 | 1.815 | 0.231 | 0.930 |
| ORF1a | nsp3 | 2424 | 2.193 | 0.249 | 0.930 |
| ORF1b | nsp13 | 1408 | 4.301 | 0.671 | 0.930 |
| Spike | Spike | 700 | 0.751 | 0.120 | 0.930 |
| Spike | Spike | 799 | 0.751 | 0.120 | 0.930 |
| Spike | Spike | 593 | 0.751 | 0.120 | 0.930 |
| Spike | Spike | 744 | 0.751 | 0.120 | 0.930 |
| ORF1b | nsp12 | 165 | 4.476 | 0.597 | 0.930 |
| ORF6 | ORF6 | 12 | 1.460 | 0.317 | 0.930 |
| ORF1a | nsp3 | 2028 | 1.023 | 0.289 | 0.930 |
| ORF1b | nsp12 | 484 | 1.164 | 0.230 | 0.930 |
| ORF1b | nsp14 | 1865 | 2.362 | 0.267 | 0.930 |
| ORF1a | nsp7 | 3883 | 1.138 | 0.171 | 0.930 |
| ORF1a | nsp8 | 3953 | 1.137 | 0.171 | 0.930 |
| ORF1a | nsp1 | 38 | 0.832 | 0.225 | 0.930 |
| ORF1a | nsp2 | 195 | 1.001 | 0.267 | 0.930 |
| ORF3a | ORF3a | 34 | 2.717 | 1.158 | 0.929 |
| ORF1b | nsp16 | 2549 | 1.438 | 0.244 | 0.929 |
| ORF1a | nsp3 | 2706 | 3.430 | 0.681 | 0.929 |
| ORF1a | nsp6 | 3692 | 1.254 | 0.284 | 0.929 |
| ORF1a | nsp5 | 3265 | 1.610 | 0.246 | 0.929 |
| ORF1a | nsp1 | 74 | 2.667 | 0.825 | 0.929 |
| ORF1a | nsp4 | 2779 | 4.862 | 1.067 | 0.928 |
| ORF1b | nsp12 | 321 | 1.429 | 0.266 | 0.928 |
| ORF1a | nsp2 | 219 | 2.238 | 0.637 | 0.928 |
| Spike | Spike | 925 | 2.391 | 0.633 | 0.928 |
| ORF1b | nsp12 | 415 | 6.432 | 1.169 | 0.928 |
| ORF1a | nsp3 | 2330 | 2.291 | 0.431 | 0.928 |
| Spike | Spike | 875 | 1.018 | 0.165 | 0.928 |
| ORF1b | nsp15 | 2093 | 1.187 | 0.210 | 0.928 |
| Spike | Spike | 186 | 2.048 | 0.645 | 0.928 |
| Spike | Spike | 1123 | 1.015 | 0.165 | 0.928 |
| ORF1a | nsp3 | 2346 | 1.707 | 0.514 | 0.928 |
| Spike | Spike | 1090 | 1.015 | 0.165 | 0.927 |
| N | N | 93 | 1.319 | 0.212 | 0.927 |
| N | N | 88 | 1.318 | 0.212 | 0.927 |
| ORF1b | nsp14 | 1809 | 0.980 | 0.239 | 0.927 |
| ORF1b | nsp13 | 1122 | 1.025 | 0.228 | 0.927 |
| Spike | Spike | 221 | 3.600 | 1.110 | 0.927 |
| Spike | Spike | 1044 | 1.587 | 0.222 | 0.927 |
| ORF1a | nsp8 | 3996 | 0.756 | 0.164 | 0.927 |
| ORF1a | nsp7 | 3907 | 0.756 | 0.164 | 0.927 |
| ORF1a | nsp3 | 2519 | 2.903 | 0.658 | 0.927 |
| Spike | Spike | 99 | 2.045 | 0.622 | 0.927 |
| ORF1b | nsp12 | 675 | 1.823 | 0.258 | 0.926 |
| ORF1b | nsp12 | 614 | 1.823 | 0.258 | 0.926 |
| ORF1b | nsp12 | 516 | 1.823 | 0.258 | 0.926 |
| ORF1b | nsp12 | 752 | 1.822 | 0.258 | 0.926 |
| ORF1b | nsp16 | 2467 | 1.741 | 0.187 | 0.926 |
| Spike | Spike | 531 | 0.750 | 0.126 | 0.926 |
| ORF1a | nsp10 | 4384 | 4.538 | 0.743 | 0.926 |
| ORF1a | nsp3 | 1734 | 2.547 | 0.826 | 0.926 |
| ORF1a | nsp3 | 1325 | 2.014 | 0.328 | 0.926 |
| ORF1b | nsp15 | 2182 | 2.006 | 0.215 | 0.925 |
| ORF1a | nsp3 | 1590 | 2.012 | 0.328 | 0.925 |
| ORF1a | nsp6 | 3732 | 0.755 | 0.166 | 0.925 |
| ORF1b | nsp12 | 563 | 2.901 | 0.535 | 0.925 |
| ORF3a | ORF3a | 79 | 0.513 | 0.090 | 0.925 |
| ORF3a | ORF3a | 56 | 0.513 | 0.090 | 0.925 |
| N | N | 386 | 2.295 | 0.482 | 0.925 |
| ORF1b | nsp12 | 845 | 1.044 | 0.167 | 0.925 |
| ORF1b | nsp13 | 1088 | 1.044 | 0.167 | 0.925 |
| Spike | Spike | 125 | 0.743 | 0.125 | 0.925 |
| Spike | Spike | 331 | 0.743 | 0.125 | 0.925 |
| Spike | Spike | 196 | 0.743 | 0.125 | 0.925 |
| Spike | Spike | 74 | 0.743 | 0.125 | 0.925 |
| ORF1b | nsp13 | 930 | 1.044 | 0.167 | 0.925 |
| N | N | 61 | 0.870 | 0.140 | 0.924 |
| N | N | 375 | 0.870 | 0.140 | 0.924 |
| ORF6 | ORF6 | 30 | 1.228 | 0.256 | 0.924 |
| Spike | Spike | 137 | 0.745 | 0.126 | 0.924 |
| ORF1a | nsp3 | 1357 | 1.427 | 0.213 | 0.924 |
| ORF1b | nsp13 | 1295 | 0.818 | 0.183 | 0.924 |
| ORF1b | nsp16 | 2448 | 1.737 | 0.191 | 0.924 |
| ORF1b | nsp12 | 834 | 1.216 | 0.179 | 0.924 |
| ORF1a | nsp5 | 3319 | 1.739 | 0.271 | 0.924 |
| ORF1b | nsp16 | 2455 | 1.689 | 0.189 | 0.924 |
| ORF1b | nsp16 | 2588 | 1.688 | 0.189 | 0.924 |
| N | N | 123 | 0.508 | 0.092 | 0.923 |
| Spike | Spike | 626 | 3.316 | 0.646 | 0.923 |
| ORF1a | nsp3 | 1169 | 0.990 | 0.244 | 0.923 |
| ORF1b | nsp13 | 1018 | 2.264 | 0.304 | 0.923 |
| ORF1a | nsp2 | 196 | 2.257 | 0.701 | 0.923 |
| ORF1b | nsp12 | 560 | 3.183 | 0.263 | 0.923 |
| ORF1b | nsp12 | 717 | 3.183 | 0.263 | 0.923 |
| ORF1a | nsp4 | 3037 | 1.026 | 0.186 | 0.923 |
| ORF1b | nsp16 | 2622 | 1.735 | 0.193 | 0.923 |
| Spike | Spike | 868 | 2.349 | 0.286 | 0.923 |
| Spike | Spike | 819 | 2.349 | 0.286 | 0.923 |
| Spike | Spike | 990 | 2.349 | 0.286 | 0.923 |
| Spike | Spike | 1072 | 2.349 | 0.286 | 0.923 |
| ORF1b | nsp13 | 1490 | 2.068 | 0.274 | 0.922 |
| ORF1b | nsp13 | 1502 | 2.065 | 0.273 | 0.922 |
| N | N | 336 | 0.556 | 0.124 | 0.922 |
| ORF1a | nsp10 | 4353 | 1.120 | 0.172 | 0.922 |
| N | N | 222 | 0.481 | 0.101 | 0.922 |
| ORF1a | nsp2 | 506 | 1.120 | 0.247 | 0.922 |
| N | N | 44 | 0.564 | 0.127 | 0.922 |
| N | N | 278 | 0.564 | 0.127 | 0.922 |
| ORF1a | nsp3 | 1713 | 0.820 | 0.150 | 0.922 |
| ORF1a | nsp3 | 1914 | 0.820 | 0.150 | 0.922 |
| ORF1a | nsp10 | 4321 | 1.198 | 0.178 | 0.922 |
| ORF1a | nsp9 | 4215 | 1.198 | 0.178 | 0.922 |
| ORF1b | nsp12 | 891 | 1.417 | 0.247 | 0.922 |
| ORF1b | nsp13 | 1086 | 1.417 | 0.247 | 0.922 |
| ORF8 | ORF8 | 6 | 0.556 | 0.122 | 0.921 |
| ORF1a | nsp8 | 4059 | 1.213 | 0.227 | 0.921 |
| ORF1a | nsp8 | 4001 | 1.213 | 0.227 | 0.921 |
| ORF1a | nsp10 | 4282 | 1.911 | 0.288 | 0.921 |
| ORF1a | nsp5 | 3404 | 0.992 | 0.159 | 0.921 |
| ORF1b | nsp12 | 677 | 1.970 | 0.214 | 0.921 |
| ORF1a | nsp3 | 819 | 2.262 | 0.444 | 0.921 |
| Spike | Spike | 122 | 0.576 | 0.121 | 0.921 |
| ORF1a | nsp1 | 26 | 1.016 | 0.219 | 0.921 |
| Spike | Spike | 497 | 0.479 | 0.086 | 0.921 |
| ORF1b | nsp14 | 2016 | 2.198 | 0.234 | 0.921 |
| ORF1b | nsp15 | 2249 | 2.196 | 0.234 | 0.921 |
| ORF1a | nsp10 | 4255 | 1.749 | 0.274 | 0.921 |
| ORF1b | nsp12 | 600 | 1.971 | 0.215 | 0.921 |
| ORF1b | nsp14 | 1866 | 0.945 | 0.181 | 0.921 |
| ORF1b | nsp14 | 1859 | 0.945 | 0.181 | 0.921 |
| N | N | 51 | 0.414 | 0.070 | 0.921 |
| N | N | 184 | 0.414 | 0.070 | 0.921 |
| ORF1a | nsp3 | 1569 | 2.018 | 0.342 | 0.920 |
| Spike | Spike | 1046 | 0.918 | 0.199 | 0.920 |
| Spike | Spike | 1171 | 0.918 | 0.199 | 0.920 |
| N | N | 170 | 0.557 | 0.127 | 0.920 |
| Spike | Spike | 984 | 1.013 | 0.176 | 0.920 |
| Spike | Spike | 84 | 1.013 | 0.176 | 0.920 |
| ORF1b | nsp13 | 1244 | 4.329 | 0.718 | 0.920 |
| ORF1b | nsp13 | 1343 | 5.972 | 0.804 | 0.920 |
| ORF1a | nsp4 | 3181 | 1.268 | 0.333 | 0.920 |
| ORF1b | nsp12 | 226 | 1.668 | 0.315 | 0.920 |
| ORF1b | nsp12 | 20 | 1.667 | 0.315 | 0.920 |
| ORF3a | ORF3a | 153 | 0.513 | 0.095 | 0.920 |
| ORF1b | nsp13 | 1496 | 0.864 | 0.165 | 0.920 |
| Spike | Spike | 1131 | 3.282 | 0.556 | 0.920 |
| ORF1a | nsp2 | 494 | 1.035 | 0.270 | 0.920 |
| ORF1a | nsp3 | 1619 | 1.541 | 0.292 | 0.920 |
| ORF1a | nsp3 | 1882 | 1.540 | 0.292 | 0.920 |
| ORF1b | nsp13 | 1394 | 0.833 | 0.193 | 0.919 |
| ORF1b | nsp15 | 2362 | 1.609 | 0.263 | 0.919 |
| ORF1b | nsp13 | 1385 | 5.963 | 0.809 | 0.919 |
| ORF1b | nsp12 | 782 | 1.816 | 0.275 | 0.919 |
| ORF1b | nsp12 | 450 | 1.816 | 0.275 | 0.919 |
| ORF1a | nsp5 | 3563 | 0.935 | 0.226 | 0.919 |
| ORF1a | nsp3 | 1331 | 1.439 | 0.292 | 0.919 |
| ORF1a | nsp3 | 1524 | 1.439 | 0.292 | 0.919 |
| Spike | Spike | 544 | 0.971 | 0.157 | 0.919 |
| Spike | Spike | 422 | 0.971 | 0.157 | 0.919 |
| Spike | Spike | 437 | 0.971 | 0.157 | 0.919 |
| Spike | Spike | 448 | 0.971 | 0.157 | 0.919 |
| N | N | 150 | 0.695 | 0.165 | 0.919 |
| ORF1a | nsp5 | 3396 | 1.737 | 0.283 | 0.919 |
| ORF1a | nsp5 | 3358 | 1.737 | 0.283 | 0.919 |
| ORF1a | nsp5 | 3477 | 1.737 | 0.283 | 0.919 |
| Spike | Spike | 546 | 0.781 | 0.139 | 0.918 |
| Spike | Spike | 763 | 0.780 | 0.139 | 0.918 |
| Spike | Spike | 650 | 0.780 | 0.139 | 0.918 |
| ORF3a | ORF3a | 73 | 0.969 | 0.398 | 0.918 |
| Spike | Spike | 542 | 0.661 | 0.154 | 0.918 |
| Spike | Spike | 266 | 0.743 | 0.134 | 0.918 |
| Spike | Spike | 369 | 0.742 | 0.134 | 0.918 |
| Spike | Spike | 204 | 0.742 | 0.134 | 0.918 |
| ORF1a | nsp3 | 1746 | 4.181 | 0.633 | 0.918 |
| Spike | Spike | 200 | 0.742 | 0.134 | 0.918 |
| ORF1a | nsp3 | 1604 | 2.119 | 0.505 | 0.918 |
| ORF1a | nsp4 | 3026 | 1.737 | 0.319 | 0.918 |
| Spike | Spike | 983 | 1.012 | 0.180 | 0.918 |
| ORF1a | nsp4 | 3199 | 1.735 | 0.319 | 0.918 |
| ORF1b | nsp15 | 2101 | 1.056 | 0.171 | 0.918 |
| ORF1b | nsp14 | 1854 | 1.887 | 0.557 | 0.918 |
| ORF1b | nsp13 | 1364 | 0.989 | 0.187 | 0.918 |
| ORF1b | nsp14 | 1535 | 0.989 | 0.187 | 0.918 |
| ORF1b | nsp12 | 140 | 2.276 | 0.639 | 0.918 |
| Spike | Spike | 10 | 0.578 | 0.125 | 0.918 |
| ORF1b | nsp12 | 166 | 2.274 | 0.639 | 0.918 |
| ORF1b | nsp12 | 656 | 3.774 | 0.301 | 0.918 |
| ORF1b | nsp12 | 594 | 3.776 | 0.301 | 0.917 |
| ORF1b | nsp12 | 499 | 3.776 | 0.301 | 0.917 |
| ORF1b | nsp12 | 584 | 3.776 | 0.301 | 0.917 |
| ORF1b | nsp13 | 1500 | 0.864 | 0.169 | 0.917 |
| ORF1b | nsp14 | 1912 | 1.939 | 0.364 | 0.917 |
| ORF1b | nsp12 | 669 | 1.523 | 0.254 | 0.917 |
| ORF1b | nsp13 | 1207 | 0.865 | 0.170 | 0.917 |
| ORF1b | nsp13 | 1368 | 0.864 | 0.170 | 0.917 |
| ORF1b | nsp12 | 588 | 1.522 | 0.254 | 0.917 |
| ORF1a | nsp3 | 2100 | 1.229 | 0.226 | 0.917 |
| ORF1a | nsp3 | 2372 | 1.228 | 0.226 | 0.917 |
| ORF1a | nsp3 | 1546 | 1.278 | 0.316 | 0.917 |
| ORF1a | nsp2 | 587 | 1.044 | 0.196 | 0.917 |
| ORF1b | nsp16 | 2480 | 1.372 | 0.184 | 0.917 |
| ORF1a | nsp6 | 3576 | 0.905 | 0.226 | 0.917 |
| ORF1b | nsp16 | 2424 | 1.375 | 0.185 | 0.916 |
| Spike | Spike | 882 | 0.937 | 0.208 | 0.916 |
| ORF1b | nsp16 | 2649 | 1.372 | 0.185 | 0.916 |
| ORF1b | nsp16 | 2671 | 1.372 | 0.185 | 0.916 |
| ORF1a | nsp3 | 1400 | 1.427 | 0.228 | 0.916 |
| ORF1b | nsp16 | 2672 | 1.369 | 0.185 | 0.916 |
| ORF1a | nsp8 | 4082 | 1.908 | 0.301 | 0.916 |
| ORF1a | nsp10 | 4315 | 1.908 | 0.301 | 0.916 |
| ORF1a | nsp8 | 4134 | 1.908 | 0.301 | 0.916 |
| Spike | Spike | 1025 | 1.012 | 0.183 | 0.916 |
| ORF1a | nsp4 | 3256 | 0.989 | 0.167 | 0.916 |
| Spike | Spike | 1022 | 1.011 | 0.183 | 0.916 |
| ORF8 | ORF8 | 28 | 0.637 | 0.131 | 0.916 |
| ORF1a | nsp8 | 4121 | 1.904 | 0.301 | 0.916 |
| Spike | Spike | 1 | 1.552 | 0.053 | 0.916 |
| ORF1b | nsp16 | 2637 | 1.370 | 0.186 | 0.915 |
| ORF1b | nsp16 | 2542 | 1.370 | 0.186 | 0.915 |
| N | N | 56 | 0.692 | 0.245 | 0.915 |
| M | M | 139 | 1.547 | 0.195 | 0.915 |
| ORF7a | ORF7a | 16 | 1.038 | 0.247 | 0.915 |
| ORF1a | nsp2 | 382 | 0.811 | 0.202 | 0.915 |
| ORF1a | nsp3 | 1327 | 0.901 | 0.255 | 0.915 |
| M | M | 187 | 1.537 | 0.195 | 0.914 |
| M | M | 88 | 1.536 | 0.195 | 0.914 |
| ORF1a | nsp3 | 1958 | 1.654 | 0.236 | 0.914 |
| ORF1a | nsp3 | 1908 | 1.654 | 0.236 | 0.914 |
| ORF1b | nsp13 | 1413 | 1.156 | 0.214 | 0.914 |
| ORF1b | nsp13 | 1080 | 2.632 | 0.518 | 0.914 |
| ORF1a | nsp4 | 2945 | 1.887 | 0.341 | 0.914 |
| ORF1b | nsp13 | 1205 | 1.794 | 0.275 | 0.914 |
| ORF1b | nsp15 | 2167 | 1.257 | 0.193 | 0.914 |
| ORF1a | nsp3 | 2752 | 2.161 | 0.395 | 0.914 |
| ORF1a | nsp3 | 2439 | 2.161 | 0.395 | 0.914 |
| ORF1a | nsp3 | 2451 | 2.159 | 0.395 | 0.913 |
| ORF1a | nsp8 | 3948 | 0.994 | 0.184 | 0.913 |
| ORF1a | nsp6 | 3639 | 0.994 | 0.184 | 0.913 |
| ORF1a | nsp8 | 3991 | 0.994 | 0.184 | 0.913 |
| ORF1a | nsp6 | 3628 | 0.994 | 0.184 | 0.913 |
| ORF1a | nsp6 | 3611 | 0.994 | 0.184 | 0.913 |
| ORF1b | nsp12 | 827 | 2.816 | 0.273 | 0.913 |
| ORF1a | nsp2 | 199 | 1.534 | 0.293 | 0.913 |
| ORF1a | nsp1 | 176 | 1.534 | 0.293 | 0.913 |
| Spike | Spike | 166 | 0.433 | 0.076 | 0.913 |
| ORF1a | nsp5 | 3561 | 4.415 | 0.626 | 0.913 |
| ORF1a | nsp3 | 1758 | 5.832 | 1.567 | 0.913 |
| ORF3a | ORF3a | 60 | 2.822 | 1.309 | 0.913 |
| ORF1b | nsp13 | 1134 | 1.065 | 0.260 | 0.913 |
| ORF1b | nsp15 | 2106 | 1.256 | 0.194 | 0.913 |
| ORF1b | nsp15 | 2173 | 1.256 | 0.194 | 0.913 |
| ORF1b | nsp14 | 2038 | 1.256 | 0.195 | 0.912 |
| Spike | Spike | 1095 | 1.979 | 0.434 | 0.912 |
| ORF1b | nsp15 | 2245 | 1.255 | 0.195 | 0.912 |
| ORF1b | nsp14 | 2030 | 1.256 | 0.195 | 0.912 |
| E | E | 38 | 2.576 | 0.360 | 0.912 |
| ORF1b | nsp12 | 356 | 2.075 | 0.286 | 0.912 |
| ORF1b | nsp12 | 296 | 2.076 | 0.286 | 0.912 |
| ORF1a | nsp6 | 3810 | 1.965 | 0.488 | 0.912 |
| ORF1b | nsp12 | 805 | 1.038 | 0.186 | 0.912 |
| M | M | 131 | 4.160 | 0.521 | 0.912 |
| ORF1a | nsp6 | 3747 | 1.137 | 0.200 | 0.912 |
| ORF1b | nsp12 | 852 | 1.036 | 0.186 | 0.912 |
| ORF1b | nsp14 | 1670 | 1.048 | 0.207 | 0.912 |
| ORF1b | nsp14 | 1955 | 1.048 | 0.207 | 0.912 |
| ORF1b | nsp12 | 22 | 1.826 | 0.271 | 0.912 |
| ORF1b | nsp12 | 389 | 1.826 | 0.271 | 0.912 |
| ORF1b | nsp16 | 2680 | 1.775 | 0.215 | 0.912 |
| ORF1b | nsp16 | 2676 | 1.775 | 0.215 | 0.912 |
| ORF1b | nsp14 | 1920 | 0.768 | 0.195 | 0.912 |
| ORF1a | nsp4 | 2949 | 2.108 | 0.694 | 0.912 |
| Spike | Spike | 163 | 1.761 | 0.580 | 0.912 |
| ORF1a | nsp3 | 1973 | 5.115 | 0.795 | 0.912 |
| ORF1a | nsp3 | 1087 | 0.707 | 0.161 | 0.912 |
| ORF1a | nsp3 | 1606 | 5.116 | 0.796 | 0.912 |
| ORF1a | nsp3 | 2657 | 1.289 | 0.275 | 0.912 |
| ORF1a | nsp3 | 2725 | 1.333 | 0.317 | 0.912 |
| ORF1b | nsp14 | 1834 | 2.565 | 0.326 | 0.912 |
| ORF1a | nsp4 | 2924 | 1.884 | 0.347 | 0.911 |
| ORF1b | nsp16 | 2674 | 4.612 | 0.599 | 0.911 |
| ORF1a | nsp1 | 71 | 0.707 | 0.206 | 0.911 |
| N | N | 43 | 5.092 | 0.968 | 0.911 |
| ORF1b | nsp15 | 2220 | 1.991 | 0.244 | 0.911 |
| ORF1a | nsp8 | 3943 | 0.817 | 0.165 | 0.911 |
| ORF1a | nsp8 | 3967 | 0.817 | 0.165 | 0.911 |
| ORF1a | nsp8 | 3958 | 0.817 | 0.165 | 0.911 |
| Spike | Spike | 144 | 7.485 | 3.116 | 0.911 |
| ORF1a | nsp4 | 2798 | 6.450 | 0.889 | 0.911 |
| ORF1a | nsp8 | 4047 | 1.390 | 0.326 | 0.911 |
| Spike | Spike | 814 | 1.277 | 0.224 | 0.910 |
| ORF1a | nsp4 | 2847 | 1.884 | 0.350 | 0.910 |
| ORF1a | nsp4 | 3159 | 1.883 | 0.350 | 0.910 |
| ORF1a | nsp3 | 825 | 0.767 | 0.177 | 0.910 |
| ORF1a | nsp4 | 3025 | 1.881 | 0.350 | 0.910 |
| ORF1a | nsp3 | 1353 | 1.055 | 0.211 | 0.910 |
| ORF1a | nsp10 | 4373 | 1.034 | 0.263 | 0.910 |
| ORF1a | nsp2 | 385 | 2.794 | 1.134 | 0.910 |
| ORF1a | nsp3 | 1917 | 1.277 | 0.365 | 0.909 |
| Spike | Spike | 1031 | 1.276 | 0.226 | 0.909 |
| ORF1a | nsp6 | 3622 | 0.818 | 0.167 | 0.909 |
| ORF1a | nsp3 | 2295 | 1.188 | 0.285 | 0.909 |
| ORF1a | nsp3 | 2117 | 1.188 | 0.285 | 0.909 |
| ORF1a | nsp8 | 3989 | 0.817 | 0.167 | 0.909 |
| Spike | Spike | 1142 | 2.375 | 0.323 | 0.909 |
| Spike | Spike | 895 | 2.375 | 0.323 | 0.909 |
| Spike | Spike | 992 | 2.375 | 0.323 | 0.909 |
| Spike | Spike | 853 | 2.374 | 0.323 | 0.909 |
| ORF1a | nsp4 | 2951 | 1.266 | 0.357 | 0.909 |
| ORF1b | nsp13 | 1448 | 0.961 | 0.197 | 0.909 |
| ORF1b | nsp13 | 1256 | 0.961 | 0.197 | 0.909 |
| Spike | Spike | 1011 | 2.384 | 0.325 | 0.909 |
| ORF1b | nsp14 | 1994 | 0.948 | 0.199 | 0.909 |
| ORF1b | nsp14 | 1754 | 0.947 | 0.199 | 0.909 |
| ORF1b | nsp14 | 1931 | 0.947 | 0.199 | 0.909 |
| ORF1b | nsp14 | 1718 | 0.947 | 0.199 | 0.909 |
| ORF1a | nsp3 | 2066 | 1.771 | 0.476 | 0.909 |
| ORF1b | nsp13 | 1451 | 0.976 | 0.239 | 0.909 |
| ORF1a | nsp10 | 4268 | 1.105 | 0.192 | 0.909 |
| ORF1a | nsp5 | 3557 | 1.045 | 0.183 | 0.909 |
| ORF1a | nsp5 | 3493 | 1.045 | 0.183 | 0.909 |
| ORF1a | nsp5 | 3448 | 1.044 | 0.183 | 0.909 |
| ORF1b | nsp12 | 342 | 1.359 | 0.333 | 0.909 |
| ORF6 | ORF6 | 7 | 2.220 | 0.542 | 0.909 |
| ORF1b | nsp12 | 307 | 1.513 | 0.321 | 0.909 |
| ORF1a | nsp2 | 796 | 1.896 | 0.423 | 0.909 |
| ORF1b | nsp12 | 198 | 1.513 | 0.321 | 0.909 |
| ORF1a | nsp3 | 2297 | 0.834 | 0.186 | 0.908 |
| ORF1a | nsp4 | 3156 | 1.683 | 0.539 | 0.908 |
| ORF1a | nsp8 | 4137 | 1.610 | 0.494 | 0.908 |
| ORF1a | nsp8 | 3999 | 0.817 | 0.169 | 0.908 |
| ORF1a | nsp3 | 2270 | 1.514 | 0.338 | 0.907 |
| ORF1a | nsp3 | 2095 | 1.797 | 0.563 | 0.907 |
| Spike | Spike | 911 | 1.008 | 0.196 | 0.907 |
| ORF1a | nsp3 | 2201 | 3.229 | 0.745 | 0.907 |
| ORF1a | nsp3 | 1376 | 2.337 | 0.406 | 0.907 |
| ORF1a | nsp3 | 1924 | 0.979 | 0.294 | 0.907 |
| ORF1a | nsp5 | 3550 | 1.244 | 0.253 | 0.907 |
| Spike | Spike | 65 | 0.433 | 0.082 | 0.907 |
| ORF1a | nsp3 | 1979 | 0.903 | 0.183 | 0.907 |
| ORF1a | nsp3 | 1690 | 0.903 | 0.183 | 0.907 |
| ORF1a | nsp3 | 1930 | 0.903 | 0.183 | 0.907 |
| ORF1a | nsp3 | 1928 | 0.903 | 0.183 | 0.907 |
| ORF1b | nsp12 | 661 | 1.147 | 0.272 | 0.907 |
| ORF1a | nsp4 | 3006 | 1.019 | 0.211 | 0.907 |
| ORF1a | nsp4 | 3074 | 2.186 | 0.694 | 0.907 |
| Spike | Spike | 1094 | 1.652 | 0.484 | 0.907 |
| ORF1b | nsp15 | 2244 | 1.103 | 0.273 | 0.906 |
| N | N | 356 | 0.510 | 0.109 | 0.906 |
| ORF1b | nsp16 | 2654 | 1.360 | 0.200 | 0.906 |
| ORF1a | nsp4 | 3138 | 2.189 | 0.735 | 0.906 |
| N | N | 284 | 0.870 | 0.166 | 0.906 |
| N | N | 310 | 0.583 | 0.151 | 0.906 |
| N | N | 176 | 0.583 | 0.151 | 0.906 |
| N | N | 143 | 1.499 | 0.205 | 0.906 |
| N | N | 233 | 1.500 | 0.205 | 0.906 |
| N | N | 266 | 1.500 | 0.205 | 0.906 |
| ORF1b | nsp16 | 2445 | 1.356 | 0.201 | 0.906 |
| ORF1b | nsp16 | 2516 | 3.199 | 0.494 | 0.906 |
| N | N | 363 | 0.722 | 0.282 | 0.906 |
| ORF1a | nsp3 | 2354 | 0.936 | 0.206 | 0.906 |
| ORF1a | nsp3 | 2390 | 0.935 | 0.206 | 0.906 |
| ORF1a | nsp3 | 2350 | 0.935 | 0.206 | 0.906 |
| ORF1b | nsp16 | 2472 | 1.706 | 0.222 | 0.906 |
| ORF1b | nsp16 | 2503 | 1.706 | 0.222 | 0.906 |
| ORF1a | nsp3 | 2328 | 0.935 | 0.206 | 0.906 |
| ORF1a | nsp3 | 2223 | 0.935 | 0.206 | 0.906 |
| ORF1b | nsp13 | 1472 | 0.818 | 0.210 | 0.906 |
| ORF1a | nsp3 | 2213 | 0.833 | 0.190 | 0.905 |
| ORF1b | nsp13 | 1309 | 0.866 | 0.187 | 0.905 |
| ORF1b | nsp13 | 1456 | 0.866 | 0.187 | 0.905 |
| ORF1b | nsp14 | 1564 | 0.866 | 0.187 | 0.905 |
| ORF3a | ORF3a | 3 | 0.471 | 0.097 | 0.905 |
| ORF1b | nsp13 | 1402 | 0.865 | 0.187 | 0.905 |
| ORF1b | nsp13 | 1372 | 0.865 | 0.187 | 0.905 |
| ORF1a | nsp3 | 2594 | 1.247 | 0.343 | 0.905 |
| ORF1b | nsp16 | 2520 | 2.082 | 0.246 | 0.905 |
| ORF1b | nsp16 | 2611 | 2.082 | 0.246 | 0.905 |
| ORF1b | nsp16 | 2650 | 2.082 | 0.246 | 0.905 |
| Spike | Spike | 1112 | 3.336 | 0.623 | 0.905 |
| ORF1b | nsp16 | 2534 | 2.084 | 0.246 | 0.905 |
| ORF1a | nsp4 | 3206 | 2.009 | 0.261 | 0.905 |
| ORF1b | nsp13 | 1503 | 2.167 | 0.697 | 0.904 |
| Spike | Spike | 880 | 1.008 | 0.200 | 0.904 |
| Spike | Spike | 1059 | 1.008 | 0.200 | 0.904 |
| Spike | Spike | 908 | 1.008 | 0.200 | 0.904 |
| ORF1b | nsp13 | 1153 | 1.203 | 0.210 | 0.904 |
| ORF1b | nsp12 | 801 | 1.203 | 0.210 | 0.904 |
| Spike | Spike | 1077 | 1.008 | 0.200 | 0.904 |
| N | N | 1 | 2.255 | 0.046 | 0.904 |
| ORF1b | nsp13 | 1370 | 2.362 | 0.345 | 0.903 |
| ORF1b | nsp14 | 1886 | 3.097 | 0.384 | 0.903 |
| ORF1a | nsp1 | 158 | 2.296 | 0.397 | 0.903 |
| ORF1b | nsp12 | 78 | 2.752 | 0.662 | 0.903 |
| ORF1b | nsp14 | 1981 | 3.096 | 0.384 | 0.903 |
| ORF1a | nsp2 | 205 | 1.015 | 0.300 | 0.903 |
| ORF1b | nsp13 | 1125 | 3.405 | 0.327 | 0.903 |
| ORF1b | nsp13 | 1069 | 3.405 | 0.327 | 0.903 |
| ORF1b | nsp13 | 1062 | 3.405 | 0.327 | 0.903 |
| ORF1b | nsp12 | 840 | 3.405 | 0.327 | 0.903 |
| ORF1b | nsp13 | 999 | 3.404 | 0.327 | 0.903 |
| ORF7a | ORF7a | 32 | 3.310 | 0.525 | 0.903 |
| ORF1a | nsp1 | 79 | 0.933 | 0.193 | 0.903 |
| ORF1b | nsp13 | 1270 | 2.363 | 0.346 | 0.903 |
| ORF1a | nsp5 | 3311 | 2.280 | 0.671 | 0.903 |
| ORF1b | nsp12 | 689 | 3.804 | 0.347 | 0.902 |
| ORF1a | nsp6 | 3744 | 0.990 | 0.270 | 0.902 |
| ORF1b | nsp15 | 2335 | 11.513 | 1.214 | 0.902 |
| ORF1a | nsp2 | 808 | 1.150 | 0.244 | 0.902 |
| ORF1b | nsp13 | 1181 | 7.180 | 3.186 | 0.902 |
| ORF1b | nsp12 | 611 | 0.967 | 0.185 | 0.902 |
| ORF1a | nsp3 | 835 | 0.995 | 0.282 | 0.902 |
| ORF1b | nsp13 | 1225 | 0.865 | 0.192 | 0.901 |
| ORF1a | nsp3 | 1963 | 1.078 | 0.266 | 0.901 |
| ORF1a | nsp3 | 2276 | 1.294 | 0.482 | 0.901 |
| ORF1a | nsp1 | 40 | 1.574 | 0.387 | 0.901 |
| N | N | 87 | 0.430 | 0.090 | 0.901 |
| Spike | Spike | 223 | 0.617 | 0.135 | 0.901 |
| ORF1a | nsp5 | 3342 | 1.954 | 0.265 | 0.901 |
| Spike | Spike | 48 | 0.617 | 0.135 | 0.901 |
| Spike | Spike | 7 | 0.617 | 0.135 | 0.901 |
| ORF1a | nsp4 | 3219 | 1.954 | 0.265 | 0.901 |
| Spike | Spike | 387 | 0.617 | 0.135 | 0.901 |
| Spike | Spike | 241 | 0.618 | 0.135 | 0.901 |
| ORF1b | nsp14 | 1980 | 2.393 | 0.341 | 0.901 |
| ORF1a | nsp8 | 4014 | 6.591 | 0.782 | 0.901 |
| Spike | Spike | 171 | 0.398 | 0.077 | 0.901 |
| ORF1a | nsp5 | 3389 | 0.931 | 0.256 | 0.901 |
| ORF1a | nsp3 | 1250 | 1.426 | 0.256 | 0.901 |
| ORF1b | nsp12 | 833 | 1.201 | 0.216 | 0.901 |
| ORF1b | nsp13 | 950 | 1.201 | 0.216 | 0.901 |
| ORF1b | nsp13 | 1035 | 1.201 | 0.216 | 0.901 |
| Spike | Spike | 671 | 0.482 | 0.105 | 0.900 |
| Spike | Spike | 749 | 0.482 | 0.105 | 0.900 |
| ORF1b | nsp14 | 2021 | 1.044 | 0.197 | 0.900 |
| ORF1b | nsp14 | 2012 | 1.044 | 0.197 | 0.900 |

iv. Gene Level Selection Over Time

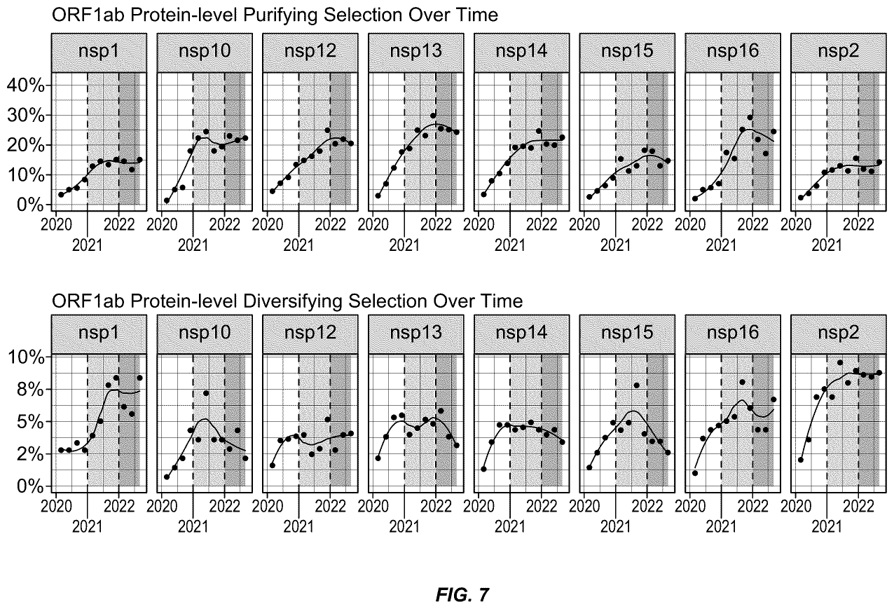

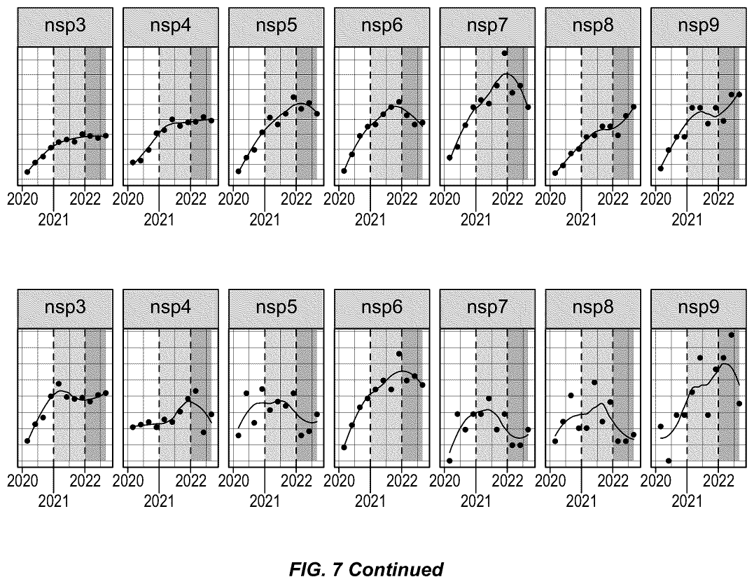

To better understand how levels of selection on SARS-CoV-2 genome have changed over time, the evolutionary analysis was repeated to generate a quarterly time series dataset. These timepoints span across three temporal windows, representative of three milestones of the recent pandemic, i.e.: 1) the pre-vaccine era (2020), 2) the post vaccine era (2021), and 3) the emergence of Omicron lineages (2022) (see Methods). At the gene level, a sharp shift in the selective forces acting on SARS-CoV-2 proteins was observed. While initially, many genes underwent an intensification of purifying selection over time (most notable for the Spike, M, N and ORF1b), this trend disappeared and often reverted with the widespread utilization of vaccination worldwide and emergence of Omicron lineages, respectively (FIG. 5C, FIG. 7). Conversely, diversification of the Spike-RBD continued to increase across the three time periods (Table 4), indicating intensification of the selective pressures promoting accumulation of mutations within this given region.

TABLE 4
RBD Sites Under Diversifying Selection over Time

| Spike Site | Pre-Vaccine | Post-Vaccine | Post-Omicron |
|---|---|---|---|
| 339 | Neutral | Neutral | Diversifying |
| 346 | Neutral | Neutral | Diversifying |
| 367 | Diversifying | Diversifying | Diversifying |
| 371 | Purifying | Neutral | Diversifying |
| 376 | Neutral | Neutral | Diversifying |
| 384 | Neutral | Diversifying | Diversifying |
| 405 | Neutral | Neutral | Diversifying |
| 408 | Neutral | Neutral | Diversifying |
| 414 | Neutral | Diversifying | Diversifying |
| 417 | Neutral | Diversifying | Diversifying |
| 439 | Diversifying | Diversifying | Neutral |
| 440 | Neutral | Diversifying | Diversifying |
| 445 | Neutral | Purifying | Diversifying |
| 446 | Diversifying | Diversifying | Diversifying |
| 449 | Neutral | Diversifying | Diversifying |
| 450 | Neutral | Neutral | Diversifying |
| 452 | Neutral | Diversifying | Diversifying |
| 460 | Neutral | Neutral | Diversifying |
| 477 | Diversifying | Diversifying | Diversifying |
| 478 | Neutral | Diversifying | Diversifying |
| 484 | Diversifying | Diversifying | Diversifying |
| 486 | Neutral | Neutral | Diversifying |
| 490 | Neutral | Diversifying | Diversifying |
| 493 | Neutral | Diversifying | Diversifying |
| 494 | Diversifying | Diversifying | Diversifying |
| 496 | Neutral | Neutral | Diversifying |
| 498 | Neutral | Diversifying | Diversifying |
| 501 | Diversifying | Diversifying | Diversifying |
| 505 | Neutral | Neutral | Diversifying |
| 522 | Diversifying | Diversifying | Diversifying |

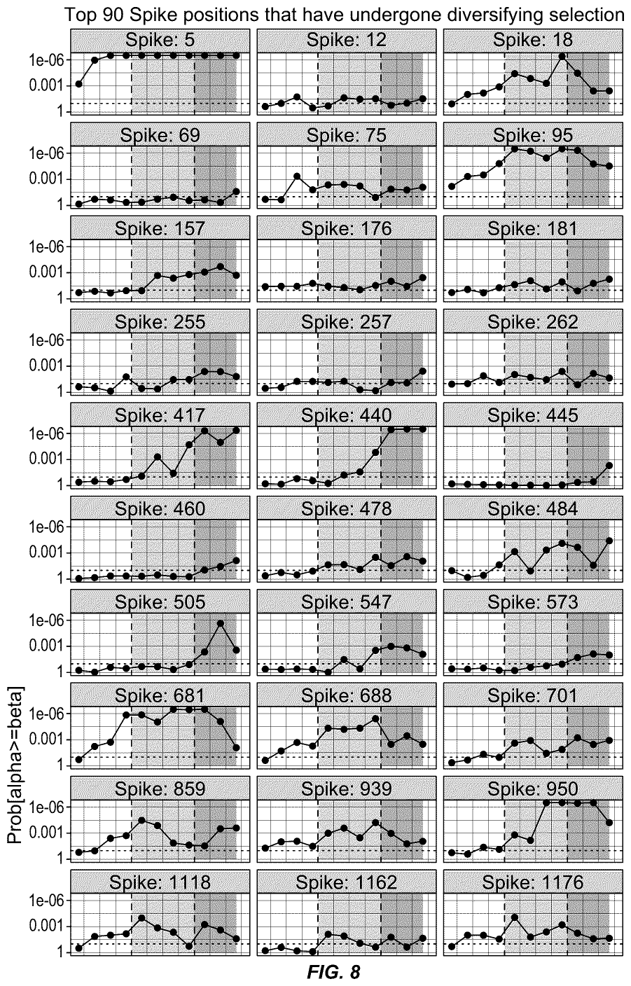

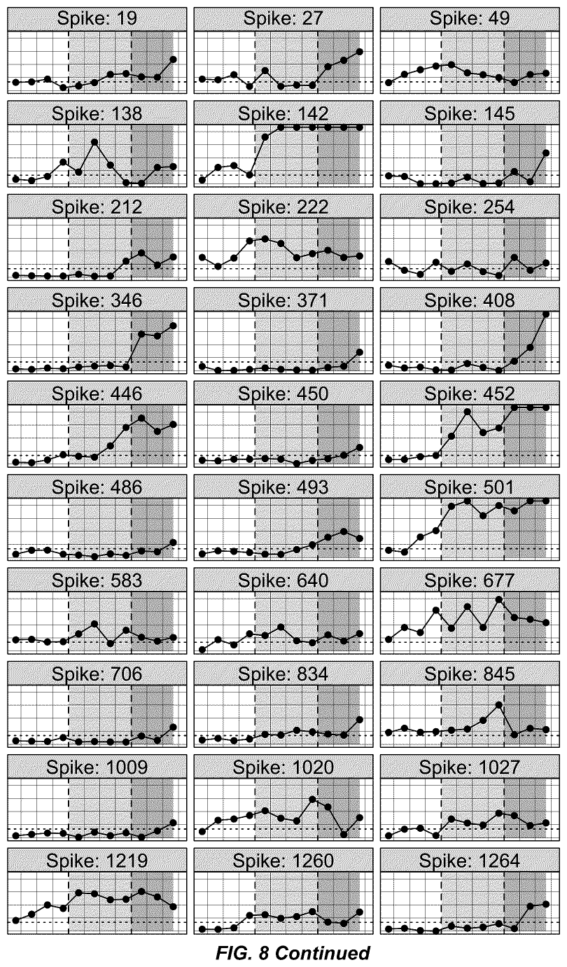

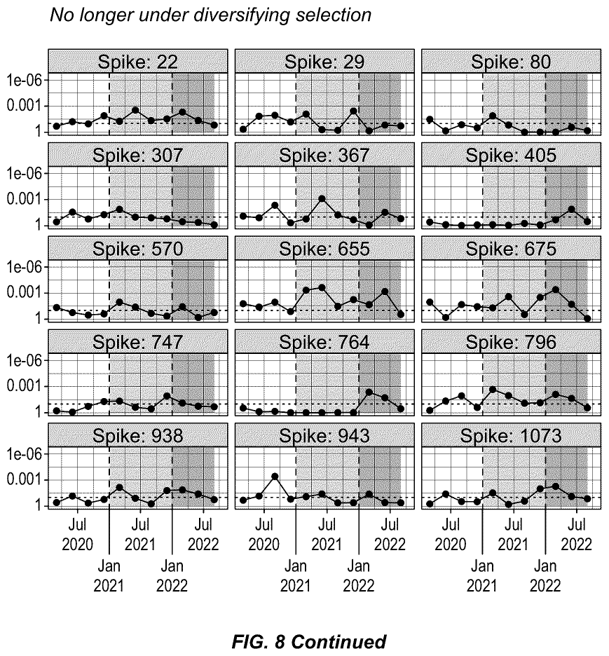

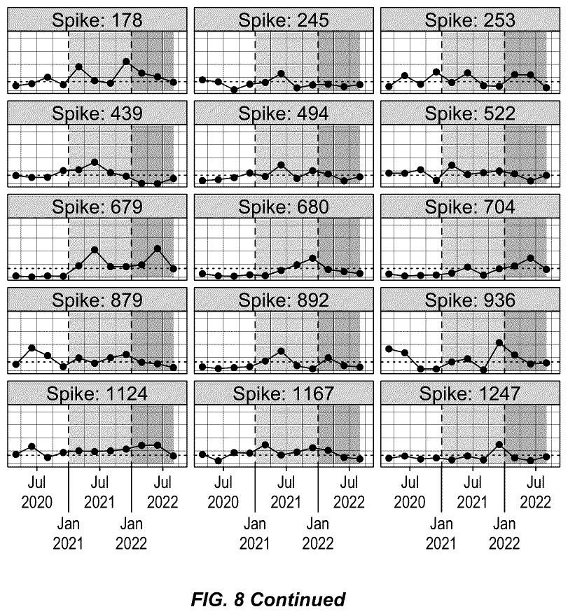

Notably, it was observed that the diversifying selection acting on variant sites that later would define Omicron lineages (417, 440, 446, 452, 501 and 484) increased at the onset of the post-vaccine era. These sites remained under strong diversifying selection even in the cases where the variants disappeared from circulation (417, 452, and 501) (FIG. 5D). This led to the transient fixation of these mutations in independent SARS-CoV-2 lineages and ultimately to their fixation by convergent evolution in Omicron lineages. Since the emergence of Omicron, new sites under diversifying selection have appeared (445, 449, and 460), indicating that the evolution of the virus is still ongoing and should be vigilantly monitored (FIG. 5D, FIG. 8). A comprehensive table detailing how selective pressures at each site genome-wide has changed over time is provided as a supplemental table (Table 5).

TABLE 5
Comprehensive list of sites under diversifying
and purifying selection over time.

| | | Pre- | Post- | Post- |
|---|---|---|---|---|
| Gene | Site | Vaccine | Vaccine | Omicron |
| E | 4 | P | P | P |
| E | 5 | D | N | N |
| E | 6 | P | P | P |
| E | 8 | N | N | P |
| E | 9 | D | D | D |
| E | 21 | D | D | D |
| E | 23 | P | P | P |
| E | 24 | N | D | N |
| E | 25 | P | P | P |
| E | 29 | N | P | P |
| E | 30 | N | D | N |
| E | 32 | N | P | P |
| E | 38 | N | P | P |
| E | 49 | N | D | D |
| E | 54 | P | P | N |
| E | 55 | N | D | D |
| E | 58 | N | D | D |
| E | 60 | P | P | P |
| E | 61 | N | N | D |
| E | 62 | D | D | D |
| E | 64 | N | P | P |
| E | 65 | P | P | P |
| E | 68 | D | D | D |
| E | 69 | N | D | N |
| E | 70 | N | D | N |
| E | 71 | D | D | D |
| E | 72 | D | D | N |
| E | 73 | D | D | D |
| E | 74 | P | P | P |
| E | 75 | P | N | P |
| M | 3 | D | D | D |
| M | 4 | N | N | P |
| M | 5 | N | P | P |
| M | 7 | N | D | D |
| M | 8 | P | P | P |
| M | 9 | P | P | P |
| M | 10 | P | N | P |
| M | 12 | P | P | P |
| M | 13 | N | P | P |
| M | 14 | N | N | P |
| M | 16 | N | P | N |
| M | 17 | D | D | D |
| M | 18 | N | P | P |
| M | 19 | N | N | D |
| M | 22 | N | P | P |
| M | 23 | D | D | D |
| M | 24 | P | P | N |
| M | 26 | P | P | P |
| M | 28 | P | P | P |
| M | 29 | N | D | D |
| M | 30 | P | P | P |
| M | 32 | N | P | P |
| M | 33 | N | P | P |
| M | 34 | N | N | D |
| M | 35 | P | P | P |
| M | 38 | N | N | P |
| M | 40 | N | D + P | D |
| M | 41 | P | P | P |
| M | 47 | N | N | P |
| M | 49 | N | P | N |
| M | 50 | N | N | P |
| M | 51 | N | P | N |
| M | 52 | N | D + P | N |
| M | 53 | P | P | P |
| M | 54 | P | P | P |
| M | 56 | P | P | P |
| M | 57 | N | P | P |
| M | 59 | P | P | P |
| M | 60 | N | P | P |
| M | 62 | N | P | N |
| M | 63 | D | D | D |
| M | 65 | N | P | P |
| M | 66 | P | P | P |
| M | 67 | N | P | N |
| M | 68 | N | P | N |
| M | 69 | N | D | D |
| M | 70 | D | D | D |
| M | 71 | P | P | P |
| M | 73 | P | N | N |
| M | 74 | D | N | N |
| M | 76 | P | N | N |
| M | 77 | P | P | P |
| M | 78 | D | D | D |
| M | 79 | P | P | P |
| M | 82 | N | D | D |
| M | 88 | P | P | P |
| M | 90 | N | P | P |
| M | 93 | P | P | P |
| M | 94 | N | D | N |
| M | 95 | N | P | N |
| M | 96 | N | P | N |
| M | 97 | P | N | N |
| M | 98 | P | D + P | N |
| M | 99 | P | P | P |
| M | 100 | P | P | P |
| M | 101 | N | N | P |
| M | 102 | P | P | P |
| M | 104 | P | P | P |
| M | 105 | N | P | N |
| M | 106 | P | P | P |
| M | 107 | D | N | N |
| M | 108 | N | P | P |
| M | 111 | N | P | P |
| M | 112 | P | P | P |
| M | 113 | P | P | P |
| M | 114 | P | P | P |
| M | 115 | N | N | P |
| M | 116 | N | P | P |
| M | 117 | P | P | P |
| M | 118 | P | P | P |
| M | 119 | N | P | P |
| M | 120 | P | P | P |
| M | 121 | P | P | P |
| M | 122 | N | P | N |
| M | 123 | N | P | P |
| M | 124 | P | P | P |
| M | 125 | D | D | D |
| M | 126 | P | N | N |
| M | 127 | N | P | P |
| M | 129 | P | P | P |
| M | 130 | P | P | P |
| M | 131 | N | N | P |
| M | 132 | P | P | P |
| M | 133 | N | P | P |
| M | 134 | P | P | P |
| M | 135 | P | P | P |
| M | 137 | N | P | P |
| M | 138 | P | P | P |
| M | 139 | P | P | P |
| M | 140 | P | P | P |
| M | 141 | N | P | P |
| M | 143 | P | P | P |
| M | 144 | N | N | P |
| M | 145 | P | P | P |
| M | 146 | N | P | P |
| M | 147 | N | P | P |
| M | 148 | N | P | P |
| M | 149 | N | P | N |
| M | 150 | P | P | P |
| M | 151 | N | P | N |
| M | 153 | N | P | N |
| M | 154 | N | P | P |
| M | 155 | D | D | N |
| M | 156 | N | P | P |
| M | 157 | N | N | P |
| M | 158 | N | P | P |
| M | 159 | N | P | N |
| M | 160 | P | P | P |
| M | 161 | P | P | P |
| M | 164 | P | P | P |
| M | 165 | N | P | P |
| M | 166 | N | P | N |
| M | 167 | N | N | P |
| M | 168 | N | N | P |
| M | 169 | N | N | P |
| M | 171 | N | P | N |
| M | 172 | P | P | P |
| M | 173 | N | N | D |
| M | 174 | N | P | N |
| M | 175 | P | P | P |
| M | 176 | N | P | N |
| M | 179 | P | P | P |
| M | 180 | N | N | P |
| M | 181 | P | P | P |
| M | 182 | N | P | P |
| M | 183 | N | P | P |
| M | 184 | P | P | P |
| M | 186 | N | P | P |
| M | 187 | P | P | P |
| M | 190 | P | P | P |
| M | 191 | N | N | P |
| M | 194 | N | P | N |
| M | 195 | P | P | P |
| M | 196 | P | P | P |
| M | 198 | N | P | N |
| M | 199 | N | N | P |
| M | 200 | N | P | N |
| M | 202 | P | P | P |
| M | 203 | P | P | P |
| M | 204 | P | P | P |
| M | 205 | P | P | P |
| M | 206 | N | P | N |
| M | 207 | P | P | P |
| M | 209 | D | D | D |
| M | 210 | N | P | P |
| M | 211 | N | P | P |
| M | 218 | N | P | P |
| M | 220 | N | P | N |
| N | 1 | P | P | P |
| N | 2 | N | D | N |
| N | 3 | D | D | D |
| N | 5 | N | D | N |
| N | 6 | N | P | N |
| N | 7 | D | D | D |
| N | 8 | D | D | N |
| N | 9 | D | D | D |
| N | 11 | D | N | D |
| N | 12 | D | D | N |
| N | 13 | D | D | D |
| N | 14 | D | D | N |
| N | 15 | N | P | N |
| N | 16 | P | P | N |
| N | 18 | D | D | D |
| N | 19 | N | P | P |
| N | 20 | N | N | P |
| N | 21 | N | P | P |
| N | 22 | D | D | D |
| N | 23 | N | P | P |
| N | 24 | D | D | D |
| N | 25 | D | D | D |
| N | 26 | N | N | D |
| N | 27 | N | P | N |
| N | 29 | N | N | D |
| N | 30 | D | P | P |
| N | 31 | P | P | P |
| N | 32 | D | D | D |
| N | 33 | D | D | D |
| N | 34 | D | D | N |
| N | 35 | P | P | P |
| N | 36 | N | P | N |
| N | 37 | N | P | P |
| N | 38 | N | P | N |
| N | 39 | N | N | P |
| N | 40 | D | D | D |
| N | 41 | P | P | P |
| N | 42 | P | P | P |
| N | 43 | N | P | P |
| N | 44 | N | N | P |
| N | 45 | N | P | P |
| N | 46 | N | D | D |
| N | 49 | N | D | D |
| N | 50 | P | N | P |
| N | 51 | P | P | P |
| N | 52 | P | P | P |
| N | 53 | P | P | P |
| N | 54 | P | P | P |
| N | 55 | D | D | D |
| N | 56 | N | P | P |
| N | 58 | P | N | N |
| N | 60 | P | P | P |
| N | 61 | N | P | P |
| N | 63 | D | D | D |
| N | 64 | P | P | P |
| N | 65 | P | P | P |
| N | 66 | P | P | P |
| N | 67 | D | D | D |
| N | 68 | N | P | N |
| N | 69 | N | P | N |
| N | 71 | P | N | P |
| N | 72 | D | D | D |
| N | 73 | P | P | P |
| N | 74 | N | P | P |
| N | 75 | N | P | P |
| N | 78 | P | P | P |
| N | 79 | N | N | P |
| N | 80 | D | N | N |
| N | 82 | N | P | P |
| N | 85 | P | P | P |
| N | 86 | P | P | P |
| N | 87 | N | P | P |
| N | 88 | N | P | P |
| N | 89 | P | P | N |
| N | 90 | D | D | D |
| N | 93 | N | N | P |
| N | 95 | N | D | N |
| N | 96 | N | D | N |
| N | 98 | P | P | P |
| N | 102 | N | P | P |
| N | 103 | D | D | N |
| N | 104 | P | P | P |
| N | 106 | P | P | P |
| N | 107 | P | P | P |
| N | 108 | P | P | P |
| N | 109 | P | P | P |
| N | 110 | P | P | P |
| N | 111 | P | P | P |
| N | 112 | P | P | P |
| N | 114 | P | P | N |
| N | 115 | P | P | P |
| N | 116 | P | P | P |
| N | 117 | P | P | P |
| N | 118 | P | N | P |
| N | 119 | D | D | D |
| N | 120 | N | D | D |
| N | 121 | P | P | P |
| N | 122 | N | D | D |
| N | 123 | N | P | P |
| N | 125 | D | N | D |
| N | 126 | P | P | P |
| N | 128 | N | P | P |
| N | 130 | N | P | N |
| N | 131 | N | P | P |
| N | 132 | P | P | P |
| N | 133 | N | P | P |
| N | 135 | D | D | D |
| N | 137 | N | P | N |
| N | 138 | N | P | N |
| N | 139 | D | D | D |
| N | 140 | N | P | N |
| N | 141 | D | D | D |
| N | 142 | D | N | N |
| N | 143 | P | P | P |
| N | 144 | D | N | D |
| N | 145 | D | N | P |
| N | 146 | P | P | P |
| N | 147 | N | N | D |
| N | 149 | P | P | P |
| N | 150 | P | P | P |
| N | 151 | D | D | D |
| N | 152 | D | D | D |
| N | 153 | P | P | P |
| N | 154 | N | P | P |
| N | 155 | D | D | D |
| N | 157 | P | P | P |
| N | 158 | P | P | P |
| N | 159 | P | P | P |
| N | 164 | D | N | P |
| N | 166 | N | D | D |
| N | 168 | D | D | D |
| N | 169 | N | N | P |
| N | 170 | N | P | P |
| N | 171 | N | P | N |
| N | 172 | P | P | P |
| N | 173 | P | P | P |
| N | 174 | P | P | P |
| N | 175 | P | P | P |
| N | 176 | N | P | P |
| N | 177 | P | P | P |
| N | 178 | N | P | N |
| N | 179 | D | D | D |
| N | 180 | D | N | N |
| N | 181 | N | N | P |
| N | 182 | D | D | D |
| N | 183 | D | D | N |
| N | 184 | N | P | P |
| N | 185 | D | D | N |
| N | 187 | D | N | N |
| N | 189 | N | N | D |
| N | 190 | D | D | D |
| N | 191 | N | P | P |
| N | 192 | P | P | P |
| N | 193 | D | D | D |
| N | 194 | N | D | D |
| N | 195 | D | P | D |
| N | 196 | N | D | N |
| N | 198 | D | N | N |
| N | 199 | D | N | N |
| N | 200 | D | D | D |
| N | 201 | D | D | N |
| N | 202 | D | D | D |
| N | 203 | P | P | P |
| N | 204 | D | D | D |
| N | 205 | D | D | D |
| N | 206 | D | D | D |
| N | 207 | D | D | D |
| N | 208 | D | D | D |
| N | 209 | D | D | D |
| N | 211 | D | D | D |
| N | 212 | D | D | D |
| N | 214 | P | P | N |
| N | 215 | D | D | D |
| N | 216 | N | P | N |
| N | 217 | D | D | D |
| N | 218 | N | D | D |
| N | 219 | N | D | N |
| N | 220 | D | D | D |
| N | 221 | P | N | N |
| N | 222 | P | P | P |
| N | 223 | P | P | P |
| N | 225 | P | P | P |
| N | 226 | N | P | P |
| N | 227 | P | P | P |
| N | 228 | P | P | P |
| N | 229 | D | D | D |
| N | 230 | N | D | D |
| N | 231 | N | N | P |
| N | 232 | D | D | N |
| N | 233 | N | P | P |
| N | 234 | D | D | N |
| N | 235 | N | D | N |
| N | 236 | D | D | D |
| N | 238 | D | D | D |
| N | 242 | N | P | D |
| N | 243 | D | D | D |
| N | 245 | N | D | D |
| N | 246 | N | P | P |
| N | 247 | D | N | D |
| N | 248 | P | P | N |
| N | 249 | N | P | P |
| N | 250 | N | P | P |
| N | 251 | D | D | D |
| N | 252 | D | D | N |
| N | 253 | N | P | P |
| N | 255 | N | P | P |
| N | 257 | P | P | P |
| N | 259 | P | P | P |
| N | 260 | P | N | N |
| N | 261 | P | P | P |
| N | 264 | N | P | N |
| N | 265 | D | D | D |
| N | 266 | N | P | P |
| N | 267 | N | N | D |
| N | 268 | P | P | P |
| N | 270 | N | D | N |
| N | 271 | D | D | D |
| N | 272 | P | N | N |
| N | 273 | N | D | N |
| N | 274 | P | P | P |
| N | 275 | P | P | P |
| N | 278 | N | P | P |
| N | 279 | D | N | N |
| N | 280 | N | P | P |
| N | 281 | N | N | D |
| N | 282 | P | P | P |
| N | 284 | P | P | P |
| N | 285 | P | P | P |
| N | 286 | P | P | P |
| N | 287 | P | P | P |
| N | 288 | N | N | P |
| N | 289 | D | N | N |
| N | 290 | N | P | N |
| N | 291 | P | P | P |
| N | 292 | P | P | P |
| N | 293 | N | P | P |
| N | 295 | P | N | N |
| N | 296 | D | D | D |
| N | 297 | N | D | N |
| N | 298 | P | P | P |
| N | 300 | N | D | D |
| N | 301 | P | P | P |
| N | 302 | P | P | P |
| N | 304 | P | P | P |
| N | 305 | N | P | P |
| N | 306 | N | D | N |
| N | 307 | P | P | P |
| N | 308 | D | N | N |
| N | 309 | P | P | P |
| N | 310 | N | P | P |
| N | 312 | P | P | P |
| N | 313 | P | P | P |
| N | 314 | P | P | P |
| N | 315 | P | P | P |
| N | 316 | P | P | P |
| N | 317 | P | N | N |
| N | 318 | P | P | P |
| N | 320 | P | N | N |
| N | 321 | N | N | P |
| N | 324 | N | N | P |
| N | 325 | D | D | D |
| N | 326 | D | D | D |
| N | 327 | P | P | P |
| N | 328 | N | P | P |
| N | 329 | P | P | P |
| N | 331 | P | P | P |
| N | 332 | N | P | P |
| N | 333 | P | P | P |
| N | 334 | D | D | D |
| N | 336 | D | N | P |
| N | 337 | P | P | P |
| N | 339 | N | P | P |
| N | 341 | P | P | P |
| N | 343 | N | P | N |
| N | 345 | N | D | N |
| N | 346 | P | P | P |
| N | 348 | D | N | N |
| N | 350 | N | N | D |
| N | 351 | N | N | P |
| N | 352 | P | P | P |
| N | 353 | P | P | P |
| N | 354 | N | P | P |
| N | 356 | N | P | P |
| N | 357 | P | N | N |
| N | 358 | P | P | P |
| N | 360 | P | P | P |
| N | 361 | N | P | N |
| N | 362 | D | D | D |
| N | 363 | P | P | P |
| N | 364 | D | D | D |
| N | 365 | D | D | D |
| N | 366 | D | D | D |
| N | 367 | P | P | P |
| N | 368 | D | D | N |
| N | 369 | N | N | P |
| N | 371 | D | N | N |
| N | 373 | D | D | D |
| N | 375 | N | N | P |
| N | 377 | D | D | D |
| N | 378 | D | D | D |
| N | 379 | D | D | D |
| N | 381 | N | P | N |
| N | 382 | N | P | N |
| N | 383 | N | N | P |
| N | 386 | N | N | P |
| N | 387 | D | N | N |
| N | 388 | N | N | P |
| N | 389 | D | N | N |
| N | 391 | D | D | D |
| N | 392 | P | P | P |
| N | 393 | D | D | D |
| N | 394 | N | P | N |
| N | 395 | P | N | N |
| N | 396 | N | P | P |
| N | 397 | N | P | N |
| N | 398 | D | D | D |
| N | 399 | D | D | N |
| N | 400 | P | P | P |
| N | 401 | D | D | D |
| N | 402 | D | D | N |
| N | 403 | P | P | P |
| N | 404 | P | P | P |
| N | 405 | N | P | P |
| N | 406 | N | P | N |
| N | 407 | P | P | P |
| N | 409 | N | P | N |
| N | 410 | P | P | P |
| N | 412 | N | P | P |
| N | 413 | D | D | D |
| N | 414 | D | D | D |
| N | 415 | P | P | P |
| N | 416 | P | P | P |
| N | 417 | N | D | D |
| N | 418 | N | D | D |
| N | 419 | N | P | P |
| ORF10 | 4 | D | D | D |
| ORF10 | 5 | N | N | D |
| ORF10 | 8 | D | D | D |
| ORF10 | 10 | D | D | D |
| ORF10 | 12 | N | N | P |
| ORF10 | 14 | N | N | D |
| ORF10 | 15 | N | N | D |
| ORF10 | 16 | N | D | N |
| ORF10 | 19 | P | P | P |
| ORF10 | 20 | N | P | N |
| ORF10 | 22 | D | D | N |
| ORF10 | 23 | D | D | D |
| ORF10 | 24 | D | D | D |
| ORF10 | 25 | D | P | P |
| ORF10 | 26 | N | P | P |
| ORF10 | 28 | D | D | N |
| ORF10 | 30 | D | D | D |
| ORF10 | 31 | D | D | D |
| ORF10 | 32 | N | P | P |
| ORF10 | 34 | N | P | N |
| ORF10 | 37 | D | D | D |
| ORF1a | 3 | P | N | P |
| ORF1a | 7 | N | D | D |
| ORF1a | 8 | N | D | N |
| ORF1a | 9 | N | N | P |
| ORF1a | 11 | N | P | P |
| ORF1a | 12 | N | P | N |
| ORF1a | 13 | N | P | N |
| ORF1a | 16 | P | P | P |
| ORF1a | 19 | N | P | P |
| ORF1a | 24 | N | P | N |
| ORF1a | 26 | P | P | P |
| ORF1a | 27 | P | P | P |
| ORF1a | 28 | N | D | D |
| ORF1a | 29 | N | N | D |
| ORF1a | 31 | N | P | P |
| ORF1a | 32 | P | P | P |
| ORF1a | 34 | N | P | N |
| ORF1a | 35 | P | P | P |
| ORF1a | 36 | N | P | N |
| ORF1a | 37 | D | D | N |
| ORF1a | 38 | N | N | P |
| ORF1a | 39 | N | P | P |
| ORF1a | 40 | P | N | P |
| ORF1a | 41 | N | N | P |
| ORF1a | 43 | N | D | N |
| ORF1a | 45 | D | N | N |
| ORF1a | 48 | N | D | N |
| ORF1a | 49 | N | D | D |
| ORF1a | 54 | N | P | P |
| ORF1a | 58 | N | N | P |
| ORF1a | 60 | N | P | N |
| ORF1a | 62 | N | D | N |
| ORF1a | 66 | P | P | N |
| ORF1a | 67 | P | P | N |
| ORF1a | 69 | N | P | P |
| ORF1a | 70 | N | P | P |
| ORF1a | 71 | N | N | P |
| ORF1a | 72 | N | P | P |
| ORF1a | 74 | P | P | P |
| ORF1a | 75 | D | D | D |
| ORF1a | 79 | N | N | P |
| ORF1a | 81 | N | N | D |
| ORF1a | 82 | N | N | D |
| ORF1a | 84 | P | P | P |
| ORF1a | 86 | N | D | N |
| ORF1a | 87 | D | D | D |
| ORF1a | 88 | P | P | P |
| ORF1a | 89 | P | P | P |
| ORF1a | 92 | D | D | N |
| ORF1a | 94 | N | D | D |
| ORF1a | 96 | N | N | P |
| ORF1a | 97 | N | P | P |
| ORF1a | 99 | N | N | D |
| ORF1a | 100 | N | D | D |
| ORF1a | 101 | N | P | N |
| ORF1a | 106 | P | P | P |
| ORF1a | 107 | N | N | D |
| ORF1a | 108 | N | P | N |
| ORF1a | 109 | P | P | P |
| ORF1a | 110 | D | D | D |
| ORF1a | 111 | P | N | N |
| ORF1a | 112 | N | N | D |
| ORF1a | 113 | N | P | N |
| ORF1a | 115 | N | P | P |
| ORF1a | 116 | N | P | P |
| ORF1a | 120 | D | D | D |
| ORF1a | 124 | D | D | D |
| ORF1a | 126 | N | P | P |
| ORF1a | 130 | P | P | N |
| ORF1a | 131 | N | N | D |
| ORF1a | 133 | N | P | P |
| ORF1a | 135 | N | N | D |
| ORF1a | 136 | P | P | P |
| ORF1a | 137 | N | D | N |
| ORF1a | 138 | D | D | D |
| ORF1a | 139 | D | N | D |
| ORF1a | 140 | P | P | P |
| ORF1a | 142 | N | P | P |
| ORF1a | 144 | N | D | D |
| ORF1a | 146 | N | P | N |
| ORF1a | 147 | D | D | N |
| ORF1a | 149 | N | P | N |
| ORF1a | 150 | N | D | N |
| ORF1a | 151 | N | P | N |
| ORF1a | 158 | N | N | P |
| ORF1a | 160 | P | P | P |
| ORF1a | 162 | N | N | P |
| ORF1a | 166 | D | D | D |
| ORF1a | 169 | P | P | P |
| ORF1a | 170 | N | D | N |
| ORF1a | 171 | N | P | N |
| ORF1a | 172 | N | P | P |
| ORF1a | 175 | N | P | N |
| ORF1a | 176 | N | N | P |
| ORF1a | 178 | N | P | N |
| ORF1a | 179 | N | N | P |
| ORF1a | 180 | N | P | P |
| ORF1a | 181 | N | P | N |
| ORF1a | 182 | D | N | N |
| ORF1a | 184 | D | D | D |
| ORF1a | 186 | P | P | P |
| ORF1a | 187 | N | P | P |
| ORF1a | 188 | P | P | P |
| ORF1a | 189 | P | P | P |
| ORF1a | 190 | P | P | P |
| ORF1a | 191 | N | N | P |
| ORF1a | 193 | D | D | D |
| ORF1a | 194 | D | N | N |
| ORF1a | 195 | N | P | P |
| ORF1a | 196 | P | P | P |
| ORF1a | 197 | D | N | N |
| ORF1a | 198 | P | N | N |
| ORF1a | 199 | N | N | P |
| ORF1a | 200 | N | N | P |
| ORF1a | 202 | N | N | P |
| ORF1a | 203 | D | D | N |
| ORF1a | 204 | D | D | D |
| ORF1a | 205 | P | P | P |
| ORF1a | 206 | N | D | N |
| ORF1a | 207 | D | D | D |
| ORF1a | 209 | N | N | D |
| ORF1a | 210 | P | P | P |
| ORF1a | 211 | N | D | N |
| ORF1a | 213 | P | N | N |
| ORF1a | 214 | N | D | N |
| ORF1a | 215 | N | P | N |
| ORF1a | 216 | P | P | P |
| ORF1a | 218 | N | P | N |
| ORF1a | 219 | P | P | P |
| ORF1a | 220 | N | N | P |
| ORF1a | 222 | D | D | D |
| ORF1a | 223 | N | P | P |
| ORF1a | 224 | D | D | D |
| ORF1a | 225 | P | N | P |
| ORF1a | 226 | N | D | D |
| ORF1a | 227 | N | D | D |
| ORF1a | 228 | P | P | P |
| ORF1a | 229 | N | D + P | N |
| ORF1a | 230 | N | N | P |
| ORF1a | 231 | P | P | P |
| ORF1a | 232 | N | N | D |
| ORF1a | 234 | N | P | N |
| ORF1a | 235 | P | P | N |
| ORF1a | 236 | N | P | P |
| ORF1a | 237 | N | P | N |
| ORF1a | 242 | P | N | P |
| ORF1a | 243 | N | P | N |
| ORF1a | 244 | N | P | P |
| ORF1a | 246 | P | N | N |
| ORF1a | 249 | N | P | N |
| ORF1a | 250 | N | N | P |
| ORF1a | 256 | N | P | N |
| ORF1a | 258 | N | P | N |
| ORF1a | 260 | N | P | N |
| ORF1a | 264 | P | P | N |
| ORF1a | 265 | N | D | N |
| ORF1a | 266 | P | P | P |
| ORF1a | 268 | N | D | D |
| ORF1a | 269 | N | P | N |
| ORF1a | 270 | N | P | N |
| ORF1a | 271 | N | D | D |
| ORF1a | 273 | N | P | N |
| ORF1a | 274 | N | P | N |
| ORF1a | 279 | N | P | P |
| ORF1a | 281 | N | P | N |
| ORF1a | 283 | N | D | N |
| ORF1a | 292 | N | D | N |
| ORF1a | 293 | N | N | D |
| ORF1a | 294 | D | N | N |
| ORF1a | 295 | N | D | N |
| ORF1a | 300 | D | N | D |
| ORF1a | 302 | D | D | D |
| ORF1a | 304 | N | P | P |
| ORF1a | 305 | N | P | N |
| ORF1a | 306 | D | D | D |
| ORF1a | 307 | P | P | P |
| ORF1a | 308 | N | P | P |
| ORF1a | 309 | D | D | D |
| ORF1a | 310 | N | P | N |
| ORF1a | 312 | N | P | N |
| ORF1a | 313 | N | P | N |
| ORF1a | 314 | N | N | D |
| ORF1a | 318 | D | D | D |
| ORF1a | 320 | N | D | N |
| ORF1a | 322 | N | D | D |
| ORF1a | 324 | D | D | N |
| ORF1a | 327 | N | D | N |
| ORF1a | 328 | N | P | N |
| ORF1a | 330 | P | P | N |
| ORF1a | 332 | N | D | N |
| ORF1a | 333 | P | P | N |
| ORF1a | 334 | N | P | N |
| ORF1a | 335 | D | D | D |
| ORF1a | 337 | N | D | D |
| ORF1a | 339 | N | D | N |
| ORF1a | 340 | N | N | D |
| ORF1a | 341 | P | P | P |
| ORF1a | 346 | D | D | D |
| ORF1a | 347 | N | D | D |
| ORF1a | 348 | N | N | D |
| ORF1a | 349 | N | P | N |
| ORF1a | 350 | D | D | D |
| ORF1a | 352 | N | N | D |
| ORF1a | 354 | D | N | N |
| ORF1a | 359 | P | P | P |
| ORF1a | 362 | N | N | D |
| ORF1a | 365 | N | N | D |
| ORF1a | 366 | D | N | N |
| ORF1a | 370 | N | P | P |
| ORF1a | 371 | N | D | D |
| ORF1a | 372 | N | D | D |
| ORF1a | 374 | P | D | N |
| ORF1a | 376 | D | D | N |
| ORF1a | 378 | P | P | N |
| ORF1a | 380 | N | D | N |
| ORF1a | 381 | P | N | N |
| ORF1a | 382 | N | N | P |
| ORF1a | 385 | N | P | P |
| ORF1a | 386 | N | P | N |
| ORF1a | 387 | P | P | P |
| ORF1a | 388 | D | D | D |
| ORF1a | 389 | D | N | N |
| ORF1a | 391 | N | N | D |
| ORF1a | 395 | N | D | N |
| ORF1a | 398 | D | D | D |
| ORF1a | 400 | D | D | D |
| ORF1a | 402 | P | P | P |
| ORF1a | 403 | D | D | D |
| ORF1a | 405 | D | N | D |
| ORF1a | 408 | N | N | P |
| ORF1a | 410 | P | P | P |
| ORF1a | 411 | N | P | P |
| ORF1a | 414 | N | P | P |
| ORF1a | 415 | N | N | D |
| ORF1a | 416 | N | P | P |
| ORF1a | 417 | D | D | D |
| ORF1a | 418 | N | P | P |
| ORF1a | 420 | N | D | N |
| ORF1a | 421 | P | P | P |
| ORF1a | 424 | P | N | P |
| ORF1a | 425 | N | P | N |
| ORF1a | 427 | N | D | N |
| ORF1a | 428 | N | D | N |
| ORF1a | 431 | N | N | D |
| ORF1a | 434 | N | P | N |
| ORF1a | 435 | N | N | P |
| ORF1a | 436 | N | N | P |
| ORF1a | 439 | D | N | P |
| ORF1a | 440 | N | N | P |
| ORF1a | 441 | N | P | P |
| ORF1a | 442 | N | D | D |
| ORF1a | 443 | N | D + P | P |
| ORF1a | 445 | D | D | D |
| ORF1a | 446 | P | N | D |
| ORF1a | 447 | P | P | P |
| ORF1a | 448 | N | N | P |
| ORF1a | 450 | N | N | D |
| ORF1a | 451 | N | D | N |
| ORF1a | 454 | P | P | P |
| ORF1a | 455 | N | N | D |
| ORF1a | 460 | D | D | N |
| ORF1a | 465 | N | D | D |
| ORF1a | 466 | D | D | N |
| ORF1a | 469 | N | D | N |
| ORF1a | 473 | P | P | P |
| ORF1a | 474 | N | N | D |
| ORF1a | 478 | N | P | P |
| ORF1a | 480 | N | N | P |
| ORF1a | 481 | N | P | N |
| ORF1a | 482 | N | N | D |
| ORF1a | 483 | N | P | N |
| ORF1a | 486 | D | D | D |
| ORF1a | 488 | N | P | P |
| ORF1a | 491 | P | P | P |
| ORF1a | 492 | N | P | P |
| ORF1a | 493 | D | N | D |
| ORF1a | 494 | P | N | P |
| ORF1a | 498 | D | D | D |
| ORF1a | 502 | P | N | N |
| ORF1a | 503 | N | N | P |
| ORF1a | 504 | N | P | P |
| ORF1a | 505 | P | N | N |
| ORF1a | 506 | N | N | P |
| ORF1a | 510 | N | N | P |
| ORF1a | 511 | N | P | N |
| ORF1a | 514 | N | N | P |
| ORF1a | 516 | N | D | D |
| ORF1a | 518 | N | P | P |
| ORF1a | 519 | D | D | D |
| ORF1a | 526 | N | N | D |
| ORF1a | 527 | P | P | N |
| ORF1a | 530 | P | N | N |
| ORF1a | 531 | N | D | N |
| ORF1a | 535 | P | P | P |
| ORF1a | 537 | P | P | P |
| ORF1a | 538 | D | N | N |
| ORF1a | 539 | N | N | P |
| ORF1a | 540 | D | D | D |
| ORF1a | 541 | D | D | N |
| ORF1a | 542 | D | D | N |
| ORF1a | 545 | N | P | P |
| ORF1a | 546 | N | P | N |
| ORF1a | 548 | N | P | P |
| ORF1a | 549 | P | P | P |
| ORF1a | 550 | D | D | D |
| ORF1a | 552 | D | N | D |
| ORF1a | 555 | N | D | D |
| ORF1a | 556 | N | N | D |
| ORF1a | 557 | N | P | N |
| ORF1a | 558 | N | N | D |
| ORF1a | 559 | N | P | N |
| ORF1a | 560 | D | D | D |
| ORF1a | 561 | D | D | D |
| ORF1a | 564 | D | N | N |
| ORF1a | 565 | P | P | P |
| ORF1a | 566 | N | D | D |
| ORF1a | 568 | D | D | D |
| ORF1a | 570 | N | P | P |
| ORF1a | 571 | D | N | D |
| ORF1a | 574 | P | P | P |
| ORF1a | 575 | N | D | D |
| ORF1a | 576 | N | N | D |
| ORF1a | 578 | N | P | N |
| ORF1a | 580 | D | D | N |
| ORF1a | 583 | N | N | D |
| ORF1a | 586 | N | P | N |
| ORF1a | 587 | N | P | P |
| ORF1a | 588 | N | P | P |
| ORF1a | 590 | D | D | D |
| ORF1a | 591 | D | D | D |
| ORF1a | 592 | D | D | D |
| ORF1a | 594 | N | P | P |
| ORF1a | 595 | N | P | P |
| ORF1a | 596 | N | P | N |
| ORF1a | 599 | P | P | P |
| ORF1a | 600 | N | N | P |
| ORF1a | 603 | N | P | N |
| ORF1a | 605 | N | P | P |
| ORF1a | 606 | N | P | P |
| ORF1a | 607 | D | D | N |
| ORF1a | 609 | D | D | D |
| ORF1a | 610 | P | N | D |
| ORF1a | 613 | N | N | P |
| ORF1a | 614 | D | D | D |
| ORF1a | 615 | P | P | P |
| ORF1a | 616 | P | P | P |
| ORF1a | 618 | N | N | P |
| ORF1a | 620 | N | N | P |
| ORF1a | 621 | D | N | N |
| ORF1a | 623 | N | P | N |
| ORF1a | 624 | P | P | N |
| ORF1a | 626 | P | P | P |
| ORF1a | 627 | D | D | D |
| ORF1a | 628 | N | D | N |
| ORF1a | 629 | N | D | D |
| ORF1a | 632 | N | P | P |
| ORF1a | 633 | N | N | D |
| ORF1a | 634 | N | D | D |
| ORF1a | 636 | P | N | N |
| ORF1a | 637 | N | P | N |
| ORF1a | 639 | N | P | N |
| ORF1a | 642 | D | D | D |
| ORF1a | 644 | P | P | P |
| ORF1a | 645 | D | N | N |
| ORF1a | 651 | N | N | P |
| ORF1a | 652 | N | P | N |
| ORF1a | 654 | N | N | P |
| ORF1a | 655 | N | N | D |
| ORF1a | 656 | N | D | D |
| ORF1a | 660 | N | P | P |
| ORF1a | 661 | N | D | D |
| ORF1a | 662 | N | P | N |
| ORF1a | 665 | D | D | D |
| ORF1a | 669 | N | N | D |
| ORF1a | 672 | P | P | N |
| ORF1a | 675 | N | P | P |
| ORF1a | 676 | D | D | D |
| ORF1a | 677 | N | P | P |
| ORF1a | 678 | N | P | N |
| ORF1a | 680 | D | D | D |
| ORF1a | 681 | D | D | D |
| ORF1a | 683 | N | N | P |
| ORF1a | 687 | N | D | N |
| ORF1a | 690 | D | D | D |
| ORF1a | 693 | N | D | D |
| ORF1a | 697 | P | P | D |
| ORF1a | 701 | P | P | N |
| ORF1a | 702 | N | D | N |
| ORF1a | 708 | D | D | D |
| ORF1a | 710 | P | P | N |
| ORF1a | 711 | P | P | P |
| ORF1a | 712 | N | P | N |
| ORF1a | 713 | N | P | P |
| ORF1a | 714 | N | P | P |
| ORF1a | 715 | N | P | P |
| ORF1a | 717 | P | P | P |
| ORF1a | 722 | P | N | N |
| ORF1a | 725 | N | P | N |
| ORF1a | 727 | D | D | D |
| ORF1a | 728 | D | D | D |
| ORF1a | 729 | P | P | P |
| ORF1a | 730 | P | P | P |
| ORF1a | 733 | N | P | P |
| ORF1a | 735 | N | P | P |
| ORF1a | 736 | P | P | P |
| ORF1a | 740 | P | P | P |
| ORF1a | 741 | P | P | P |
| ORF1a | 742 | P | P | P |
| ORF1a | 744 | P | P | P |
| ORF1a | 747 | P | N | N |
| ORF1a | 748 | P | P | P |
| ORF1a | 749 | N | P | N |
| ORF1a | 751 | N | P | N |
| ORF1a | 753 | N | D | D |
| ORF1a | 754 | P | D | D |
| ORF1a | 755 | D | P | N |
| ORF1a | 756 | N | P | N |
| ORF1a | 757 | P | P | P |
| ORF1a | 761 | N | P | N |
| ORF1a | 762 | D | N | N |
| ORF1a | 765 | N | P | P |
| ORF1a | 766 | P | N | N |
| ORF1a | 768 | N | N | D |
| ORF1a | 769 | N | D | N |
| ORF1a | 770 | P | N | N |
| ORF1a | 775 | P | N | N |
| ORF1a | 777 | P | P | P |
| ORF1a | 778 | P | P | N |
| ORF1a | 781 | N | N | D |
| ORF1a | 782 | N | P | P |
| ORF1a | 786 | N | P | P |
| ORF1a | 787 | N | P | P |
| ORF1a | 790 | N | P | N |
| ORF1a | 791 | P | P | P |
| ORF1a | 792 | N | N | D |
| ORF1a | 793 | P | P | P |
| ORF1a | 794 | P | P | P |
| ORF1a | 795 | D | D | N |
| ORF1a | 796 | N | P | P |
| ORF1a | 798 | D | N | N |
| ORF1a | 799 | N | P | N |
| ORF1a | 802 | N | N | P |
| ORF1a | 804 | D | D | D |
| ORF1a | 808 | N | N | P |
| ORF1a | 809 | P | P | P |
| ORF1a | 810 | P | P | P |
| ORF1a | 811 | P | P | P |
| ORF1a | 813 | N | P | P |
| ORF1a | 814 | D | D | N |
| ORF1a | 815 | P | P | P |
| ORF1a | 816 | P | N | N |
| ORF1a | 817 | N | P | P |
| ORF1a | 819 | N | N | P |
| ORF1a | 820 | N | N | P |
| ORF1a | 822 | P | P | N |
| ORF1a | 823 | P | N | N |
| ORF1a | 825 | N | N | P |
| ORF1a | 828 | N | D | P |
| ORF1a | 830 | P | P | P |
| ORF1a | 833 | P | P | P |
| ORF1a | 835 | N | N | P |
| ORF1a | 836 | N | P | P |
| ORF1a | 839 | P | P | P |
| ORF1a | 841 | N | N | P |
| ORF1a | 842 | N | N | D |
| ORF1a | 843 | N | N | P |
| ORF1a | 845 | N | P | N |
| ORF1a | 846 | N | P | P |
| ORF1a | 848 | D | N | P |
| ORF1a | 850 | N | P | P |
| ORF1a | 854 | N | P | N |
| ORF1a | 856 | N | D | D |
| ORF1a | 857 | P | P | P |
| ORF1a | 859 | N | D | D |
| ORF1a | 860 | P | P | P |
| ORF1a | 861 | N | P | N |
| ORF1a | 863 | N | P | P |
| ORF1a | 864 | N | N | P |
| ORF1a | 866 | N | P | N |
| ORF1a | 868 | P | P | N |
| ORF1a | 869 | N | D | D |
| ORF1a | 871 | N | P | N |
| ORF1a | 872 | N | N | D |
| ORF1a | 875 | N | P | P |
| ORF1a | 877 | N | N | D |
| ORF1a | 879 | N | P | P |
| ORF1a | 882 | D | D | D |
| ORF1a | 885 | N | N | D |
| ORF1a | 886 | N | P | P |
| ORF1a | 888 | N | N | P |
| ORF1a | 890 | N | N | P |
| ORF1a | 891 | N | D | D |
| ORF1a | 892 | D | D | D |
| ORF1a | 893 | N | P | N |
| ORF1a | 894 | D | N | N |
| ORF1a | 897 | N | N | P |
| ORF1a | 898 | N | P | N |
| ORF1a | 903 | D | D | D |
| ORF1a | 904 | P | N | P |
| ORF1a | 905 | N | D | N |
| ORF1a | 908 | N | N | P |
| ORF1a | 913 | D | D | D |
| ORF1a | 914 | N | P | P |
| ORF1a | 916 | P | P | P |
| ORF1a | 917 | N | N | P |
| ORF1a | 919 | N | D | N |
| ORF1a | 922 | N | P | N |
| ORF1a | 923 | N | P | P |
| ORF1a | 924 | P | P | P |
| ORF1a | 925 | N | P | N |
| ORF1a | 927 | N | D | P |
| ORF1a | 928 | P | N | N |
| ORF1a | 929 | N | P | P |
| ORF1a | 930 | D | D | N |
| ORF1a | 932 | N | P | N |
| ORF1a | 934 | N | N | D |
| ORF1a | 935 | N | D | D |
| ORF1a | 936 | N | P | N |
| ORF1a | 940 | D | N | D |
| ORF1a | 942 | N | D | N |
| ORF1a | 944 | D | D | D |
| ORF1a | 950 | D | N | N |
| ORF1a | 951 | D | D | D |
| ORF1a | 952 | D | N | N |
| ORF1a | 953 | N | D | D |
| ORF1a | 956 | D | N | N |
| ORF1a | 959 | D | D | D |
| ORF1a | 960 | N | P | P |
| ORF1a | 961 | N | P | N |
| ORF1a | 962 | N | P | N |
| ORF1a | 963 | D | D | D |
| ORF1a | 964 | N | D | N |
| ORF1a | 966 | P | N | P |
| ORF1a | 967 | N | D | D |
| ORF1a | 971 | D | D | D |
| ORF1a | 973 | N | P | N |
| ORF1a | 975 | P | P | N |
| ORF1a | 981 | N | D | N |
| ORF1a | 983 | D | N | D |
| ORF1a | 985 | D | D | D |
| ORF1a | 987 | N | P | N |
| ORF1a | 989 | D | D | D |
| ORF1a | 992 | N | D | N |
| ORF1a | 993 | D | D | D |
| ORF1a | 995 | D | D | D |
| ORF1a | 996 | D | D | D |
| ORF1a | 997 | P | P | N |
| ORF1a | 998 | D | D | D |
| ORF1a | 999 | N | D | D |
| ORF1a | 1000 | D | D | D |
| ORF1a | 1001 | D | D | D |
| ORF1a | 1007 | D | N | N |
| ORF1a | 1008 | P | N | N |
| ORF1a | 1013 | D | D | D |
| ORF1a | 1018 | N | N | D |
| ORF1a | 1020 | N | D | N |
| ORF1a | 1021 | D | D | D |
| ORF1a | 1022 | N | D | N |
| ORF1a | 1024 | N | D + P | N |
| ORF1a | 1025 | P | P | P |
| ORF1a | 1026 | N | D | N |
| ORF1a | 1031 | N | N | P |
| ORF1a | 1036 | D | D | D |
| ORF1a | 1038 | N | P | P |
| ORF1a | 1043 | N | N | D |
| ORF1a | 1046 | P | P | P |
| ORF1a | 1047 | D | N | D |
| ORF1a | 1049 | D | D | D |
| ORF1a | 1051 | N | P | N |
| ORF1a | 1054 | D | N | N |
| ORF1a | 1055 | D | D | D |
| ORF1a | 1056 | D | D | N |
| ORF1a | 1060 | N | P | N |
| ORF1a | 1061 | N | N | D |
| ORF1a | 1070 | P | N | N |
| ORF1a | 1072 | P | P | P |
| ORF1a | 1073 | D | N | D |
| ORF1a | 1074 | N | D | N |
| ORF1a | 1076 | N | N | P |
| ORF1a | 1077 | N | N | P |
| ORF1a | 1079 | N | N | P |
| ORF1a | 1084 | P | P | P |
| ORF1a | 1086 | N | P | P |
| ORF1a | 1087 | N | P | P |
| ORF1a | 1093 | D | D | D |
| ORF1a | 1094 | P | P | N |
| ORF1a | 1097 | N | N | P |
| ORF1a | 1099 | N | P | N |
| ORF1a | 1100 | D | N | N |
| ORF1a | 1102 | D | D | D |
| ORF1a | 1103 | N | P | P |
| ORF1a | 1108 | N | D | D |
| ORF1a | 1109 | P | P | P |
| ORF1a | 1111 | P | N | N |
| ORF1a | 1112 | P | N | N |
| ORF1a | 1113 | D | D | D |
| ORF1a | 1118 | P | P | P |
| ORF1a | 1119 | N | D | D |
| ORF1a | 1120 | P | P | P |
| ORF1a | 1123 | P | N | N |
| ORF1a | 1125 | D | D | D |
| ORF1a | 1130 | D | D | D |
| ORF1a | 1131 | D | N | N |
| ORF1a | 1135 | N | N | P |
| ORF1a | 1137 | N | N | D |
| ORF1a | 1138 | N | P | N |
| ORF1a | 1139 | P | N | P |
| ORF1a | 1142 | D | D | N |
| ORF1a | 1143 | D | D | D |
| ORF1a | 1144 | P | P | P |
| ORF1a | 1147 | P | N | P |
| ORF1a | 1148 | N | N | P |
| ORF1a | 1151 | N | D | D |
| ORF1a | 1155 | D | D | D |
| ORF1a | 1156 | P | P | P |
| ORF1a | 1157 | P | P | P |
| ORF1a | 1158 | D | D | D |
| ORF1a | 1160 | D | D | D |
| ORF1a | 1161 | N | N | P |
| ORF1a | 1166 | N | P | N |
| ORF1a | 1167 | P | P | N |
| ORF1a | 1168 | D | D | D |
| ORF1a | 1169 | N | N | P |
| ORF1a | 1170 | D | D | D |
| ORF1a | 1173 | P | P | P |
| ORF1a | 1174 | P | P | P |
| ORF1a | 1176 | N | N | D |
| ORF1a | 1178 | N | N | P |
| ORF1a | 1182 | P | D + P | N |
| ORF1a | 1184 | P | P | P |
| ORF1a | 1188 | D | D | D |
| ORF1a | 1189 | N | N | D |
| ORF1a | 1190 | N | P | N |
| ORF1a | 1191 | N | N | D + P |
| ORF1a | 1192 | D | D | N |
| ORF1a | 1196 | N | P | N |
| ORF1a | 1197 | P | N | N |
| ORF1a | 1198 | D | N | N |
| ORF1a | 1202 | D | D | D |
| ORF1a | 1203 | N | P | P |
| ORF1a | 1205 | P | N | P |
| ORF1a | 1206 | N | N | D |
| ORF1a | 1207 | D | D | D |
| ORF1a | 1208 | N | N | P |
| ORF1a | 1211 | N | D | D |
| ORF1a | 1216 | N | D | N |
| ORF1a | 1217 | P | N | N |
| ORF1a | 1220 | N | D | D |
| ORF1a | 1221 | N | P | N |
| ORF1a | 1222 | P | N | N |
| ORF1a | 1225 | P | N | N |
| ORF1a | 1226 | N | N | P |
| ORF1a | 1227 | N | N | D |
| ORF1a | 1228 | N | D | D |
| ORF1a | 1230 | D | D | D |
| ORF1a | 1232 | N | N | P |
| ORF1a | 1235 | N | P | N |
| ORF1a | 1237 | P | N | N |
| ORF1a | 1242 | D | D | N |
| ORF1a | 1243 | P | P | P |
| ORF1a | 1244 | N | N | P |
| ORF1a | 1246 | D | D | N |
| ORF1a | 1248 | N | P | N |
| ORF1a | 1250 | N | P | P |
| ORF1a | 1252 | N | P | P |
| ORF1a | 1254 | N | P | N |
| ORF1a | 1256 | N | P | P |
| ORF1a | 1259 | N | D | N |
| ORF1a | 1260 | N | P | P |
| ORF1a | 1261 | N | P | P |
| ORF1a | 1262 | N | P | N |
| ORF1a | 1263 | P | P | P |
| ORF1a | 1264 | P | P | P |
| ORF1a | 1265 | N | P | N |
| ORF1a | 1267 | N | P | P |
| ORF1a | 1268 | N | D | N |
| ORF1a | 1273 | P | P | P |
| ORF1a | 1274 | N | P | N |
| ORF1a | 1275 | N | P | P |
| ORF1a | 1276 | N | D | N |
| ORF1a | 1278 | N | P | P |
| ORF1a | 1279 | P | P | N |
| ORF1a | 1280 | D | D | D |
| ORF1a | 1282 | N | P | N |
| ORF1a | 1283 | D | D | D |
| ORF1a | 1284 | N | P | P |
| ORF1a | 1290 | N | N | P |
| ORF1a | 1291 | N | D | D |
| ORF1a | 1292 | D | N | P |
| ORF1a | 1295 | N | N | D |
| ORF1a | 1298 | D | D | D |
| ORF1a | 1299 | P | P | P |
| ORF1a | 1302 | N | P | P |
| ORF1a | 1305 | D | N | D |
| ORF1a | 1307 | N | N | D |
| ORF1a | 1309 | N | P | P |
| ORF1a | 1310 | P | N | P |
| ORF1a | 1311 | D | N | N |
| ORF1a | 1313 | P | P | P |
| ORF1a | 1314 | N | P | P |
| ORF1a | 1319 | N | P | N |
| ORF1a | 1320 | P | N | P |
| ORF1a | 1321 | N | P | N |
| ORF1a | 1322 | D | N | N |
| ORF1a | 1324 | N | P | P |
| ORF1a | 1325 | N | P | P |
| ORF1a | 1327 | P | P | P |
| ORF1a | 1328 | N | N | P |
| ORF1a | 1329 | N | P | N |
| ORF1a | 1330 | P | P | P |
| ORF1a | 1331 | N | P | P |
| ORF1a | 1332 | N | P | P |
| ORF1a | 1335 | N | P | N |
| ORF1a | 1337 | P | P | P |
| ORF1a | 1338 | N | P | N |
| ORF1a | 1340 | N | N | P |
| ORF1a | 1342 | P | P | P |
| ORF1a | 1344 | P | P | N |
| ORF1a | 1345 | P | P | P |
| ORF1a | 1346 | N | N | P |
| ORF1a | 1353 | P | P | P |
| ORF1a | 1355 | N | N | P |
| ORF1a | 1356 | P | P | P |
| ORF1a | 1357 | P | P | P |
| ORF1a | 1358 | N | P | P |
| ORF1a | 1359 | N | P | N |
| ORF1a | 1362 | P | P | P |
| ORF1a | 1368 | N | P | P |
| ORF1a | 1369 | P | P | P |
| ORF1a | 1370 | N | N | P |
| ORF1a | 1371 | N | P | P |
| ORF1a | 1372 | N | N | P |
| ORF1a | 1376 | P | P | P |
| ORF1a | 1379 | P | P | P |
| ORF1a | 1380 | N | P | P |
| ORF1a | 1385 | N | P | P |
| ORF1a | 1386 | N | D + P | P |
| ORF1a | 1387 | N | P | P |
| ORF1a | 1388 | N | P | P |
| ORF1a | 1390 | N | P | N |
| ORF1a | 1391 | N | P | P |
| ORF1a | 1392 | N | P | N |
| ORF1a | 1393 | P | P | P |
| ORF1a | 1397 | N | N | P |
| ORF1a | 1400 | N | N | P |
| ORF1a | 1404 | N | D | N |
| ORF1a | 1406 | N | P | N |
| ORF1a | 1407 | P | N | N |
| ORF1a | 1415 | P | P | P |
| ORF1a | 1421 | P | P | N |
| ORF1a | 1422 | N | N | P |
| ORF1a | 1423 | P | P | P |
| ORF1a | 1424 | N | P | N |
| ORF1a | 1425 | P | P | P |
| ORF1a | 1426 | P | P | P |
| ORF1a | 1429 | D | D | N |
| ORF1a | 1431 | N | P | N |
| ORF1a | 1432 | N | P | N |
| ORF1a | 1433 | N | P | P |
| ORF1a | 1434 | N | N | P |
| ORF1a | 1435 | P | P | N |
| ORF1a | 1437 | P | P | P |
| ORF1a | 1438 | P | P | P |
| ORF1a | 1439 | P | P | P |
| ORF1a | 1440 | N | D | N |
| ORF1a | 1441 | P | P | P |
| ORF1a | 1445 | N | P | N |
| ORF1a | 1447 | N | P | P |
| ORF1a | 1449 | N | N | P |
| ORF1a | 1450 | N | P | P |
| ORF1a | 1451 | N | P | N |
| ORF1a | 1453 | P | N | N |
| ORF1a | 1454 | N | P | N |
| ORF1a | 1455 | N | P | P |
| ORF1a | 1456 | N | P | P |
| ORF1a | 1457 | P | P | P |
| ORF1a | 1459 | P | P | P |
| ORF1a | 1462 | N | N | P |
| ORF1a | 1463 | P | N | N |
| ORF1a | 1465 | P | D | N |
| ORF1a | 1467 | N | P | N |
| ORF1a | 1468 | N | D | N |
| ORF1a | 1469 | N | P | P |
| ORF1a | 1470 | N | P | N |
| ORF1a | 1473 | D | D | D |
| ORF1a | 1474 | P | N | N |
| ORF1a | 1476 | N | N | P |
| ORF1a | 1478 | N | D | N |
| ORF1a | 1479 | N | P | P |
| ORF1a | 1480 | N | P | N |
| ORF1a | 1482 | P | N | N |
| ORF1a | 1483 | P | N | N |
| ORF1a | 1484 | N | P | P |
| ORF1a | 1485 | P | P | P |
| ORF1a | 1486 | N | N | P |
| ORF1a | 1489 | P | P | P |
| ORF1a | 1490 | N | N | P |
| ORF1a | 1492 | P | N | N |
| ORF1a | 1493 | N | P | P |
| ORF1a | 1497 | D | D | D |
| ORF1a | 1499 | P | P | N |
| ORF1a | 1500 | N | D | D |
| ORF1a | 1501 | P | P | N |
| ORF1a | 1502 | N | P | N |
| ORF1a | 1504 | P | N | N |
| ORF1a | 1505 | P | P | P |
| ORF1a | 1506 | N | N | P |
| ORF1a | 1507 | N | D | N |
| ORF1a | 1509 | N | P | N |
| ORF1a | 1510 | P | N | N |
| ORF1a | 1515 | N | N | P |
| ORF1a | 1520 | N | N | D |
| ORF1a | 1523 | P | P | P |
| ORF1a | 1524 | N | N | P |
| ORF1a | 1526 | N | P | N |
| ORF1a | 1528 | N | N | P |
| ORF1a | 1530 | N | N | P |
| ORF1a | 1532 | N | N | D |
| ORF1a | 1535 | P | P | P |
| ORF1a | 1538 | N | D | D |
| ORF1a | 1540 | P | P | P |
| ORF1a | 1541 | N | N | P |
| ORF1a | 1542 | D | N | P |
| ORF1a | 1543 | D | D | D |
| ORF1a | 1544 | P | P | P |
| ORF1a | 1546 | P | P | P |
| ORF1a | 1547 | D | D | D |
| ORF1a | 1553 | P | P | N |
| ORF1a | 1554 | P | P | P |
| ORF1a | 1558 | N | P | P |
| ORF1a | 1562 | P | N | N |
| ORF1a | 1563 | N | P | N |
| ORF1a | 1565 | P | P | P |
| ORF1a | 1567 | D | D | D |
| ORF1a | 1569 | N | N | P |
| ORF1a | 1570 | N | P | P |
| ORF1a | 1572 | N | P | N |
| ORF1a | 1573 | P | P | P |
| ORF1a | 1575 | N | N | P |
| ORF1a | 1576 | P | P | P |
| ORF1a | 1578 | P | P | P |
| ORF1a | 1579 | N | N | P |
| ORF1a | 1581 | P | P | P |
| ORF1a | 1582 | D | N | N |
| ORF1a | 1583 | N | D | N |
| ORF1a | 1584 | P | N | N |
| ORF1a | 1585 | N | N | P |
| ORF1a | 1590 | P | P | P |
| ORF1a | 1592 | N | P | N |
| ORF1a | 1594 | N | P | P |
| ORF1a | 1597 | D | D | D |
| ORF1a | 1599 | N | N | P |
| ORF1a | 1601 | N | P | N |
| ORF1a | 1602 | P | P | P |
| ORF1a | 1604 | P | P | P |
| ORF1a | 1605 | N | N | D |
| ORF1a | 1606 | N | N | P |
| ORF1a | 1608 | N | N | P |
| ORF1a | 1614 | D | N | N |
| ORF1a | 1617 | N | P | N |
| ORF1a | 1618 | N | P | N |
| ORF1a | 1619 | N | P | P |
| ORF1a | 1621 | P | P | P |
| ORF1a | 1625 | N | D | P |
| ORF1a | 1626 | N | P | N |
| ORF1a | 1627 | P | P | P |
| ORF1a | 1628 | D | N | N |
| ORF1a | 1629 | N | P | N |
| ORF1a | 1630 | P | P | P |
| ORF1a | 1632 | N | P | N |
| ORF1a | 1633 | N | D | D |
| ORF1a | 1635 | P | P | P |
| ORF1a | 1636 | P | P | P |
| ORF1a | 1637 | D | N | N |
| ORF1a | 1638 | D | N | N |
| ORF1a | 1640 | D | D | D |
| ORF1a | 1643 | P | P | P |
| ORF1a | 1645 | P | N | P |
| ORF1a | 1646 | N | P | P |
| ORF1a | 1648 | N | P | P |
| ORF1a | 1649 | N | P | N |
| ORF1a | 1650 | N | P | N |
| ORF1a | 1651 | N | P | P |
| ORF1a | 1652 | P | P | P |
| ORF1a | 1655 | D | D | D |
| ORF1a | 1658 | P | P | P |
| ORF1a | 1664 | N | P | N |
| ORF1a | 1666 | N | P | N |
| ORF1a | 1670 | N | N | P |
| ORF1a | 1673 | P | P | P |
| ORF1a | 1674 | N | N | P |
| ORF1a | 1675 | P | P | P |
| ORF1a | 1676 | N | P | P |
| ORF1a | 1679 | N | N | P |
| ORF1a | 1680 | P | P | P |
| ORF1a | 1682 | D | D | D |
| ORF1a | 1684 | P | N | N |
| ORF1a | 1690 | N | P | P |
| ORF1a | 1691 | P | P | N |
| ORF1a | 1692 | D | N | N |
| ORF1a | 1693 | N | P | N |
| ORF1a | 1694 | N | P | N |
| ORF1a | 1696 | N | P | P |
| ORF1a | 1697 | N | P | N |
| ORF1a | 1700 | P | P | P |
| ORF1a | 1702 | N | P | P |
| ORF1a | 1703 | P | P | P |
| ORF1a | 1704 | N | P | N |
| ORF1a | 1707 | P | N | P |
| ORF1a | 1708 | N | D | N |
| ORF1a | 1709 | P | P | P |
| ORF1a | 1712 | N | P | P |
| ORF1a | 1713 | N | N | P |
| ORF1a | 1716 | N | N | D |
| ORF1a | 1717 | N | P | N |
| ORF1a | 1720 | D | P | D |
| ORF1a | 1722 | N | P | P |
| ORF1a | 1724 | D | N | D |
| ORF1a | 1728 | N | P | P |
| ORF1a | 1729 | N | N | P |
| ORF1a | 1733 | N | D | D |
| ORF1a | 1734 | P | P | P |
| ORF1a | 1735 | N | P | N |
| ORF1a | 1738 | N | D | P |
| ORF1a | 1739 | N | P | P |
| ORF1a | 1741 | P | P | N |
| ORF1a | 1744 | P | P | P |
| ORF1a | 1746 | N | N | P |
| ORF1a | 1747 | N | P | N |
| ORF1a | 1749 | P | P | P |
| ORF1a | 1750 | N | P | P |
| ORF1a | 1751 | P | P | N |
| ORF1a | 1754 | D | D | D |
| ORF1a | 1756 | P | P | N |
| ORF1a | 1758 | P | P | P |
| ORF1a | 1759 | N | D | N |
| ORF1a | 1760 | N | N | D |
| ORF1a | 1761 | D | N | N |
| ORF1a | 1763 | D | D | D |
| ORF1a | 1765 | P | P | P |
| ORF1a | 1767 | P | P | P |
| ORF1a | 1770 | N | P | P |
| ORF1a | 1772 | N | P | P |
| ORF1a | 1773 | N | P | P |
| ORF1a | 1775 | N | P | N |
| ORF1a | 1778 | N | P | N |
| ORF1a | 1781 | N | P | P |
| ORF1a | 1783 | N | P | N |
| ORF1a | 1784 | N | D | D |
| ORF1a | 1786 | D | D | D |
| ORF1a | 1787 | N | N | P |
| ORF1a | 1788 | P | P | P |
| ORF1a | 1791 | N | N | P |
| ORF1a | 1793 | N | P | P |
| ORF1a | 1796 | P | P | P |
| ORF1a | 1797 | P | P | P |
| ORF1a | 1798 | N | P | N |
| ORF1a | 1799 | N | N | P |
| ORF1a | 1800 | P | P | P |
| ORF1a | 1803 | D | D | D |
| ORF1a | 1808 | N | P | P |
| ORF1a | 1811 | N | N | P |
| ORF1a | 1812 | D | N | N |
| ORF1a | 1813 | D | N | N |
| ORF1a | 1816 | N | P | N |
| ORF1a | 1817 | D | D | N |
| ORF1a | 1818 | P | D | N |
| ORF1a | 1820 | P | N | N |
| ORF1a | 1821 | P | P | N |
| ORF1a | 1822 | D | D | D |
| ORF1a | 1827 | N | P | P |
| ORF1a | 1828 | D | D | D |
| ORF1a | 1832 | P | N | N |
| ORF1a | 1835 | N | P | N |
| ORF1a | 1836 | N | N | P |
| ORF1a | 1840 | D | D | D |
| ORF1a | 1844 | N | N | D |
| ORF1a | 1847 | P | P | P |
| ORF1a | 1849 | P | P | P |
| ORF1a | 1853 | D | D | D |
| ORF1a | 1854 | D | D | D |
| ORF1a | 1855 | N | P | P |
| ORF1a | 1856 | P | P | N |
| ORF1a | 1859 | D | N | D |
| ORF1a | 1862 | D | D | D |
| ORF1a | 1864 | N | P | P |
| ORF1a | 1867 | N | P | N |
| ORF1a | 1868 | P | P | P |
| ORF1a | 1871 | D | D | N |
| ORF1a | 1873 | P | P | P |
| ORF1a | 1874 | N | D | N |
| ORF1a | 1879 | N | P | N |
| ORF1a | 1881 | D | D | D |
| ORF1a | 1882 | N | N | P |
| ORF1a | 1885 | N | D | N |
| ORF1a | 1887 | D | N | D |
| ORF1a | 1894 | N | N | P |
| ORF1a | 1895 | D | D | D |
| ORF1a | 1896 | N | P | N |
| ORF1a | 1898 | D | D | N |
| ORF1a | 1900 | N | N | P |
| ORF1a | 1903 | P | P | P |
| ORF1a | 1905 | N | D | N |
| ORF1a | 1907 | P | P | P |
| ORF1a | 1908 | N | P | P |
| ORF1a | 1910 | N | P | P |
| ORF1a | 1911 | N | P | N |
| ORF1a | 1913 | N | N | P |
| ORF1a | 1914 | P | P | P |
| ORF1a | 1916 | P | P | P |
| ORF1a | 1917 | N | N | P |
| ORF1a | 1919 | N | P | P |
| ORF1a | 1920 | P | P | N |
| ORF1a | 1921 | D | D | D |
| ORF1a | 1922 | P | P | P |
| ORF1a | 1923 | D | D | D |
| ORF1a | 1924 | N | N | P |
| ORF1a | 1925 | P | P | P |
| ORF1a | 1926 | N | P | N |
| ORF1a | 1928 | N | P | P |
| ORF1a | 1930 | N | P | P |
| ORF1a | 1931 | P | P | P |
| ORF1a | 1932 | N | P | N |
| ORF1a | 1934 | N | P | N |
| ORF1a | 1935 | P | P | P |
| ORF1a | 1937 | N | N | P |
| ORF1a | 1938 | N | N | P |
| ORF1a | 1939 | P | N | D |
| ORF1a | 1940 | N | P | N |
| ORF1a | 1943 | D | D | N |
| ORF1a | 1948 | N | N | P |
| ORF1a | 1952 | N | P | D |
| ORF1a | 1954 | P | N | N |
| ORF1a | 1958 | N | P | P |
| ORF1a | 1959 | N | P | P |
| ORF1a | 1960 | P | P | P |
| ORF1a | 1961 | N | P | P |
| ORF1a | 1963 | N | N | P |
| ORF1a | 1965 | N | N | P |
| ORF1a | 1967 | N | P | P |
| ORF1a | 1968 | N | P | P |
| ORF1a | 1973 | N | N | P |
| ORF1a | 1977 | P | P | P |
| ORF1a | 1978 | D | N | N |
| ORF1a | 1979 | N | N | P |
| ORF1a | 1980 | N | P | N |
| ORF1a | 1981 | N | P | N |
| ORF1a | 1982 | P | P | P |
| ORF1a | 1984 | N | P | N |
| ORF1a | 1985 | N | P | P |
| ORF1a | 1987 | N | N | P |
| ORF1a | 1988 | N | N | P |
| ORF1a | 1991 | P | N | N |
| ORF1a | 1995 | N | N | D |
| ORF1a | 1997 | D | D | D |
| ORF1a | 1998 | N | N | D |
| ORF1a | 1999 | P | N | N |
| ORF1a | 2000 | N | N | P |
| ORF1a | 2002 | N | N | P |
| ORF1a | 2003 | P | N | N |
| ORF1a | 2006 | N | P | P |
| ORF1a | 2011 | N | N | P |
| ORF1a | 2013 | P | P | P |
| ORF1a | 2015 | N | P | N |
| ORF1a | 2016 | D | D | N |
| ORF1a | 2020 | P | N | P |
| ORF1a | 2021 | N | P | N |
| ORF1a | 2024 | P | P | N |
| ORF1a | 2028 | P | P | P |
| ORF1a | 2029 | D | D | N |
| ORF1a | 2031 | N | P | P |
| ORF1a | 2033 | N | D | N |
| ORF1a | 2034 | N | D | D |
| ORF1a | 2037 | N | P | N |
| ORF1a | 2038 | N | P | N |
| ORF1a | 2043 | P | N | N |
| ORF1a | 2044 | N | P | P |
| ORF1a | 2045 | P | N | N |
| ORF1a | 2046 | D | D | N |
| ORF1a | 2047 | N | D | N |
| ORF1a | 2050 | N | P | N |
| ORF1a | 2051 | N | P | N |
| ORF1a | 2052 | P | P | P |
| ORF1a | 2053 | N | P | N |
| ORF1a | 2054 | N | N | D |
| ORF1a | 2058 | N | D | N |
| ORF1a | 2060 | P | N | N |
| ORF1a | 2061 | D | D | D |
| ORF1a | 2062 | D | D | D |
| ORF1a | 2065 | N | D | D |
| ORF1a | 2066 | N | P | P |
| ORF1a | 2067 | P | N | N |
| ORF1a | 2073 | N | P | N |
| ORF1a | 2077 | N | N | P |
| ORF1a | 2083 | N | D | N |
| ORF1a | 2084 | N | N | D |
| ORF1a | 2085 | N | N | P |
| ORF1a | 2087 | N | D | N |
| ORF1a | 2088 | N | D | N |
| ORF1a | 2090 | N | P | P |
| ORF1a | 2095 | N | P | P |
| ORF1a | 2097 | N | D | N |
| ORF1a | 2100 | N | P | P |
| ORF1a | 2103 | D | D | D |
| ORF1a | 2106 | N | D | D |
| ORF1a | 2108 | N | N | P |
| ORF1a | 2113 | N | P | P |
| ORF1a | 2114 | N | D | D |
| ORF1a | 2116 | P | P | P |
| ORF1a | 2117 | P | P | P |
| ORF1a | 2120 | N | D | N |
| ORF1a | 2122 | D | D | N |
| ORF1a | 2123 | N | D | N |
| ORF1a | 2124 | D | N | N |
| ORF1a | 2125 | D | D | N |
| ORF1a | 2127 | N | P | P |
| ORF1a | 2128 | D | N | P |
| ORF1a | 2129 | N | D | D |
| ORF1a | 2132 | D | P | N |
| ORF1a | 2133 | N | P | N |
| ORF1a | 2139 | N | D | N |
| ORF1a | 2143 | N | P | N |
| ORF1a | 2144 | D | D | N |
| ORF1a | 2145 | N | N | P |
| ORF1a | 2146 | N | D | D |
| ORF1a | 2147 | P | P | N |
| ORF1a | 2152 | N | D | N |
| ORF1a | 2153 | N | D | D |
| ORF1a | 2154 | D | D | D |
| ORF1a | 2155 | P | P | P |
| ORF1a | 2159 | N | D | N |
| ORF1a | 2161 | N | N | P |
| ORF1a | 2162 | N | N | D |
| ORF1a | 2164 | N | P | N |
| ORF1a | 2165 | N | D | D |
| ORF1a | 2166 | N | D | D |
| ORF1a | 2168 | N | P | P |
| ORF1a | 2170 | N | P | P |
| ORF1a | 2172 | P | P | P |
| ORF1a | 2175 | P | P | P |
| ORF1a | 2177 | N | P | P |
| ORF1a | 2183 | N | D | N |
| ORF1a | 2192 | N | P | N |
| ORF1a | 2193 | N | D | D |
| ORF1a | 2195 | N | N | P |
| ORF1a | 2197 | N | P | N |
| ORF1a | 2198 | N | P | P |
| ORF1a | 2200 | N | D | D |
| ORF1a | 2201 | P | P | P |
| ORF1a | 2203 | P | N | N |
| ORF1a | 2206 | P | P | P |
| ORF1a | 2207 | N | D | D |
| ORF1a | 2211 | P | P | P |
| ORF1a | 2212 | N | P | N |
| ORF1a | 2213 | N | N | P |
| ORF1a | 2214 | N | P | P |
| ORF1a | 2215 | N | P | N |
| ORF1a | 2218 | P | N | N |
| ORF1a | 2219 | N | P | N |
| ORF1a | 2220 | N | P | N |
| ORF1a | 2223 | N | P | P |
| ORF1a | 2224 | D | D | D |
| ORF1a | 2226 | P | P | P |
| ORF1a | 2229 | N | P | N |
| ORF1a | 2233 | N | P | N |
| ORF1a | 2234 | N | P | P |
| ORF1a | 2235 | P | P | P |
| ORF1a | 2236 | N | P | P |
| ORF1a | 2238 | N | N | P |
| ORF1a | 2239 | P | P | P |
| ORF1a | 2240 | N | P | N |
| ORF1a | 2241 | N | N | P |
| ORF1a | 2242 | D | D | D |
| ORF1a | 2243 | N | N | P |
| ORF1a | 2247 | P | N | P |
| ORF1a | 2249 | D | D | D |
| ORF1a | 2252 | N | N | P |
| ORF1a | 2258 | D | D | N |
| ORF1a | 2265 | N | N | D |
| ORF1a | 2269 | N | N | D |
| ORF1a | 2270 | N | N | P |
| ORF1a | 2271 | N | P | N |
| ORF1a | 2272 | P | N | P |
| ORF1a | 2274 | D | D | D |
| ORF1a | 2276 | N | P | P |
| ORF1a | 2277 | P | N | N |
| ORF1a | 2281 | N | P | N |
| ORF1a | 2283 | D | D | D |
| ORF1a | 2284 | N | P | N |
| ORF1a | 2285 | D | D | N |
| ORF1a | 2287 | N | N | D |
| ORF1a | 2290 | N | P | N |
| ORF1a | 2292 | N | P | P |
| ORF1a | 2295 | P | P | P |
| ORF1a | 2296 | P | P | P |
| ORF1a | 2297 | N | N | P |
| ORF1a | 2299 | P | P | N |
| ORF1a | 2301 | N | P | P |
| ORF1a | 2302 | N | P | P |
| ORF1a | 2303 | N | D | N |
| ORF1a | 2310 | P | P | N |
| ORF1a | 2312 | P | P | P |
| ORF1a | 2313 | P | N | N |
| ORF1a | 2314 | N | P | N |
| ORF1a | 2318 | N | P | N |
| ORF1a | 2319 | N | N | P |
| ORF1a | 2320 | D | D | D |
| ORF1a | 2324 | N | P | N |
| ORF1a | 2325 | P | P | N |
| ORF1a | 2326 | N | P | N |
| ORF1a | 2328 | N | P | P |
| ORF1a | 2330 | N | N | P |
| ORF1a | 2331 | P | P | P |
| ORF1a | 2333 | P | P | N |
| ORF1a | 2334 | P | P | P |
| ORF1a | 2338 | P | P | P |
| ORF1a | 2341 | P | P | P |
| ORF1a | 2342 | N | N | P |
| ORF1a | 2343 | P | N | N |
| ORF1a | 2344 | N | N | D |
| ORF1a | 2345 | N | D | N |
| ORF1a | 2346 | P | P | P |
| ORF1a | 2349 | N | N | P |
| ORF1a | 2350 | N | N | P |
| ORF1a | 2351 | P | P | P |
| ORF1a | 2352 | P | N | N |
| ORF1a | 2353 | P | P | N |
| ORF1a | 2354 | N | P | P |
| ORF1a | 2357 | N | D | N |
| ORF1a | 2362 | N | P | P |
| ORF1a | 2364 | N | P | N |
| ORF1a | 2369 | N | N | P |
| ORF1a | 2371 | P | P | N |
| ORF1a | 2372 | N | P | P |
| ORF1a | 2373 | N | P | N |
| ORF1a | 2375 | P | P | N |
| ORF1a | 2376 | P | P | P |
| ORF1a | 2378 | N | P | P |
| ORF1a | 2379 | N | N | P |
| ORF1a | 2385 | P | P | P |
| ORF1a | 2386 | P | P | P |
| ORF1a | 2388 | N | P | P |
| ORF1a | 2390 | N | P | P |
| ORF1a | 2391 | P | P | N |
| ORF1a | 2393 | N | P | P |
| ORF1a | 2398 | N | P | P |
| ORF1a | 2399 | N | P | P |
| ORF1a | 2401 | N | P | N |
| ORF1a | 2402 | P | P | N |
| ORF1a | 2405 | D | D | D |
| ORF1a | 2406 | P | P | N |
| ORF1a | 2408 | N | N | D |
| ORF1a | 2413 | P | P | N |
| ORF1a | 2414 | N | P | N |
| ORF1a | 2416 | N | N | P |
| ORF1a | 2418 | N | P | P |
| ORF1a | 2421 | P | P | P |
| ORF1a | 2422 | N | P | N |
| ORF1a | 2423 | P | P | N |
| ORF1a | 2424 | P | P | P |
| ORF1a | 2425 | N | P | P |
| ORF1a | 2426 | N | P | N |
| ORF1a | 2432 | P | P | P |
| ORF1a | 2433 | P | P | P |
| ORF1a | 2436 | P | P | P |
| ORF1a | 2439 | N | P | P |
| ORF1a | 2444 | N | P | N |
| ORF1a | 2445 | P | P | P |
| ORF1a | 2446 | N | N | P |
| ORF1a | 2447 | P | P | P |
| ORF1a | 2448 | P | P | P |
| ORF1a | 2451 | N | N | P |
| ORF1a | 2453 | N | N | P |
| ORF1a | 2455 | N | N | P |
| ORF1a | 2456 | P | P | P |
| ORF1a | 2457 | P | P | P |
| ORF1a | 2458 | P | P | P |
| ORF1a | 2460 | N | D | N |
| ORF1a | 2461 | P | P | N |
| ORF1a | 2463 | P | P | N |
| ORF1a | 2467 | N | P | P |
| ORF1a | 2470 | P | P | P |
| ORF1a | 2471 | P | N | N |
| ORF1a | 2474 | N | N | P |
| ORF1a | 2475 | P | P | P |
| ORF1a | 2477 | N | P | P |
| ORF1a | 2478 | N | N | P |
| ORF1a | 2479 | P | N | P |
| ORF1a | 2480 | P | P | P |
| ORF1a | 2483 | N | D | P |
| ORF1a | 2484 | N | P | P |
| ORF1a | 2487 | N | N | P |
| ORF1a | 2488 | D | D | D |
| ORF1a | 2489 | P | P | P |
| ORF1a | 2490 | P | P | P |
| ORF1a | 2492 | N | N | D |
| ORF1a | 2493 | D | D | D |
| ORF1a | 2494 | N | N | P |
| ORF1a | 2495 | N | N | D |
| ORF1a | 2497 | D | D | D |
| ORF1a | 2500 | P | N | D |
| ORF1a | 2502 | N | P | P |
| ORF1a | 2506 | N | P | N |
| ORF1a | 2507 | N | P | N |
| ORF1a | 2510 | N | N | D |
| ORF1a | 2511 | D | D | D |
| ORF1a | 2512 | N | D | N |
| ORF1a | 2513 | N | P | P |
| ORF1a | 2514 | N | P | N |
| ORF1a | 2516 | N | P | P |
| ORF1a | 2518 | N | N | P |
| ORF1a | 2519 | P | P | P |
| ORF1a | 2520 | N | N | P |
| ORF1a | 2522 | P | P | P |
| ORF1a | 2523 | P | P | P |
| ORF1a | 2525 | P | P | N |
| ORF1a | 2526 | P | P | P |
| ORF1a | 2527 | P | P | P |
| ORF1a | 2529 | D | D | D |
| ORF1a | 2531 | D | N | N |
| ORF1a | 2532 | N | N | D |
| ORF1a | 2537 | P | N | N |
| ORF1a | 2544 | P | P | P |
| ORF1a | 2546 | N | P | N |
| ORF1a | 2547 | N | P | N |
| ORF1a | 2548 | N | P | P |
| ORF1a | 2550 | N | P | N |
| ORF1a | 2551 | N | N | P |
| ORF1a | 2552 | N | N | P |
| ORF1a | 2554 | N | D | D |
| ORF1a | 2556 | P | P | N |
| ORF1a | 2557 | P | P | P |
| ORF1a | 2558 | N | P | P |
| ORF1a | 2559 | N | P | P |
| ORF1a | 2560 | P | P | P |
| ORF1a | 2561 | P | P | P |
| ORF1a | 2564 | N | P | P |
| ORF1a | 2567 | N | P | N |
| ORF1a | 2570 | P | N | N |
| ORF1a | 2571 | P | N | N |
| ORF1a | 2572 | N | N | P |
| ORF1a | 2573 | N | P | P |
| ORF1a | 2575 | N | P | N |
| ORF1a | 2576 | P | N | N |
| ORF1a | 2577 | N | P | P |
| ORF1a | 2578 | N | P | P |
| ORF1a | 2582 | N | N | P |
| ORF1a | 2583 | N | P | N |
| ORF1a | 2584 | P | P | P |
| ORF1a | 2585 | P | P | N |
| ORF1a | 2586 | D | N | P |
| ORF1a | 2589 | N | N | D |
| ORF1a | 2592 | N | P | N |
| ORF1a | 2593 | N | N | D |
| ORF1a | 2594 | N | P | P |
| ORF1a | 2597 | P | P | P |
| ORF1a | 2599 | P | P | P |
| ORF1a | 2602 | N | P | N |
| ORF1a | 2603 | D | N | D |
| ORF1a | 2605 | N | P | N |
| ORF1a | 2609 | P | N | N |
| ORF1a | 2610 | N | N | P |
| ORF1a | 2611 | P | N | P |
| ORF1a | 2612 | P | N | N |
| ORF1a | 2613 | D | D | D |
| ORF1a | 2616 | N | N | P |
| ORF1a | 2618 | N | N | D |
| ORF1a | 2620 | N | D | N |
| ORF1a | 2621 | N | P | P |
| ORF1a | 2622 | D | D | D |
| ORF1a | 2623 | P | N | P |
| ORF1a | 2624 | P | P | P |
| ORF1a | 2625 | P | P | P |
| ORF1a | 2626 | N | P | N |
| ORF1a | 2627 | P | N | P |
| ORF1a | 2628 | P | P | N |
| ORF1a | 2629 | N | P | P |
| ORF1a | 2630 | P | P | N |
| ORF1a | 2633 | N | N | P |
| ORF1a | 2635 | N | P | P |
| ORF1a | 2637 | D | N | N |
| ORF1a | 2638 | P | P | P |
| ORF1a | 2639 | N | D | N |
| ORF1a | 2640 | N | P | N |
| ORF1a | 2641 | N | P | N |
| ORF1a | 2643 | N | N | P |
| ORF1a | 2644 | P | N | P |
| ORF1a | 2645 | N | N | P |
| ORF1a | 2646 | P | N | P |
| ORF1a | 2648 | D | D | D |
| ORF1a | 2657 | N | N | P |
| ORF1a | 2658 | P | P | P |
| ORF1a | 2659 | D | N | N |
| ORF1a | 2660 | P | N | N |
| ORF1a | 2661 | D | D | N |
| ORF1a | 2662 | P | P | P |
| ORF1a | 2663 | P | N | N |
| ORF1a | 2666 | N | P | N |
| ORF1a | 2667 | N | P | D + P |
| ORF1a | 2671 | P | P | P |
| ORF1a | 2673 | P | P | P |
| ORF1a | 2675 | P | P | P |
| ORF1a | 2676 | P | P | P |
| ORF1a | 2677 | P | P | P |
| ORF1a | 2678 | P | P | P |
| ORF1a | 2679 | P | P | P |
| ORF1a | 2680 | N | N | P |
| ORF1a | 2681 | N | N | P |
| ORF1a | 2682 | N | N | P |
| ORF1a | 2685 | P | N | D |
| ORF1a | 2686 | N | P | P |
| ORF1a | 2687 | P | P | P |
| ORF1a | 2688 | N | D | N |
| ORF1a | 2692 | N | P | N |
| ORF1a | 2693 | P | P | P |
| ORF1a | 2696 | P | P | P |
| ORF1a | 2697 | N | P | N |
| ORF1a | 2700 | N | P | N |
| ORF1a | 2701 | P | P | P |
| ORF1a | 2702 | D | D | D |
| ORF1a | 2703 | P | P | P |
| ORF1a | 2706 | N | P | P |
| ORF1a | 2707 | P | P | P |
| ORF1a | 2708 | N | P | P |
| ORF1a | 2710 | N | N | D |
| ORF1a | 2714 | N | P | P |
| ORF1a | 2715 | N | N | P |
| ORF1a | 2716 | N | P | N |
| ORF1a | 2718 | N | P | P |
| ORF1a | 2720 | N | P | P |
| ORF1a | 2721 | N | P | P |
| ORF1a | 2722 | N | P | N |
| ORF1a | 2725 | N | P | P |
| ORF1a | 2727 | N | P | N |
| ORF1a | 2733 | N | P | P |
| ORF1a | 2736 | P | P | P |
| ORF1a | 2739 | D | D | D |
| ORF1a | 2742 | N | P | N |
| ORF1a | 2743 | N | P | N |
| ORF1a | 2748 | N | P | N |
| ORF1a | 2749 | P | P | P |
| ORF1a | 2752 | N | P | P |
| ORF1a | 2754 | P | P | N |
| ORF1a | 2755 | N | P | N |
| ORF1a | 2756 | P | P | P |
| ORF1a | 2757 | P | P | N |
| ORF1a | 2760 | N | P | N |
| ORF1a | 2761 | P | N | N |
| ORF1a | 2763 | N | P | N |
| ORF1a | 2767 | N | N | P |
| ORF1a | 2773 | P | N | N |
| ORF1a | 2775 | P | N | N |
| ORF1a | 2776 | N | N | P |
| ORF1a | 2778 | P | N | N |
| ORF1a | 2779 | N | P | P |
| ORF1a | 2780 | N | N | P |
| ORF1a | 2783 | N | D | N |
| ORF1a | 2784 | N | P | N |
| ORF1a | 2785 | D | D | D |
| ORF1a | 2787 | P | P | P |
| ORF1a | 2797 | D | D | D |
| ORF1a | 2798 | P | P | P |
| ORF1a | 2800 | N | D | D |
| ORF1a | 2805 | N | P | N |
| ORF1a | 2806 | P | P | P |
| ORF1a | 2808 | N | P | N |
| ORF1a | 2809 | P | P | N |
| ORF1a | 2812 | N | N | P |
| ORF1a | 2816 | N | N | P |
| ORF1a | 2817 | N | P | N |
| ORF1a | 2821 | N | N | P |
| ORF1a | 2822 | D | N | N |
| ORF1a | 2823 | N | D | D |
| ORF1a | 2824 | N | P | P |
| ORF1a | 2825 | N | N | D |
| ORF1a | 2826 | N | N | P |
| ORF1a | 2828 | N | D | N |
| ORF1a | 2829 | P | P | P |
| ORF1a | 2830 | N | P | P |
| ORF1a | 2832 | N | D | N |
| ORF1a | 2834 | P | N | N |
| ORF1a | 2835 | N | P | N |
| ORF1a | 2836 | P | P | N |
| ORF1a | 2839 | P | P | P |
| ORF1a | 2842 | N | N | P |
| ORF1a | 2845 | N | P | P |
| ORF1a | 2846 | D | D | D |
| ORF1a | 2847 | N | P | P |
| ORF1a | 2848 | P | P | N |
| ORF1a | 2849 | N | P | N |
| ORF1a | 2851 | P | P | P |
| ORF1a | 2852 | P | P | P |
| ORF1a | 2853 | N | P | N |
| ORF1a | 2855 | N | N | D |
| ORF1a | 2856 | N | P | N |
| ORF1a | 2857 | P | D + P | D |
| ORF1a | 2859 | N | P | D |
| ORF1a | 2860 | P | P | P |
| ORF1a | 2863 | N | P | N |
| ORF1a | 2865 | N | P | P |
| ORF1a | 2866 | N | N | D |
| ORF1a | 2867 | N | P | P |
| ORF1a | 2868 | N | P | N |
| ORF1a | 2870 | N | P | P |
| ORF1a | 2871 | N | P | N |
| ORF1a | 2872 | N | D | N |
| ORF1a | 2873 | D | N | N |
| ORF1a | 2874 | D | N | N |
| ORF1a | 2878 | P | N | N |
| ORF1a | 2880 | P | P | N |
| ORF1a | 2881 | N | N | P |
| ORF1a | 2882 | N | P | N |
| ORF1a | 2883 | P | P | P |
| ORF1a | 2884 | P | P | P |
| ORF1a | 2886 | N | N | P |
| ORF1a | 2887 | N | N | P |
| ORF1a | 2891 | N | N | D |
| ORF1a | 2892 | D | N | P |
| ORF1a | 2894 | N | P | P |
| ORF1a | 2895 | P | P | P |
| ORF1a | 2897 | P | P | P |
| ORF1a | 2898 | N | P | N |
| ORF1a | 2899 | P | P | N |
| ORF1a | 2900 | D | D | D |
| ORF1a | 2902 | N | P | N |
| ORF1a | 2903 | P | N | N |
| ORF1a | 2905 | N | P | P |
| ORF1a | 2906 | N | N | D + P |
| ORF1a | 2907 | P | P | P |
| ORF1a | 2908 | D | N | N |
| ORF1a | 2909 | N | N | D |
| ORF1a | 2911 | N | N | P |
| ORF1a | 2912 | N | N | P |
| ORF1a | 2914 | P | P | P |
| ORF1a | 2915 | N | N | P |
| ORF1a | 2916 | P | N | N |
| ORF1a | 2917 | N | P | N |
| ORF1a | 2918 | N | P | N |
| ORF1a | 2920 | P | P | N |
| ORF1a | 2924 | N | P | P |
| ORF1a | 2926 | N | D | N |
| ORF1a | 2929 | P | N | N |
| ORF1a | 2930 | N | D | D |
| ORF1a | 2931 | P | P | P |
| ORF1a | 2935 | P | P | P |
| ORF1a | 2936 | P | P | P |
| ORF1a | 2938 | P | P | N |
| ORF1a | 2939 | N | P | P |
| ORF1a | 2940 | P | P | P |
| ORF1a | 2942 | N | P | P |
| ORF1a | 2945 | N | P | P |
| ORF1a | 2949 | N | N | P |
| ORF1a | 2950 | N | N | P |
| ORF1a | 2951 | N | P | P |
| ORF1a | 2954 | P | P | P |
| ORF1a | 2955 | P | P | P |
| ORF1a | 2956 | N | P | P |
| ORF1a | 2958 | P | N | P |
| ORF1a | 2959 | P | P | P |
| ORF1a | 2961 | N | N | P |
| ORF1a | 2962 | P | P | P |
| ORF1a | 2964 | P | P | P |
| ORF1a | 2965 | N | P | P |
| ORF1a | 2966 | N | D | N |
| ORF1a | 2967 | D | D | N |
| ORF1a | 2968 | N | P | N |
| ORF1a | 2970 | N | P | N |
| ORF1a | 2972 | N | D | D |
| ORF1a | 2974 | P | P | N |
| ORF1a | 2975 | P | P | P |
| ORF1a | 2976 | P | N | N |
| ORF1a | 2980 | N | D | D |
| ORF1a | 2982 | N | D | N |
| ORF1a | 2983 | N | P | P |
| ORF1a | 2985 | P | P | P |
| ORF1a | 2986 | P | P | P |
| ORF1a | 2989 | N | P | N |
| ORF1a | 2991 | P | P | P |
| ORF1a | 2992 | P | P | P |
| ORF1a | 2994 | D | D | D |
| ORF1a | 2998 | P | P | P |
| ORF1a | 2999 | N | N | P |
| ORF1a | 3003 | P | P | P |
| ORF1a | 3005 | N | P | P |
| ORF1a | 3006 | N | P | P |
| ORF1a | 3007 | P | P | P |
| ORF1a | 3008 | N | P | N |
| ORF1a | 3011 | N | P | P |
| ORF1a | 3013 | N | N | P |
| ORF1a | 3015 | N | P | P |
| ORF1a | 3016 | N | P | P |
| ORF1a | 3017 | N | P | N |
| ORF1a | 3018 | P | P | P |
| ORF1a | 3021 | P | P | N |
| ORF1a | 3023 | P | N | N |
| ORF1a | 3024 | N | N | P |
| ORF1a | 3025 | N | N | P |
| ORF1a | 3026 | N | N | P |
| ORF1a | 3027 | D | D | D |
| ORF1a | 3029 | P | P | N |
| ORF1a | 3031 | P | P | N |
| ORF1a | 3033 | N | P | P |
| ORF1a | 3034 | N | N | P |
| ORF1a | 3037 | N | P | P |
| ORF1a | 3041 | N | N | P |
| ORF1a | 3042 | N | N | D |
| ORF1a | 3044 | P | P | P |
| ORF1a | 3046 | N | N | P |
| ORF1a | 3047 | P | N | N |
| ORF1a | 3048 | P | P | P |
| ORF1a | 3053 | P | P | P |
| ORF1a | 3055 | P | P | P |
| ORF1a | 3056 | D | N | N |
| ORF1a | 3057 | D | P | N |
| ORF1a | 3058 | N | D | D |
| ORF1a | 3061 | N | D | N |
| ORF1a | 3062 | N | P | P |
| ORF1a | 3063 | N | P | N |
| ORF1a | 3068 | P | N | P |
| ORF1a | 3069 | P | P | P |
| ORF1a | 3070 | D | D | D |
| ORF1a | 3072 | D | D | D |
| ORF1a | 3073 | D | P | N |
| ORF1a | 3074 | N | P | P |
| ORF1a | 3076 | D | D | D |
| ORF1a | 3081 | N | P | P |
| ORF1a | 3083 | P | P | N |
| ORF1a | 3084 | P | N | P |
| ORF1a | 3085 | N | N | P |
| ORF1a | 3086 | D | N | D |
| ORF1a | 3088 | P | P | P |
| ORF1a | 3089 | P | P | P |
| ORF1a | 3090 | D | N | D |
| ORF1a | 3092 | P | P | P |
| ORF1a | 3095 | N | P | P |
| ORF1a | 3096 | P | P | P |
| ORF1a | 3098 | P | P | P |
| ORF1a | 3100 | P | P | P |
| ORF1a | 3101 | P | N | N |
| ORF1a | 3102 | N | P | P |
| ORF1a | 3106 | N | P | N |
| ORF1a | 3111 | N | P | P |
| ORF1a | 3113 | N | P | N |
| ORF1a | 3116 | N | N | D |
| ORF1a | 3119 | N | N | P |
| ORF1a | 3123 | D | P | N |
| ORF1a | 3124 | N | P | P |
| ORF1a | 3125 | N | P | P |
| ORF1a | 3132 | P | P | P |
| ORF1a | 3136 | P | P | P |
| ORF1a | 3137 | N | P | P |
| ORF1a | 3138 | N | P | P |
| ORF1a | 3141 | P | P | P |
| ORF1a | 3143 | D | D | D |
| ORF1a | 3144 | N | N | P |
| ORF1a | 3149 | D | P | N |
| ORF1a | 3152 | N | P | P |
| ORF1a | 3153 | P | P | P |
| ORF1a | 3156 | P | P | P |
| ORF1a | 3158 | N | N | D |
| ORF1a | 3159 | N | N | P |
| ORF1a | 3160 | N | P | P |
| ORF1a | 3161 | N | P | P |
| ORF1a | 3162 | D | D | D |
| ORF1a | 3164 | N | N | D |
| ORF1a | 3165 | N | P | N |
| ORF1a | 3166 | N | N | P |
| ORF1a | 3171 | P | P | P |
| ORF1a | 3177 | P | N | N |
| ORF1a | 3179 | P | P | P |
| ORF1a | 3180 | P | P | P |
| ORF1a | 3181 | P | P | P |
| ORF1a | 3182 | N | P | N |
| ORF1a | 3184 | P | P | P |
| ORF1a | 3187 | N | P | N |
| ORF1a | 3191 | P | P | P |
| ORF1a | 3195 | D | D | D |
| ORF1a | 3196 | N | P | N |
| ORF1a | 3197 | P | P | P |
| ORF1a | 3198 | P | P | P |
| ORF1a | 3199 | P | P | P |
| ORF1a | 3200 | N | P | P |
| ORF1a | 3201 | D | D | D |
| ORF1a | 3202 | N | P | N |
| ORF1a | 3206 | P | P | P |
| ORF1a | 3208 | N | P | P |
| ORF1a | 3209 | D | D | D |
| ORF1a | 3213 | P | N | N |
| ORF1a | 3214 | P | P | P |
| ORF1a | 3215 | N | P | P |
| ORF1a | 3218 | N | P | P |
| ORF1a | 3219 | P | N | P |
| ORF1a | 3220 | D | N | N |
| ORF1a | 3223 | N | N | P |
| ORF1a | 3224 | N | D | N |
| ORF1a | 3225 | N | P | P |
| ORF1a | 3226 | P | P | P |
| ORF1a | 3227 | N | P | P |
| ORF1a | 3228 | N | N | P |
| ORF1a | 3229 | N | P | N |
| ORF1a | 3232 | N | N | P |
| ORF1a | 3234 | P | P | P |
| ORF1a | 3235 | P | N | N |
| ORF1a | 3236 | P | N | N |
| ORF1a | 3237 | P | P | P |
| ORF1a | 3238 | P | P | P |
| ORF1a | 3239 | P | P | N |
| ORF1a | 3240 | N | P | N |
| ORF1a | 3241 | P | P | P |
| ORF1a | 3242 | N | P | P |
| ORF1a | 3243 | N | N | P |
| ORF1a | 3244 | D | D | N |
| ORF1a | 3245 | N | N | P |
| ORF1a | 3246 | N | P | N |
| ORF1a | 3248 | N | N | P |
| ORF1a | 3249 | N | P | P |
| ORF1a | 3250 | P | P | P |
| ORF1a | 3251 | N | N | P |
| ORF1a | 3252 | P | P | P |
| ORF1a | 3253 | N | P | P |
| ORF1a | 3255 | D | D | D |
| ORF1a | 3256 | N | N | P |
| ORF1a | 3257 | P | P | P |
| ORF1a | 3258 | P | P | P |
| ORF1a | 3259 | P | P | P |
| ORF1a | 3260 | N | P | P |
| ORF1a | 3261 | N | P | P |
| ORF1a | 3265 | N | P | P |
| ORF1a | 3266 | N | P | N |
| ORF1a | 3267 | N | P | N |
| ORF1a | 3270 | P | P | N |
| ORF1a | 3271 | P | P | P |
| ORF1a | 3272 | N | P | P |
| ORF1a | 3273 | N | P | P |
| ORF1a | 3275 | N | P | P |
| ORF1a | 3276 | N | P | P |
| ORF1a | 3277 | P | N | P |
| ORF1a | 3278 | D | D | N |
| ORF1a | 3281 | P | P | P |
| ORF1a | 3283 | N | N | P |
| ORF1a | 3284 | D | D | D |
| ORF1a | 3286 | N | D | N |
| ORF1a | 3287 | N | D | D |
| ORF1a | 3288 | P | N | N |
| ORF1a | 3289 | N | P | N |
| ORF1a | 3290 | P | P | P |
| ORF1a | 3291 | P | P | P |
| ORF1a | 3293 | N | P | N |
| ORF1a | 3295 | P | P | N |
| ORF1a | 3296 | P | P | P |
| ORF1a | 3297 | P | P | P |
| ORF1a | 3300 | N | N | P |
| ORF1a | 3302 | P | P | P |
| ORF1a | 3303 | N | N | P |
| ORF1a | 3304 | N | N | P |
| ORF1a | 3305 | P | P | P |
| ORF1a | 3306 | N | P | P |
| ORF1a | 3307 | N | P | P |
| ORF1a | 3308 | P | P | N |
| ORF1a | 3310 | D | N | N |
| ORF1a | 3311 | P | P | P |
| ORF1a | 3314 | N | P | P |
| ORF1a | 3315 | N | P | P |
| ORF1a | 3318 | N | P | N |
| ORF1a | 3319 | N | N | P |
| ORF1a | 3320 | P | N | N |
| ORF1a | 3321 | P | P | P |
| ORF1a | 3322 | N | N | P |
| ORF1a | 3323 | D | N | N |
| ORF1a | 3325 | N | P | P |
| ORF1a | 3326 | P | N | N |
| ORF1a | 3327 | N | P | N |
| ORF1a | 3328 | N | P | P |
| ORF1a | 3329 | P | P | P |
| ORF1a | 3330 | D | N | N |
| ORF1a | 3331 | N | P | P |
| ORF1a | 3332 | N | D | N |
| ORF1a | 3334 | D | N | N |
| ORF1a | 3336 | P | N | N |
| ORF1a | 3338 | P | P | P |
| ORF1a | 3340 | N | P | N |
| ORF1a | 3341 | D | D | N |
| ORF1a | 3342 | P | P | P |
| ORF1a | 3348 | N | P | N |
| ORF1a | 3349 | N | N | P |
| ORF1a | 3351 | D | D | D |
| ORF1a | 3352 | D | D | N |
| ORF1a | 3353 | D | D | D |
| ORF1a | 3355 | N | P | P |
| ORF1a | 3357 | D | N | N |
| ORF1a | 3358 | P | N | P |
| ORF1a | 3359 | D | D | D |
| ORF1a | 3360 | N | N | P |
| ORF1a | 3361 | N | P | P |
| ORF1a | 3362 | N | P | P |
| ORF1a | 3363 | N | D | N |
| ORF1a | 3365 | P | P | P |
| ORF1a | 3366 | N | P | N |
| ORF1a | 3368 | P | P | P |
| ORF1a | 3371 | D | D | D |
| ORF1a | 3373 | N | N | P |
| ORF1a | 3376 | N | P | N |
| ORF1a | 3377 | P | P | P |
| ORF1a | 3378 | N | P | P |
| ORF1a | 3381 | P | P | P |
| ORF1a | 3384 | N | P | N |
| ORF1a | 3385 | P | P | P |
| ORF1a | 3386 | N | P | P |
| ORF1a | 3387 | N | N | P |
| ORF1a | 3388 | N | P | N |
| ORF1a | 3389 | N | N | P |
| ORF1a | 3392 | N | D | N |
| ORF1a | 3394 | P | P | P |
| ORF1a | 3395 | D | D | N |
| ORF1a | 3396 | P | P | P |
| ORF1a | 3397 | P | P | P |
| ORF1a | 3399 | N | N | P |
| ORF1a | 3400 | N | P | P |
| ORF1a | 3402 | N | P | P |
| ORF1a | 3403 | P | P | P |
| ORF1a | 3404 | P | N | P |
| ORF1a | 3405 | D + P | P | P |
| ORF1a | 3407 | P | P | P |
| ORF1a | 3413 | N | P | N |
| ORF1a | 3414 | P | P | P |
| ORF1a | 3417 | N | P | N |
| ORF1a | 3418 | P | P | N |
| ORF1a | 3420 | P | N | N |
| ORF1a | 3421 | N | P | N |
| ORF1a | 3424 | P | P | P |
| ORF1a | 3426 | P | P | P |
| ORF1a | 3427 | N | P | P |
| ORF1a | 3429 | P | P | P |
| ORF1a | 3430 | N | P | P |
| ORF1a | 3432 | N | P | N |
| ORF1a | 3435 | N | P | N |
| ORF1a | 3436 | N | P | P |
| ORF1a | 3438 | N | P | N |
| ORF1a | 3439 | P | P | P |
| ORF1a | 3441 | N | P | P |
| ORF1a | 3443 | P | P | P |
| ORF1a | 3444 | N | P | N |
| ORF1a | 3445 | N | P | P |
| ORF1a | 3447 | D | D | D |
| ORF1a | 3448 | N | P | P |
| ORF1a | 3450 | P | P | P |
| ORF1a | 3451 | N | P | N |
| ORF1a | 3452 | N | P | N |
| ORF1a | 3454 | D | D | D |
| ORF1a | 3456 | N | D | N |
| ORF1a | 3457 | N | P | N |
| ORF1a | 3459 | P | P | P |
| ORF1a | 3460 | N | P | P |
| ORF1a | 3463 | N | P | N |
| ORF1a | 3464 | N | P | P |
| ORF1a | 3465 | N | P | N |
| ORF1a | 3466 | N | P | N |
| ORF1a | 3472 | P | P | P |
| ORF1a | 3474 | N | P | P |
| ORF1a | 3475 | N | D | D |
| ORF1a | 3477 | N | N | P |
| ORF1a | 3478 | N | P | P |
| ORF1a | 3479 | P | P | P |
| ORF1a | 3480 | D | P | N |
| ORF1a | 3482 | N | P | N |
| ORF1a | 3483 | D | N | N |
| ORF1a | 3485 | N | P | P |
| ORF1a | 3487 | N | N | P |
| ORF1a | 3492 | P | P | P |
| ORF1a | 3493 | N | P | P |
| ORF1a | 3494 | P | P | P |
| ORF1a | 3495 | N | P | N |
| ORF1a | 3496 | N | P | P |
| ORF1a | 3497 | D | D | N |
| ORF1a | 3499 | D | D | N |
| ORF1a | 3500 | N | P | P |
| ORF1a | 3502 | N | P | P |
| ORF1a | 3504 | D | D | D |
| ORF1a | 3505 | P | P | N |
| ORF1a | 3507 | P | N | P |
| ORF1a | 3508 | P | P | P |
| ORF1a | 3509 | D | D | D |
| ORF1a | 3511 | N | P | N |
| ORF1a | 3513 | P | P | P |
| ORF1a | 3514 | N | N | P |
| ORF1a | 3515 | N | P | P |
| ORF1a | 3516 | N | P | P |
| ORF1a | 3517 | N | P | P |
| ORF1a | 3518 | N | P | N |
| ORF1a | 3519 | N | N | P |
| ORF1a | 3521 | N | P | P |
| ORF1a | 3523 | D | D | D |
| ORF1a | 3524 | D | N | N |
| ORF1a | 3525 | N | P | P |
| ORF1a | 3526 | N | D | N |
| ORF1a | 3529 | D | N | N |
| ORF1a | 3530 | N | P | P |
| ORF1a | 3532 | N | P | N |
| ORF1a | 3535 | P | P | P |
| ORF1a | 3537 | D | D | D |
| ORF1a | 3540 | P | N | P |
| ORF1a | 3546 | N | N | D |
| ORF1a | 3547 | N | N | D |
| ORF1a | 3549 | N | P | P |
| ORF1a | 3550 | N | N | P |
| ORF1a | 3552 | N | N | P |
| ORF1a | 3553 | N | P | N |
| ORF1a | 3555 | P | N | P |
| ORF1a | 3557 | N | P | P |
| ORF1a | 3561 | N | P | P |
| ORF1a | 3563 | P | P | P |
| ORF1a | 3564 | N | P | N |
| ORF1a | 3565 | N | P | N |
| ORF1a | 3568 | P | P | P |
| ORF1a | 3571 | P | P | N |
| ORF1a | 3572 | P | P | P |
| ORF1a | 3574 | N | N | D |
| ORF1a | 3575 | N | P | N |
| ORF1a | 3576 | N | P | P |
| ORF1a | 3577 | D | N | N |
| ORF1a | 3579 | N | D | N |
| ORF1a | 3580 | D | D | D |
| ORF1a | 3581 | P | P | P |
| ORF1a | 3583 | N | P | P |
| ORF1a | 3584 | N | P | P |
| ORF1a | 3585 | P | P | P |
| ORF1a | 3586 | N | P | P |
| ORF1a | 3588 | P | N | N |
| ORF1a | 3590 | P | P | N |
| ORF1a | 3592 | N | P | P |
| ORF1a | 3593 | D | D | N |
| ORF1a | 3595 | P | N | N |
| ORF1a | 3599 | N | N | P |
| ORF1a | 3602 | N | N | P |
| ORF1a | 3603 | P | P | P |
| ORF1a | 3604 | P | P | N |
| ORF1a | 3606 | D | D | D |
| ORF1a | 3608 | N | D | N |
| ORF1a | 3609 | P | N | N |
| ORF1a | 3610 | P | P | N |
| ORF1a | 3611 | N | P | P |
| ORF1a | 3613 | N | N | D |
| ORF1a | 3614 | N | P | P |
| ORF1a | 3615 | D | D | D |
| ORF1a | 3617 | N | N | D |
| ORF1a | 3618 | D | D | D |
| ORF1a | 3619 | N | P | N |
| ORF1a | 3620 | D | D | D |
| ORF1a | 3622 | N | N | P |
| ORF1a | 3624 | N | N | P |
| ORF1a | 3625 | N | N | D |
| ORF1a | 3628 | N | N | P |
| ORF1a | 3629 | P | P | P |
| ORF1a | 3631 | N | N | P |
| ORF1a | 3633 | N | P | N |
| ORF1a | 3635 | N | N | P |
| ORF1a | 3636 | P | P | P |
| ORF1a | 3637 | N | P | P |
| ORF1a | 3639 | P | P | P |
| ORF1a | 3640 | N | N | P |
| ORF1a | 3642 | P | P | N |
| ORF1a | 3643 | N | N | P |
| ORF1a | 3644 | D | D | D |
| ORF1a | 3645 | P | D + P | P |
| ORF1a | 3646 | N | D | D |
| ORF1a | 3647 | N | P | N |
| ORF1a | 3648 | D | N | N |
| ORF1a | 3650 | N | P | N |
| ORF1a | 3653 | P | D | N |
| ORF1a | 3654 | N | P | P |
| ORF1a | 3656 | P | P | P |
| ORF1a | 3657 | N | D | D |
| ORF1a | 3658 | N | P | P |
| ORF1a | 3660 | N | D | N |
| ORF1a | 3665 | P | N | N |
| ORF1a | 3668 | N | P | P |
| ORF1a | 3671 | P | P | N |
| ORF1a | 3674 | N | N | D |
| ORF1a | 3675 | N | D | N |
| ORF1a | 3676 | N | D | D |
| ORF1a | 3677 | N | D | D |
| ORF1a | 3679 | P | P | P |
| ORF1a | 3682 | N | N | P |
| ORF1a | 3689 | N | P | P |
| ORF1a | 3690 | P | P | P |
| ORF1a | 3692 | P | P | P |
| ORF1a | 3693 | N | N | P |
| ORF1a | 3694 | D | N | N |
| ORF1a | 3696 | N | P | P |
| ORF1a | 3697 | P | N | N |
| ORF1a | 3699 | N | P | N |
| ORF1a | 3700 | P | P | P |
| ORF1a | 3701 | N | P | N |
| ORF1a | 3702 | N | P | N |
| ORF1a | 3703 | N | P | P |
| ORF1a | 3705 | D | D | D |
| ORF1a | 3706 | N | P | P |
| ORF1a | 3707 | N | P | P |
| ORF1a | 3708 | P | P | N |
| ORF1a | 3710 | N | N | P |
| ORF1a | 3711 | D | D | N |
| ORF1a | 3714 | N | P | P |
| ORF1a | 3715 | P | N | N |
| ORF1a | 3716 | N | N | P |
| ORF1a | 3717 | P | N | P |
| ORF1a | 3718 | D | D | D |
| ORF1a | 3719 | N | P | P |
| ORF1a | 3722 | N | D | D |
| ORF1a | 3725 | D | N | N |
| ORF1a | 3727 | N | P | N |
| ORF1a | 3728 | N | P | P |
| ORF1a | 3729 | N | D | D |
| ORF1a | 3730 | P | P | P |
| ORF1a | 3731 | N | N | D |
| ORF1a | 3732 | N | D | P |
| ORF1a | 3735 | N | P | N |
| ORF1a | 3736 | N | D | N |
| ORF1a | 3738 | P | P | P |
| ORF1a | 3739 | N | P | P |
| ORF1a | 3740 | N | P | N |
| ORF1a | 3741 | N | P | P |
| ORF1a | 3742 | N | P | P |
| ORF1a | 3743 | N | N | P |
| ORF1a | 3744 | P | P | P |
| ORF1a | 3745 | P | P | N |
| ORF1a | 3746 | N | P | N |
| ORF1a | 3747 | N | P | P |
| ORF1a | 3750 | D | D | D |
| ORF1a | 3751 | N | P | N |
| ORF1a | 3753 | N | N | P |
| ORF1a | 3754 | N | P | N |
| ORF1a | 3755 | P | P | P |
| ORF1a | 3756 | P | P | P |
| ORF1a | 3758 | N | D | D |
| ORF1a | 3759 | D | N | N |
| ORF1a | 3766 | P | P | N |
| ORF1a | 3767 | N | D | N |
| ORF1a | 3768 | N | P | N |
| ORF1a | 3769 | P | P | P |
| ORF1a | 3770 | P | P | P |
| ORF1a | 3772 | P | P | N |
| ORF1a | 3774 | P | P | P |
| ORF1a | 3777 | D | D | D |
| ORF1a | 3778 | P | P | N |
| ORF1a | 3781 | N | P | N |
| ORF1a | 3784 | P | P | N |
| ORF1a | 3785 | P | P | P |
| ORF1a | 3786 | P | N | P |
| ORF1a | 3787 | N | P | P |
| ORF1a | 3788 | N | P | P |
| ORF1a | 3791 | P | P | P |
| ORF1a | 3792 | P | P | P |
| ORF1a | 3793 | P | P | P |
| ORF1a | 3794 | N | P | N |
| ORF1a | 3795 | P | P | P |
| ORF1a | 3796 | P | P | P |
| ORF1a | 3798 | N | P | P |
| ORF1a | 3800 | N | N | P |
| ORF1a | 3801 | N | N | P |
| ORF1a | 3802 | N | N | D |
| ORF1a | 3803 | P | P | P |
| ORF1a | 3804 | N | P | N |
| ORF1a | 3805 | N | P | N |
| ORF1a | 3807 | N | P | N |
| ORF1a | 3810 | N | N | P |
| ORF1a | 3811 | N | P | N |
| ORF1a | 3812 | P | N | N |
| ORF1a | 3813 | P | P | P |
| ORF1a | 3814 | N | P | N |
| ORF1a | 3815 | N | P | N |
| ORF1a | 3816 | N | P | N |
| ORF1a | 3817 | P | P | N |
| ORF1a | 3818 | P | P | P |
| ORF1a | 3821 | N | P | P |
| ORF1a | 3822 | N | P | P |
| ORF1a | 3824 | P | N | N |
| ORF1a | 3825 | P | P | P |
| ORF1a | 3826 | N | D | N |
| ORF1a | 3827 | N | P | P |
| ORF1a | 3828 | P | P | P |
| ORF1a | 3829 | N | D | D |
| ORF1a | 3830 | P | P | P |
| ORF1a | 3831 | P | P | P |
| ORF1a | 3832 | D | D | D |
| ORF1a | 3833 | P | N | N |
| ORF1a | 3834 | P | P | P |
| ORF1a | 3835 | N | N | P |
| ORF1a | 3836 | P | N | N |
| ORF1a | 3837 | P | P | P |
| ORF1a | 3838 | P | P | P |
| ORF1a | 3839 | P | P | P |
| ORF1a | 3840 | N | P | N |
| ORF1a | 3841 | N | P | N |
| ORF1a | 3842 | N | P | N |
| ORF1a | 3843 | N | P | N |
| ORF1a | 3844 | P | P | N |
| ORF1a | 3845 | N | P | P |
| ORF1a | 3848 | N | P | N |
| ORF1a | 3849 | P | P | P |
| ORF1a | 3851 | P | P | N |
| ORF1a | 3852 | N | P | N |
| ORF1a | 3853 | P | P | P |
| ORF1a | 3855 | P | P | P |
| ORF1a | 3856 | N | P | P |
| ORF1a | 3857 | P | P | N |
| ORF1a | 3858 | P | P | N |
| ORF1a | 3859 | P | P | P |
| ORF1a | 3860 | N | N | P |
| ORF1a | 3863 | N | P | P |
| ORF1a | 3866 | N | N | P |
| ORF1a | 3867 | P | P | P |
| ORF1a | 3868 | N | N | P |
| ORF1a | 3869 | P | P | N |
| ORF1a | 3870 | N | P | P |
| ORF1a | 3871 | P | P | P |
| ORF1a | 3872 | N | N | P |
| ORF1a | 3873 | N | P | P |
| ORF1a | 3875 | P | P | P |
| ORF1a | 3879 | N | P | P |
| ORF1a | 3881 | P | P | P |
| ORF1a | 3883 | N | P | P |
| ORF1a | 3884 | D | D | N |
| ORF1a | 3885 | D | D | N |
| ORF1a | 3886 | P | P | P |
| ORF1a | 3887 | N | P | N |
| ORF1a | 3889 | N | P | N |
| ORF1a | 3891 | P | P | P |
| ORF1a | 3892 | P | P | P |
| ORF1a | 3894 | N | P | P |
| ORF1a | 3895 | N | P | P |
| ORF1a | 3897 | P | P | P |
| ORF1a | 3898 | N | P | N |
| ORF1a | 3899 | P | P | P |
| ORF1a | 3900 | N | P | P |
| ORF1a | 3903 | P | P | P |
| ORF1a | 3906 | P | P | P |
| ORF1a | 3907 | P | P | P |
| ORF1a | 3908 | N | N | P |
| ORF1a | 3909 | D | D | N |
| ORF1a | 3912 | N | P | N |
| ORF1a | 3913 | P | P | P |
| ORF1a | 3914 | N | P | P |
| ORF1a | 3915 | D | D | D |
| ORF1a | 3917 | N | N | P |
| ORF1a | 3918 | N | P | P |
| ORF1a | 3920 | P | P | P |
| ORF1a | 3922 | N | N | D |
| ORF1a | 3923 | N | P | P |
| ORF1a | 3926 | P | P | P |
| ORF1a | 3927 | N | P | N |
| ORF1a | 3928 | P | P | P |
| ORF1a | 3930 | D | D | D |
| ORF1a | 3931 | P | N | N |
| ORF1a | 3932 | N | P | N |
| ORF1a | 3935 | P | P | P |
| ORF1a | 3936 | P | P | N |
| ORF1a | 3937 | P | P | P |
| ORF1a | 3938 | N | P | N |
| ORF1a | 3939 | N | N | P |
| ORF1a | 3940 | P | P | P |
| ORF1a | 3941 | P | P | N |
| ORF1a | 3943 | P | P | P |
| ORF1a | 3945 | P | P | P |
| ORF1a | 3946 | P | P | P |
| ORF1a | 3947 | N | N | P |
| ORF1a | 3948 | N | P | P |
| ORF1a | 3949 | N | P | N |
| ORF1a | 3950 | P | P | P |
| ORF1a | 3951 | D | D | N |
| ORF1a | 3952 | P | P | P |
| ORF1a | 3953 | N | P | P |
| ORF1a | 3954 | N | P | P |
| ORF1a | 3955 | P | N | P |
| ORF1a | 3958 | P | P | P |
| ORF1a | 3959 | N | D | N |
| ORF1a | 3960 | P | P | P |
| ORF1a | 3965 | P | P | P |
| ORF1a | 3966 | N | D | N |
| ORF1a | 3967 | N | N | P |
| ORF1a | 3973 | N | P | P |
| ORF1a | 3976 | D | D | D |
| ORF1a | 3977 | N | N | P |
| ORF1a | 3978 | P | N | N |
| ORF1a | 3979 | D | D | N |
| ORF1a | 3980 | N | P | N |
| ORF1a | 3983 | N | D | N |
| ORF1a | 3985 | N | P | N |
| ORF1a | 3986 | P | P | P |
| ORF1a | 3989 | N | P | P |
| ORF1a | 3991 | N | N | P |
| ORF1a | 3992 | N | P | P |
| ORF1a | 3993 | D | D | N |
| ORF1a | 3994 | N | P | N |
| ORF1a | 3995 | N | P | P |
| ORF1a | 3996 | N | N | P |
| ORF1a | 3999 | N | P | P |
| ORF1a | 4001 | N | P | P |
| ORF1a | 4003 | N | N | P |
| ORF1a | 4006 | P | P | P |
| ORF1a | 4010 | P | P | P |
| ORF1a | 4013 | N | P | P |
| ORF1a | 4014 | N | P | P |
| ORF1a | 4015 | N | P | P |
| ORF1a | 4016 | N | D | D |
| ORF1a | 4017 | P | P | N |
| ORF1a | 4018 | N | N | P |
| ORF1a | 4019 | P | P | P |
| ORF1a | 4020 | N | P | P |
| ORF1a | 4022 | N | N | P |
| ORF1a | 4023 | N | P | P |
| ORF1a | 4024 | N | P | N |
| ORF1a | 4030 | N | N | P |
| ORF1a | 4031 | N | D | N |
| ORF1a | 4033 | N | N | P |
| ORF1a | 4034 | P | P | P |
| ORF1a | 4037 | D | N | N |
| ORF1a | 4040 | N | P | N |
| ORF1a | 4041 | P | P | P |
| ORF1a | 4045 | P | P | P |
| ORF1a | 4046 | N | P | N |
| ORF1a | 4047 | P | P | P |
| ORF1a | 4049 | D | D | N |
| ORF1a | 4050 | N | P | N |
| ORF1a | 4052 | N | P | P |
| ORF1a | 4053 | N | P | N |
| ORF1a | 4054 | N | P | N |
| ORF1a | 4056 | N | N | P |
| ORF1a | 4057 | N | P | P |
| ORF1a | 4058 | P | P | P |
| ORF1a | 4059 | N | N | P |
| ORF1a | 4060 | N | P | N |
| ORF1a | 4062 | N | P | P |
| ORF1a | 4063 | N | P | P |
| ORF1a | 4064 | N | D | N |
| ORF1a | 4065 | D | D | D |
| ORF1a | 4066 | P | N | N |
| ORF1a | 4067 | N | P | P |
| ORF1a | 4068 | N | P | P |
| ORF1a | 4069 | N | N | P |
| ORF1a | 4070 | P | P | P |
| ORF1a | 4073 | P | P | P |
| ORF1a | 4075 | D | N | N |
| ORF1a | 4077 | N | P | P |
| ORF1a | 4080 | D | N | N |
| ORF1a | 4082 | P | P | P |
| ORF1a | 4083 | P | P | P |
| ORF1a | 4087 | D | D | D |
| ORF1a | 4089 | N | N | P |
| ORF1a | 4090 | D | D | D |
| ORF1a | 4091 | N | P | P |
| ORF1a | 4092 | N | P | P |
| ORF1a | 4093 | P | P | P |
| ORF1a | 4097 | N | D | N |
| ORF1a | 4098 | N | P | N |
| ORF1a | 4100 | D | D | N |
| ORF1a | 4102 | N | N | P |
| ORF1a | 4104 | P | P | N |
| ORF1a | 4119 | N | P | N |
| ORF1a | 4121 | N | P | P |
| ORF1a | 4123 | N | P | P |
| ORF1a | 4125 | N | P | P |
| ORF1a | 4128 | P | N | N |
| ORF1a | 4129 | N | D | D |
| ORF1a | 4132 | P | P | P |
| ORF1a | 4134 | N | N | P |
| ORF1a | 4135 | N | N | P |
| ORF1a | 4137 | P | P | P |
| ORF1a | 4143 | P | P | P |
| ORF1a | 4144 | N | P | N |
| ORF1a | 4146 | N | P | P |
| ORF1a | 4147 | N | D | N |
| ORF1a | 4148 | P | N | P |
| ORF1a | 4149 | N | P | P |
| ORF1a | 4150 | N | N | P |
| ORF1a | 4153 | P | P | N |
| ORF1a | 4155 | P | N | N |
| ORF1a | 4156 | P | P | P |
| ORF1a | 4157 | P | N | N |
| ORF1a | 4158 | N | N | D |
| ORF1a | 4159 | N | D | D |
| ORF1a | 4161 | D | D | D |
| ORF1a | 4163 | P | P | P |
| ORF1a | 4164 | D | D | D |
| ORF1a | 4165 | N | N | D |
| ORF1a | 4166 | N | P | N |
| ORF1a | 4168 | P | P | P |
| ORF1a | 4169 | N | P | P |
| ORF1a | 4170 | N | P | P |
| ORF1a | 4171 | N | P | P |
| ORF1a | 4172 | P | P | P |
| ORF1a | 4173 | P | P | P |
| ORF1a | 4174 | N | D | D |
| ORF1a | 4175 | D | D | D |
| ORF1a | 4176 | N | D | N |
| ORF1a | 4177 | D | N | P |
| ORF1a | 4178 | N | D | D |
| ORF1a | 4181 | N | N | P |
| ORF1a | 4182 | N | N | D |
| ORF1a | 4183 | N | N | P |
| ORF1a | 4184 | P | P | P |
| ORF1a | 4185 | P | P | P |
| ORF1a | 4186 | P | P | P |
| ORF1a | 4188 | N | P | P |
| ORF1a | 4191 | N | P | N |
| ORF1a | 4192 | N | P | P |
| ORF1a | 4195 | N | P | P |
| ORF1a | 4196 | N | P | P |
| ORF1a | 4197 | D | D | D |
| ORF1a | 4200 | P | P | P |
| ORF1a | 4205 | P | P | P |
| ORF1a | 4206 | P | N | P |
| ORF1a | 4207 | D | P | P |
| ORF1a | 4208 | N | N | P |
| ORF1a | 4209 | N | P | P |
| ORF1a | 4211 | P | N | N |
| ORF1a | 4212 | N | P | P |
| ORF1a | 4214 | P | N | P |
| ORF1a | 4215 | N | P | P |
| ORF1a | 4217 | N | N | P |
| ORF1a | 4218 | P | N | N |
| ORF1a | 4219 | N | P | N |
| ORF1a | 4220 | D | D | D |
| ORF1a | 4221 | N | N | P |
| ORF1a | 4223 | N | N | D |
| ORF1a | 4225 | N | P | N |
| ORF1a | 4227 | N | P | P |
| ORF1a | 4228 | N | N | P |
| ORF1a | 4229 | N | P | P |
| ORF1a | 4233 | P | P | P |
| ORF1a | 4234 | N | P | N |
| ORF1a | 4235 | P | P | P |
| ORF1a | 4236 | P | P | P |
| ORF1a | 4237 | P | P | P |
| ORF1a | 4238 | N | P | N |
| ORF1a | 4242 | P | P | N |
| ORF1a | 4243 | N | P | N |
| ORF1a | 4246 | N | P | N |
| ORF1a | 4247 | N | P | P |
| ORF1a | 4249 | N | D | D |
| ORF1a | 4250 | N | P | N |
| ORF1a | 4251 | N | N | P |
| ORF1a | 4252 | P | P | P |
| ORF1a | 4253 | N | N | P |
| ORF1a | 4254 | N | P | P |
| ORF1a | 4255 | N | N | P |
| ORF1a | 4256 | N | D | N |
| ORF1a | 4257 | P | P | N |
| ORF1a | 4258 | N | P | P |
| ORF1a | 4260 | P | P | P |
| ORF1a | 4261 | N | P | N |
| ORF1a | 4262 | P | P | P |
| ORF1a | 4264 | N | P | P |
| ORF1a | 4265 | D | D | D |
| ORF1a | 4266 | P | P | P |
| ORF1a | 4267 | N | P | N |
| ORF1a | 4268 | N | P | P |
| ORF1a | 4269 | P | P | P |
| ORF1a | 4270 | N | P | N |
| ORF1a | 4271 | N | P | P |
| ORF1a | 4272 | N | P | N |
| ORF1a | 4274 | N | P | P |
| ORF1a | 4275 | N | P | P |
| ORF1a | 4276 | N | P | N |
| ORF1a | 4277 | N | N | P |
| ORF1a | 4279 | N | P | N |
| ORF1a | 4280 | N | P | P |
| ORF1a | 4281 | N | N | P |
| ORF1a | 4282 | N | N | P |
| ORF1a | 4283 | P | P | P |
| ORF1a | 4284 | P | P | P |
| ORF1a | 4285 | D | D | D |
| ORF1a | 4287 | P | P | P |
| ORF1a | 4288 | N | N | P |
| ORF1a | 4290 | N | P | N |
| ORF1a | 4291 | N | P | P |
| ORF1a | 4292 | N | D | N |
| ORF1a | 4298 | P | N | N |
| ORF1a | 4299 | P | P | P |
| ORF1a | 4300 | N | N | P |
| ORF1a | 4301 | N | P | P |
| ORF1a | 4303 | N | P | N |
| ORF1a | 4304 | D | D | D |
| ORF1a | 4305 | N | P | N |
| ORF1a | 4307 | P | P | P |
| ORF1a | 4309 | P | N | N |
| ORF1a | 4310 | N | P | P |
| ORF1a | 4311 | N | D | N |
| ORF1a | 4312 | P | P | P |
| ORF1a | 4314 | N | N | P |
| ORF1a | 4315 | N | N | P |
| ORF1a | 4317 | P | P | N |
| ORF1a | 4318 | N | N | P |
| ORF1a | 4319 | N | N | P |
| ORF1a | 4320 | N | N | P |
| ORF1a | 4321 | N | P | P |
| ORF1a | 4324 | N | P | P |
| ORF1a | 4325 | P | P | P |
| ORF1a | 4327 | N | P | N |
| ORF1a | 4328 | P | P | P |
| ORF1a | 4330 | P | N | P |
| ORF1a | 4331 | P | N | N |
| ORF1a | 4332 | P | N | P |
| ORF1a | 4333 | N | P | P |
| ORF1a | 4334 | N | N | P |
| ORF1a | 4335 | N | D + P | N |
| ORF1a | 4336 | N | P | P |
| ORF1a | 4337 | D | D | D |
| ORF1a | 4340 | N | P | N |
| ORF1a | 4341 | N | P | N |
| ORF1a | 4344 | P | P | N |
| ORF1a | 4345 | N | P | N |
| ORF1a | 4348 | D | N | N |
| ORF1a | 4349 | N | N | P |
| ORF1a | 4350 | P | N | N |
| ORF1a | 4353 | N | P | P |
| ORF1a | 4355 | D | D | D |
| ORF1a | 4357 | D | D | D |
| ORF1a | 4358 | P | P | P |
| ORF1a | 4360 | N | P | P |
| ORF1a | 4361 | P | P | P |
| ORF1a | 4363 | N | P | N |
| ORF1a | 4364 | N | D | D |
| ORF1a | 4365 | N | P | N |
| ORF1a | 4367 | P | P | P |
| ORF1a | 4369 | P | P | P |
| ORF1a | 4370 | N | N | P |
| ORF1a | 4371 | P | P | P |
| ORF1a | 4372 | P | P | P |
| ORF1a | 4373 | P | P | P |
| ORF1a | 4377 | N | P | P |
| ORF1a | 4379 | N | N | P |
| ORF1a | 4380 | N | P | P |
| ORF1a | 4384 | N | P | P |
| ORF1a | 4386 | N | N | P |
| ORF1a | 4387 | N | D | N |
| ORF1a | 4388 | N | P | N |
| ORF1a | 4393 | N | P | P |
| ORF1a | 4394 | N | D | D |
| ORF1a | 4395 | D | N | N |
| ORF1a | 4396 | N | N | P |
| ORF1a | 4398 | P | P | P |
| ORF1b | 3 | P | N | N |
| ORF1b | 5 | P | P | P |
| ORF1b | 7 | D | D + P | D |
| ORF1b | 11 | N | P | N |
| ORF1b | 12 | P | N | N |
| ORF1b | 13 | N | P | N |
| ORF1b | 15 | P | P | N |
| ORF1b | 16 | D | N | N |
| ORF1b | 17 | D | D | D |
| ORF1b | 18 | N | P | N |
| ORF1b | 20 | P | N | P |
| ORF1b | 22 | P | P | P |
| ORF1b | 23 | P | P | P |
| ORF1b | 25 | N | P | N |
| ORF1b | 27 | P | P | P |
| ORF1b | 28 | P | P | P |
| ORF1b | 29 | P | P | P |
| ORF1b | 31 | N | P | N |
| ORF1b | 33 | P | P | P |
| ORF1b | 34 | N | P | N |
| ORF1b | 35 | N | D | N |
| ORF1b | 37 | N | P | N |
| ORF1b | 39 | P | P | P |
| ORF1b | 40 | N | P | N |
| ORF1b | 44 | P | N | N |
| ORF1b | 46 | N | P | P |
| ORF1b | 49 | N | P | P |
| ORF1b | 50 | D | N | N |
| ORF1b | 51 | N | P | P |
| ORF1b | 53 | N | D | N |
| ORF1b | 54 | N | N | D |
| ORF1b | 57 | N | N | P |
| ORF1b | 60 | N | P | P |
| ORF1b | 62 | P | P | P |
| ORF1b | 64 | N | N | P |
| ORF1b | 65 | N | P | N |
| ORF1b | 66 | P | P | P |
| ORF1b | 67 | D | N | D |
| ORF1b | 71 | N | N | P |
| ORF1b | 76 | D | D | D |
| ORF1b | 78 | N | P | P |
| ORF1b | 82 | D | D | D |
| ORF1b | 83 | N | N | D |
| ORF1b | 85 | N | D | N |
| ORF1b | 86 | N | D | N |
| ORF1b | 87 | N | D | N |
| ORF1b | 88 | D | D | D |
| ORF1b | 90 | P | P | N |
| ORF1b | 96 | N | P | P |
| ORF1b | 98 | N | N | D |
| ORF1b | 99 | D | D | D |
| ORF1b | 100 | N | D | N |
| ORF1b | 102 | N | P | N |
| ORF1b | 104 | P | P | P |
| ORF1b | 106 | N | P | P |
| ORF1b | 107 | P | N | P |
| ORF1b | 108 | N | N | P |
| ORF1b | 114 | N | P | N |
| ORF1b | 116 | P | P | P |
| ORF1b | 117 | N | N | P |
| ORF1b | 118 | N | P | P |
| ORF1b | 120 | N | N | P |
| ORF1b | 126 | N | P | N |
| ORF1b | 127 | N | P | N |
| ORF1b | 129 | N | P | P |
| ORF1b | 131 | P | P | N |
| ORF1b | 132 | D | D | D |
| ORF1b | 133 | N | P | N |
| ORF1b | 139 | N | P | N |
| ORF1b | 140 | P | P | P |
| ORF1b | 144 | D | D | D |
| ORF1b | 145 | N | N | D |
| ORF1b | 146 | N | D | D |
| ORF1b | 147 | N | D | N |
| ORF1b | 151 | N | D | N |
| ORF1b | 152 | N | N | D |
| ORF1b | 154 | N | N | P |
| ORF1b | 155 | N | P | P |
| ORF1b | 156 | N | P | N |
| ORF1b | 157 | N | N | P |
| ORF1b | 159 | P | P | P |
| ORF1b | 160 | N | P | N |
| ORF1b | 161 | N | D | N |
| ORF1b | 163 | N | P | N |
| ORF1b | 164 | D | D | D |
| ORF1b | 165 | P | P | P |
| ORF1b | 166 | N | P | P |
| ORF1b | 167 | N | P | P |
| ORF1b | 170 | D | D | N |
| ORF1b | 174 | P | P | P |
| ORF1b | 176 | D | D | D |
| ORF1b | 180 | N | P | N |
| ORF1b | 181 | P | P | P |
| ORF1b | 183 | P | P | N |
| ORF1b | 188 | N | N | D |
| ORF1b | 189 | N | P | P |
| ORF1b | 190 | N | N | P |
| ORF1b | 192 | N | D | D |
| ORF1b | 196 | P | P | P |
| ORF1b | 198 | N | N | P |
| ORF1b | 199 | N | P | P |
| ORF1b | 200 | N | P | P |
| ORF1b | 202 | P | P | P |
| ORF1b | 203 | N | P | N |
| ORF1b | 208 | N | N | P |
| ORF1b | 209 | P | N | N |
| ORF1b | 210 | P | P | P |
| ORF1b | 211 | P | P | P |
| ORF1b | 212 | P | P | P |
| ORF1b | 213 | N | P | N |
| ORF1b | 217 | P | P | P |
| ORF1b | 218 | D | N | N |
| ORF1b | 219 | D | D | N |
| ORF1b | 220 | N | D | D |
| ORF1b | 221 | N | P | N |
| ORF1b | 222 | P | N | N |
| ORF1b | 223 | N | P | P |
| ORF1b | 225 | N | P | N |
| ORF1b | 226 | N | P | P |
| ORF1b | 227 | N | P | P |
| ORF1b | 230 | N | P | N |
| ORF1b | 231 | N | N | P |
| ORF1b | 234 | N | N | P |
| ORF1b | 237 | P | P | P |
| ORF1b | 239 | P | P | P |
| ORF1b | 240 | N | P | N |
| ORF1b | 242 | N | P | P |
| ORF1b | 244 | N | P | N |
| ORF1b | 246 | N | P | N |
| ORF1b | 247 | N | P | N |
| ORF1b | 248 | D | D | D |
| ORF1b | 249 | N | D | D |
| ORF1b | 251 | P | P | P |
| ORF1b | 252 | D | D | D |
| ORF1b | 253 | D | N | N |
| ORF1b | 254 | N | N | P |
| ORF1b | 255 | N | N | D |
| ORF1b | 256 | P | N | N |
| ORF1b | 257 | P | N | P |
| ORF1b | 260 | N | N | D |
| ORF1b | 263 | N | P | N |
| ORF1b | 265 | N | P | P |
| ORF1b | 266 | N | P | P |
| ORF1b | 267 | N | N | P |
| ORF1b | 268 | N | P | N |
| ORF1b | 269 | N | N | P |
| ORF1b | 270 | N | N | D |
| ORF1b | 273 | N | P | N |
| ORF1b | 275 | D | D | D |
| ORF1b | 282 | P | P | P |
| ORF1b | 284 | N | N | D |
| ORF1b | 285 | P | P | P |
| ORF1b | 287 | N | P | P |
| ORF1b | 292 | N | N | P |
| ORF1b | 294 | D + P | N | N |
| ORF1b | 295 | N | N | P |
| ORF1b | 296 | N | N | P |
| ORF1b | 297 | P | P | P |
| ORF1b | 298 | N | N | P |
| ORF1b | 299 | N | P | P |
| ORF1b | 307 | N | P | P |
| ORF1b | 309 | N | P | P |
| ORF1b | 311 | N | P | P |
| ORF1b | 312 | N | P | P |
| ORF1b | 313 | N | P | P |
| ORF1b | 314 | D | D | D |
| ORF1b | 315 | N | P | N |
| ORF1b | 316 | N | D | N |
| ORF1b | 317 | N | P | P |
| ORF1b | 320 | N | P | N |
| ORF1b | 321 | N | P | P |
| ORF1b | 323 | N | P | N |
| ORF1b | 327 | N | P | N |
| ORF1b | 328 | N | N | P |
| ORF1b | 329 | P | N | N |
| ORF1b | 330 | N | P | P |
| ORF1b | 332 | N | N | P |
| ORF1b | 333 | N | P | N |
| ORF1b | 334 | N | N | P |
| ORF1b | 336 | N | P | P |
| ORF1b | 337 | D | N | P |
| ORF1b | 339 | N | P | P |
| ORF1b | 340 | N | P | N |
| ORF1b | 342 | P | P | P |
| ORF1b | 345 | P | P | P |
| ORF1b | 346 | N | N | P |
| ORF1b | 347 | P | P | P |
| ORF1b | 348 | N | N | D |
| ORF1b | 349 | P | N | N |
| ORF1b | 350 | N | P | N |
| ORF1b | 356 | N | N | P |
| ORF1b | 360 | N | P | P |
| ORF1b | 362 | N | N | P |
| ORF1b | 364 | N | P | P |
| ORF1b | 365 | N | P | N |
| ORF1b | 367 | P | P | P |
| ORF1b | 368 | N | P | P |
| ORF1b | 369 | N | P | N |
| ORF1b | 370 | N | D | D |
| ORF1b | 372 | P | P | P |
| ORF1b | 373 | D | D | N |
| ORF1b | 375 | N | N | P |
| ORF1b | 378 | P | P | P |
| ORF1b | 379 | N | P | N |
| ORF1b | 380 | N | P | N |
| ORF1b | 381 | N | P | P |
| ORF1b | 382 | P | N | N |
| ORF1b | 383 | N | P | P |
| ORF1b | 384 | N | P | P |
| ORF1b | 385 | N | P | N |
| ORF1b | 386 | P | P | P |
| ORF1b | 387 | N | N | P |
| ORF1b | 389 | P | P | P |
| ORF1b | 390 | N | P | P |
| ORF1b | 392 | N | P | P |
| ORF1b | 393 | N | P | P |
| ORF1b | 394 | P | P | P |
| ORF1b | 395 | N | N | P |
| ORF1b | 396 | D | D | N |
| ORF1b | 397 | N | P | N |
| ORF1b | 401 | P | P | P |
| ORF1b | 402 | P | N | N |
| ORF1b | 403 | P | P | P |
| ORF1b | 407 | P | P | P |
| ORF1b | 408 | P | P | P |
| ORF1b | 409 | N | P | N |
| ORF1b | 410 | P | P | P |
| ORF1b | 411 | P | N | P |
| ORF1b | 413 | N | N | P |
| ORF1b | 414 | D | D | D |
| ORF1b | 415 | P | P | P |
| ORF1b | 416 | N | P | N |
| ORF1b | 418 | P | P | P |
| ORF1b | 419 | P | P | P |
| ORF1b | 422 | N | P | N |
| ORF1b | 424 | P | N | N |
| ORF1b | 425 | N | D | N |
| ORF1b | 427 | N | P | P |
| ORF1b | 428 | N | N | P |
| ORF1b | 430 | N | P | N |
| ORF1b | 431 | N | P | P |
| ORF1b | 432 | N | P | N |
| ORF1b | 433 | N | P | P |
| ORF1b | 434 | D | D | N |
| ORF1b | 435 | D | N | P |
| ORF1b | 436 | D + P | D | N |
| ORF1b | 439 | N | P | N |
| ORF1b | 440 | D | D | D |
| ORF1b | 441 | P | P | P |
| ORF1b | 442 | N | D | N |
| ORF1b | 443 | N | P | N |
| ORF1b | 446 | P | P | P |
| ORF1b | 447 | N | P | P |
| ORF1b | 449 | N | P | P |
| ORF1b | 450 | N | N | P |
| ORF1b | 451 | N | P | P |
| ORF1b | 456 | N | P | N |
| ORF1b | 457 | N | D | N |
| ORF1b | 458 | P | P | P |
| ORF1b | 459 | N | N | P |
| ORF1b | 461 | P | N | N |
| ORF1b | 462 | N | P | P |
| ORF1b | 463 | N | P | N |
| ORF1b | 464 | D | D | D |
| ORF1b | 469 | D | D | D |
| ORF1b | 470 | P | P | P |
| ORF1b | 474 | N | P | P |
| ORF1b | 476 | N | P | P |
| ORF1b | 477 | P | P | P |
| ORF1b | 481 | P | N | N |
| ORF1b | 482 | D | N | P |
| ORF1b | 484 | N | P | P |
| ORF1b | 485 | N | N | P |
| ORF1b | 486 | P | P | P |
| ORF1b | 487 | N | P | N |
| ORF1b | 488 | P | P | P |
| ORF1b | 489 | P | P | P |
| ORF1b | 490 | P | P | P |
| ORF1b | 491 | N | P | P |
| ORF1b | 492 | N | P | P |
| ORF1b | 495 | N | P | P |
| ORF1b | 496 | P | P | P |
| ORF1b | 497 | N | P | N |
| ORF1b | 498 | P | P | N |
| ORF1b | 499 | P | P | P |
| ORF1b | 502 | P | N | N |
| ORF1b | 503 | N | N | P |
| ORF1b | 504 | P | P | P |
| ORF1b | 505 | D | N | N |
| ORF1b | 507 | P | N | P |
| ORF1b | 508 | N | P | N |
| ORF1b | 509 | P | P | N |
| ORF1b | 511 | N | D | N |
| ORF1b | 512 | N | N | D |
| ORF1b | 514 | P | N | P |
| ORF1b | 515 | N | P | P |
| ORF1b | 516 | N | N | P |
| ORF1b | 517 | D | D | D |
| ORF1b | 518 | N | P | N |
| ORF1b | 519 | P | P | P |
| ORF1b | 520 | D | D | D |
| ORF1b | 521 | N | P | P |
| ORF1b | 522 | P | P | P |
| ORF1b | 523 | P | P | N |
| ORF1b | 525 | N | N | P |
| ORF1b | 527 | P | P | N |
| ORF1b | 532 | N | P | N |
| ORF1b | 541 | N | N | P |
| ORF1b | 543 | P | P | P |
| ORF1b | 546 | N | P | N |
| ORF1b | 547 | N | P | P |
| ORF1b | 548 | N | P | P |
| ORF1b | 549 | N | P | P |
| ORF1b | 551 | P | P | P |
| ORF1b | 552 | N | P | N |
| ORF1b | 554 | N | P | N |
| ORF1b | 555 | N | P | N |
| ORF1b | 558 | P | P | P |
| ORF1b | 559 | N | N | P |
| ORF1b | 560 | N | N | P |
| ORF1b | 562 | N | N | P |
| ORF1b | 563 | N | P | P |
| ORF1b | 564 | N | N | P |
| ORF1b | 565 | P | P | P |
| ORF1b | 566 | P | P | P |
| ORF1b | 567 | P | P | P |
| ORF1b | 568 | N | P | P |
| ORF1b | 569 | N | P | P |
| ORF1b | 571 | N | P | P |
| ORF1b | 572 | N | D | N |
| ORF1b | 574 | P | P | P |
| ORF1b | 575 | P | P | P |
| ORF1b | 576 | N | D | N |
| ORF1b | 578 | N | P | N |
| ORF1b | 579 | N | N | P |
| ORF1b | 580 | N | P | N |
| ORF1b | 582 | N | P | N |
| ORF1b | 583 | P | P | P |
| ORF1b | 584 | P | P | P |
| ORF1b | 585 | P | P | P |
| ORF1b | 586 | N | P | N |
| ORF1b | 587 | N | N | D |
| ORF1b | 588 | N | N | P |
| ORF1b | 590 | P | P | P |
| ORF1b | 591 | P | P | P |
| ORF1b | 594 | P | P | P |
| ORF1b | 597 | N | N | P |
| ORF1b | 598 | D | N | N |
| ORF1b | 599 | N | P | P |
| ORF1b | 600 | N | P | P |
| ORF1b | 601 | N | N | P |
| ORF1b | 603 | P | P | P |
| ORF1b | 604 | N | D | D |
| ORF1b | 610 | N | P | N |
| ORF1b | 611 | N | P | P |
| ORF1b | 612 | N | N | P |
| ORF1b | 614 | N | P | P |
| ORF1b | 615 | P | P | N |
| ORF1b | 616 | N | P | N |
| ORF1b | 618 | N | P | P |
| ORF1b | 619 | P | P | P |
| ORF1b | 621 | N | P | N |
| ORF1b | 622 | N | P | P |
| ORF1b | 625 | P | P | P |
| ORF1b | 626 | P | N | N |
| ORF1b | 629 | D | D | D |
| ORF1b | 630 | P | N | N |
| ORF1b | 631 | N | P | P |
| ORF1b | 632 | P | P | N |
| ORF1b | 633 | P | P | P |
| ORF1b | 634 | N | N | D |
| ORF1b | 635 | P | P | P |
| ORF1b | 638 | D | D | D |
| ORF1b | 639 | P | N | N |
| ORF1b | 640 | N | N | P |
| ORF1b | 642 | P | N | N |
| ORF1b | 643 | N | P | P |
| ORF1b | 644 | N | P | P |
| ORF1b | 645 | N | P | P |
| ORF1b | 646 | P | P | P |
| ORF1b | 647 | D | D | D |
| ORF1b | 652 | N | N | P |
| ORF1b | 653 | P | P | P |
| ORF1b | 654 | N | P | P |
| ORF1b | 656 | P | P | P |
| ORF1b | 658 | N | N | P |
| ORF1b | 661 | P | P | P |
| ORF1b | 662 | D | D | D |
| ORF1b | 663 | N | P | P |
| ORF1b | 666 | N | N | P |
| ORF1b | 667 | P | P | P |
| ORF1b | 668 | P | P | P |
| ORF1b | 669 | N | P | P |
| ORF1b | 670 | P | P | P |
| ORF1b | 671 | P | P | P |
| ORF1b | 672 | P | P | P |
| ORF1b | 673 | P | P | P |
| ORF1b | 674 | P | P | P |
| ORF1b | 675 | N | N | P |
| ORF1b | 676 | N | P | N |
| ORF1b | 677 | P | P | P |
| ORF1b | 679 | N | N | P |
| ORF1b | 680 | N | P | N |
| ORF1b | 681 | P | N | N |
| ORF1b | 685 | D | D | D |
| ORF1b | 688 | N | N | P |
| ORF1b | 689 | N | N | P |
| ORF1b | 690 | D | N | N |
| ORF1b | 691 | P | P | P |
| ORF1b | 692 | P | P | P |
| ORF1b | 693 | N | P | N |
| ORF1b | 695 | N | N | P |
| ORF1b | 699 | N | P | N |
| ORF1b | 700 | N | P | N |
| ORF1b | 702 | D | P | N |
| ORF1b | 704 | P | P | P |
| ORF1b | 705 | N | N | P |
| ORF1b | 707 | P | P | P |
| ORF1b | 709 | D | D | N |
| ORF1b | 714 | N | P | N |
| ORF1b | 717 | N | P | P |
| ORF1b | 719 | N | P | P |
| ORF1b | 720 | D | N | N |
| ORF1b | 724 | N | N | P |
| ORF1b | 727 | N | D | N |
| ORF1b | 729 | N | N | P |
| ORF1b | 730 | D | D | D |
| ORF1b | 732 | N | N | P |
| ORF1b | 734 | N | N | D |
| ORF1b | 735 | D | D | D |
| ORF1b | 738 | P | P | N |
| ORF1b | 739 | N | P | N |
| ORF1b | 742 | N | N | P |
| ORF1b | 743 | N | P | P |
| ORF1b | 744 | N | P | P |
| ORF1b | 745 | N | N | P |
| ORF1b | 749 | P | P | P |
| ORF1b | 751 | P | P | P |
| ORF1b | 752 | N | P | P |
| ORF1b | 753 | N | P | N |
| ORF1b | 755 | P | P | P |
| ORF1b | 757 | P | P | P |
| ORF1b | 758 | N | P | P |
| ORF1b | 761 | N | P | P |
| ORF1b | 762 | P | P | P |
| ORF1b | 763 | N | N | D |
| ORF1b | 765 | N | D | N |
| ORF1b | 766 | P | P | P |
| ORF1b | 767 | D | N | N |
| ORF1b | 771 | N | D | N |
| ORF1b | 773 | N | P | N |
| ORF1b | 774 | N | P | N |
| ORF1b | 775 | P | P | P |
| ORF1b | 776 | N | P | N |
| ORF1b | 779 | N | P | N |
| ORF1b | 781 | P | P | P |
| ORF1b | 782 | N | P | P |
| ORF1b | 784 | N | N | P |
| ORF1b | 789 | N | P | P |
| ORF1b | 792 | D | N | N |
| ORF1b | 793 | P | P | N |
| ORF1b | 794 | D | D | D |
| ORF1b | 796 | N | P | N |
| ORF1b | 797 | D | D | D |
| ORF1b | 798 | N | N | P |
| ORF1b | 799 | N | P | N |
| ORF1b | 801 | N | P | P |
| ORF1b | 802 | P | P | P |
| ORF1b | 804 | N | P | N |
| ORF1b | 805 | N | P | P |
| ORF1b | 807 | P | P | P |
| ORF1b | 808 | P | P | N |
| ORF1b | 810 | N | P | N |
| ORF1b | 813 | D | D | D |
| ORF1b | 814 | D | D | D |
| ORF1b | 815 | N | N | D |
| ORF1b | 818 | P | P | P |
| ORF1b | 819 | P | P | P |
| ORF1b | 820 | N | D | N |
| ORF1b | 822 | P | N | P |
| ORF1b | 823 | P | P | P |
| ORF1b | 824 | P | P | P |
| ORF1b | 825 | P | P | P |
| ORF1b | 826 | P | P | P |
| ORF1b | 827 | P | N | P |
| ORF1b | 828 | P | P | P |
| ORF1b | 829 | P | P | P |
| ORF1b | 830 | P | P | P |
| ORF1b | 831 | P | P | P |
| ORF1b | 832 | N | P | P |
| ORF1b | 833 | N | P | P |
| ORF1b | 834 | N | P | P |
| ORF1b | 835 | P | P | P |
| ORF1b | 836 | N | P | P |
| ORF1b | 837 | P | N | N |
| ORF1b | 838 | N | P | P |
| ORF1b | 840 | N | N | P |
| ORF1b | 843 | P | P | P |
| ORF1b | 844 | N | P | N |
| ORF1b | 845 | N | P | P |
| ORF1b | 848 | P | P | P |
| ORF1b | 849 | P | P | P |
| ORF1b | 850 | P | P | P |
| ORF1b | 851 | N | N | P |
| ORF1b | 852 | N | N | P |
| ORF1b | 853 | N | P | N |
| ORF1b | 854 | N | N | P |
| ORF1b | 855 | N | N | P |
| ORF1b | 857 | N | N | P |
| ORF1b | 858 | N | N | P |
| ORF1b | 859 | P | P | P |
| ORF1b | 860 | P | N | N |
| ORF1b | 861 | D | D | D |
| ORF1b | 862 | P | P | N |
| ORF1b | 863 | N | N | P |
| ORF1b | 864 | N | N | P |
| ORF1b | 865 | P | P | P |
| ORF1b | 866 | P | N | D |
| ORF1b | 867 | D | N | N |
| ORF1b | 869 | N | D | N |
| ORF1b | 870 | D | D | D |
| ORF1b | 871 | N | N | P |
| ORF1b | 872 | P | P | P |
| ORF1b | 874 | P | P | N |
| ORF1b | 875 | P | P | P |
| ORF1b | 876 | N | N | P |
| ORF1b | 877 | N | P | N |
| ORF1b | 882 | P | P | P |
| ORF1b | 883 | N | P | P |
| ORF1b | 884 | D | D | N |
| ORF1b | 887 | P | P | P |
| ORF1b | 888 | P | P | P |
| ORF1b | 891 | N | P | P |
| ORF1b | 894 | N | N | P |
| ORF1b | 898 | N | P | P |
| ORF1b | 899 | D | N | N |
| ORF1b | 901 | N | N | P |
| ORF1b | 902 | N | P | P |
| ORF1b | 903 | P | P | P |
| ORF1b | 904 | D | N | N |
| ORF1b | 908 | P | N | N |
| ORF1b | 909 | D | D | D |
| ORF1b | 911 | N | P | P |
| ORF1b | 912 | N | N | P |
| ORF1b | 913 | D | N | N |
| ORF1b | 914 | N | D | N |
| ORF1b | 916 | N | P | N |
| ORF1b | 917 | N | P | N |
| ORF1b | 918 | P | P | P |
| ORF1b | 919 | P | P | P |
| ORF1b | 920 | N | P | P |
| ORF1b | 922 | N | N | P |
| ORF1b | 923 | D | N | N |
| ORF1b | 924 | N | P | P |
| ORF1b | 925 | P | P | P |
| ORF1b | 926 | P | P | N |
| ORF1b | 927 | D | N | N |
| ORF1b | 928 | N | P | P |
| ORF1b | 930 | P | P | P |
| ORF1b | 931 | P | P | P |
| ORF1b | 933 | N | P | P |
| ORF1b | 934 | N | P | P |
| ORF1b | 935 | N | N | P |
| ORF1b | 936 | N | P | P |
| ORF1b | 938 | N | P | P |
| ORF1b | 941 | D | D | D |
| ORF1b | 942 | P | P | P |
| ORF1b | 943 | N | P | N |
| ORF1b | 945 | P | P | P |
| ORF1b | 946 | P | P | P |
| ORF1b | 947 | P | P | P |
| ORF1b | 948 | N | P | N |
| ORF1b | 949 | N | P | N |
| ORF1b | 950 | P | P | P |
| ORF1b | 951 | N | P | P |
| ORF1b | 952 | P | P | P |
| ORF1b | 953 | N | P | N |
| ORF1b | 954 | P | P | P |
| ORF1b | 955 | P | P | P |
| ORF1b | 956 | P | P | P |
| ORF1b | 957 | P | P | P |
| ORF1b | 958 | N | N | P |
| ORF1b | 959 | P | N | D |
| ORF1b | 960 | N | P | N |
| ORF1b | 962 | N | P | P |
| ORF1b | 963 | N | N | P |
| ORF1b | 964 | N | P | N |
| ORF1b | 965 | P | P | P |
| ORF1b | 968 | N | P | P |
| ORF1b | 969 | N | P | P |
| ORF1b | 970 | P | P | P |
| ORF1b | 973 | N | P | P |
| ORF1b | 974 | N | N | P |
| ORF1b | 975 | N | N | D |
| ORF1b | 976 | D | N | D |
| ORF1b | 977 | N | D | N |
| ORF1b | 978 | P | P | P |
| ORF1b | 979 | N | N | P |
| ORF1b | 981 | P | N | P |
| ORF1b | 986 | P | P | P |
| ORF1b | 987 | P | P | P |
| ORF1b | 988 | N | P | P |
| ORF1b | 989 | P | P | N |
| ORF1b | 992 | N | P | N |
| ORF1b | 993 | P | N | N |
| ORF1b | 995 | N | P | P |
| ORF1b | 997 | N | D | D |
| ORF1b | 998 | P | P | P |
| ORF1b | 999 | N | N | P |
| ORF1b | 1000 | D | D | D |
| ORF1b | 1001 | N | D | N |
| ORF1b | 1002 | N | D | N |
| ORF1b | 1003 | D | D | D |
| ORF1b | 1004 | P | P | P |
| ORF1b | 1005 | N | P | P |
| ORF1b | 1007 | N | P | P |
| ORF1b | 1009 | N | P | N |
| ORF1b | 1013 | P | P | P |
| ORF1b | 1014 | P | N | N |
| ORF1b | 1015 | P | P | P |
| ORF1b | 1017 | N | P | N |
| ORF1b | 1018 | P | N | P |
| ORF1b | 1019 | N | N | P |
| ORF1b | 1020 | N | P | P |
| ORF1b | 1021 | N | D | N |
| ORF1b | 1023 | D | D | N |
| ORF1b | 1024 | N | N | P |
| ORF1b | 1027 | N | P | N |
| ORF1b | 1028 | D | D | N |
| ORF1b | 1029 | P | P | N |
| ORF1b | 1031 | N | N | P |
| ORF1b | 1032 | P | P | P |
| ORF1b | 1033 | P | N | P |
| ORF1b | 1034 | P | P | P |
| ORF1b | 1035 | N | P | P |
| ORF1b | 1036 | P | P | P |
| ORF1b | 1043 | N | P | P |
| ORF1b | 1046 | N | P | P |
| ORF1b | 1048 | N | P | N |
| ORF1b | 1049 | N | P | P |
| ORF1b | 1050 | D | D | D |
| ORF1b | 1052 | D | N | N |
| ORF1b | 1053 | P | P | P |
| ORF1b | 1054 | N | P | N |
| ORF1b | 1055 | P | P | P |
| ORF1b | 1056 | P | P | P |
| ORF1b | 1057 | P | N | P |
| ORF1b | 1058 | N | P | P |
| ORF1b | 1059 | N | P | P |
| ORF1b | 1060 | P | P | P |
| ORF1b | 1061 | P | P | P |
| ORF1b | 1062 | P | N | P |
| ORF1b | 1063 | P | P | P |
| ORF1b | 1064 | N | D | N |
| ORF1b | 1065 | N | P | P |
| ORF1b | 1066 | N | N | P |
| ORF1b | 1067 | N | P | P |
| ORF1b | 1068 | N | P | P |
| ORF1b | 1069 | N | P | P |
| ORF1b | 1070 | N | N | P |
| ORF1b | 1072 | N | P | P |
| ORF1b | 1074 | N | N | D |
| ORF1b | 1075 | N | P | P |
| ORF1b | 1076 | D | N | N |
| ORF1b | 1078 | N | N | D |
| ORF1b | 1079 | N | N | P |
| ORF1b | 1080 | N | P | P |
| ORF1b | 1081 | N | P | N |
| ORF1b | 1082 | N | N | P |
| ORF1b | 1084 | P | P | N |
| ORF1b | 1085 | N | P | P |
| ORF1b | 1086 | N | P | P |
| ORF1b | 1087 | D | D | D |
| ORF1b | 1088 | N | P | P |
| ORF1b | 1089 | D + P | D | N |
| ORF1b | 1092 | D | D | D |
| ORF1b | 1093 | N | N | D |
| ORF1b | 1094 | N | N | P |
| ORF1b | 1095 | N | D | D |
| ORF1b | 1096 | N | P | N |
| ORF1b | 1097 | P | P | P |
| ORF1b | 1098 | N | P | N |
| ORF1b | 1100 | P | P | P |
| ORF1b | 1101 | N | P | P |
| ORF1b | 1105 | P | N | N |
| ORF1b | 1108 | N | P | N |
| ORF1b | 1109 | N | P | P |
| ORF1b | 1110 | D | D | N |
| ORF1b | 1113 | N | N | P |
| ORF1b | 1115 | P | N | N |
| ORF1b | 1117 | N | P | N |
| ORF1b | 1119 | P | P | P |
| ORF1b | 1120 | N | P | P |
| ORF1b | 1121 | N | P | N |
| ORF1b | 1122 | N | P | P |
| ORF1b | 1123 | P | P | N |
| ORF1b | 1124 | N | P | N |
| ORF1b | 1125 | N | P | P |
| ORF1b | 1127 | D | D | D |
| ORF1b | 1129 | D | D | D |
| ORF1b | 1130 | P | P | N |
| ORF1b | 1131 | D | N | N |
| ORF1b | 1134 | N | P | P |
| ORF1b | 1135 | P | P | P |
| ORF1b | 1137 | D | D | N |
| ORF1b | 1138 | P | P | P |
| ORF1b | 1140 | P | P | P |
| ORF1b | 1141 | N | P | N |
| ORF1b | 1147 | N | P | N |
| ORF1b | 1148 | P | P | N |
| ORF1b | 1149 | D | N | N |
| ORF1b | 1150 | P | P | P |
| ORF1b | 1151 | P | P | P |
| ORF1b | 1152 | P | P | P |
| ORF1b | 1153 | P | P | P |
| ORF1b | 1154 | P | P | P |
| ORF1b | 1155 | P | P | P |
| ORF1b | 1157 | P | P | P |
| ORF1b | 1158 | N | P | P |
| ORF1b | 1160 | D | N | N |
| ORF1b | 1161 | N | N | P |
| ORF1b | 1162 | D | N | N |
| ORF1b | 1163 | P | P | P |
| ORF1b | 1164 | N | P | P |
| ORF1b | 1165 | P | P | P |
| ORF1b | 1166 | N | P | P |
| ORF1b | 1170 | N | D | N |
| ORF1b | 1171 | D | P | N |
| ORF1b | 1172 | N | P | N |
| ORF1b | 1173 | D | D | N |
| ORF1b | 1175 | P | P | P |
| ORF1b | 1176 | D | D | D |
| ORF1b | 1177 | N | N | P |
| ORF1b | 1178 | P | N | N |
| ORF1b | 1179 | P | P | P |
| ORF1b | 1180 | P | P | P |
| ORF1b | 1181 | P | P | P |
| ORF1b | 1182 | D | D | N |
| ORF1b | 1183 | D | D | D |
| ORF1b | 1184 | D | D | D |
| ORF1b | 1190 | P | N | N |
| ORF1b | 1191 | N | P | P |
| ORF1b | 1194 | N | N | P |
| ORF1b | 1202 | N | P | P |
| ORF1b | 1204 | N | P | P |
| ORF1b | 1205 | N | P | P |
| ORF1b | 1207 | N | P | P |
| ORF1b | 1209 | N | N | P |
| ORF1b | 1212 | N | P | N |
| ORF1b | 1213 | D | D | D |
| ORF1b | 1216 | N | P | N |
| ORF1b | 1217 | N | P | P |
| ORF1b | 1218 | N | P | N |
| ORF1b | 1219 | D | D | D |
| ORF1b | 1220 | D | D | D |
| ORF1b | 1221 | N | N | P |
| ORF1b | 1222 | N | P | N |
| ORF1b | 1223 | D | D | D |
| ORF1b | 1225 | P | P | P |
| ORF1b | 1227 | P | N | N |
| ORF1b | 1228 | N | N | P |
| ORF1b | 1229 | N | P | P |
| ORF1b | 1232 | N | N | P |
| ORF1b | 1233 | N | P | P |
| ORF1b | 1234 | N | P | P |
| ORF1b | 1235 | P | P | P |
| ORF1b | 1236 | P | P | P |
| ORF1b | 1237 | P | P | P |
| ORF1b | 1238 | D | N | N |
| ORF1b | 1240 | P | P | N |
| ORF1b | 1241 | N | P | P |
| ORF1b | 1242 | N | D | D |
| ORF1b | 1243 | N | P | N |
| ORF1b | 1244 | P | P | P |
| ORF1b | 1246 | P | P | P |
| ORF1b | 1249 | N | P | N |
| ORF1b | 1252 | N | N | P |
| ORF1b | 1255 | N | P | P |
| ORF1b | 1256 | N | P | P |
| ORF1b | 1258 | N | P | P |
| ORF1b | 1260 | P | P | P |
| ORF1b | 1263 | P | N | P |
| ORF1b | 1264 | D | D | D |
| ORF1b | 1265 | P | P | P |
| ORF1b | 1266 | N | N | P |
| ORF1b | 1269 | P | P | P |
| ORF1b | 1270 | N | P | P |
| ORF1b | 1271 | D | N | N |
| ORF1b | 1273 | D | N | D |
| ORF1b | 1274 | D | N | N |
| ORF1b | 1275 | N | P | N |
| ORF1b | 1278 | N | P | N |
| ORF1b | 1279 | P | P | P |
| ORF1b | 1281 | P | P | N |
| ORF1b | 1283 | P | P | N |
| ORF1b | 1285 | N | P | N |
| ORF1b | 1286 | N | P | N |
| ORF1b | 1287 | N | P | N |
| ORF1b | 1288 | N | D | D |
| ORF1b | 1289 | P | P | P |
| ORF1b | 1290 | N | P | N |
| ORF1b | 1291 | D | D | D |
| ORF1b | 1292 | N | N | P |
| ORF1b | 1295 | N | P | P |
| ORF1b | 1296 | N | P | N |
| ORF1b | 1298 | N | P | N |
| ORF1b | 1300 | P | P | P |
| ORF1b | 1302 | P | P | P |
| ORF1b | 1303 | N | P | N |
| ORF1b | 1307 | N | P | N |
| ORF1b | 1308 | N | P | N |
| ORF1b | 1309 | N | P | P |
| ORF1b | 1310 | N | N | P |
| ORF1b | 1312 | N | P | N |
| ORF1b | 1313 | P | P | P |
| ORF1b | 1314 | P | P | P |
| ORF1b | 1315 | D | D | D |
| ORF1b | 1316 | P | P | N |
| ORF1b | 1320 | P | P | P |
| ORF1b | 1321 | P | P | N |
| ORF1b | 1322 | N | P | P |
| ORF1b | 1323 | N | P | P |
| ORF1b | 1324 | P | P | P |
| ORF1b | 1325 | D | P | P |
| ORF1b | 1326 | N | P | N |
| ORF1b | 1328 | N | P | P |
| ORF1b | 1329 | P | P | P |
| ORF1b | 1330 | N | P | P |
| ORF1b | 1331 | P | P | P |
| ORF1b | 1332 | P | P | P |
| ORF1b | 1333 | N | P | N |
| ORF1b | 1334 | N | P | P |
| ORF1b | 1335 | P | P | P |
| ORF1b | 1336 | D | D | N |
| ORF1b | 1337 | P | P | P |
| ORF1b | 1340 | P | P | P |
| ORF1b | 1341 | N | P | N |
| ORF1b | 1342 | N | P | N |
| ORF1b | 1343 | N | P | P |
| ORF1b | 1345 | P | P | P |
| ORF1b | 1346 | N | N | P |
| ORF1b | 1347 | N | P | P |
| ORF1b | 1348 | N | P | N |
| ORF1b | 1350 | N | P | P |
| ORF1b | 1351 | D | D | D |
| ORF1b | 1353 | N | N | P |
| ORF1b | 1354 | N | D | N |
| ORF1b | 1355 | N | P | N |
| ORF1b | 1357 | P | P | P |
| ORF1b | 1360 | P | P | N |
| ORF1b | 1361 | P | P | P |
| ORF1b | 1362 | N | P | N |
| ORF1b | 1364 | N | N | P |
| ORF1b | 1365 | P | P | P |
| ORF1b | 1366 | N | P | P |
| ORF1b | 1367 | N | P | P |
| ORF1b | 1368 | N | P | P |
| ORF1b | 1369 | D | N | N |
| ORF1b | 1370 | N | N | P |
| ORF1b | 1372 | N | P | P |
| ORF1b | 1373 | N | P | P |
| ORF1b | 1374 | P | N | P |
| ORF1b | 1375 | P | P | P |
| ORF1b | 1380 | P | P | P |
| ORF1b | 1382 | N | N | P |
| ORF1b | 1383 | D | D | D |
| ORF1b | 1384 | N | P | P |
| ORF1b | 1385 | N | N | P |
| ORF1b | 1386 | P | N | N |
| ORF1b | 1387 | P | N | N |
| ORF1b | 1389 | N | N | D |
| ORF1b | 1390 | N | P | P |
| ORF1b | 1391 | D | N | N |
| ORF1b | 1394 | P | P | P |
| ORF1b | 1395 | N | P | P |
| ORF1b | 1399 | P | N | N |
| ORF1b | 1400 | N | P | N |
| ORF1b | 1402 | N | N | P |
| ORF1b | 1403 | P | P | P |
| ORF1b | 1404 | D | N | N |
| ORF1b | 1405 | P | P | P |
| ORF1b | 1407 | N | N | P |
| ORF1b | 1408 | P | P | P |
| ORF1b | 1409 | N | P | P |
| ORF1b | 1410 | P | P | P |
| ORF1b | 1411 | N | P | P |
| ORF1b | 1412 | N | P | P |
| ORF1b | 1413 | N | P | P |
| ORF1b | 1414 | N | P | N |
| ORF1b | 1416 | P | P | N |
| ORF1b | 1417 | N | P | P |
| ORF1b | 1418 | P | P | P |
| ORF1b | 1419 | N | P | N |
| ORF1b | 1420 | N | P | P |
| ORF1b | 1421 | P | P | P |
| ORF1b | 1422 | P | P | P |
| ORF1b | 1424 | N | N | P |
| ORF1b | 1425 | P | N | N |
| ORF1b | 1426 | P | P | P |
| ORF1b | 1427 | D | D | D |
| ORF1b | 1428 | N | N | D |
| ORF1b | 1430 | P | P | P |
| ORF1b | 1431 | N | N | P |
| ORF1b | 1432 | D | P | N |
| ORF1b | 1433 | P | P | P |
| ORF1b | 1435 | N | P | N |
| ORF1b | 1437 | N | N | P |
| ORF1b | 1438 | P | P | P |
| ORF1b | 1439 | N | P | P |
| ORF1b | 1440 | N | P | N |
| ORF1b | 1441 | D | D | D |
| ORF1b | 1442 | P | P | N |
| ORF1b | 1443 | N | D | N |
| ORF1b | 1444 | P | P | P |
| ORF1b | 1445 | N | P | N |
| ORF1b | 1446 | N | P | P |
| ORF1b | 1447 | P | N | P |
| ORF1b | 1448 | N | P | P |
| ORF1b | 1449 | N | P | N |
| ORF1b | 1451 | P | P | P |
| ORF1b | 1455 | N | P | N |
| ORF1b | 1456 | N | P | P |
| ORF1b | 1458 | P | P | P |
| ORF1b | 1459 | P | P | P |
| ORF1b | 1460 | N | P | P |
| ORF1b | 1461 | P | P | P |
| ORF1b | 1462 | N | P | P |
| ORF1b | 1463 | N | N | P |
| ORF1b | 1464 | P | N | N |
| ORF1b | 1465 | N | D | D |
| ORF1b | 1466 | N | P | N |
| ORF1b | 1469 | P | P | N |
| ORF1b | 1470 | N | N | P |
| ORF1b | 1471 | N | P | N |
| ORF1b | 1472 | N | P | P |
| ORF1b | 1473 | N | P | N |
| ORF1b | 1475 | P | P | P |
| ORF1b | 1476 | N | P | P |
| ORF1b | 1478 | N | P | P |
| ORF1b | 1479 | N | N | P |
| ORF1b | 1481 | N | P | N |
| ORF1b | 1482 | P | P | N |
| ORF1b | 1483 | P | P | P |
| ORF1b | 1485 | N | P | N |
| ORF1b | 1486 | N | P | N |
| ORF1b | 1487 | P | P | P |
| ORF1b | 1488 | N | N | P |
| ORF1b | 1489 | P | P | P |
| ORF1b | 1490 | N | P | P |
| ORF1b | 1491 | N | P | P |
| ORF1b | 1492 | N | P | N |
| ORF1b | 1494 | N | N | P |
| ORF1b | 1496 | N | P | P |
| ORF1b | 1500 | N | N | P |
| ORF1b | 1501 | N | P | N |
| ORF1b | 1502 | N | N | P |
| ORF1b | 1503 | N | N | P |
| ORF1b | 1504 | D | D | D |
| ORF1b | 1505 | N | P | N |
| ORF1b | 1506 | N | N | P |
| ORF1b | 1508 | N | P | N |
| ORF1b | 1510 | N | P | N |
| ORF1b | 1511 | D | D | D |
| ORF1b | 1513 | N | P | P |
| ORF1b | 1516 | P | N | N |
| ORF1b | 1517 | N | P | P |
| ORF1b | 1518 | N | P | N |
| ORF1b | 1520 | P | P | P |
| ORF1b | 1521 | D | D | D |
| ORF1b | 1522 | D | D | D |
| ORF1b | 1525 | D | D | N |
| ORF1b | 1527 | N | P | P |
| ORF1b | 1528 | N | P | P |
| ORF1b | 1529 | N | P | N |
| ORF1b | 1530 | N | N | P |
| ORF1b | 1531 | P | P | P |
| ORF1b | 1532 | N | P | N |
| ORF1b | 1533 | N | P | P |
| ORF1b | 1535 | N | P | P |
| ORF1b | 1536 | N | P | N |
| ORF1b | 1538 | N | D | N |
| ORF1b | 1539 | D | D | N |
| ORF1b | 1540 | D | D | D |
| ORF1b | 1541 | N | P | N |
| ORF1b | 1542 | N | P | N |
| ORF1b | 1543 | N | P | P |
| ORF1b | 1544 | N | N | P |
| ORF1b | 1545 | N | P | P |
| ORF1b | 1546 | N | D | N |
| ORF1b | 1547 | P | P | N |
| ORF1b | 1548 | P | P | N |
| ORF1b | 1549 | N | P | P |
| ORF1b | 1550 | D | D | D |
| ORF1b | 1551 | N | P | P |
| ORF1b | 1553 | N | P | P |
| ORF1b | 1554 | P | P | P |
| ORF1b | 1555 | D | D | D |
| ORF1b | 1556 | N | N | P |
| ORF1b | 1557 | N | P | P |
| ORF1b | 1558 | P | P | N |
| ORF1b | 1559 | P | P | P |
| ORF1b | 1560 | N | P | N |
| ORF1b | 1561 | D | D | N |
| ORF1b | 1562 | N | P | P |
| ORF1b | 1564 | N | N | P |
| ORF1b | 1567 | D | N | D |
| ORF1b | 1570 | D | D | D |
| ORF1b | 1571 | N | P | P |
| ORF1b | 1572 | N | P | N |
| ORF1b | 1574 | N | P | P |
| ORF1b | 1576 | N | P | P |
| ORF1b | 1577 | N | P | N |
| ORF1b | 1585 | N | P | N |
| ORF1b | 1588 | N | P | P |
| ORF1b | 1593 | P | P | P |
| ORF1b | 1594 | N | D | N |
| ORF1b | 1595 | P | P | P |
| ORF1b | 1596 | N | D | N |
| ORF1b | 1597 | P | P | N |
| ORF1b | 1598 | D | D | D |
| ORF1b | 1599 | N | P | P |
| ORF1b | 1600 | N | P | P |
| ORF1b | 1601 | N | N | P |
| ORF1b | 1602 | N | N | P |
| ORF1b | 1605 | N | P | N |
| ORF1b | 1607 | P | P | N |
| ORF1b | 1608 | P | N | P |
| ORF1b | 1609 | N | N | P |
| ORF1b | 1611 | N | P | N |
| ORF1b | 1612 | N | N | P |
| ORF1b | 1614 | P | P | P |
| ORF1b | 1615 | P | P | P |
| ORF1b | 1616 | P | P | P |
| ORF1b | 1617 | P | P | P |
| ORF1b | 1620 | N | D | N |
| ORF1b | 1621 | N | D | D |
| ORF1b | 1622 | N | P | P |
| ORF1b | 1626 | N | P | N |
| ORF1b | 1627 | P | P | P |
| ORF1b | 1628 | N | P | N |
| ORF1b | 1630 | N | P | P |
| ORF1b | 1631 | N | P | P |
| ORF1b | 1632 | P | P | N |
| ORF1b | 1633 | P | P | P |
| ORF1b | 1634 | N | P | N |
| ORF1b | 1635 | N | P | N |
| ORF1b | 1636 | N | P | P |
| ORF1b | 1637 | D | D | D |
| ORF1b | 1641 | N | N | P |
| ORF1b | 1642 | N | P | N |
| ORF1b | 1643 | D | D | D |
| ORF1b | 1644 | N | P | N |
| ORF1b | 1645 | N | P | P |
| ORF1b | 1646 | P | P | P |
| ORF1b | 1649 | D | D | D |
| ORF1b | 1650 | P | P | P |
| ORF1b | 1651 | N | P | N |
| ORF1b | 1653 | D | D | N |
| ORF1b | 1654 | N | N | P |
| ORF1b | 1655 | P | N | N |
| ORF1b | 1656 | N | P | N |
| ORF1b | 1657 | N | P | N |
| ORF1b | 1658 | N | P | N |
| ORF1b | 1659 | N | N | P |
| ORF1b | 1661 | N | D | D |
| ORF1b | 1664 | N | N | D |
| ORF1b | 1665 | P | P | P |
| ORF1b | 1666 | D | N | N |
| ORF1b | 1667 | P | P | P |
| ORF1b | 1668 | P | P | P |
| ORF1b | 1669 | N | P | P |
| ORF1b | 1670 | N | N | P |
| ORF1b | 1671 | N | N | P |
| ORF1b | 1672 | P | P | N |
| ORF1b | 1673 | P | P | P |
| ORF1b | 1675 | P | N | P |
| ORF1b | 1676 | P | N | D |
| ORF1b | 1678 | P | P | P |
| ORF1b | 1679 | N | N | P |
| ORF1b | 1680 | N | N | P |
| ORF1b | 1681 | D | D | D |
| ORF1b | 1685 | P | N | P |
| ORF1b | 1686 | P | P | P |
| ORF1b | 1687 | D | D | D |
| ORF1b | 1688 | N | N | P |
| ORF1b | 1689 | P | N | N |
| ORF1b | 1690 | D | N | N |
| ORF1b | 1696 | P | P | P |
| ORF1b | 1700 | N | N | P |
| ORF1b | 1701 | D | N | N |
| ORF1b | 1702 | P | P | N |
| ORF1b | 1703 | N | P | N |
| ORF1b | 1704 | N | P | N |
| ORF1b | 1705 | N | P | P |
| ORF1b | 1706 | N | D + P | P |
| ORF1b | 1707 | N | P | N |
| ORF1b | 1708 | N | P | P |
| ORF1b | 1709 | N | P | N |
| ORF1b | 1712 | P | P | N |
| ORF1b | 1713 | P | N | N |
| ORF1b | 1714 | N | P | N |
| ORF1b | 1717 | N | P | P |
| ORF1b | 1718 | N | N | P |
| ORF1b | 1721 | N | P | P |
| ORF1b | 1723 | N | P | N |
| ORF1b | 1726 | N | N | P |
| ORF1b | 1727 | D | D | D |
| ORF1b | 1728 | D | D | N |
| ORF1b | 1729 | D | D + P | N |
| ORF1b | 1730 | P | P | P |
| ORF1b | 1731 | N | P | N |
| ORF1b | 1733 | N | P | N |
| ORF1b | 1734 | P | N | N |
| ORF1b | 1737 | D | N | N |
| ORF1b | 1738 | N | P | P |
| ORF1b | 1740 | P | P | P |
| ORF1b | 1742 | N | P | P |
| ORF1b | 1743 | D | N | N |
| ORF1b | 1745 | P | P | P |
| ORF1b | 1752 | N | N | P |
| ORF1b | 1753 | N | P | N |
| ORF1b | 1754 | N | N | P |
| ORF1b | 1756 | N | P | P |
| ORF1b | 1759 | P | P | P |
| ORF1b | 1760 | P | P | P |
| ORF1b | 1761 | N | N | P |
| ORF1b | 1763 | P | P | P |
| ORF1b | 1764 | N | P | P |
| ORF1b | 1768 | N | N | P |
| ORF1b | 1772 | D | N | D |
| ORF1b | 1774 | D | D | D |
| ORF1b | 1776 | N | N | P |
| ORF1b | 1777 | N | P | P |
| ORF1b | 1779 | D | D | N |
| ORF1b | 1780 | P | N | N |
| ORF1b | 1782 | P | N | N |
| ORF1b | 1783 | P | P | P |
| ORF1b | 1785 | P | P | N |
| ORF1b | 1786 | P | N | N |
| ORF1b | 1787 | D + P | P | P |
| ORF1b | 1788 | P | P | N |
| ORF1b | 1790 | N | N | P |
| ORF1b | 1791 | P | P | P |
| ORF1b | 1793 | P | P | N |
| ORF1b | 1794 | N | P | N |
| ORF1b | 1797 | P | P | N |
| ORF1b | 1802 | P | N | P |
| ORF1b | 1803 | N | P | P |
| ORF1b | 1804 | P | P | P |
| ORF1b | 1805 | D | N | N |
| ORF1b | 1807 | N | P | D |
| ORF1b | 1809 | N | P | P |
| ORF1b | 1810 | N | P | P |
| ORF1b | 1813 | N | N | D |
| ORF1b | 1814 | D | D | N |
| ORF1b | 1820 | P | P | P |
| ORF1b | 1821 | D | D | D |
| ORF1b | 1823 | N | P | N |
| ORF1b | 1825 | N | P | N |
| ORF1b | 1827 | P | P | P |
| ORF1b | 1830 | P | N | P |
| ORF1b | 1831 | N | N | P |
| ORF1b | 1832 | N | P | N |
| ORF1b | 1834 | N | N | P |
| ORF1b | 1837 | N | N | P |
| ORF1b | 1838 | P | P | P |
| ORF1b | 1840 | D | D | D |
| ORF1b | 1845 | P | P | P |
| ORF1b | 1846 | P | P | N |
| ORF1b | 1848 | D + P | D | N |
| ORF1b | 1849 | N | P | P |
| ORF1b | 1850 | P | P | P |
| ORF1b | 1851 | N | P | N |
| ORF1b | 1853 | N | P | P |
| ORF1b | 1854 | P | P | P |
| ORF1b | 1855 | P | P | P |
| ORF1b | 1856 | N | P | P |
| ORF1b | 1857 | N | P | N |
| ORF1b | 1859 | N | N | P |
| ORF1b | 1861 | N | P | P |
| ORF1b | 1862 | N | P | N |
| ORF1b | 1864 | N | P | N |
| ORF1b | 1865 | P | N | P |
| ORF1b | 1866 | N | P | P |
| ORF1b | 1869 | D | D | D |
| ORF1b | 1870 | N | P | P |
| ORF1b | 1873 | D | D | D |
| ORF1b | 1874 | N | N | P |
| ORF1b | 1876 | N | P | P |
| ORF1b | 1877 | P | N | N |
| ORF1b | 1878 | N | D | D |
| ORF1b | 1879 | D | P | N |
| ORF1b | 1881 | N | D | N |
| ORF1b | 1882 | N | N | P |
| ORF1b | 1883 | N | P | P |
| ORF1b | 1884 | D | D | D |
| ORF1b | 1886 | N | N | P |
| ORF1b | 1889 | N | P | N |
| ORF1b | 1890 | N | P | P |
| ORF1b | 1893 | D | N | N |
| ORF1b | 1894 | P | N | N |
| ORF1b | 1895 | N | D | D |
| ORF1b | 1899 | P | P | P |
| ORF1b | 1900 | P | P | N |
| ORF1b | 1901 | P | P | P |
| ORF1b | 1902 | P | N | N |
| ORF1b | 1903 | N | P | D |
| ORF1b | 1905 | P | P | P |
| ORF1b | 1906 | P | P | P |
| ORF1b | 1907 | P | P | P |
| ORF1b | 1912 | N | N | P |
| ORF1b | 1913 | N | P | N |
| ORF1b | 1914 | P | N | D |
| ORF1b | 1915 | P | P | P |
| ORF1b | 1916 | N | P | N |
| ORF1b | 1918 | N | D | D |
| ORF1b | 1919 | N | P | N |
| ORF1b | 1920 | N | N | P |
| ORF1b | 1924 | P | P | P |
| ORF1b | 1925 | N | P | N |
| ORF1b | 1926 | N | P | P |
| ORF1b | 1927 | N | P | N |
| ORF1b | 1928 | N | P | N |
| ORF1b | 1929 | P | P | P |
| ORF1b | 1930 | P | P | P |
| ORF1b | 1931 | N | N | P |
| ORF1b | 1932 | P | P | P |
| ORF1b | 1934 | P | P | P |
| ORF1b | 1935 | N | N | P |
| ORF1b | 1936 | D | D | D |
| ORF1b | 1939 | P | N | N |
| ORF1b | 1940 | P | N | N |
| ORF1b | 1941 | N | P | P |
| ORF1b | 1943 | N | P | N |
| ORF1b | 1945 | N | P | P |
| ORF1b | 1946 | N | P | P |
| ORF1b | 1947 | N | N | P |
| ORF1b | 1948 | N | P | P |
| ORF1b | 1949 | N | P | P |
| ORF1b | 1951 | N | P | N |
| ORF1b | 1952 | N | P | P |
| ORF1b | 1953 | N | P | P |
| ORF1b | 1954 | N | N | P |
| ORF1b | 1955 | N | N | P |
| ORF1b | 1964 | P | N | N |
| ORF1b | 1966 | N | P | P |
| ORF1b | 1969 | P | P | P |
| ORF1b | 1970 | N | N | P |
| ORF1b | 1971 | N | P | P |
| ORF1b | 1972 | P | P | P |
| ORF1b | 1974 | N | D | N |
| ORF1b | 1975 | N | P | N |
| ORF1b | 1976 | N | P | N |
| ORF1b | 1977 | D | D | N |
| ORF1b | 1978 | N | P | P |
| ORF1b | 1979 | D | N | N |
| ORF1b | 1980 | N | P | P |
| ORF1b | 1981 | N | P | P |
| ORF1b | 1983 | N | P | P |
| ORF1b | 1984 | P | P | N |
| ORF1b | 1985 | N | P | N |
| ORF1b | 1986 | N | P | P |
| ORF1b | 1991 | N | P | P |
| ORF1b | 1994 | P | P | P |
| ORF1b | 1995 | N | P | P |
| ORF1b | 1996 | P | P | N |
| ORF1b | 1999 | P | P | P |
| ORF1b | 2000 | N | P | N |
| ORF1b | 2002 | P | P | P |
| ORF1b | 2003 | P | P | P |
| ORF1b | 2007 | P | P | P |
| ORF1b | 2009 | N | P | P |
| ORF1b | 2011 | N | P | P |
| ORF1b | 2012 | N | P | P |
| ORF1b | 2013 | N | N | P |
| ORF1b | 2014 | P | P | N |
| ORF1b | 2015 | P | P | P |
| ORF1b | 2016 | N | P | P |
| ORF1b | 2017 | D | D | N |
| ORF1b | 2018 | P | P | P |
| ORF1b | 2019 | P | P | P |
| ORF1b | 2020 | D + P | D | D |
| ORF1b | 2021 | P | N | P |
| ORF1b | 2022 | N | N | P |
| ORF1b | 2026 | N | P | N |
| ORF1b | 2027 | N | P | N |
| ORF1b | 2028 | D | D | D |
| ORF1b | 2029 | P | P | P |
| ORF1b | 2030 | N | P | P |
| ORF1b | 2031 | N | N | P |
| ORF1b | 2032 | N | P | N |
| ORF1b | 2035 | P | P | P |
| ORF1b | 2036 | N | P | N |
| ORF1b | 2037 | N | N | P |
| ORF1b | 2038 | N | N | P |
| ORF1b | 2039 | P | P | P |
| ORF1b | 2040 | D | D | D |
| ORF1b | 2041 | N | P | P |
| ORF1b | 2042 | N | P | P |
| ORF1b | 2043 | N | P | N |
| ORF1b | 2045 | P | P | P |
| ORF1b | 2046 | N | N | P |
| ORF1b | 2047 | N | P | N |
| ORF1b | 2049 | N | P | N |
| ORF1b | 2055 | N | N | P |
| ORF1b | 2056 | N | N | P |
| ORF1b | 2058 | N | P | P |
| ORF1b | 2060 | N | D | N |
| ORF1b | 2062 | N | P | P |
| ORF1b | 2063 | D | N | D |
| ORF1b | 2064 | N | N | P |
| ORF1b | 2065 | P | P | P |
| ORF1b | 2066 | N | N | P |
| ORF1b | 2069 | N | P | N |
| ORF1b | 2070 | D | D | D |
| ORF1b | 2071 | N | P | P |
| ORF1b | 2073 | D | D | D |
| ORF1b | 2074 | N | P | N |
| ORF1b | 2076 | N | P | N |
| ORF1b | 2077 | D | N | N |
| ORF1b | 2079 | P | N | N |
| ORF1b | 2081 | N | P | N |
| ORF1b | 2084 | D | N | N |
| ORF1b | 2085 | N | P | N |
| ORF1b | 2086 | N | D | N |
| ORF1b | 2087 | N | N | P |
| ORF1b | 2090 | D | D | D |
| ORF1b | 2091 | P | P | P |
| ORF1b | 2092 | N | P | N |
| ORF1b | 2093 | N | P | P |
| ORF1b | 2096 | N | P | N |
| ORF1b | 2097 | N | P | N |
| ORF1b | 2098 | N | P | N |
| ORF1b | 2101 | N | P | P |
| ORF1b | 2104 | P | P | P |
| ORF1b | 2105 | N | P | P |
| ORF1b | 2106 | N | P | P |
| ORF1b | 2107 | N | P | N |
| ORF1b | 2108 | P | P | P |
| ORF1b | 2110 | N | P | P |
| ORF1b | 2111 | N | N | P |
| ORF1b | 2112 | N | P | P |
| ORF1b | 2117 | D | D | D |
| ORF1b | 2118 | P | P | P |
| ORF1b | 2120 | P | N | P |
| ORF1b | 2124 | P | P | P |
| ORF1b | 2126 | N | P | P |
| ORF1b | 2128 | N | P | N |
| ORF1b | 2129 | N | P | P |
| ORF1b | 2131 | N | D | D |
| ORF1b | 2132 | D | D | D |
| ORF1b | 2133 | P | P | P |
| ORF1b | 2135 | P | P | P |
| ORF1b | 2136 | P | P | P |
| ORF1b | 2138 | N | P | P |
| ORF1b | 2139 | P | P | P |
| ORF1b | 2140 | N | P | N |
| ORF1b | 2141 | N | P | P |
| ORF1b | 2144 | P | P | N |
| ORF1b | 2145 | N | P | N |
| ORF1b | 2146 | N | P | P |
| ORF1b | 2148 | N | P | N |
| ORF1b | 2149 | N | P | N |
| ORF1b | 2153 | P | P | N |
| ORF1b | 2156 | N | N | P |
| ORF1b | 2159 | N | P | P |
| ORF1b | 2160 | D | D | D |
| ORF1b | 2162 | P | N | P |
| ORF1b | 2163 | D | D | D |
| ORF1b | 2165 | P | P | P |
| ORF1b | 2167 | N | N | P |
| ORF1b | 2169 | P | P | P |
| ORF1b | 2170 | N | P | N |
| ORF1b | 2171 | N | N | P |
| ORF1b | 2172 | P | P | P |
| ORF1b | 2173 | N | N | P |
| ORF1b | 2174 | N | P | P |
| ORF1b | 2177 | N | P | P |
| ORF1b | 2178 | D | D | D |
| ORF1b | 2179 | D | D | N |
| ORF1b | 2180 | D | D | N |
| ORF1b | 2181 | N | N | P |
| ORF1b | 2182 | N | N | P |
| ORF1b | 2183 | D | P | N |
| ORF1b | 2186 | N | P | P |
| ORF1b | 2187 | P | P | P |
| ORF1b | 2188 | P | P | N |
| ORF1b | 2190 | N | P | N |
| ORF1b | 2191 | N | P | N |
| ORF1b | 2194 | N | P | N |
| ORF1b | 2195 | N | N | P |
| ORF1b | 2196 | P | P | P |
| ORF1b | 2198 | D | N | N |
| ORF1b | 2199 | N | D | N |
| ORF1b | 2201 | D | D | N |
| ORF1b | 2202 | N | P | N |
| ORF1b | 2204 | N | N | P |
| ORF1b | 2206 | N | P | P |
| ORF1b | 2208 | P | P | P |
| ORF1b | 2209 | N | P | N |
| ORF1b | 2212 | D | D | N |
| ORF1b | 2213 | D | D | D |
| ORF1b | 2215 | N | N | P |
| ORF1b | 2216 | P | N | N |
| ORF1b | 2219 | N | P | P |
| ORF1b | 2220 | N | N | P |
| ORF1b | 2222 | N | D | D |
| ORF1b | 2223 | D | D | D |
| ORF1b | 2225 | P | P | P |
| ORF1b | 2227 | P | P | P |
| ORF1b | 2232 | P | P | P |
| ORF1b | 2234 | N | P | N |
| ORF1b | 2237 | P | P | P |
| ORF1b | 2241 | N | P | P |
| ORF1b | 2244 | N | P | P |
| ORF1b | 2245 | N | P | P |
| ORF1b | 2246 | N | P | P |
| ORF1b | 2247 | D | D | N |
| ORF1b | 2249 | N | P | P |
| ORF1b | 2250 | P | N | N |
| ORF1b | 2254 | N | P | P |
| ORF1b | 2256 | D | D | D |
| ORF1b | 2263 | N | N | P |
| ORF1b | 2264 | N | P | N |
| ORF1b | 2265 | P | P | P |
| ORF1b | 2266 | N | P | N |
| ORF1b | 2267 | P | P | P |
| ORF1b | 2268 | D | D | D |
| ORF1b | 2270 | D | D | D |
| ORF1b | 2272 | N | P | P |
| ORF1b | 2273 | D | D | N |
| ORF1b | 2275 | N | P | P |
| ORF1b | 2277 | P | N | N |
| ORF1b | 2281 | N | P | P |
| ORF1b | 2283 | N | P | P |
| ORF1b | 2288 | P | N | N |
| ORF1b | 2289 | N | P | N |
| ORF1b | 2290 | N | P | N |
| ORF1b | 2291 | N | N | P |
| ORF1b | 2293 | N | N | P |
| ORF1b | 2295 | N | D + P | P |
| ORF1b | 2296 | P | P | P |
| ORF1b | 2297 | N | N | P |
| ORF1b | 2298 | P | P | P |
| ORF1b | 2299 | N | P | P |
| ORF1b | 2300 | P | P | P |
| ORF1b | 2302 | N | N | P |
| ORF1b | 2304 | N | P | N |
| ORF1b | 2305 | N | P | P |
| ORF1b | 2306 | D | D | D |
| ORF1b | 2308 | D | N | N |
| ORF1b | 2310 | N | D | N |
| ORF1b | 2311 | N | P | N |
| ORF1b | 2312 | N | D | N |
| ORF1b | 2313 | D | D | D |
| ORF1b | 2317 | N | P | P |
| ORF1b | 2319 | N | P | P |
| ORF1b | 2321 | N | N | D |
| ORF1b | 2323 | P | P | P |
| ORF1b | 2324 | N | D | N |
| ORF1b | 2325 | P | P | N |
| ORF1b | 2326 | P | P | N |
| ORF1b | 2328 | P | P | N |
| ORF1b | 2330 | N | P | N |
| ORF1b | 2332 | N | P | P |
| ORF1b | 2333 | N | N | D |
| ORF1b | 2335 | N | N | P |
| ORF1b | 2337 | N | N | D |
| ORF1b | 2339 | N | D | N |
| ORF1b | 2340 | N | D | D |
| ORF1b | 2341 | N | D | N |
| ORF1b | 2348 | P | P | N |
| ORF1b | 2352 | N | P | P |
| ORF1b | 2353 | N | P | P |
| ORF1b | 2355 | N | N | P |
| ORF1b | 2358 | N | P | P |
| ORF1b | 2359 | P | P | P |
| ORF1b | 2360 | N | P | N |
| ORF1b | 2361 | N | P | N |
| ORF1b | 2362 | P | P | P |
| ORF1b | 2363 | D | N | N |
| ORF1b | 2364 | N | P | N |
| ORF1b | 2365 | N | D | N |
| ORF1b | 2366 | N | P | P |
| ORF1b | 2371 | N | D | N |
| ORF1b | 2372 | N | N | P |
| ORF1b | 2375 | P | P | P |
| ORF1b | 2376 | N | D | N |
| ORF1b | 2382 | N | N | P |
| ORF1b | 2386 | N | N | P |
| ORF1b | 2387 | N | P | P |
| ORF1b | 2388 | N | D | D |
| ORF1b | 2389 | P | P | N |
| ORF1b | 2393 | N | N | P |
| ORF1b | 2394 | P | P | N |
| ORF1b | 2397 | N | N | P |
| ORF1b | 2398 | N | P | P |
| ORF1b | 2401 | P | P | P |
| ORF1b | 2404 | P | P | P |
| ORF1b | 2405 | N | P | P |
| ORF1b | 2406 | N | P | N |
| ORF1b | 2409 | N | N | P |
| ORF1b | 2412 | P | P | P |
| ORF1b | 2413 | N | P | N |
| ORF1b | 2416 | N | P | N |
| ORF1b | 2418 | P | P | P |
| ORF1b | 2419 | N | N | P |
| ORF1b | 2420 | N | N | P |
| ORF1b | 2421 | N | D | N |
| ORF1b | 2422 | N | P | N |
| ORF1b | 2423 | D | N | N |
| ORF1b | 2424 | N | P | P |
| ORF1b | 2425 | D | D | D |
| ORF1b | 2427 | P | P | P |
| ORF1b | 2428 | N | P | N |
| ORF1b | 2429 | N | D | P |
| ORF1b | 2430 | D | N | N |
| ORF1b | 2431 | D | D | D |
| ORF1b | 2432 | D | D | D |
| ORF1b | 2434 | N | P | N |
| ORF1b | 2440 | N | P | N |
| ORF1b | 2442 | N | P | P |
| ORF1b | 2443 | N | P | N |
| ORF1b | 2444 | P | P | P |
| ORF1b | 2445 | N | N | P |
| ORF1b | 2447 | N | P | P |
| ORF1b | 2448 | N | P | P |
| ORF1b | 2451 | N | N | P |
| ORF1b | 2452 | P | P | P |
| ORF1b | 2453 | N | D | N |
| ORF1b | 2455 | N | P | P |
| ORF1b | 2457 | N | P | P |
| ORF1b | 2459 | P | P | P |
| ORF1b | 2461 | N | P | N |
| ORF1b | 2463 | N | N | P |
| ORF1b | 2464 | N | P | N |
| ORF1b | 2466 | N | P | P |
| ORF1b | 2467 | N | P | P |
| ORF1b | 2469 | N | P | P |
| ORF1b | 2470 | P | N | N |
| ORF1b | 2471 | N | P | P |
| ORF1b | 2472 | N | P | P |
| ORF1b | 2473 | N | P | N |
| ORF1b | 2476 | N | N | P |
| ORF1b | 2477 | N | P | P |
| ORF1b | 2478 | N | P | N |
| ORF1b | 2479 | N | P | P |
| ORF1b | 2480 | N | P | P |
| ORF1b | 2481 | N | P | N |
| ORF1b | 2482 | N | N | P |
| ORF1b | 2483 | N | P | P |
| ORF1b | 2484 | N | P | P |
| ORF1b | 2486 | N | P | P |
| ORF1b | 2487 | N | P | P |
| ORF1b | 2488 | P | D | D |
| ORF1b | 2489 | N | N | D |
| ORF1b | 2490 | N | P | N |
| ORF1b | 2491 | N | P | P |
| ORF1b | 2492 | N | N | P |
| ORF1b | 2493 | P | P | P |
| ORF1b | 2494 | N | P | P |
| ORF1b | 2496 | N | P | P |
| ORF1b | 2498 | N | P | P |
| ORF1b | 2499 | N | D | N |
| ORF1b | 2502 | N | P | N |
| ORF1b | 2503 | N | P | P |
| ORF1b | 2504 | P | P | P |
| ORF1b | 2506 | N | P | N |
| ORF1b | 2507 | N | D | N |
| ORF1b | 2509 | N | P | P |
| ORF1b | 2510 | D | D | D |
| ORF1b | 2511 | N | N | P |
| ORF1b | 2512 | N | P | N |
| ORF1b | 2513 | N | N | D |
| ORF1b | 2514 | N | N | D |
| ORF1b | 2515 | N | P | N |
| ORF1b | 2516 | P | P | P |
| ORF1b | 2517 | N | D | N |
| ORF1b | 2518 | N | N | P |
| ORF1b | 2519 | P | P | P |
| ORF1b | 2520 | N | P | P |
| ORF1b | 2522 | N | P | N |
| ORF1b | 2523 | D | D | D |
| ORF1b | 2526 | N | P | P |
| ORF1b | 2527 | N | P | P |
| ORF1b | 2529 | N | P | P |
| ORF1b | 2530 | P | P | P |
| ORF1b | 2531 | D | D | D |
| ORF1b | 2534 | N | P | P |
| ORF1b | 2535 | P | N | N |
| ORF1b | 2536 | P | N | P |
| ORF1b | 2537 | N | D | D |
| ORF1b | 2538 | N | D | N |
| ORF1b | 2539 | N | D | N |
| ORF1b | 2540 | N | P | P |
| ORF1b | 2542 | N | P | P |
| ORF1b | 2543 | P | P | N |
| ORF1b | 2544 | N | N | P |
| ORF1b | 2545 | N | N | P |
| ORF1b | 2547 | P | P | P |
| ORF1b | 2548 | D | D | D |
| ORF1b | 2549 | N | N | P |
| ORF1b | 2552 | P | P | P |
| ORF1b | 2557 | D | D | D |
| ORF1b | 2558 | N | P | P |
| ORF1b | 2560 | D | D | D |
| ORF1b | 2561 | N | P | P |
| ORF1b | 2564 | D | N | N |
| ORF1b | 2565 | N | D | D |
| ORF1b | 2567 | N | N | P |
| ORF1b | 2568 | P | N | N |
| ORF1b | 2569 | N | P | N |
| ORF1b | 2571 | N | D | N |
| ORF1b | 2572 | N | P | P |
| ORF1b | 2574 | N | P | N |
| ORF1b | 2575 | D | N | N |
| ORF1b | 2576 | N | D | D |
| ORF1b | 2577 | N | N | P |
| ORF1b | 2578 | P | P | P |
| ORF1b | 2579 | D | D | D |
| ORF1b | 2580 | D | P | N |
| ORF1b | 2582 | P | P | P |
| ORF1b | 2583 | N | D | N |
| ORF1b | 2584 | P | P | P |
| ORF1b | 2585 | N | N | P |
| ORF1b | 2588 | N | P | P |
| ORF1b | 2589 | N | P | N |
| ORF1b | 2591 | N | P | N |
| ORF1b | 2593 | N | P | N |
| ORF1b | 2594 | P | P | P |
| ORF1b | 2595 | P | P | N |
| ORF1b | 2596 | P | P | P |
| ORF1b | 2597 | N | P | P |
| ORF1b | 2598 | N | P | P |
| ORF1b | 2599 | N | P | P |
| ORF1b | 2601 | N | P | P |
| ORF1b | 2602 | N | N | P |
| ORF1b | 2605 | N | P | P |
| ORF1b | 2608 | N | P | N |
| ORF1b | 2609 | N | P | P |
| ORF1b | 2610 | D | D | D |
| ORF1b | 2611 | N | N | P |
| ORF1b | 2612 | D | D | D |
| ORF1b | 2613 | D | D | D |
| ORF1b | 2615 | N | P | N |
| ORF1b | 2616 | N | P | N |
| ORF1b | 2617 | N | N | P |
| ORF1b | 2622 | N | P | P |
| ORF1b | 2624 | N | P | N |
| ORF1b | 2625 | N | P | P |
| ORF1b | 2626 | N | P | N |
| ORF1b | 2629 | N | P | N |
| ORF1b | 2630 | N | P | N |
| ORF1b | 2632 | N | P | N |
| ORF1b | 2633 | D | D | D |
| ORF1b | 2634 | P | P | P |
| ORF1b | 2635 | D | D | D |
| ORF1b | 2636 | N | P | N |
| ORF1b | 2637 | N | N | P |
| ORF1b | 2640 | N | P | N |
| ORF1b | 2641 | P | P | P |
| ORF1b | 2642 | N | P | P |
| ORF1b | 2643 | N | P | P |
| ORF1b | 2646 | N | P | P |
| ORF1b | 2647 | N | P | P |
| ORF1b | 2648 | N | N | D |
| ORF1b | 2649 | N | N | P |
| ORF1b | 2650 | N | P | P |
| ORF1b | 2651 | N | P | P |
| ORF1b | 2653 | N | P | N |
| ORF1b | 2654 | N | P | P |
| ORF1b | 2661 | N | D | N |
| ORF1b | 2662 | N | D | N |
| ORF1b | 2666 | P | N | N |
| ORF1b | 2668 | N | P | N |
| ORF1b | 2669 | N | P | N |
| ORF1b | 2670 | N | N | P |
| ORF1b | 2671 | N | P | P |
| ORF1b | 2672 | N | N | P |
| ORF1b | 2674 | N | P | P |
| ORF1b | 2676 | N | P | P |
| ORF1b | 2679 | N | P | N |
| ORF1b | 2680 | N | P | P |
| ORF1b | 2682 | N | N | P |
| ORF1b | 2683 | P | P | P |
| ORF1b | 2684 | P | N | N |
| ORF1b | 2685 | N | N | D |
| ORF1b | 2688 | D | N | N |
| ORF1b | 2689 | D | N | P |
| ORF1b | 2690 | D | N | N |
| ORF1b | 2691 | N | D | N |
| ORF1b | 2692 | N | N | P |
| ORF1b | 2693 | N | P | N |
| ORF1b | 2694 | P | P | P |
| ORF1b | 2695 | D | D | N |
| ORF3a | 3 | P | N | P |
| ORF3a | 4 | N | N | P |
| ORF3a | 6 | P | N | P |
| ORF3a | 7 | P | P | P |
| ORF3a | 8 | P | P | P |
| ORF3a | 11 | N | N | D |
| ORF3a | 12 | D | D | D |
| ORF3a | 13 | D | D | P |
| ORF3a | 14 | D | D | D |
| ORF3a | 15 | D | D | D |
| ORF3a | 16 | D | D | D |
| ORF3a | 17 | N | N | P |
| ORF3a | 18 | D | D | D |
| ORF3a | 19 | D | N | N |
| ORF3a | 20 | P | P | P |
| ORF3a | 21 | D | D | D |
| ORF3a | 22 | D | D | D |
| ORF3a | 23 | D | D | D |
| ORF3a | 24 | D | D | D |
| ORF3a | 25 | N | D | D |
| ORF3a | 26 | N | D | D |
| ORF3a | 27 | N | D | D |
| ORF3a | 29 | N | D | N |
| ORF3a | 30 | N | N | D |
| ORF3a | 31 | N | N | D |
| ORF3a | 32 | D | D | D |
| ORF3a | 34 | P | P | P |
| ORF3a | 35 | D | N | N |
| ORF3a | 36 | P | P | P |
| ORF3a | 38 | D | D | N |
| ORF3a | 39 | N | N | D |
| ORF3a | 40 | D | N | D |
| ORF3a | 41 | N | D | D |
| ORF3a | 42 | D | D | D |
| ORF3a | 43 | P | P | P |
| ORF3a | 46 | N | D | D |
| ORF3a | 47 | N | D | D |
| ORF3a | 48 | D | N | N |
| ORF3a | 49 | N | D | D |
| ORF3a | 50 | N | D | D |
| ORF3a | 52 | D | D | D |
| ORF3a | 53 | D | D | D |
| ORF3a | 54 | D | D | D |
| ORF3a | 55 | D | D | D |
| ORF3a | 56 | N | N | P |
| ORF3a | 57 | D | D | D |
| ORF3a | 58 | D | D | D |
| ORF3a | 59 | N | D | D |
| ORF3a | 60 | P | P | P |
| ORF3a | 61 | D | D | D |
| ORF3a | 62 | N | N | P |
| ORF3a | 63 | D | D | N |
| ORF3a | 64 | P | P | P |
| ORF3a | 65 | P | P | P |
| ORF3a | 67 | D | D | D |
| ORF3a | 68 | D | D | N |
| ORF3a | 71 | P | P | P |
| ORF3a | 72 | N | D | D |
| ORF3a | 73 | N | P | P |
| ORF3a | 74 | P | P | P |
| ORF3a | 77 | D | D | D |
| ORF3a | 78 | D | D | D |
| ORF3a | 79 | P | P | P |
| ORF3a | 81 | N | P | N |
| ORF3a | 82 | N | P | P |
| ORF3a | 83 | N | D | D |
| ORF3a | 84 | P | P | P |
| ORF3a | 85 | D | D | D |
| ORF3a | 86 | N | D | N |
| ORF3a | 87 | N | P | N |
| ORF3a | 89 | D | D | D |
| ORF3a | 90 | D | D | D |
| ORF3a | 91 | P | P | P |
| ORF3a | 92 | D | N | N |
| ORF3a | 93 | D | N | D |
| ORF3a | 94 | D | D | N |
| ORF3a | 95 | N | N | D |
| ORF3a | 96 | P | P | P |
| ORF3a | 97 | D | D | D |
| ORF3a | 99 | D | D | D |
| ORF3a | 100 | D | D | D |
| ORF3a | 101 | N | D | D |
| ORF3a | 102 | N | N | D |
| ORF3a | 104 | D | D | D |
| ORF3a | 107 | D | N | D |
| ORF3a | 108 | D | D | D |
| ORF3a | 110 | D | D | D |
| ORF3a | 111 | N | P | P |
| ORF3a | 112 | N | N | P |
| ORF3a | 113 | P | P | P |
| ORF3a | 119 | N | D | D |
| ORF3a | 121 | N | P | P |
| ORF3a | 122 | N | N | D |
| ORF3a | 129 | D | D | D |
| ORF3a | 130 | P | P | P |
| ORF3a | 131 | N | D | N |
| ORF3a | 133 | P | P | P |
| ORF3a | 134 | D | D | D |
| ORF3a | 135 | P | P | P |
| ORF3a | 138 | N | P | P |
| ORF3a | 139 | N | N | P |
| ORF3a | 140 | D | D | D |
| ORF3a | 141 | N | N | P |
| ORF3a | 143 | D | N | D |
| ORF3a | 144 | N | D | N |
| ORF3a | 145 | N | P | N |
| ORF3a | 147 | D | D | N |
| ORF3a | 148 | P | P | P |
| ORF3a | 151 | D | D | D |
| ORF3a | 153 | P | P | P |
| ORF3a | 154 | P | P | N |
| ORF3a | 155 | D | D | D |
| ORF3a | 161 | N | P | N |
| ORF3a | 165 | D | D | D |
| ORF3a | 166 | N | D | D |
| ORF3a | 168 | N | N | P |
| ORF3a | 169 | P | P | P |
| ORF3a | 171 | D | D | D |
| ORF3a | 172 | D | D | D |
| ORF3a | 173 | D | D | D |
| ORF3a | 174 | D | D | D |
| ORF3a | 175 | D | D | D |
| ORF3a | 176 | D | D | D |
| ORF3a | 177 | N | D | D |
| ORF3a | 179 | N | N | D |
| ORF3a | 181 | N | N | D |
| ORF3a | 182 | D | D | D |
| ORF3a | 184 | N | D | N |
| ORF3a | 185 | D | D | D |
| ORF3a | 187 | N | N | P |
| ORF3a | 188 | N | D | N |
| ORF3a | 190 | N | N | D |
| ORF3a | 192 | N | P | N |
| ORF3a | 193 | P | P | P |
| ORF3a | 194 | P | N | D + P |
| ORF3a | 195 | D | N | D |
| ORF3a | 196 | N | N | P |
| ORF3a | 197 | P | P | P |
| ORF3a | 200 | N | P | P |
| ORF3a | 202 | D | N | N |
| ORF3a | 206 | P | P | P |
| ORF3a | 207 | P | P | P |
| ORF3a | 209 | N | P | N |
| ORF3a | 210 | P | P | P |
| ORF3a | 212 | P | P | P |
| ORF3a | 213 | D | N | D |
| ORF3a | 215 | N | D | N |
| ORF3a | 217 | N | N | D |
| ORF3a | 220 | N | N | D |
| ORF3a | 221 | P | N | N |
| ORF3a | 222 | D + P | P | P |
| ORF3a | 223 | D | D | D |
| ORF3a | 224 | D | D | D |
| ORF3a | 225 | D | D | D |
| ORF3a | 228 | N | P | P |
| ORF3a | 229 | D | D | D |
| ORF3a | 230 | N | P | P |
| ORF3a | 231 | N | N | P |
| ORF3a | 232 | P | N | N |
| ORF3a | 233 | N | P | N |
| ORF3a | 234 | N | P | N |
| ORF3a | 235 | N | D | N |
| ORF3a | 239 | D | N | N |
| ORF3a | 240 | D | D | D |
| ORF3a | 242 | N | P | P |
| ORF3a | 244 | P | P | P |
| ORF3a | 249 | P | P | P |
| ORF3a | 251 | D | D | D |
| ORF3a | 252 | P | P | P |
| ORF3a | 253 | P | P | P |
| ORF3a | 254 | D | D | D |
| ORF3a | 255 | N | D | N |
| ORF3a | 258 | N | P | N |
| ORF3a | 259 | N | P | P |
| ORF3a | 263 | N | N | P |
| ORF3a | 265 | N | P | P |
| ORF3a | 266 | N | D | N |
| ORF3a | 268 | D | D | D |
| ORF3a | 269 | N | P | D |
| ORF3a | 270 | N | D | D |
| ORF3a | 272 | P | P | P |
| ORF3a | 273 | P | P | P |
| ORF3a | 274 | N | N | P |
| ORF3a | 275 | N | D | D |
| ORF6 | 3 | N | D | P |
| ORF6 | 4 | P | P | P |
| ORF6 | 5 | D | N | D |
| ORF6 | 6 | N | D | D |
| ORF6 | 7 | P | P | P |
| ORF6 | 8 | D + P | N | N |
| ORF6 | 9 | D | N | N |
| ORF6 | 11 | N | P | N |
| ORF6 | 12 | N | P | P |
| ORF6 | 16 | P | P | P |
| ORF6 | 19 | N | N | P |
| ORF6 | 20 | P | P | P |
| ORF6 | 21 | N | D | D |
| ORF6 | 24 | N | D | N |
| ORF6 | 25 | N | N | P |
| ORF6 | 30 | N | P | P |
| ORF6 | 31 | P | P | P |
| ORF6 | 32 | P | P | P |
| ORF6 | 33 | N | D | D |
| ORF6 | 34 | N | D | N |
| ORF6 | 35 | N | D | D |
| ORF6 | 39 | N | D | N |
| ORF6 | 40 | N | P | N |
| ORF6 | 42 | D | N | N |
| ORF6 | 43 | N | P | N |
| ORF6 | 48 | N | D | N |
| ORF6 | 49 | N | N | D |
| ORF6 | 50 | N | P | N |
| ORF6 | 51 | P | P | P |
| ORF6 | 53 | N | D | N |
| ORF6 | 57 | N | P | P |
| ORF6 | 61 | P | P | P |
| ORF7a | 2 | N | P | P |
| ORF7a | 4 | N | D | N |
| ORF7a | 5 | N | D | D |
| ORF7a | 6 | N | N | D |
| ORF7a | 7 | N | P | P |
| ORF7a | 11 | N | D | N |
| ORF7a | 13 | N | D | N |
| ORF7a | 14 | D | D | D |
| ORF7a | 15 | N | P | P |
| ORF7a | 16 | N | N | P |
| ORF7a | 17 | N | D | N |
| ORF7a | 18 | P | N | P |
| ORF7a | 21 | P | P | P |
| ORF7a | 24 | D | D | D |
| ORF7a | 25 | P | N | P |
| ORF7a | 28 | D | D | D |
| ORF7a | 30 | P | P | P |
| ORF7a | 31 | P | P | P |
| ORF7a | 32 | N | P | P |
| ORF7a | 34 | D | D | D |
| ORF7a | 35 | P | P | P |
| ORF7a | 37 | P | N | P |
| ORF7a | 38 | D | N | P |
| ORF7a | 39 | D | D | N |
| ORF7a | 40 | P | P | P |
| ORF7a | 42 | N | D | D |
| ORF7a | 44 | N | P | N |
| ORF7a | 45 | N | D | D |
| ORF7a | 46 | P | P | N |
| ORF7a | 47 | N | D | D |
| ORF7a | 49 | N | P | P |
| ORF7a | 50 | N | N | D |
| ORF7a | 52 | P | P | P |
| ORF7a | 56 | P | P | P |
| ORF7a | 60 | N | D | N |
| ORF7a | 61 | N | D | N |
| ORF7a | 62 | D | D | D |
| ORF7a | 65 | N | N | P |
| ORF7a | 66 | N | D | D |
| ORF7a | 69 | P | P | P |
| ORF7a | 70 | N | P | P |
| ORF7a | 71 | N | D | N |
| ORF7a | 73 | D | D | D |
| ORF7a | 75 | P | P | P |
| ORF7a | 78 | N | D | D |
| ORF7a | 79 | P | P | P |
| ORF7a | 80 | N | N | D |
| ORF7a | 82 | N | D | D |
| ORF7a | 83 | N | D | N |
| ORF7a | 84 | N | D | D |
| ORF7a | 86 | P | P | P |
| ORF7a | 87 | N | P | P |
| ORF7a | 89 | P | N | N |
| ORF7a | 92 | P | P | N |
| ORF7a | 93 | D | D | D |
| ORF7a | 94 | D | D | D |
| ORF7a | 96 | N | D | D |
| ORF7a | 97 | P | P | P |
| ORF7a | 98 | N | D | N |
| ORF7a | 99 | D | D | D |
| ORF7a | 101 | N | N | P |
| ORF7a | 104 | D | D | D |
| ORF7a | 105 | D | N | D |
| ORF7a | 106 | N | P | P |
| ORF7a | 109 | N | N | P |
| ORF7a | 113 | P | P | P |
| ORF7a | 114 | P | P | P |
| ORF7a | 115 | D | D | N |
| ORF7a | 116 | P | P | P |
| ORF7a | 117 | N | N | P |
| ORF7a | 120 | D | D | N |
| ORF7a | 121 | P | N | N |
| ORF8 | 2 | N | P | N |
| ORF8 | 5 | N | D | N |
| ORF8 | 6 | P | P | P |
| ORF8 | 7 | N | N | P |
| ORF8 | 8 | D | D | D |
| ORF8 | 9 | P | P | P |
| ORF8 | 10 | P | N | P |
| ORF8 | 11 | D | D | D |
| ORF8 | 12 | N | N | P |
| ORF8 | 13 | N | P | N |
| ORF8 | 15 | P | P | P |
| ORF8 | 16 | N | P | N |
| ORF8 | 17 | P | N | P |
| ORF8 | 26 | D | D | D |
| ORF8 | 28 | N | P | P |
| ORF8 | 32 | D | D | D |
| ORF8 | 35 | D | N | N |
| ORF8 | 36 | P | P | P |
| ORF8 | 37 | N | D | N |
| ORF8 | 38 | N | D | D |
| ORF8 | 40 | D | D | D |
| ORF8 | 41 | N | N | D |
| ORF8 | 42 | N | P | N |
| ORF8 | 43 | N | N | P |
| ORF8 | 45 | N | D | N |
| ORF8 | 46 | N | P | N |
| ORF8 | 48 | N | P | N |
| ORF8 | 49 | N | P | P |
| ORF8 | 50 | P | N | N |
| ORF8 | 51 | D | D | D |
| ORF8 | 52 | D | D | D |
| ORF8 | 54 | N | D | D |
| ORF8 | 55 | N | D | D |
| ORF8 | 61 | P | N | N |
| ORF8 | 62 | D | N | D + P |
| ORF8 | 64 | N | N | D |
| ORF8 | 65 | D | D | D |
| ORF8 | 66 | N | D | D |
| ORF8 | 67 | D | D | D |
| ORF8 | 68 | D | D | D |
| ORF8 | 69 | N | N | D |
| ORF8 | 72 | N | D | D |
| ORF8 | 73 | N | D | D |
| ORF8 | 74 | N | P | N |
| ORF8 | 76 | N | N | D |
| ORF8 | 77 | D | D | D |
| ORF8 | 80 | P | N | P |
| ORF8 | 82 | P | N | D + P |
| ORF8 | 83 | P | P | N |
| ORF8 | 85 | N | D | D |
| ORF8 | 86 | N | P | N |
| ORF8 | 87 | D | D | D |
| ORF8 | 90 | P | P | P |
| ORF8 | 93 | N | D | D |
| ORF8 | 94 | N | P | N |
| ORF8 | 95 | D | N | N |
| ORF8 | 102 | N | D | N |
| ORF8 | 103 | P | N | N |
| ORF8 | 105 | N | N | P |
| ORF8 | 107 | N | P | N |
| ORF8 | 109 | N | P | P |
| ORF8 | 110 | N | D | N |
| ORF8 | 111 | N | P | N |
| ORF8 | 113 | N | P | P |
| ORF8 | 115 | N | D | D |
| ORF8 | 118 | N | D | D |
| ORF8 | 119 | N | D | N |
| ORF8 | 120 | P | P | P |
| ORF8 | 121 | N | D | D |
| ORF8 | 122 | N | D | D |
| Spike | 1 | N | P | P |
| Spike | 4 | N | P | N |
| Spike | 5 | D | D | D |
| Spike | 6 | N | D | D |
| Spike | 7 | P | P | P |
| Spike | 8 | N | D | D |
| Spike | 10 | P | P | P |
| Spike | 11 | P | P | P |
| Spike | 12 | D | D | D |
| Spike | 13 | D | D | D |
| Spike | 14 | N | D | D |
| Spike | 16 | N | D | N |
| Spike | 18 | D | D | D |
| Spike | 19 | D | D | D |
| Spike | 20 | P | D | P |
| Spike | 22 | D | D | D |
| Spike | 24 | N | N | P |
| Spike | 25 | N | P | P |
| Spike | 26 | D | D | D |
| Spike | 27 | D | D | D |
| Spike | 28 | P | P | P |
| Spike | 29 | D | D | N |
| Spike | 30 | N | P | N |
| Spike | 31 | N | N | P |
| Spike | 32 | P | P | P |
| Spike | 33 | N | D | N |
| Spike | 34 | P | P | N |
| Spike | 37 | P | P | P |
| Spike | 38 | P | P | P |
| Spike | 39 | N | P | P |
| Spike | 40 | P | P | P |
| Spike | 41 | N | P | P |
| Spike | 43 | P | P | P |
| Spike | 44 | N | P | N |
| Spike | 45 | N | P | P |
| Spike | 46 | N | P | P |
| Spike | 48 | P | P | P |
| Spike | 49 | D | D | D |
| Spike | 50 | D | N | N |
| Spike | 53 | P | P | P |
| Spike | 54 | D | D | D |
| Spike | 55 | P | P | P |
| Spike | 56 | N | P | N |
| Spike | 57 | N | N | P |
| Spike | 58 | P | P | P |
| Spike | 59 | P | N | P |
| Spike | 60 | P | P | P |
| Spike | 61 | P | P | P |
| Spike | 63 | N | P | N |
| Spike | 64 | N | N | P |
| Spike | 65 | N | P | P |
| Spike | 66 | N | P | N |
| Spike | 67 | D | D | D |
| Spike | 68 | P | P | P |
| Spike | 69 | N | N | D |
| Spike | 70 | P | N | N |
| Spike | 71 | P | N | N |
| Spike | 72 | D | D | D |
| Spike | 73 | N | P | P |
| Spike | 74 | N | N | P |
| Spike | 75 | D | D | D |
| Spike | 76 | D | D | D |
| Spike | 78 | N | D + P | N |
| Spike | 79 | N | N | P |
| Spike | 80 | D | D | N |
| Spike | 82 | P | P | N |
| Spike | 83 | P | P | P |
| Spike | 84 | P | P | P |
| Spike | 85 | N | P | N |
| Spike | 86 | P | P | P |
| Spike | 87 | P | P | P |
| Spike | 88 | N | N | P |
| Spike | 90 | D | N | N |
| Spike | 91 | N | P | P |
| Spike | 92 | P | P | N |
| Spike | 93 | N | P | P |
| Spike | 94 | P | N | N |
| Spike | 95 | D | D | D |
| Spike | 96 | N | D | D |
| Spike | 98 | D | D | D |
| Spike | 99 | P | P | P |
| Spike | 103 | N | P | P |
| Spike | 106 | P | P | P |
| Spike | 107 | P | P | P |
| Spike | 108 | P | P | P |
| Spike | 109 | P | N | P |
| Spike | 110 | N | P | P |
| Spike | 111 | N | P | P |
| Spike | 113 | P | P | P |
| Spike | 114 | N | P | P |
| Spike | 115 | N | P | P |
| Spike | 116 | P | P | P |
| Spike | 117 | N | P | P |
| Spike | 118 | N | N | P |
| Spike | 119 | N | N | P |
| Spike | 121 | P | P | P |
| Spike | 122 | N | P | P |
| Spike | 123 | N | P | N |
| Spike | 124 | N | P | P |
| Spike | 125 | N | P | P |
| Spike | 128 | N | P | N |
| Spike | 129 | N | N | P |
| Spike | 130 | P | P | P |
| Spike | 131 | N | N | P |
| Spike | 133 | N | P | P |
| Spike | 134 | N | P | N |
| Spike | 137 | N | P | P |
| Spike | 138 | D | D | D |
| Spike | 139 | P | P | P |
| Spike | 140 | P | P | N |
| Spike | 142 | D | D | D |
| Spike | 143 | D | N | N |
| Spike | 144 | P | D | P |
| Spike | 145 | N | N | D |
| Spike | 146 | N | P | P |
| Spike | 147 | N | N | D |
| Spike | 148 | N | N | D |
| Spike | 149 | N | P | P |
| Spike | 151 | D | D | N |
| Spike | 154 | N | D | N |
| Spike | 155 | N | N | P |
| Spike | 156 | D | N | N |
| Spike | 157 | N | D | D |
| Spike | 158 | N | D | D |
| Spike | 159 | P | P | N |
| Spike | 160 | N | P | P |
| Spike | 161 | N | P | N |
| Spike | 163 | P | P | P |
| Spike | 164 | N | N | D |
| Spike | 165 | N | P | N |
| Spike | 166 | N | N | P |
| Spike | 167 | N | P | P |
| Spike | 168 | N | P | P |
| Spike | 171 | N | P | P |
| Spike | 172 | P | P | P |
| Spike | 173 | N | D | D |
| Spike | 175 | N | P | N |
| Spike | 176 | D | D | D |
| Spike | 178 | D | D | D |
| Spike | 180 | N | N | D |
| Spike | 181 | D | D | D |
| Spike | 182 | N | N | D |
| Spike | 183 | D | N | N |
| Spike | 184 | P | N | N |
| Spike | 186 | P | P | P |
| Spike | 191 | N | P | P |
| Spike | 192 | N | P | P |
| Spike | 193 | N | N | P |
| Spike | 195 | P | P | P |
| Spike | 196 | N | N | P |
| Spike | 199 | N | P | N |
| Spike | 200 | P | P | P |
| Spike | 202 | N | P | P |
| Spike | 203 | N | P | P |
| Spike | 204 | N | P | P |
| Spike | 205 | N | P | N |
| Spike | 206 | P | P | P |
| Spike | 210 | N | N | D |
| Spike | 212 | N | D | D |
| Spike | 214 | D | D + P | N |
| Spike | 215 | D | D | D |
| Spike | 217 | N | P | P |
| Spike | 220 | N | P | N |
| Spike | 221 | P | P | P |
| Spike | 222 | D | D | D |
| Spike | 223 | P | P | P |
| Spike | 225 | P | P | P |
| Spike | 226 | P | P | P |
| Spike | 227 | N | N | P |
| Spike | 228 | N | N | P |
| Spike | 229 | N | D | N |
| Spike | 230 | P | P | P |
| Spike | 231 | P | P | P |
| Spike | 234 | P | P | P |
| Spike | 235 | N | P | P |
| Spike | 236 | N | P | P |
| Spike | 238 | P | P | P |
| Spike | 240 | N | N | D |
| Spike | 241 | P | P | P |
| Spike | 243 | P | N | N |
| Spike | 245 | D | D | N |
| Spike | 247 | N | N | P |
| Spike | 248 | N | P | D |
| Spike | 249 | N | N | P |
| Spike | 250 | N | P | D |
| Spike | 251 | P | N | N |
| Spike | 252 | N | D | N |
| Spike | 253 | D | D | D |
| Spike | 254 | D | D | D |
| Spike | 255 | D | D | D |
| Spike | 256 | D | D | N |
| Spike | 257 | D | D | D |
| Spike | 259 | P | N | N |
| Spike | 260 | P | P | N |
| Spike | 261 | D | D | D |
| Spike | 262 | D | D | D |
| Spike | 263 | P | P | N |
| Spike | 264 | N | P | N |
| Spike | 266 | N | N | P |
| Spike | 267 | P | P | P |
| Spike | 270 | N | N | P |
| Spike | 273 | N | N | P |
| Spike | 274 | P | P | P |
| Spike | 275 | P | P | N |
| Spike | 276 | P | P | P |
| Spike | 277 | P | P | P |
| Spike | 279 | N | P | P |
| Spike | 280 | N | N | P |
| Spike | 283 | N | P | P |
| Spike | 285 | N | P | N |
| Spike | 287 | N | N | P |
| Spike | 288 | N | P | P |
| Spike | 290 | P | P | P |
| Spike | 291 | N | P | P |
| Spike | 292 | P | P | N |
| Spike | 293 | P | P | P |
| Spike | 294 | P | P | P |
| Spike | 295 | P | P | P |
| Spike | 296 | P | P | P |
| Spike | 297 | N | P | P |
| Spike | 298 | N | P | P |
| Spike | 299 | N | D | N |
| Spike | 301 | N | P | P |
| Spike | 302 | P | P | P |
| Spike | 303 | P | P | N |
| Spike | 304 | N | P | P |
| Spike | 305 | P | P | N |
| Spike | 306 | P | P | P |
| Spike | 307 | D | D | N |
| Spike | 308 | D | D | D |
| Spike | 309 | N | D | N |
| Spike | 310 | N | P | P |
| Spike | 311 | P | P | N |
| Spike | 312 | P | P | P |
| Spike | 313 | N | P | P |
| Spike | 314 | D | D | N |
| Spike | 315 | N | P | P |
| Spike | 316 | N | N | P |
| Spike | 320 | N | P | P |
| Spike | 321 | P | P | P |
| Spike | 322 | P | N | N |
| Spike | 324 | N | P | N |
| Spike | 325 | N | N | P |
| Spike | 328 | P | P | P |
| Spike | 329 | N | P | P |
| Spike | 330 | P | N | N |
| Spike | 331 | N | N | P |
| Spike | 333 | N | P | P |
| Spike | 334 | N | P | P |
| Spike | 335 | N | P | N |
| Spike | 336 | N | P | P |
| Spike | 337 | P | P | P |
| Spike | 338 | N | P | P |
| Spike | 339 | N | N | D |
| Spike | 340 | N | N | P |
| Spike | 341 | N | P | N |
| Spike | 342 | P | P | P |
| Spike | 343 | P | P | P |
| Spike | 344 | N | N | P |
| Spike | 345 | P | P | P |
| Spike | 346 | N | N | D |
| Spike | 348 | P | N | P |
| Spike | 349 | N | P | N |
| Spike | 351 | N | P | P |
| Spike | 352 | P | P | N |
| Spike | 354 | P | P | P |
| Spike | 355 | P | P | P |
| Spike | 358 | N | P | P |
| Spike | 359 | N | P | P |
| Spike | 360 | N | P | P |
| Spike | 361 | P | P | P |
| Spike | 363 | N | N | P |
| Spike | 364 | N | P | P |
| Spike | 365 | P | N | P |
| Spike | 366 | P | P | P |
| Spike | 367 | D | D | D |
| Spike | 368 | N | P | N |
| Spike | 369 | P | P | P |
| Spike | 371 | P | N | D |
| Spike | 372 | P | N | N |
| Spike | 374 | N | P | P |
| Spike | 375 | P | P | N |
| Spike | 376 | N | N | D |
| Spike | 377 | P | P | P |
| Spike | 379 | N | N | P |
| Spike | 380 | N | P | P |
| Spike | 381 | N | P | P |
| Spike | 382 | P | P | P |
| Spike | 383 | N | P | P |
| Spike | 384 | N | D | D |
| Spike | 386 | N | P | P |
| Spike | 387 | N | N | P |
| Spike | 388 | N | N | P |
| Spike | 389 | N | P | P |
| Spike | 390 | N | P | N |
| Spike | 391 | N | P | P |
| Spike | 392 | P | P | P |
| Spike | 393 | N | P | P |
| Spike | 394 | P | P | N |
| Spike | 395 | P | P | P |
| Spike | 396 | N | P | P |
| Spike | 397 | N | P | P |
| Spike | 398 | N | P | N |
| Spike | 399 | N | P | P |
| Spike | 400 | N | P | N |
| Spike | 401 | N | P | N |
| Spike | 402 | N | N | P |
| Spike | 403 | P | N | P |
| Spike | 404 | N | P | N |
| Spike | 405 | N | N | D |
| Spike | 407 | P | P | P |
| Spike | 408 | N | N | D |
| Spike | 409 | N | P | N |
| Spike | 410 | N | P | P |
| Spike | 411 | N | P | P |
| Spike | 412 | P | P | P |
| Spike | 413 | P | P | P |
| Spike | 414 | N | D | D |
| Spike | 416 | N | P | P |
| Spike | 417 | N | D | D |
| Spike | 418 | N | N | P |
| Spike | 419 | N | P | N |
| Spike | 420 | N | P | P |
| Spike | 421 | N | P | P |
| Spike | 422 | N | N | P |
| Spike | 423 | N | P | P |
| Spike | 425 | P | P | N |
| Spike | 426 | N | P | P |
| Spike | 427 | N | N | P |
| Spike | 428 | P | P | P |
| Spike | 429 | P | N | P |
| Spike | 431 | N | P | P |
| Spike | 432 | P | P | P |
| Spike | 433 | N | P | P |
| Spike | 436 | N | P | P |
| Spike | 437 | N | P | P |
| Spike | 438 | N | P | N |
| Spike | 439 | D | D | N |
| Spike | 440 | N | D | D |
| Spike | 441 | N | P | N |
| Spike | 442 | N | P | P |
| Spike | 443 | N | P | N |
| Spike | 444 | P | P | N |
| Spike | 445 | N | P | D |
| Spike | 446 | D | D | D |
| Spike | 447 | N | P | N |
| Spike | 448 | N | P | P |
| Spike | 449 | N | D | D |
| Spike | 450 | N | N | D |
| Spike | 452 | N | D | D |
| Spike | 453 | N | N | P |
| Spike | 454 | N | N | P |
| Spike | 456 | N | N | P |
| Spike | 457 | P | P | P |
| Spike | 458 | N | P | P |
| Spike | 460 | N | N | D |
| Spike | 461 | P | P | P |
| Spike | 462 | N | P | P |
| Spike | 463 | N | P | P |
| Spike | 464 | N | N | P |
| Spike | 465 | N | N | P |
| Spike | 466 | P | P | P |
| Spike | 467 | N | N | P |
| Spike | 468 | N | P | N |
| Spike | 469 | N | P | P |
| Spike | 472 | P | P | P |
| Spike | 473 | P | P | P |
| Spike | 474 | N | P | P |
| Spike | 475 | P | P | P |
| Spike | 477 | D | D | D |
| Spike | 478 | N | D | D |
| Spike | 480 | N | N | P |
| Spike | 481 | N | P | P |
| Spike | 482 | N | N | P |
| Spike | 484 | D | D | D |
| Spike | 486 | N | N | D |
| Spike | 487 | N | P | N |
| Spike | 488 | N | P | P |
| Spike | 489 | P | P | P |
| Spike | 490 | N | D | D |
| Spike | 491 | N | P | N |
| Spike | 492 | N | P | P |
| Spike | 493 | N | D | D |
| Spike | 494 | D | D | D |
| Spike | 495 | P | P | P |
| Spike | 496 | N | N | D |
| Spike | 497 | P | P | P |
| Spike | 498 | N | D | D |
| Spike | 499 | N | P | N |
| Spike | 500 | N | N | P |
| Spike | 501 | D | D | D |
| Spike | 502 | N | P | N |
| Spike | 503 | N | N | P |
| Spike | 504 | N | N | P |
| Spike | 505 | N | N | D |
| Spike | 507 | P | P | P |
| Spike | 508 | P | P | P |
| Spike | 510 | P | P | P |
| Spike | 511 | P | P | N |
| Spike | 512 | P | P | P |
| Spike | 513 | P | P | P |
| Spike | 514 | N | P | P |
| Spike | 517 | N | P | N |
| Spike | 518 | P | P | P |
| Spike | 519 | N | N | P |
| Spike | 521 | N | P | P |
| Spike | 522 | D | D | D |
| Spike | 523 | N | P | N |
| Spike | 524 | N | P | N |
| Spike | 525 | P | P | P |
| Spike | 527 | P | P | P |
| Spike | 528 | P | P | N |
| Spike | 530 | N | P | P |
| Spike | 531 | N | P | P |
| Spike | 532 | N | P | P |
| Spike | 533 | P | P | P |
| Spike | 534 | P | N | N |
| Spike | 535 | P | P | P |
| Spike | 536 | N | P | N |
| Spike | 538 | N | N | P |
| Spike | 539 | N | P | P |
| Spike | 540 | N | N | P |
| Spike | 541 | P | P | P |
| Spike | 542 | P | P | P |
| Spike | 543 | P | P | P |
| Spike | 544 | N | P | P |
| Spike | 545 | N | P | N |
| Spike | 546 | N | P | P |
| Spike | 547 | N | D | D |
| Spike | 549 | P | P | N |
| Spike | 550 | N | P | N |
| Spike | 551 | N | P | N |
| Spike | 554 | N | D | N |
| Spike | 555 | P | P | P |
| Spike | 556 | N | P | P |
| Spike | 557 | N | P | N |
| Spike | 559 | P | P | P |
| Spike | 560 | N | N | P |
| Spike | 561 | P | N | P |
| Spike | 562 | P | P | P |
| Spike | 563 | P | N | P |
| Spike | 564 | N | P | N |
| Spike | 567 | N | P | P |
| Spike | 569 | N | N | P |
| Spike | 570 | D | D | D |
| Spike | 571 | N | P | P |
| Spike | 572 | D | D | D |
| Spike | 573 | N | N | D |
| Spike | 574 | D | N | D |
| Spike | 575 | N | N | P |
| Spike | 576 | N | P | P |
| Spike | 577 | N | N | P |
| Spike | 578 | P | P | P |
| Spike | 579 | P | P | P |
| Spike | 580 | N | P | P |
| Spike | 581 | N | P | P |
| Spike | 582 | N | P | P |
| Spike | 583 | D | D | D |
| Spike | 584 | N | P | N |
| Spike | 585 | N | N | P |
| Spike | 586 | N | P | P |
| Spike | 587 | P | P | P |
| Spike | 589 | P | P | P |
| Spike | 590 | P | P | P |
| Spike | 591 | N | P | P |
| Spike | 592 | P | P | N |
| Spike | 593 | N | P | P |
| Spike | 595 | N | N | P |
| Spike | 596 | N | P | N |
| Spike | 597 | N | P | N |
| Spike | 598 | N | N | P |
| Spike | 599 | N | P | N |
| Spike | 600 | N | P | P |
| Spike | 601 | P | P | P |
| Spike | 602 | P | P | P |
| Spike | 603 | P | P | P |
| Spike | 605 | N | P | P |
| Spike | 606 | P | P | P |
| Spike | 608 | N | P | N |
| Spike | 609 | N | N | P |
| Spike | 610 | N | N | P |
| Spike | 611 | N | P | P |
| Spike | 612 | N | P | P |
| Spike | 613 | N | D | D |
| Spike | 614 | D | D | D |
| Spike | 616 | P | N | N |
| Spike | 617 | P | P | P |
| Spike | 618 | P | P | P |
| Spike | 619 | N | P | N |
| Spike | 620 | P | P | P |
| Spike | 622 | D | D | D |
| Spike | 623 | D | D | N |
| Spike | 624 | N | P | N |
| Spike | 625 | N | P | N |
| Spike | 626 | D | N | P |
| Spike | 627 | P | P | P |
| Spike | 629 | P | P | N |
| Spike | 631 | N | N | P |
| Spike | 633 | N | P | P |
| Spike | 634 | N | P | N |
| Spike | 636 | N | P | P |
| Spike | 637 | N | N | P |
| Spike | 638 | N | P | P |
| Spike | 640 | D | D | D |
| Spike | 643 | P | N | N |
| Spike | 645 | N | P | P |
| Spike | 648 | P | P | P |
| Spike | 649 | P | P | P |
| Spike | 650 | N | P | P |
| Spike | 652 | P | P | P |
| Spike | 653 | N | D | D |
| Spike | 654 | D | N | N |
| Spike | 655 | D | D | D |
| Spike | 656 | N | P | P |
| Spike | 657 | P | P | P |
| Spike | 658 | N | D | D |
| Spike | 659 | P | P | P |
| Spike | 660 | N | N | P |
| Spike | 661 | N | D | N |
| Spike | 662 | P | P | P |
| Spike | 663 | P | P | P |
| Spike | 665 | P | P | P |
| Spike | 666 | N | P | P |
| Spike | 668 | N | P | P |
| Spike | 669 | N | P | P |
| Spike | 671 | P | P | P |
| Spike | 672 | D | N | N |
| Spike | 673 | P | N | N |
| Spike | 674 | N | P | P |
| Spike | 675 | D | D | D |
| Spike | 676 | P | D | D |
| Spike | 677 | D | D | D |
| Spike | 678 | N | D | D |
| Spike | 679 | N | D | D |
| Spike | 680 | N | D | N |
| Spike | 681 | D | D | D |
| Spike | 682 | P | P | P |
| Spike | 683 | P | P | P |
| Spike | 685 | N | N | P |
| Spike | 688 | D | D | D |
| Spike | 691 | P | P | P |
| Spike | 692 | P | P | P |
| Spike | 693 | P | P | N |
| Spike | 695 | N | P | P |
| Spike | 697 | N | N | P |
| Spike | 699 | N | P | P |
| Spike | 700 | N | P | P |
| Spike | 701 | D | D | D |
| Spike | 702 | N | N | P |
| Spike | 703 | N | N | P |
| Spike | 704 | N | D | D |
| Spike | 705 | N | P | N |
| Spike | 706 | N | N | D |
| Spike | 707 | P | P | P |
| Spike | 708 | N | P | P |
| Spike | 709 | N | P | P |
| Spike | 711 | P | P | P |
| Spike | 713 | N | P | P |
| Spike | 715 | P | P | P |
| Spike | 716 | D | D | D |
| Spike | 717 | N | P | P |
| Spike | 718 | N | P | P |
| Spike | 721 | N | P | N |
| Spike | 723 | N | N | P |
| Spike | 724 | P | P | P |
| Spike | 725 | P | N | N |
| Spike | 726 | N | P | N |
| Spike | 727 | N | P | N |
| Spike | 728 | P | P | N |
| Spike | 729 | N | P | P |
| Spike | 732 | P | P | N |
| Spike | 733 | N | P | P |
| Spike | 734 | N | P | P |
| Spike | 735 | N | N | P |
| Spike | 736 | N | P | P |
| Spike | 737 | N | N | P |
| Spike | 738 | N | P | P |
| Spike | 739 | P | P | P |
| Spike | 740 | N | N | P |
| Spike | 742 | N | P | N |
| Spike | 743 | N | P | P |
| Spike | 744 | N | P | P |
| Spike | 746 | P | N | P |
| Spike | 747 | D | D | D |
| Spike | 748 | P | P | N |
| Spike | 749 | N | P | P |
| Spike | 750 | N | N | P |
| Spike | 751 | N | P | N |
| Spike | 752 | P | P | N |
| Spike | 753 | N | P | P |
| Spike | 755 | N | P | N |
| Spike | 757 | N | P | N |
| Spike | 759 | N | N | P |
| Spike | 760 | N | P | P |
| Spike | 761 | N | P | N |
| Spike | 762 | N | P | P |
| Spike | 763 | N | P | P |
| Spike | 764 | N | P | D |
| Spike | 766 | N | P | P |
| Spike | 767 | N | N | P |
| Spike | 768 | N | P | N |
| Spike | 769 | D | D | D |
| Spike | 770 | N | N | P |
| Spike | 771 | D | N | N |
| Spike | 773 | P | N | N |
| Spike | 774 | N | P | P |
| Spike | 775 | N | P | P |
| Spike | 776 | P | N | N |
| Spike | 777 | P | P | N |
| Spike | 778 | N | P | P |
| Spike | 779 | P | N | N |
| Spike | 780 | N | D | N |
| Spike | 781 | N | P | P |
| Spike | 782 | P | P | P |
| Spike | 783 | N | N | P |
| Spike | 785 | N | N | P |
| Spike | 786 | N | N | P |
| Spike | 788 | N | P | N |
| Spike | 789 | P | P | P |
| Spike | 790 | N | P | N |
| Spike | 791 | N | D | D |
| Spike | 792 | P | N | N |
| Spike | 793 | N | P | P |
| Spike | 795 | P | P | P |
| Spike | 796 | D | D | D |
| Spike | 799 | N | N | P |
| Spike | 800 | N | P | N |
| Spike | 801 | N | P | N |
| Spike | 803 | N | P | N |
| Spike | 806 | N | P | P |
| Spike | 807 | P | P | P |
| Spike | 808 | N | P | P |
| Spike | 809 | P | P | N |
| Spike | 810 | P | P | P |
| Spike | 811 | P | N | P |
| Spike | 812 | D | N | D |
| Spike | 813 | N | D | N |
| Spike | 814 | N | P | P |
| Spike | 815 | P | P | P |
| Spike | 816 | P | P | P |
| Spike | 817 | N | P | N |
| Spike | 818 | N | D | N |
| Spike | 819 | P | P | P |
| Spike | 820 | P | P | P |
| Spike | 821 | P | P | P |
| Spike | 822 | D | D | N |
| Spike | 823 | P | P | P |
| Spike | 824 | P | P | P |
| Spike | 825 | N | P | P |
| Spike | 826 | N | P | P |
| Spike | 828 | N | P | P |
| Spike | 830 | N | N | P |
| Spike | 832 | N | P | N |
| Spike | 834 | N | D | D |
| Spike | 835 | P | P | P |
| Spike | 837 | N | P | N |
| Spike | 838 | P | P | N |
| Spike | 839 | D + P | N | N |
| Spike | 840 | P | P | P |
| Spike | 841 | N | P | P |
| Spike | 843 | N | P | P |
| Spike | 844 | N | N | D |
| Spike | 845 | D | D | D |
| Spike | 846 | D | D | D |
| Spike | 848 | P | P | P |
| Spike | 849 | N | P | P |
| Spike | 851 | N | P | P |
| Spike | 853 | P | P | P |
| Spike | 854 | N | P | P |
| Spike | 855 | N | P | P |
| Spike | 856 | P | P | P |
| Spike | 857 | N | P | P |
| Spike | 858 | P | N | P |
| Spike | 859 | D | D | D |
| Spike | 861 | N | P | N |
| Spike | 862 | P | P | N |
| Spike | 863 | N | P | N |
| Spike | 864 | N | P | P |
| Spike | 866 | N | P | P |
| Spike | 867 | N | N | P |
| Spike | 868 | P | P | P |
| Spike | 869 | N | P | N |
| Spike | 870 | N | N | P |
| Spike | 872 | P | P | N |
| Spike | 873 | P | P | P |
| Spike | 874 | N | N | P |
| Spike | 875 | N | P | P |
| Spike | 876 | N | P | N |
| Spike | 877 | P | P | P |
| Spike | 878 | N | P | P |
| Spike | 879 | D | D | N |
| Spike | 880 | P | P | P |
| Spike | 881 | P | P | P |
| Spike | 882 | N | P | P |
| Spike | 883 | N | P | N |
| Spike | 884 | N | N | P |
| Spike | 886 | N | P | N |
| Spike | 887 | P | P | P |
| Spike | 888 | N | N | P |
| Spike | 889 | N | P | P |
| Spike | 890 | P | P | N |
| Spike | 891 | N | P | N |
| Spike | 892 | N | D | D |
| Spike | 893 | P | N | P |
| Spike | 894 | N | P | N |
| Spike | 895 | P | N | P |
| Spike | 897 | P | P | P |
| Spike | 898 | N | P | P |
| Spike | 900 | N | P | N |
| Spike | 901 | N | N | P |
| Spike | 902 | N | P | N |
| Spike | 903 | N | N | P |
| Spike | 904 | N | P | P |
| Spike | 905 | P | P | P |
| Spike | 906 | N | P | P |
| Spike | 907 | N | N | P |
| Spike | 908 | N | P | P |
| Spike | 909 | N | P | P |
| Spike | 910 | P | P | P |
| Spike | 911 | N | P | P |
| Spike | 912 | N | P | P |
| Spike | 913 | P | P | N |
| Spike | 914 | N | P | N |
| Spike | 915 | N | P | N |
| Spike | 917 | P | P | P |
| Spike | 918 | P | P | P |
| Spike | 919 | N | P | P |
| Spike | 921 | P | P | P |
| Spike | 922 | P | N | N |
| Spike | 923 | N | P | P |
| Spike | 924 | P | P | P |
| Spike | 925 | P | P | P |
| Spike | 927 | P | N | N |
| Spike | 928 | N | P | N |
| Spike | 929 | D | N | N |
| Spike | 930 | N | P | P |
| Spike | 931 | N | N | P |
| Spike | 932 | P | P | N |
| Spike | 933 | N | P | N |
| Spike | 934 | N | P | P |
| Spike | 935 | N | P | N |
| Spike | 936 | D | D | D |
| Spike | 937 | N | P | P |
| Spike | 938 | D | D | D |
| Spike | 939 | D | D | D |
| Spike | 940 | P | P | P |
| Spike | 941 | N | P | P |
| Spike | 942 | N | N | P |
| Spike | 943 | D | D | D |
| Spike | 944 | N | N | P |
| Spike | 945 | N | P | P |
| Spike | 946 | P | N | N |
| Spike | 947 | N | P | N |
| Spike | 948 | N | P | P |
| Spike | 949 | P | N | N |
| Spike | 950 | D | D | D |
| Spike | 951 | P | P | P |
| Spike | 952 | P | P | P |
| Spike | 953 | N | P | N |
| Spike | 954 | D | N | D |
| Spike | 955 | N | P | N |
| Spike | 956 | N | P | P |
| Spike | 957 | N | D | P |
| Spike | 958 | P | P | P |
| Spike | 959 | N | P | N |
| Spike | 960 | P | P | P |
| Spike | 961 | N | P | N |
| Spike | 964 | P | N | N |
| Spike | 966 | N | N | P |
| Spike | 967 | N | N | P |
| Spike | 968 | N | P | P |
| Spike | 970 | N | N | P |
| Spike | 971 | N | P | P |
| Spike | 972 | N | P | P |
| Spike | 973 | N | P | P |
| Spike | 974 | N | P | P |
| Spike | 975 | N | N | P |
| Spike | 976 | N | N | P |
| Spike | 979 | P | P | P |
| Spike | 980 | P | P | P |
| Spike | 981 | N | N | D |
| Spike | 982 | N | D | N |
| Spike | 983 | N | P | P |
| Spike | 984 | N | N | P |
| Spike | 986 | N | P | N |
| Spike | 987 | N | P | P |
| Spike | 988 | N | P | N |
| Spike | 989 | N | P | P |
| Spike | 990 | P | P | P |
| Spike | 991 | N | P | P |
| Spike | 992 | N | P | P |
| Spike | 993 | N | P | P |
| Spike | 994 | P | P | P |
| Spike | 995 | N | P | P |
| Spike | 996 | N | N | P |
| Spike | 997 | N | P | N |
| Spike | 998 | N | P | P |
| Spike | 999 | N | P | N |
| Spike | 1000 | N | P | N |
| Spike | 1001 | N | P | P |
| Spike | 1002 | N | P | P |
| Spike | 1003 | N | P | N |
| Spike | 1004 | N | P | P |
| Spike | 1005 | N | P | N |
| Spike | 1006 | N | P | P |
| Spike | 1007 | N | P | P |
| Spike | 1008 | P | P | P |
| Spike | 1009 | N | N | D |
| Spike | 1010 | N | P | N |
| Spike | 1011 | N | N | P |
| Spike | 1012 | N | P | N |
| Spike | 1014 | N | P | P |
| Spike | 1015 | N | P | N |
| Spike | 1016 | P | P | N |
| Spike | 1017 | N | P | N |
| Spike | 1018 | P | P | P |
| Spike | 1020 | D | D | D |
| Spike | 1022 | N | N | P |
| Spike | 1023 | N | P | N |
| Spike | 1024 | N | P | P |
| Spike | 1025 | N | P | P |
| Spike | 1026 | N | N | P |
| Spike | 1027 | D | D | D |
| Spike | 1028 | P | N | N |
| Spike | 1029 | N | P | N |
| Spike | 1030 | N | P | P |
| Spike | 1031 | N | N | P |
| Spike | 1032 | P | P | N |
| Spike | 1033 | P | N | N |
| Spike | 1034 | N | P | N |
| Spike | 1035 | N | N | P |
| Spike | 1037 | P | P | P |
| Spike | 1038 | P | N | N |
| Spike | 1039 | N | P | N |
| Spike | 1040 | N | P | P |
| Spike | 1041 | N | P | N |
| Spike | 1042 | N | P | P |
| Spike | 1043 | N | P | P |
| Spike | 1044 | P | P | P |
| Spike | 1045 | N | P | N |
| Spike | 1046 | N | P | P |
| Spike | 1048 | N | P | P |
| Spike | 1051 | N | P | P |
| Spike | 1053 | N | P | P |
| Spike | 1054 | N | N | P |
| Spike | 1055 | P | P | P |
| Spike | 1056 | P | P | P |
| Spike | 1057 | N | P | P |
| Spike | 1058 | N | N | P |
| Spike | 1059 | N | P | P |
| Spike | 1060 | P | P | N |
| Spike | 1061 | P | P | P |
| Spike | 1062 | P | P | P |
| Spike | 1064 | N | P | N |
| Spike | 1065 | P | P | P |
| Spike | 1066 | N | P | P |
| Spike | 1067 | P | P | P |
| Spike | 1069 | N | P | P |
| Spike | 1070 | D | D | D |
| Spike | 1071 | D | D | N |
| Spike | 1072 | N | P | P |
| Spike | 1073 | D | D | D |
| Spike | 1074 | N | D | N |
| Spike | 1075 | P | P | P |
| Spike | 1077 | P | P | P |
| Spike | 1078 | D | D | D |
| Spike | 1079 | N | P | N |
| Spike | 1080 | N | N | P |
| Spike | 1082 | P | P | P |
| Spike | 1083 | N | P | N |
| Spike | 1085 | D | N | N |
| Spike | 1086 | N | N | P |
| Spike | 1087 | D | D | N |
| Spike | 1089 | N | N | P |
| Spike | 1090 | N | P | P |
| Spike | 1091 | N | D | P |
| Spike | 1092 | P | P | N |
| Spike | 1093 | N | P | P |
| Spike | 1094 | P | P | P |
| Spike | 1095 | N | P | P |
| Spike | 1097 | P | P | P |
| Spike | 1098 | N | P | N |
| Spike | 1100 | P | P | P |
| Spike | 1101 | N | D + P | N |
| Spike | 1102 | N | P | N |
| Spike | 1104 | D | D | N |
| Spike | 1105 | N | N | P |
| Spike | 1106 | N | P | N |
| Spike | 1107 | N | P | P |
| Spike | 1110 | P | P | P |
| Spike | 1112 | N | P | P |
| Spike | 1114 | P | P | P |
| Spike | 1115 | N | N | P |
| Spike | 1116 | P | N | N |
| Spike | 1118 | D | D | D |
| Spike | 1119 | N | P | P |
| Spike | 1120 | P | N | N |
| Spike | 1121 | P | P | P |
| Spike | 1122 | P | P | P |
| Spike | 1123 | N | P | P |
| Spike | 1124 | D | D | D |
| Spike | 1126 | N | N | P |
| Spike | 1127 | N | N | D |
| Spike | 1131 | P | P | P |
| Spike | 1133 | D | N | N |
| Spike | 1135 | N | N | P |
| Spike | 1136 | N | P | P |
| Spike | 1138 | P | P | P |
| Spike | 1139 | P | D | N |
| Spike | 1140 | P | P | P |
| Spike | 1142 | P | P | P |
| Spike | 1143 | N | N | D |
| Spike | 1145 | N | P | N |
| Spike | 1146 | P | P | P |
| Spike | 1147 | N | P | P |
| Spike | 1148 | P | P | P |
| Spike | 1149 | P | P | P |
| Spike | 1150 | N | P | N |
| Spike | 1152 | P | P | P |
| Spike | 1153 | N | D | D |
| Spike | 1154 | N | N | P |
| Spike | 1158 | P | P | P |
| Spike | 1159 | N | P | P |
| Spike | 1160 | N | P | P |
| Spike | 1161 | N | P | N |
| Spike | 1162 | N | D | D |
| Spike | 1163 | D | D | D |
| Spike | 1166 | N | P | N |
| Spike | 1167 | D | D | D |
| Spike | 1168 | N | N | D |
| Spike | 1169 | P | P | N |
| Spike | 1170 | P | P | N |
| Spike | 1171 | N | N | P |
| Spike | 1172 | N | P | P |
| Spike | 1173 | N | P | P |
| Spike | 1174 | P | P | P |
| Spike | 1175 | N | P | P |
| Spike | 1176 | D | D | D |
| Spike | 1178 | P | P | P |
| Spike | 1179 | N | N | P |
| Spike | 1180 | N | D | N |
| Spike | 1181 | P | P | P |
| Spike | 1182 | N | P | N |
| Spike | 1183 | N | P | P |
| Spike | 1184 | N | P | N |
| Spike | 1185 | N | P | N |
| Spike | 1187 | N | P | P |
| Spike | 1188 | P | N | N |
| Spike | 1190 | N | P | P |
| Spike | 1191 | D | D | D |
| Spike | 1193 | P | P | P |
| Spike | 1194 | P | P | N |
| Spike | 1196 | P | P | P |
| Spike | 1197 | P | P | P |
| Spike | 1198 | N | P | P |
| Spike | 1199 | N | P | N |
| Spike | 1200 | N | P | P |
| Spike | 1201 | N | D | N |
| Spike | 1202 | D | D | D |
| Spike | 1203 | N | N | P |
| Spike | 1204 | N | P | P |
| Spike | 1205 | N | D | N |
| Spike | 1206 | N | P | P |
| Spike | 1207 | D | D | N |
| Spike | 1213 | P | N | P |
| Spike | 1215 | P | P | P |
| Spike | 1218 | N | P | P |
| Spike | 1219 | D | D | D |
| Spike | 1220 | N | P | P |
| Spike | 1222 | N | P | P |
| Spike | 1226 | N | P | P |
| Spike | 1228 | D | D | D |
| Spike | 1230 | P | P | N |
| Spike | 1232 | N | N | P |
| Spike | 1235 | P | D | D |
| Spike | 1238 | P | P | P |
| Spike | 1240 | P | P | N |
| Spike | 1242 | N | N | P |
| Spike | 1244 | N | P | N |
| Spike | 1245 | N | P | P |
| Spike | 1246 | P | P | P |
| Spike | 1247 | N | D | N |
| Spike | 1250 | D | N | D |
| Spike | 1251 | N | D | P |
| Spike | 1252 | D + P | D | D |
| Spike | 1253 | N | P | N |
| Spike | 1254 | N | N | P |
| Spike | 1255 | P | P | N |
| Spike | 1256 | N | N | P |
| Spike | 1257 | P | P | P |
| Spike | 1259 | P | P | P |
| Spike | 1260 | D | D | D |
| Spike | 1261 | N | P | N |
| Spike | 1263 | D | D | N |
| Spike | 1264 | N | N | D |
| Spike | 1266 | N | D | N |
| Spike | 1268 | P | P | N |
| Spike | 1269 | N | P | P |
| Spike | 1272 | N | P | P |

P = purifying selection (at least one timepoint with purifying selection within the time period).
D = diversifying selection (at least one timepoint with diversifying selection in time period).
D + P = at least one timepoint with purifying selection and another timepoint with diversifying selection within the time period.
N = neutral selection (no timepoints with either diversifying nor purifying selection within the time period).
Pre-Vaccine Timepoints: 2020-03-31, 2020-06-30, 2020-09-30, 2020-12-31.
Post-Vaccine Timepoints: 2021-03-31, 2021-06-30, 2021-09-30, 2021-12-31.
Post-Omicron Timepoints: 2022-03-31, 2022-06-30, 2022-09-30.

v. Impact of Selective Forces on SARS-CoV-2 Infectivity and Antibody Neutralization

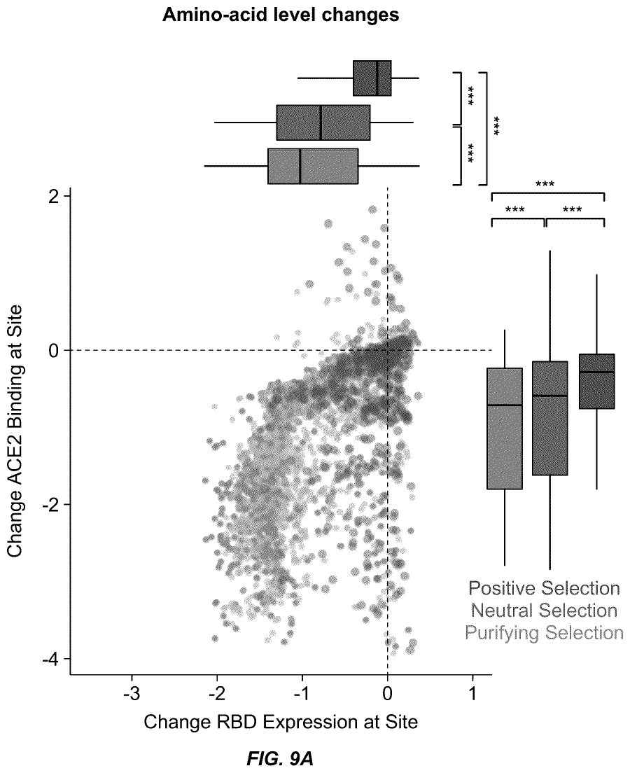

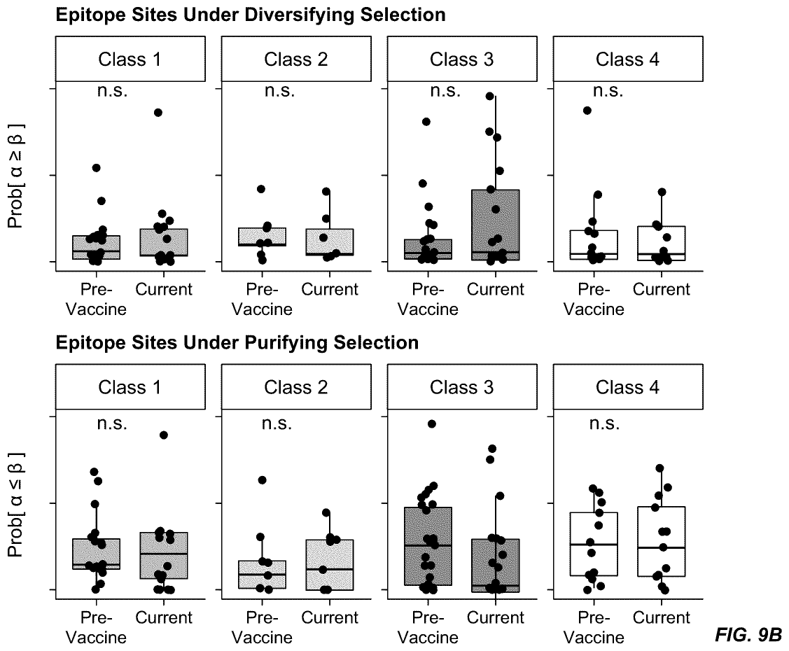

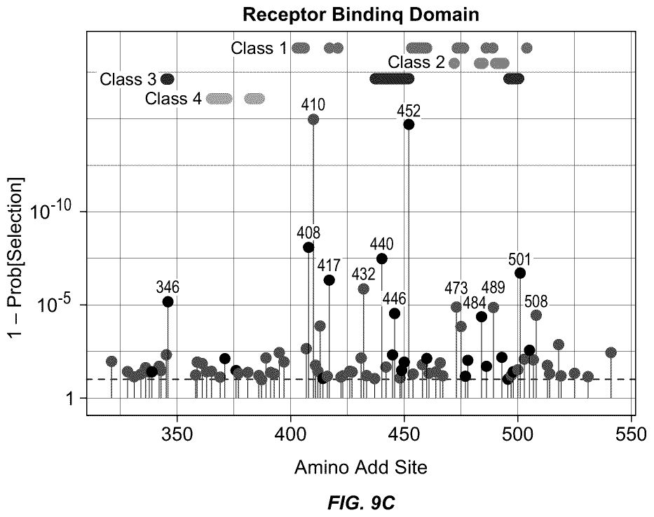

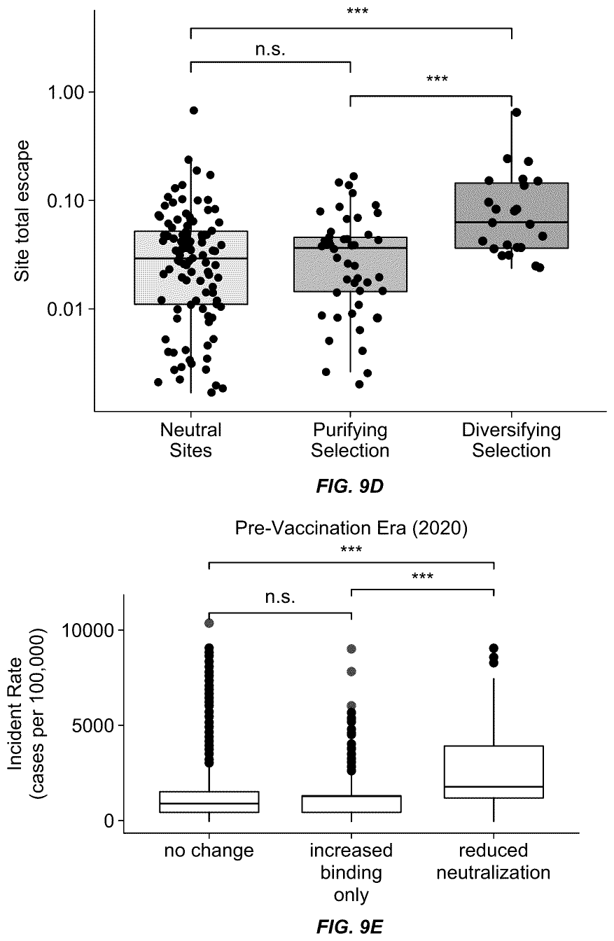

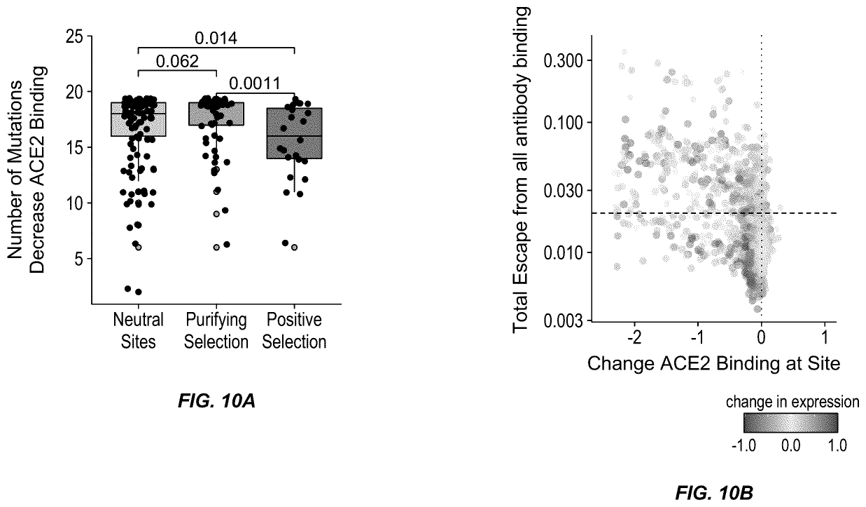

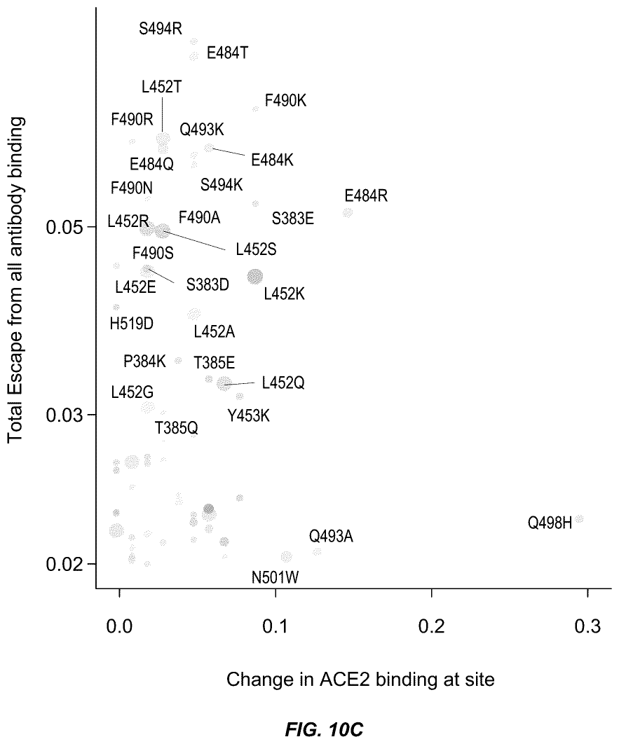

To further characterize how sites under selection within the RBD may relate to the pressures of the host environment, the impact of variants under selection on infectivity were first determined. The functional constraints of ACE2 binding were assessed using previously published deep mutational screens that measured the impact of mutations on affinity for ACE2 binding and protein folding stability, as estimated by RBD expression. Substitutions at sites under purifying selection (88.4%) had the strongest negative impact on ACE2 binding affinity and protein stability (FIG. 9A, FIG. 10A). Conversely, sites under diversifying selection had less effect on binding and stability (61.1% of sites) (FIG. 9A). Since this screen was limited at estimating increased binding affinity due to global epistasis estimation, N501Y was the only HFV in the RBD that demonstrated a substantial increase ACE2 binding affinity in this analysis. However, additional HFV have been reported to enhance binding affinity (N440K, L452R, S477N, S477I, T478K, and S494P), and these changes have been linked to increased infectivity and transmissibility.

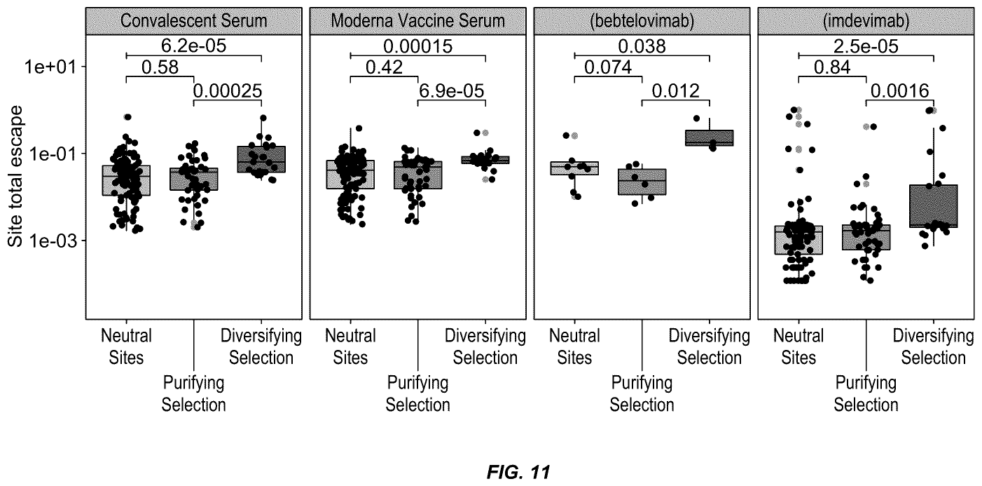

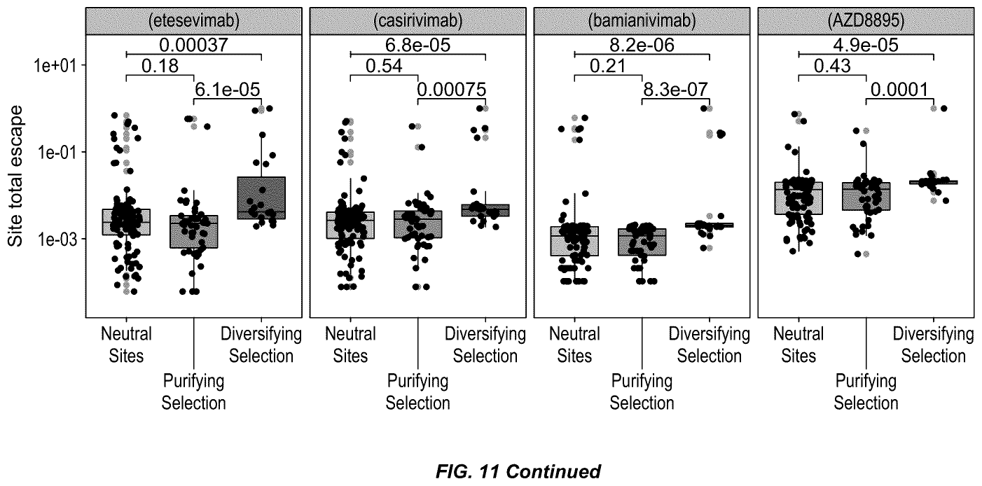

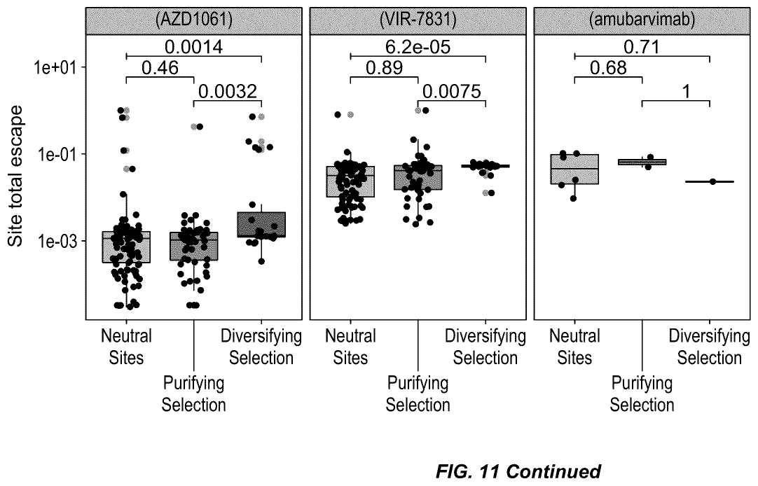

It was next assessed how sites under selection impacted antibody neutralization. It was compared how the site-level estimates of selection related to the four major classes (binding regions) of SARS-CoV-2 neutralizing antibodies. Each class varies in its ability to block ACE2 (class 1, 2) and its binding configuration to the RBD (class 1, 4: “up”-only; class 2, 3: “up” and “down”). While purifying selection applied to epitopes from all antibody classes, it had the strongest impact on epitopes from class 1 and class 4 antibodies (p=0.0169) (FIG. 9B) and the lowest impact on epitopes from class 3 antibodies. Overall, diversifying selection acting on class 3 antibody epitopes increased during the post-vaccine era (FIG. 9B). However, epitopes from each of these classes contained at least one site under strong diversifying selection which have been shown to impact therapeutic antibodies (FIG. 9C). Indeed, class 1 (e.g. casirivimab, etesevimab), class 2 (e.g. bamlanivimab) and class 3 antibodies (e.g. C110) have been reported to be negatively impacted by mutations at the 417, 484, and 452 sites respectively. The sites under diversifying selection significantly decreased antibody neutralization when compared to both sites under purifying (p=2.5×10⁴) and neutral selection (p=6.2×10⁵) (FIG. 9D, FIG. 11).

Finally, the impact of RBD HFVs was analyzed at sites under diversifying selection and it was found that many of these mutations have an impact on ACE2 binding affinity (e.g. N501Y), antibody neutralization (e.g. E484K/Q), or both (L452R/Q). Interestingly, many of these mutations have arisen at the same sites (R346, K417, L452, T478, E484, and N501) independently across multiple lineages (Table 1), indicating a common selective pressure acting on independent isolates. Mutations at site 417 (K417N/Beta+Omicron; K417T/Gamma) and 501 were physically linked in 77% of isolates (5,658,304/7,338,018). Because mutations at site 417 reduced antibody neutralization at a cost of ACE2 binding, and N501Y increased ACE2 affinity, the data indicated that some mutations can combine to alleviate the detrimental effect of individual variants.

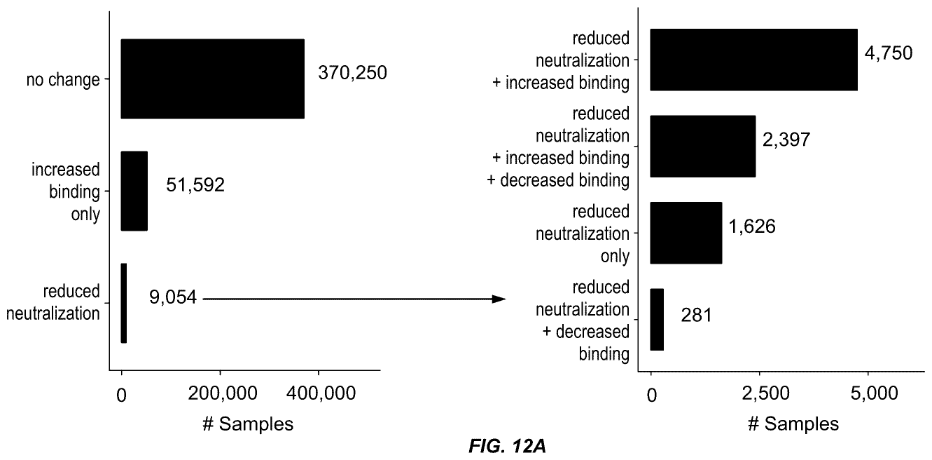

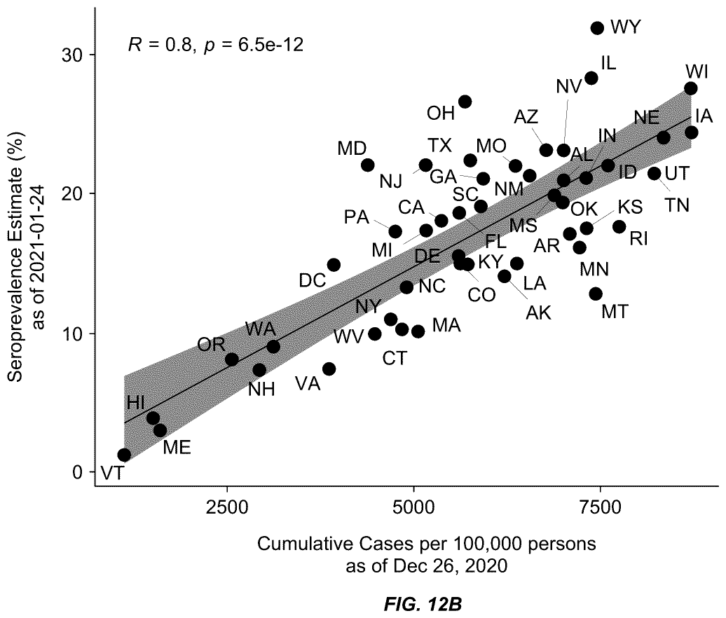

Each of the currently characterized major variants contain at least one of the RBD HFVs that are associated with increased infectivity. Additionally, all except Alpha also include a mutation that reduces antibody neutralization. The impact of the K417N/T and E484K on convalescent sera may have enabled the Beta and Gamma variants to spread in South Africa and Brazil despite high levels of seroprevalence in the late 2020 to early 2021 periods. To further investigate this, each isolate collected in 2020 was annotated with the incident rate (cases per 100,000) estimated from the geographic region and month from which the sample was collected, which was found to be a strong correlate with seroprevalence in the pre-vaccine era (FIG. 12). The variants that reduced antibody neutralization were found in areas with significantly higher seroprevalence (FIG. 9E). The Beta and Gamma, and later Omicron, variants have led to the loss of efficacy of both therapeutic antibodies (e.g., bamlanivimab, etesevimab) and specific vaccines (e.g., ChAdOx1). Altogether, this indicates that the mutations associated with reduced neutralization have serious implications for public health.

vi. Selective Pressures within Candidate Variable T Cell Epitopes

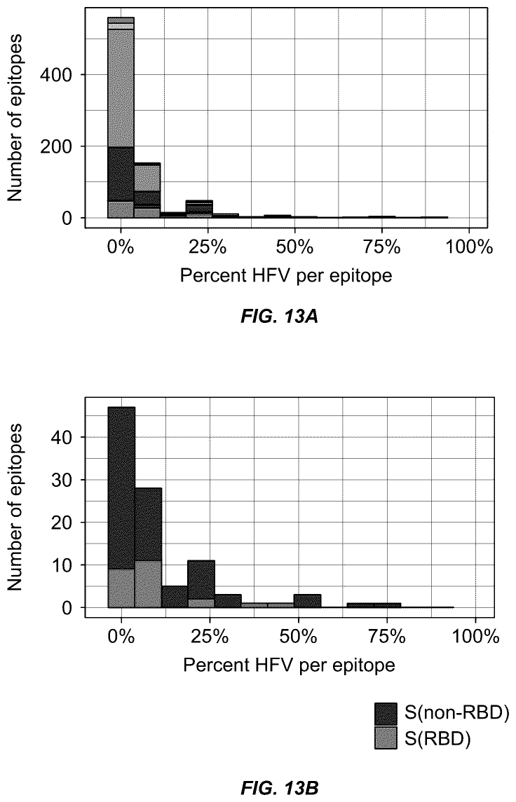

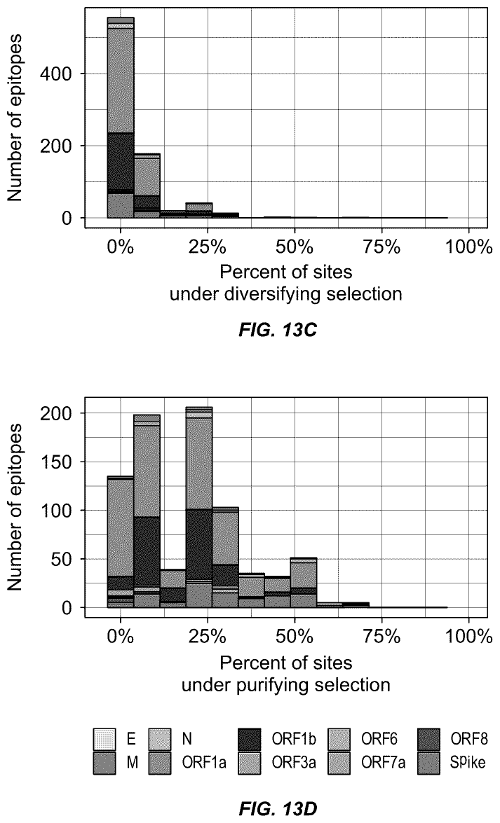

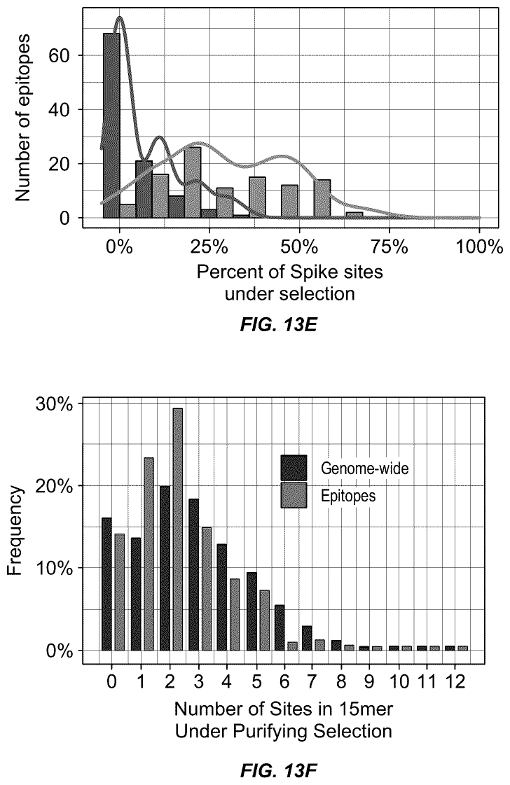

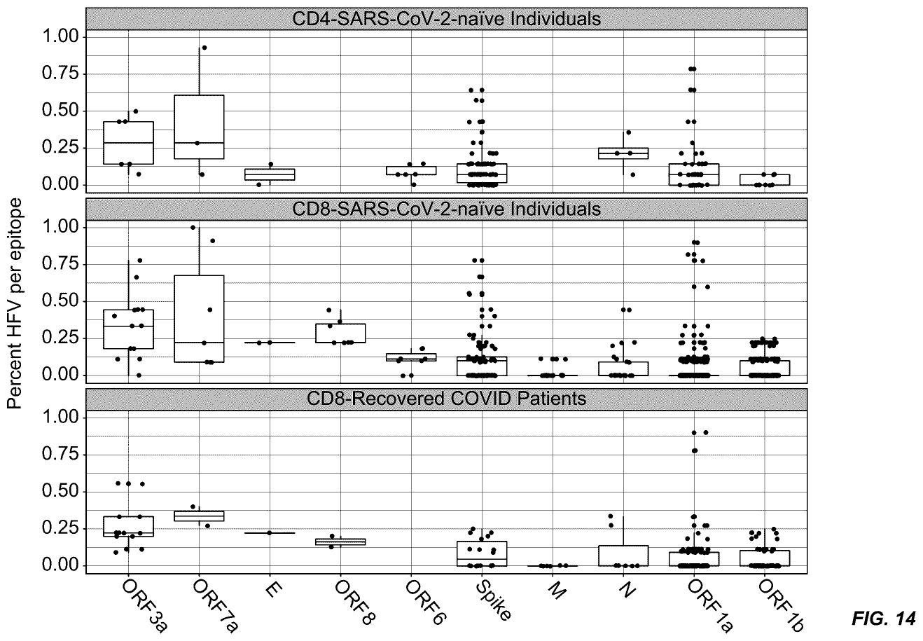

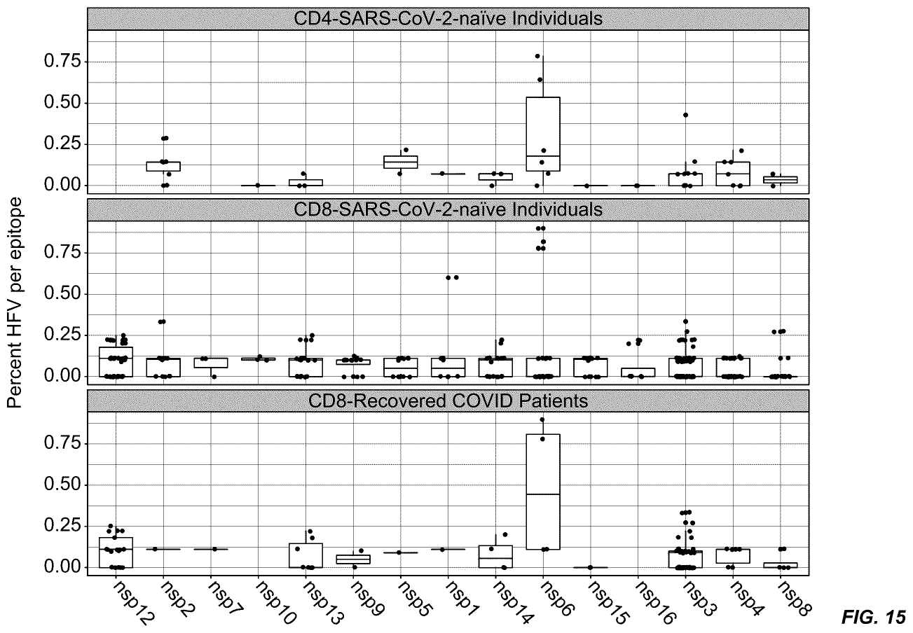

In order to determine if SARS-CoV-2 mutations occur in regions targeted by the human immune system, the distribution of amino acid mutations across 142 previously reported putative memory CD4+ T cell epitopes from SARS-CoV-2-naïve individuals and 637 previously reported candidate memory CD8+ T cell epitopes from both SARS-CoV-2-naïve individuals and recovered COVID patients were examined. For each peptide, all amino acid mutations observed in SARS-CoV-2 genomes present in GISAID were determined. While all peptides had each amino acid position mutated in at least one SARS-CoV-2 isolate, the vast majority of epitopes had very little overlap with HFV (FIG. 13A-B, FIG. 14-FIG. 15). Interestingly, the exception to this was ORF3a, which had large overlap (>25% of sites) with HFV in 92% (12/13) of peptides.

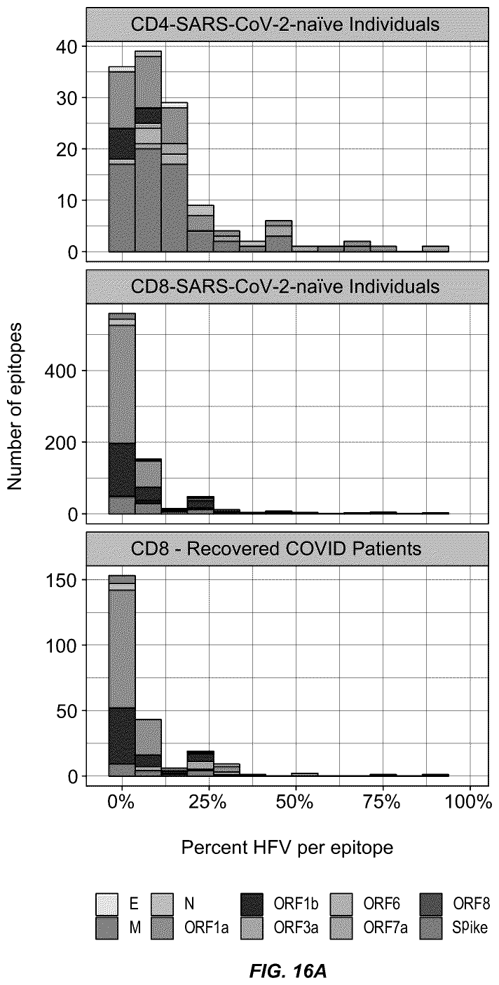

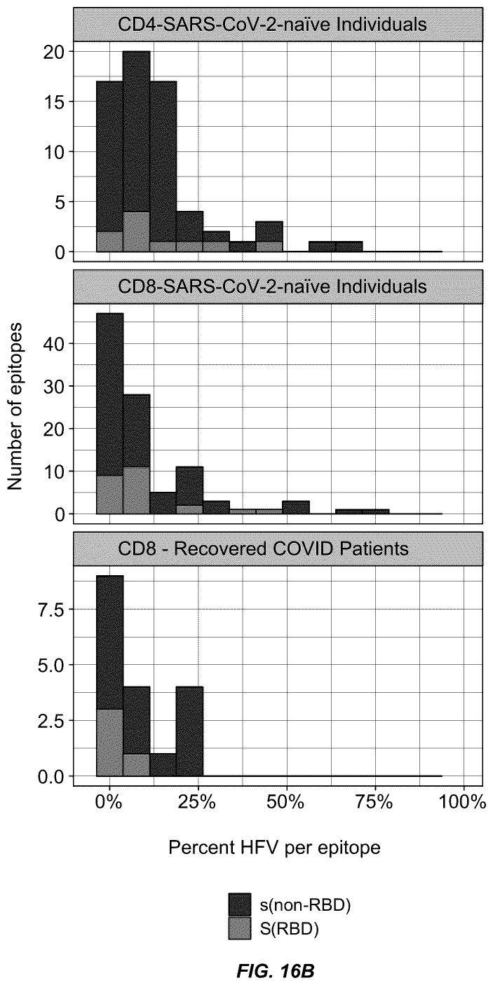

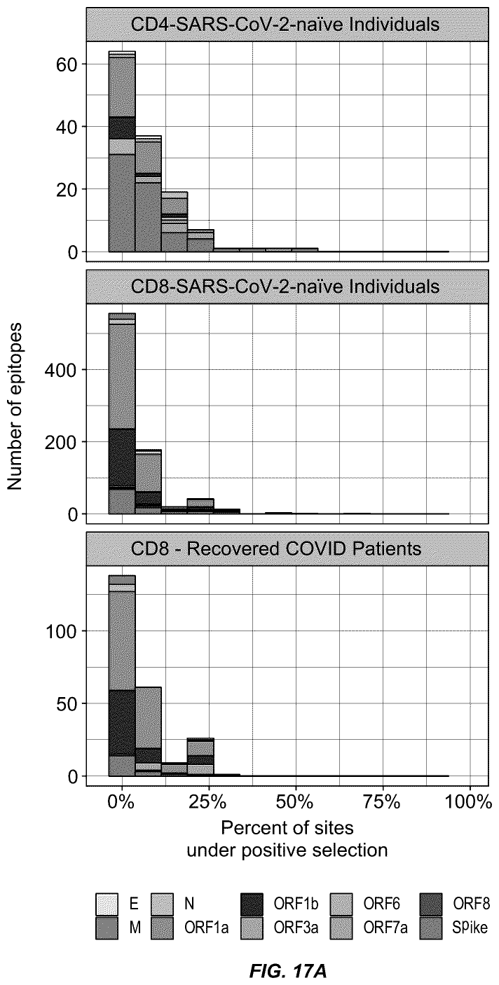

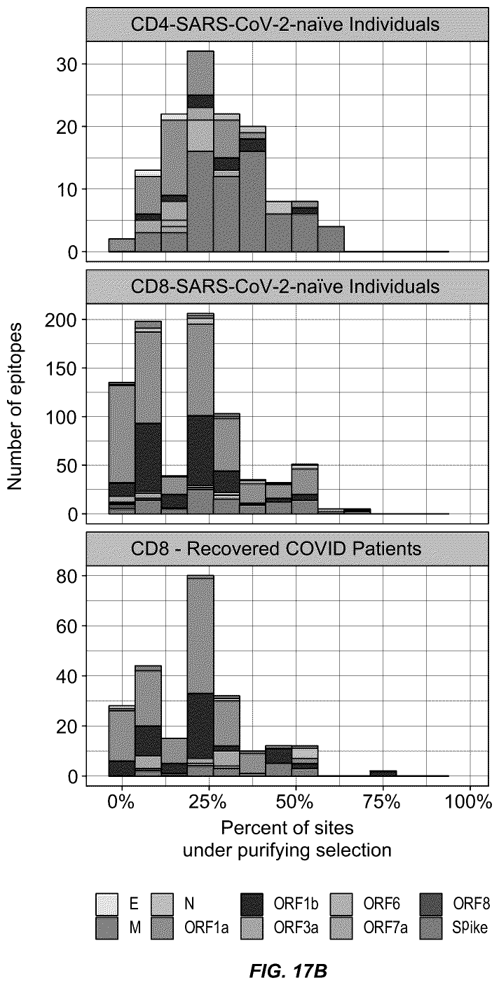

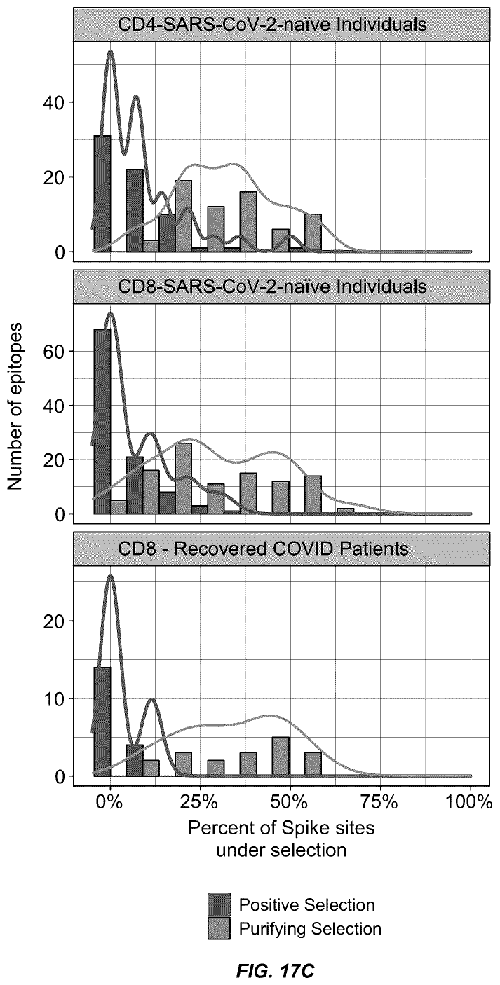

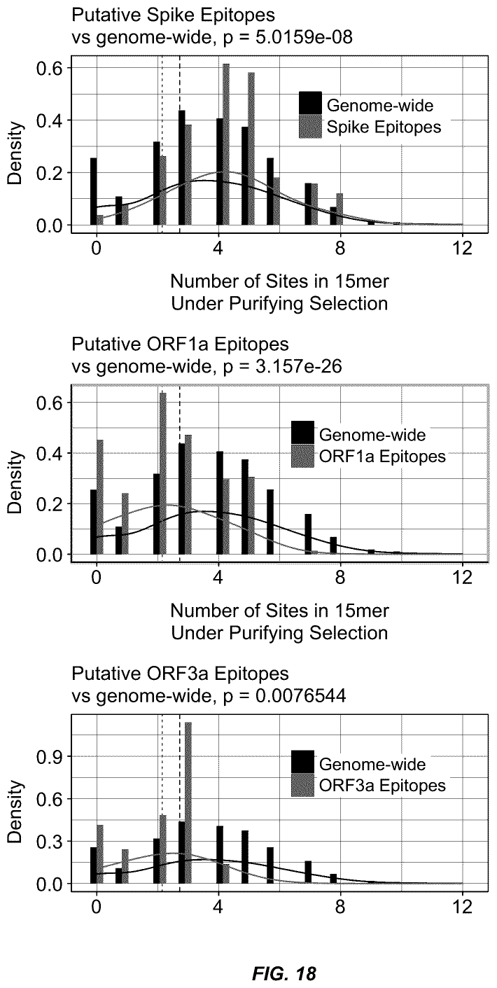

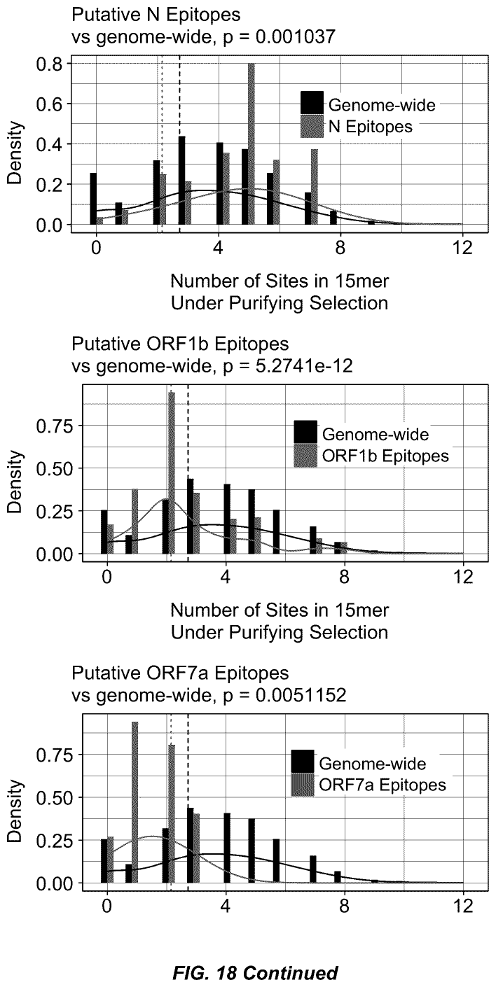

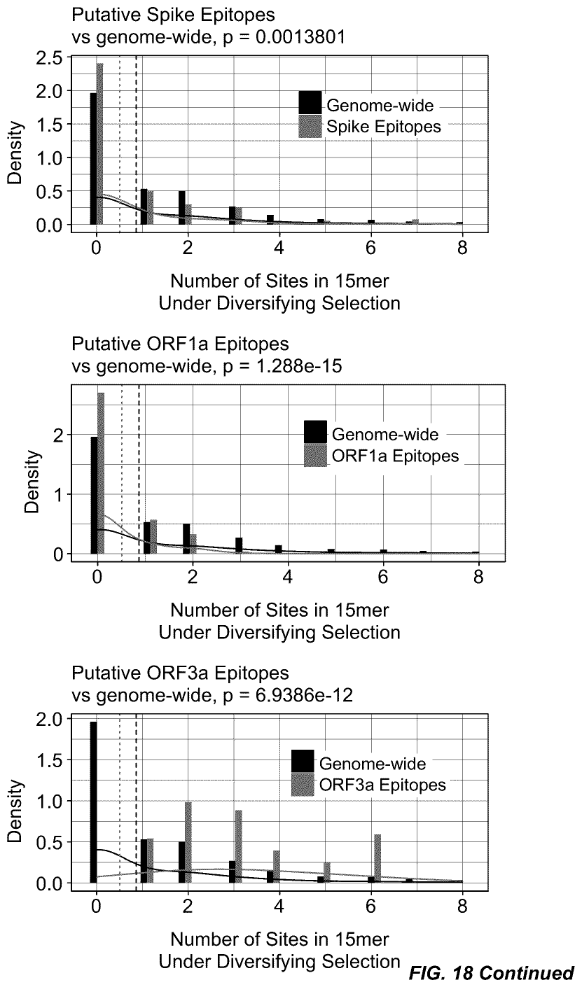

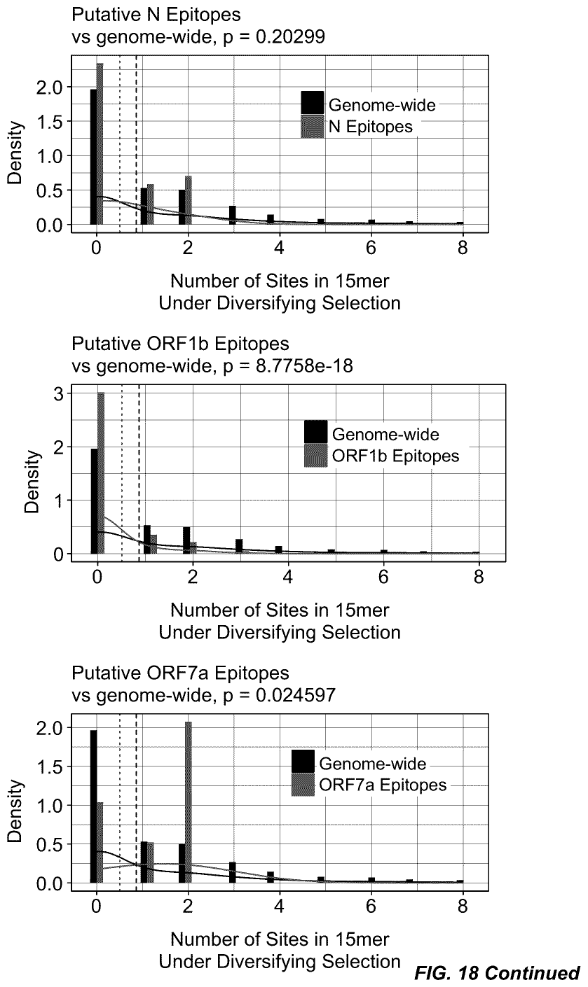

To determine whether these putative T cell epitope sequences in SARS-CoV-2 strains were under selective pressure, the number of sites under selection in the peptide sequences to background genome-wide levels of selection were compared. Overall, it was found that T cell epitope sequences were significantly enriched for sites under purifying selection (p=2.6×10⁴), with a single epitope sequence on average containing 3% (n=0-1) of their sites under diversifying selection and 20% (n=2-3) of their sites under purifying selection (FIG. 13C-F). Notably, there were no significant differences in patterns of selection between either CD4 and CD8 epitopes nor between SARS-CoV-2-naïve and recovered COVID patients (FIG. 16-FIG. 17). In addition to the absence of diversifying mutations, the spike, ORF1a, and N epitope sequences were significantly enriched for sites under purifying selection compared to the full genome (spike: p=1.3×10², ORF1b: p=0.04, N: p=0.005) (FIG. 18). Overall, the data indicates that T cell epitope sequences are under strong purifying selective pressures.

### 2. Discussion

The Omicron variant is the most successful SARS-CoV-2 lineage to date as demonstrated by its rapid spread and dominance across the world, starting at the end of 2021. Its success was explained by the accumulation of RBD mutations in the binding footprint of the virus's main entry host receptor (ACE2). Presence of such mutations within the RBD are known to potentially provide an evolutionary advantage by strengthening the virus infectivity, or by avoiding detection by neutralizing antibodies. One year following Omicron emergence, its origin remains unclear. Phylogenetic analysis has not identified any intermediary sequences between Omicron and its closest relatives and it did not reveal any straightforward evolutionary exit (frameshift or mutational profile) that could indicate that it descends from the Alpha, Beta, Delta or Gamma variants. However, defining mutations in Omicron lineages have been identified independently in strains early on during the pandemic. In this study, it was shown that the majority of these mutations targeted sites that quickly became under diversifying selection months before the emergence of Omicron (sites: 417, 440, 446, 452, 501, and 484). More specifically, diversifying forces acting on these sites intensified during the early utilization of vaccination worldwide. Importantly, this demonstrates that sites identified under selective forces represent early clues to predict the genome makeup of the next wave of emerging SARS-COV-2 strains. With that in mind, the analysis showed that in addition to an accumulation of RBD sites remaining under strong diversifying selection, new RBD sites have also recently been targeted by newly arising diversifying selection (including 445, 449, and 460), indicating that the virus evolution is not slowing down and indicating that it will lead to new SARS-CoV-2 variants.

The SARS-CoV-2 RBD is both the region mediating viral entry into the cell and the main target of most potent neutralizing anti-spike antibodies. RBD sites under strong levels of diversification were often associated with increased ACE2 binding and infectivity, as well as reduced antibody neutralization consistent with selective pressure imposed by the immune system on this domain. Fixation of such mutations comes at a fitness cost for the virus which can be compensated for by compensatory neutral mutations. For instance, while K417N/T variants in the RBD increased immune escape, it occurred at the fitness cost of reduced ACE2 binding affinity. Interestingly, it was found that mutations at this site have not been successful in spreading without the addition of the compensatory mutation N501Y. Experimental studies have confirmed that while K417N alone results in a significant drop in ACE2 binding affinity, its combination with N501Y resulted in 3-fold stronger binding than wild-type (although still 2-fold less compared to N501Y alone).

Neutralizing monoclonal antibodies have also been isolated that target non-RBD epitopes (Chi X., et al., 2020; Liu et al., 2020). For example, antibodies 4A8 and 1-68 have been reported to neutralize SARS-CoV-2 by targeting the NTD, which was characterized by elevated levels of diversifying selection. Variants including the 144 deletion found in the Alpha variant, the 242-244 deletions in the Beta variant, and multiple NTD mutations found in the Delta variant have demonstrated reduced sensitivity to anti-NTD neutralizing antibodies.

Another significant finding of the present study is the identification of a substantial number of sites under both diversifying and purifying selection outside of the spike protein. Elevated and intensified levels of diversification were found in the N, ORF3a, ORF7a, ORF8, and ORF10 genes. This can be explained by the roles these genes have been described to play in interacting with the immune system to block the innate immune response. Additionally, the N R203M mutation initially associated with the Delta variant was described to significantly increase mRNA production and delivery in to host cells and thus explain the increased transmissibility of this successful variant. In the analysis, R203M mutation site was identified as being under selection, demonstrating that identification of sites under selection and subsequent phenotypic characterization of mutations occurring at these sites can help better understand the keys of SARS-CoV-2 success. Altogether, this indicates that the pressures outside of the spike are also important for fitness and warrant closer examination.

Experimentally verified peptide epitopes derived from unexposed donors and recovered COVID patients were examined for sequence variants in SARS-CoV-2 and found that the vast majority were under strong conservation and did not accumulate sites under diversifying selection. In uninfected donors, SARS-CoV-2-reactive T cells can exhibit different patterns of immunodominance and frequently target ORF1a coding sequence, nsp7. In the present study, it was shown that nsp7 is hyper-conserved and has very few sites actively under diversifying selection. This observation is coherent with the high level of identity between orthologous sequences of nsp7 across multiple coronaviruses and helps explain why nsp7-specific T cells are detected and often dominant in unexposed donors as they may derive from exposure to other coronaviruses. Overall, data show that CD8⁺ and CD4⁺ T cell epitopes are still unaffected in Omicron genomes, indicating that Omicron does not appear to escape T cell responses.

To understand the future burden of COVID-19 it is imperative to carefully monitor the mechanisms generating highly antigenically divergent variants and the circumstances underlying their emergence. New dominating variants impose a relentless burden on the immune system which in turn imposes pressure on the virus to select the next variant, which can be as antigenically different from previous variants as possible to overcome host immunity. This study informs patterns of antigenic evolution in infected and vaccinated individuals and gives the tools to enable to more reliably identify the currently forming variants that may yet emerge.

### 3. Methods

i. Sequence Analysis, Filtering, and Alignment

There were approximately 13 million complete genome sequences deposited in GISAID as of Nov. 12, 2022, which were downloaded along with their variant calls and accompanying metadata. In order to reduce the number of genomes to a computationally tractable size, a two-stage down-sampling algorithm was developed to reduce the number of sequences to 2 million. The first step in this process was collecting 100 randomly sampled accessions for each for each pango lineages. In cases where there less than 100 sequences for a lineage, all available sequences were used. The balance of the 2 million sequences were composed of randomly sampled sequences, irrespective of lineage, with sampling dates normalized by using 1000 sampled accessions for each month, where available, between December 2019 and October 2022. After the date-based sampling, the resulting subset was topped up to 2 million by randomly selecting the remaining accessions with no lineage or date constraints. The 2 million sequences were then filtered to meet the following quality control (QC) criteria: complete accession metadata (no missing location or sample collection date); less than 5% ambiguous DNA (N) nucleotide or translated amino (X) sequences; no runs of 6 or more consecutive Ns in the DNA or Xs in the protein sequence; the entire set of the canonical gene-based open reading frames (ORFs) for the coding genome with full length BLAST hits when searched against the reference genome; no ORFs truncated by premature stop codons. The premature stop codon constraint was relaxed for ORF8, which has a stop codon at amino acid position 27 in several lineages. 820,056 sequences met all quality control filters, which were used as the population sampled for downstream analysis steps.

For selection analysis, more computationally feasible subsets of approximately 10,000 genomes were further down-sampled. The rapid spread of emerging variants of concern (VOCs) resulted in the majority of sampled genomes being from a dominant VOC lineage at any point in time. The sampling date distribution was simulated for the 10,000 sequence data sets by fitting the randomly sampled, QC-passed sequence count for each month of the pandemic to a log curve, favoring more recently samples genomes. Prior to this step, at least one randomly sampled representative of each lineage was preserved, with the balance of sequences composed of the date log curve-fitted random subsample. Using this method, up to 10,000 sequence sets were selected for timepoints. In some cases, sequences for a particular date range numbered less than 10,000 and all were retained. For time points with greater than 10,000 QC-passed sequences available, the data were down-sampled to approximately 10,000 sequences. The following timepoints were used: 2020-03-31, 2020-06-30, 2020-09-30, 2020-12-31, 2021-03-31, 2021-06-30, 2021-09-30, 2021-12-31, 2022-03-31, 2022-06-30, 2022-09-30. Each timepoint represents the end of a 3-month quarter. Accessions sampled prior the quarter beginning of the first quarter were included in the 2020-03-30 subset. Available samples collected later than 2022-09-30 but available in the 2022-11-12 GISAID download were included in the 2022-09-30 subset. Six replicate down-sampled 10,000 sequence sets were generated for the final timepoint to evaluate reproducibility of selection analysis of the sequences sets resulting from the down-sampling methodology. For each of the timepoints, coding sequences of each full-length gene-based open reading frame were aligned with the codon-aware multiple sequence aligner virulign. The aligned ORF sequences were then assembled into a full-length coding genomes.

ii. Phylogenetics

To guide the selection analysis (described below) for each of the timepoint data subsets, a whole-genome phylogenetic tree was inferred using fasttree with arguments -noml -nome -fastest. To create the phylogeny annotated in FIG. 1, a smaller subset of 2,000 QC-passed sequences was generated with unconstrained random sampling, except for preservation of at least 1 sequence of each lineage in the data subset. A more rigorous maximum-likelihood phylogenetic tree was inferred using fasttree for the 2,000 genomes set. The tree was outgroup rooted using a GISAID accession EPI_ISL_402125 from December 2019. The maximum likelihood tree was annotated with geographic, variant and mutation metadata using the ETE3 Python toolkit⁷¹. Images of annotated phylogenetic trees were rendered using the Python API to iTOL, the interactive Tree of Life.

iii. Natural Selection Analysis

The selection test was performed using Fast Unconstrained Bayesian AppRoximation (FUBAR) method from the hypothesis testing using phylogenies (HyPhy) Suite (https://stevenweaver.github.io/hyphy-site). The analysis was conducted for each single gene. The prepared alignment and corresponding whole-genome phylogenies described above were inputted into FUBAR to infer nonsynonymous (dN) and synonymous substitution (dS) rates at a per-site basis and test whether dN was significantly different from dS. Sites with probability values >0.9 were considered to be under non-neutral selection. This was repeated at the historical time points to understand the changes over time. For the purposes of interpretation, the historical quarters were binned into pre-vaccine (2020-03-31, 2020-06-30, 2020-09-30, 2020-12-31), post-vaccine (2021-03-31, 2021-06-30, 2021-09-30, 2021-12-31) and post-Omicron (2022-03-31, 2022-06-30, 2022-09-30) periods.

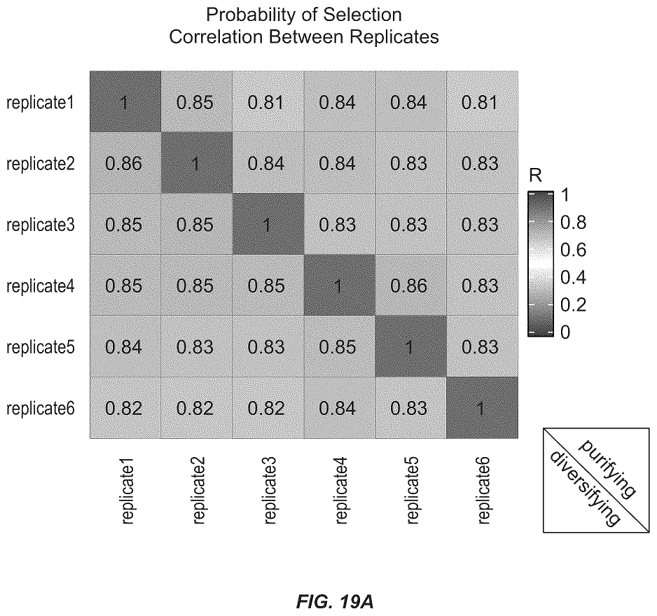

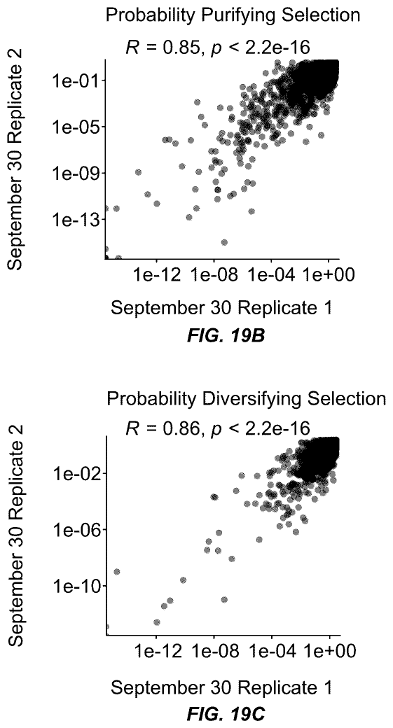

To assess whether the genome subsets were representative of the larger data set and that any results would be robust and reproducible, this analysis was repeated across six different replicates of the above-described down-sampling procedure. The consistency of the results was assessed using Pearson correlation of the log(1—probabilities) of diversifying and purifying selection across the full SARS-CoV-2 genome. There was a high degree of concordance among replicates (FIG. 19), indicating that these down-sampled subsets were sufficiently representative of the larger dataset and could be used to further investigate the evolution of the SARS-CoV-2 genome.

iv. Analysis of Deep Mutation Scan Data

Data from deep mutational scans were downloaded from the following sources: single mut effects.csv file from https://github.com/jbloomlab/SARS-CoV-2-RBD_DMS (RBD binding, expression constraints), final variant scores.csv from https://github.com/jbloomlab/SARS-CoV-2-RBD_DMS_Omicron (RBD binding, expression constraints), and the escape data.csv file from https://github.com/jbloomlab/SARS2_RBD_Ab_escape_maps (escape maps). The escape value was taken to be the average of “site total_escape” across all convalescent plasma samples.

v. Seroprevalence Analysis

CDC seroprevalence estimates were downloaded from https://covid.cdc.gov/covid-data-tracker/#serology-surveillance. The John Hopkins daily incident rate estimates were downloaded from https://github.com/CSSEGISandData/COVID-19. For each US state, the relationship between seroprevalence estimates and incident rates was evaluated using Pearson's correlation coefficient. Since these were highly correlated (R=0.8), the incident rate was used as a proxy for seroprevalence. For each sample in GISAID, the incident rate at the month (Collection Date) and highest-resolution place (county when available, otherwise state/country) was annotated. The incident rates were used to compare trends where different sets of mutations are spreading. This analysis was restricted to the pre-vaccination era of sequences, as defined by the samples collected between June 2020 (when incident rates became reliable) and Dec. 31, 2020 (when vaccinations began to ramp up in the United States).

vi. Analysis of CD4+ and CD8+ T Cell Epitope Sequences

The list of 142 previously published memory CD4+ T cell epitope sequences and genome coordinates was downloaded from Supplementary Table 1 of Mateus et al., 2020. The list of 637 previously published CD8+ T cell epitope sequences were downloaded from Supplementary Tables 4 and 7 of Saini et al., 2021 and each peptide sequence was aligned against the reference sequence using BLAST (accession: MN908947.3) to determine genome coordinates.

For each gene, the total numbers of unique sites that were covered by epitope sequences and under diversifying/purifying selection were counted. Significance was assessed using a Chi-square test comparing the number of sites under selection within sites targeted by candidate epitopes to genome-wide levels of selection. The number of sites under diversifying/purifying selection were computed for all 15-mers genome-wide and compared to epitope sequences using the one-sided Wilcoxon rank-sum test for each gene. The number of sites under diversifying/purifying selection were also computed for all 15-mers genome-wide and compared to epitope sequences using the one-sided Wilcoxon rank-sum test for each gene.

vii. Data Availability

Sequences downloaded from the GISAID EpiCoV database are not available due to data-sharing restrictions. Data from deep mutational scans was downloaded from github.com/jbloomlab/SARS-CoV-2-RBD_DMS_Omicron (RBD binding, expression constraints) and github.com/jbloomlab/SARS2_RBD_Ab_escape_maps (escape maps).

CDC seroprevalence estimates were downloaded from covid.cdc.gov/covid-data-tracker/#serology-surveillance.

The John Hopkins daily incident rate estimates were downloaded from github.com/CSSEGISandData/COVID-19.

Previously published T cell epitope sequences were downloaded from Supplementary Table 1 of Mateus et al., 2020 and supplementary Tables 4 and 7 of Saini et al., 2021.

### B. Example 2: The Selective Landscape of the SARS-CoV-2 Genome at a Snapshot in Time can be Used to Predict Future Variants with High Accuracy

Majority of Spike mutations contained in current emerging variants were identified as under selection over a year ago.

Out of the 95 spike sites that have been identified under strong diversifying selection, 90.5% (86/95) contained high frequency variants (HFV), which were defined to be mutations that have been observed in at least 10,000 isolates. Similarly, 95.2% (20/21) of the sites identified in the receptor binding domain (RBD) under strong diversifying selection were associated with HFV.

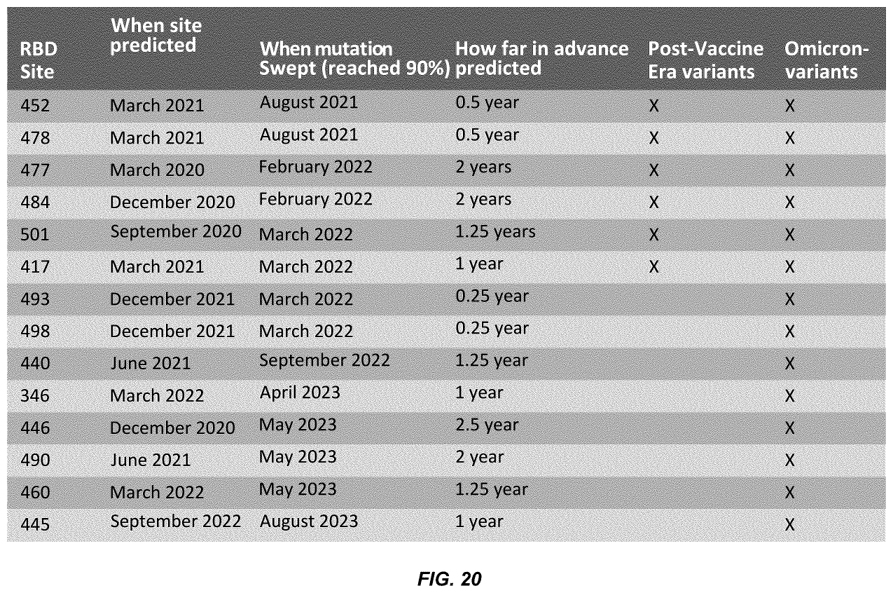

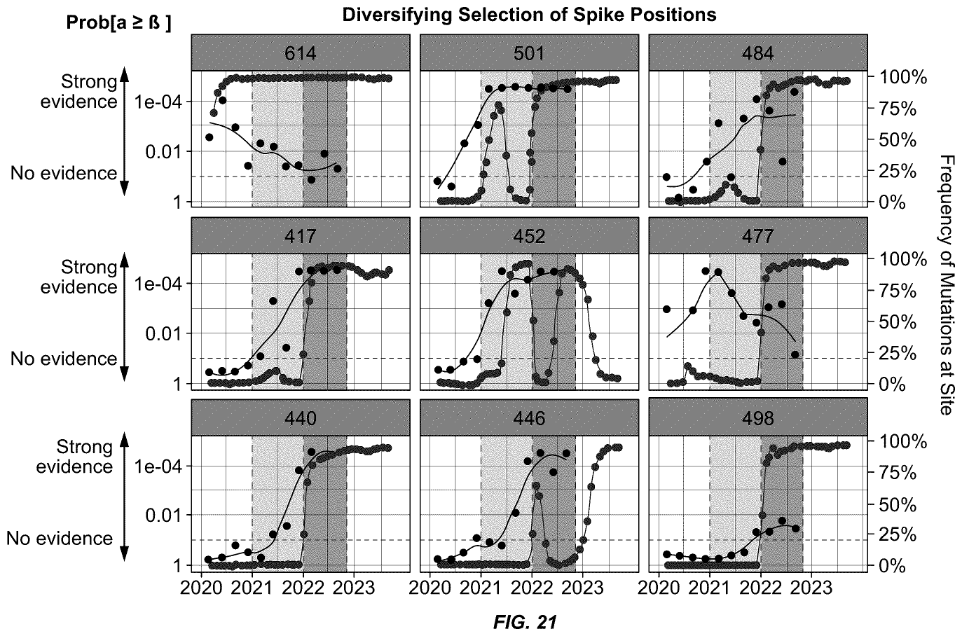

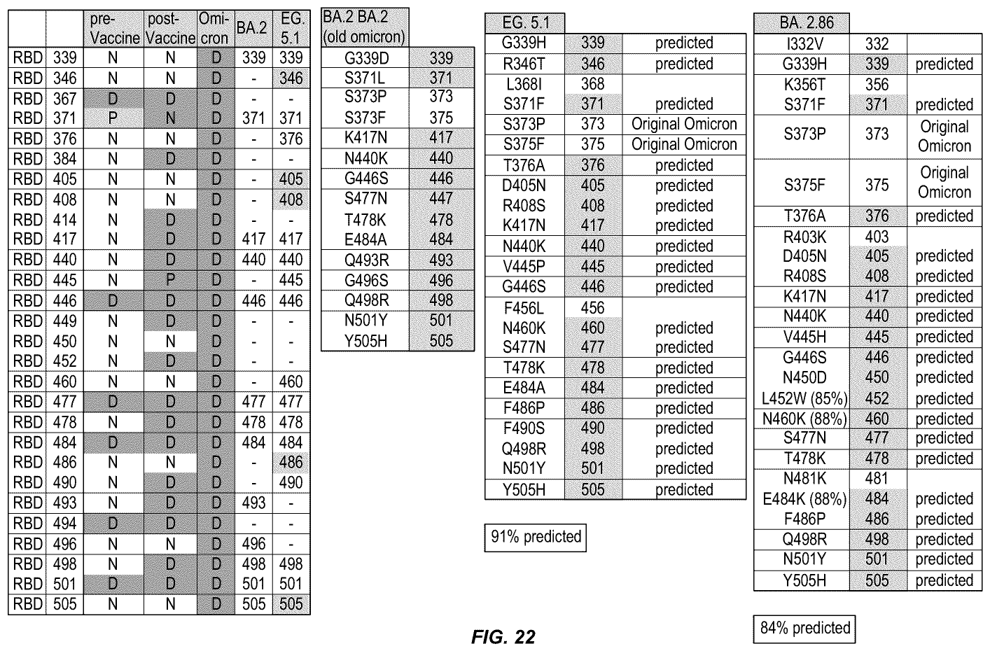

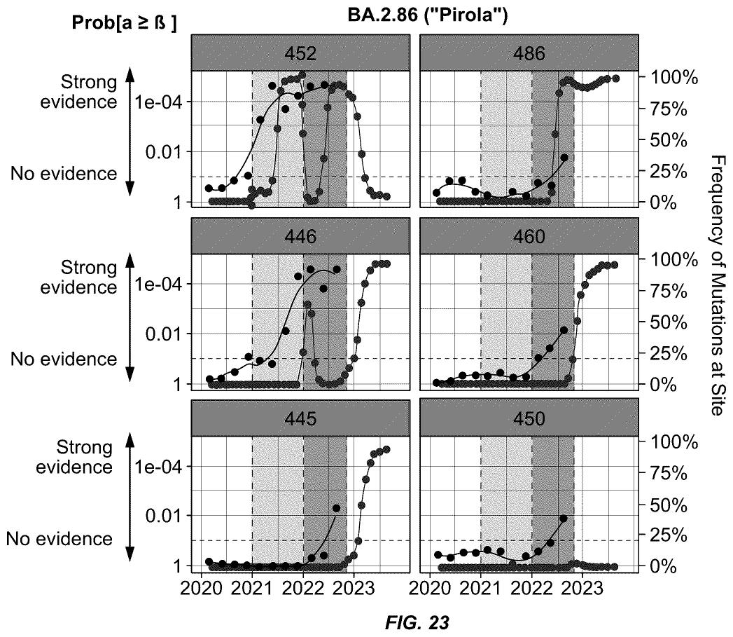

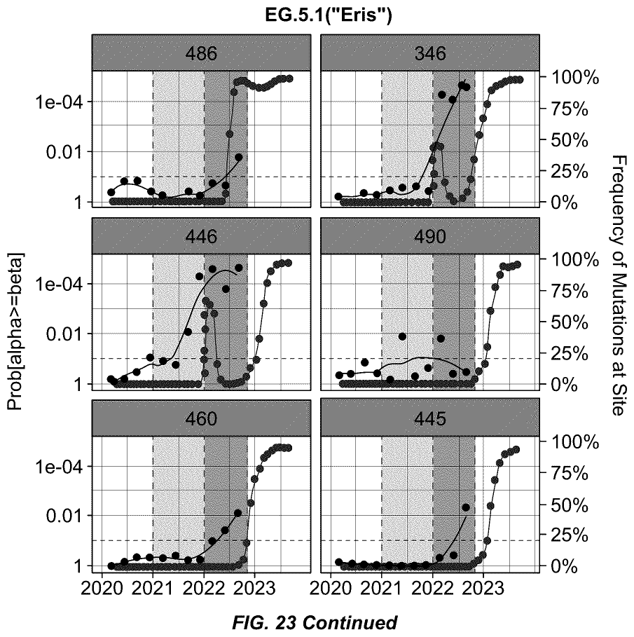

Among the spike variants that have swept through the global population (i.e. variants found in >90% of isolates collected within a month), the majority (>70%) were under intensifying positive selection before the sweep occurred (FIG. 20 and FIG. 21). Furthermore, in the cases of the recently identified BA.2.86 and EG.5.1 variants, the defining mutations in the RBD sites were predicted with 84% and 91% accuracy, respectively. (FIG. 22 and FIG. 23).

Furthermore, only few sites that have been identified under strong purifying selection have ever resulted in HFV. Indeed, out of the 219 Spike sites that have ever been under purifying selection, only 14 sites (6.4%) have ever contained HFV and only one site has contained a mutation that swept through the population. In the RBD, only 1% (1/33) of sites under strong purifying selection contained HFV and none had mutations that swept through the population.

Altogether, this suggests that characterizing the selective landscape is highly predictive of the evolutionary trajectory of the SARS-CoV-2 genome.

This has applications in the design and evaluation of novel and existing preventative and therapeutic interventions, including monoclonal antibodies, vaccines, or any therapeutic that specifically targets SARS-CoV-2 proteins.

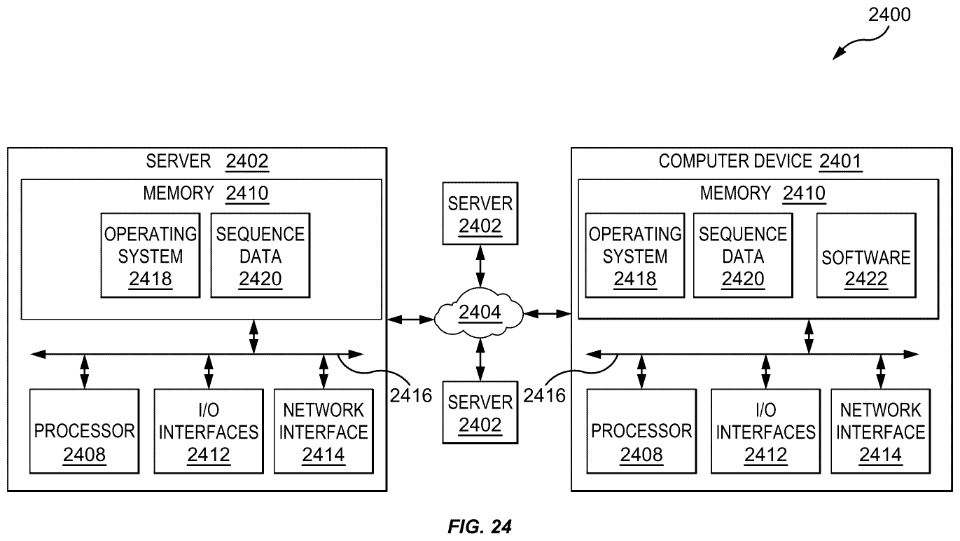

FIG. 24 is a block diagram depicting an environment 2400 comprising non-limiting examples of a computing device 2401 and a server 2402 connected through a network 2404. In an aspect, some or all steps of any described method may be performed on a computing device as described herein, including the method 100 of FIG. 1. The computing device 2401 can include one or multiple computers configured to store one or more of sequence data 2420 (e.g., viral genome sequence data, full-length coding genomes, and the like), software 2422 (e.g., down-sampling software, alignment software, mapping software, software to perform the method 100 of FIG. 1, and the like), and the like. The server 2402 can include one or multiple computers configured to store the sequence data 2420. Multiple servers 2402 can communicate with the computing device 2401 via the through the network 2404.

The computing device 2401 and the server 2402 can be a digital computer that, in terms of hardware architecture, generally includes a processor 2408, memory system 2410, input/output (I/O) interfaces 2412, and network interfaces 2414. These components (2408, 2410, 2412, and 2414) are communicatively coupled via a local interface 2416. The local interface 2416 can be, for example, but not limited to, one or more buses or other wired or wireless connections, as is known in the art. The local interface 2416 can have additional elements, which are omitted for simplicity, such as controllers, buffers (caches), drivers, repeaters, and receivers, to enable communications. Further, the local interface may include address, control, and/or data connections to enable appropriate communications among the aforementioned components.

The processor 2408 can be a hardware device for executing software, particularly that stored in memory system 2410. The processor 2408 can be any custom made or commercially available processor, a central processing unit (CPU), an auxiliary processor among several processors associated with the computing device 2401 and the server 2402, a semiconductor-based microprocessor (in the form of a microchip or chip set), or generally any device for executing software instructions. When the computing device 2401 and/or the server 2402 is in operation, the processor 2408 can be configured to execute software stored within the memory system 2410, to communicate data to and from the memory system 2410, and to generally control operations of the computing device 2401 and the server 2402 pursuant to the software.

The I/O interfaces 2412 can be used to receive user input from, and/or for providing system output to, one or more devices or components. User input can be provided via, for example, a keyboard and/or a mouse. System output can be provided via a display device and a printer (not shown). I/O interfaces 2412 can include, for example, a serial port, a parallel port, a Small Computer System Interface (SCSI), an infrared (IR) interface, a radio frequency (RF) interface, and/or a universal serial bus (USB) interface.

The network interface 2414 can be used to transmit and receive from the computing device 2401 and/or the server 2402 on the network 2404. The network interface 2414 may include, for example, a 10BaseT Ethernet Adaptor, a 100BaseT Ethernet Adaptor, a LAN PHY Ethernet Adaptor, a Token Ring Adaptor, a wireless network adapter (e.g., WiFi, cellular, satellite), or any other suitable network interface device. The network interface 2414 may include address, control, and/or data connections to enable appropriate communications on the network 2404.

The memory system 2410 can include any one or combination of volatile memory elements (e.g., random access memory (RAM, such as DRAM, SRAM, SDRAM, etc.)) and nonvolatile memory elements (e.g., ROM, hard drive, tape, CDROM, DVDROM, etc.). Moreover, the memory system 2410 may incorporate electronic, magnetic, optical, and/or other types of storage media. Note that the memory system 2410 can have a distributed architecture, where various components are situated remote from one another, but can be accessed by the processor 2408.

The software in memory system 2410 may include one or more software programs, each of which includes an ordered listing of executable instructions for implementing logical functions. In the example of FIG. 24, the software in the memory system 2410 of the computing device 2401 can include the sequence data 2420, the software 2422, and a suitable operating system (O/S) 2418. In the example of FIG. 24, the software in the memory system 2410 of the server 2402 can include, the sequence data 2420, and a suitable operating system (O/S) 2418. The operating system 2418 essentially controls the execution of other computer programs and provides scheduling, input-output control, file and data management, memory management, and communication control and related services.

For purposes of illustration, application programs and other executable program components such as the operating system 2418 are illustrated herein as discrete blocks, although it is recognized that such programs and components can reside at various times in different storage components of the computing device 2401 and/or the server 2402. An implementation of the software 2422 can be stored on or transmitted across some form of computer readable media. Any of the disclosed methods can be performed by computer readable instructions embodied on computer readable media. Computer readable media can be any available media that can be accessed by a computer. By way of example and not meant to be limiting, computer readable media can include “computer storage media” and “communications media.” “Computer storage media” can include volatile and non-volatile, removable and non-removable media implemented in any methods or technology for storage of information such as computer readable instructions, data structures, program modules, or other data. Exemplary computer storage media can include RAM, ROM, EEPROM, flash memory or other memory technology, CD-ROM, digital versatile disks (DVD) or other optical storage, magnetic cassettes, magnetic tape, magnetic disk storage or other magnetic storage devices, or any other medium which can be used to store the desired information and which can be accessed by a computer.

In an embodiment, the software 2422 may be configured to perform the methods disclosed herein, including the method 100 of FIG. 1. The methods disclosed herein may be performed in whole or in part by a single computing device, a plurality of electronic devices, and the like.

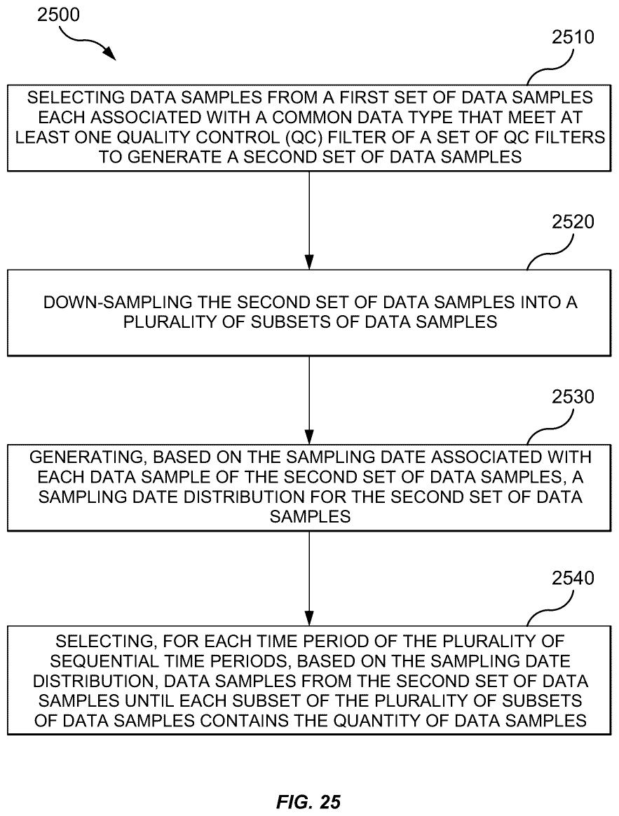

FIG. 25 shows an example method 2500 to sample data in accordance with embodiments of the disclosure. The method 2500 may be performed by the server 2402 and/or the computing device 2401 of FIG. 24, in some examples.

The method 2500 includes selecting data samples from a first set of data samples that meet at least one quality control (QC) criterion to generate a second set of data samples, at 2510. Each of the first set of data samples is associated with a sampling date. In some examples, a quantity of data samples in the first set of data samples includes at least two million data samples. The at least one QC criterion may include one or more of: requiring data source information, requiring the sampling date, requiring lack of data errors, requiring lack of missing data, requiring a complete data sample, requiring data samples from a particular source, or excluding data samples from a particular source. In some examples, the plurality of subsets of data samples are configured for selection analysis, and the selection analysis is facilitated by a computationally feasible number of data samples within each subset of the plurality of subsets of data samples. In some examples, the first set of data samples is associated with a common data type. In some examples, the common data type includes a common viral species. In some examples, the common viral species includes SARS-CoV-2. In some examples, the common data type is selected from at least one of: genomic data; data used to track genetic variations in endangered species; disease data used to monitor disease progression; data used to analyze drug response; disease data used to monitor ecosystem; data used to monitor genetic changes in plants over time; data used for data compression for storing and transmitting large datasets; data used for early diagnosis of genetic disorders; data used for constructing phylogenetic trees; genetic data from multiple sources that can be integrated together; or genetic data used for training or testing machine learning models. In some examples, the common data type is selected from stock or financial market data, data used for fraud detection, data used for network traffic analysis.

The method 2500 includes down-sampling the second set of data samples into a plurality of subsets of data samples, at 2520. Each subset of the plurality of subsets of data samples contains a predetermined quantity of data samples, and each subset of the plurality of subsets of data samples is associated with a different time period of a plurality of sequential time periods. In some examples, down-sampling the second set of data samples into the plurality of subsets of data samples is via a machine learning model trained using labeled data to down-sample data samples according to one or more predefined criteria. In some examples, down-sampling the second set of data samples into the plurality of subsets of data samples is via a large language model trained using labeled data to down-sample data samples according to one or more predefined criteria. The machine learning model and/or the large language model may be implemented using the software 2422 of FIG. 24.

Down-sampling the second set of data samples into a plurality of subsets of data samples includes generating, based on the sampling date associated with each data sample of the second set of data samples, a sampling date distribution for the second set of data samples, at 2530, and selecting, for each time period of the plurality of sequential time periods, based on the sampling date distribution, data samples from the second set of data samples until each subset of the plurality of subsets of data samples contains the predetermined quantity of data samples, at 2540. The method 2500 may further include selecting, for each time period of the plurality of sequential time periods, a representative data sample from the second set of data samples of each data subtype of a plurality of data subtype for each subset of the plurality of subsets of data samples. In some examples, the predetermined quantity of data samples includes at least ten thousand data samples. In some examples, generating the sampling date distribution may include fitting a quantity of data samples from the second set of data samples to an exponential curve. In some examples, fitting the quantity of data samples from the second set of data samples to the exponential curve may include generating the sampling date distribution by fitting the quantity of data samples from the second set of data samples for each of a second time period to the exponential curve. In other examples, generating the sampling date distribution includes fitting a quantity of data samples from the second set of data samples to a predetermined statistical curve or trend.

In some examples, selecting, for each time period of the plurality of sequential time periods, based on the sampling date distribution, data samples from the second set of data samples until each subset contains the predetermined quantity of data samples may include for any time period of the plurality of sequential time periods with more than the predetermined quantity of data samples available, randomly selecting data samples from the second set of data samples until the subset of the plurality of subsets of data samples associated with the time period contains the predetermined quantity of data samples, and for any time period of the plurality of sequential time periods with less than the predetermined quantity of data samples available, retaining all data samples for the subset of the plurality of subsets of data samples associated with the time period. In some examples, each time period of the plurality of sequential time periods represents a second, a minute, an hour, a day, a month, a predetermined quantity of months, a year, or a predetermined quantity of years.

In some examples, the method 2500 may further include analyzing a characteristic of the data samples within each subset of the plurality of subsets of data samples to generate analysis data, and predicting a trend or change in future data samples based on the analysis data. In some examples, the method 2500 may further include performing, based on the predicted trend or change in future data samples, an action as appropriate for the data prediction and the type of data.

### Examples

Example 1 is a method comprising: receiving a first set of viral genome sequences of the same viral species, wherein each viral genome sequence is associated with a sampling date; down-sampling the first set of viral genome sequences to contain a first predetermined quantity of viral genome sequences, comprising: i) randomly selecting viral genome sequences from the first set of viral genome sequences for each lineage of a plurality of lineages, and ii) randomly selecting viral genome sequences from the first set of viral genome sequences and from any of the plurality of lineages until the first predetermined quantity is reached; selecting viral genome sequences from the first set of viral genome sequences that meet at least one quality control (QC) filter of a set of QC filters to generate a second set of viral genome sequences; down-sampling the second set of viral genome sequences into a plurality of subsets of viral genome sequences, wherein each subset contains a second predetermined quantity of viral genome sequences, and wherein each subset is associated with a different timepoint of a plurality of timepoints, comprising: i) selecting, for each timepoint of the plurality of timepoints, at least one representative viral genome sequence from the second set of viral genome sequences of each lineage for each subset, ii) generating, based on the sampling dates associated with each viral genome sequence of the second set of viral genome sequences, a sampling date distribution for the second set of viral genome sequences, and iii) selecting, for each timepoint of the plurality of timepoints, based on the sampling date distribution, viral genome sequences from the second set of viral genome sequences until each subset contains the second predetermined quantity of viral genome sequences; within each subset, aligning coding sequences of each full-length gene-based open reading frame; for each subset, assembling the aligned open reading frame sequences into a full-length coding genome; identifying, based on the full-length coding genomes, one or more sites of the full-length coding genomes under diversifying selection; and predicting one or more future mutations at the one or more sites of the full-length coding genomes under diversifying selection.

In Example 2, the subject matter of Example 1 includes, wherein the viral species is SARS-CoV-2.

In Example 3, the subject matter of Examples 1-2 includes, wherein the first set of viral genome sequences includes tens of millions of genome sequences.

In Example 4, the subject matter of Examples 1-3 includes, wherein the first predetermined quantity of viral genome sequences includes at most two million viral genome sequences.

In Example 5, the subject matter of Examples 1-4 includes, wherein randomly selecting viral genome sequences from the first set of viral genome sequences for each lineage of a plurality of lineages includes randomly selection at most one hundred viral genome sequences.

In Example 6, the subject matter of Examples 1-5 includes, wherein the plurality of lineages include a plurality of Phylogenetic Assignment of Named Global Outbreak (Pango) viral lineages.

In Example 7, the subject matter of Examples 1-6 includes, wherein randomly selecting viral genome sequences from the first set of viral genome sequences for each lineage of a plurality of lineages randomly sampling genome sequences via accession numbers.

In Example 8, the subject matter of Examples 1-7 includes, wherein randomly selecting viral genome sequences from the first set of viral genome sequences for each lineage of a plurality of lineages includes, in cases where there are less than one hundred viral genome sequences for a lineage, selecting all available viral genome sequences for that lineage.

In Example 9, the subject matter of Examples 1-8 includes, wherein randomly selecting viral genome sequences from the first set of viral genome sequences and from any of the plurality of lineages until the first predetermined quantity is reached includes randomly selecting viral genome sequences per time period for each of a plurality of time periods.

In Example 10, the subject matter of Examples 1-9 includes, wherein randomly selecting viral genome sequences from the first set of viral genome sequences and from any of the plurality of lineages until the first predetermined quantity is reached includes randomly selecting viral genome sequences without a lineage constraint or a time period constraint.

In Example 11, the subject matter of Examples 1-10 includes.

In Example 12, the subject matter of Examples 1-11 includes, wherein the second predetermined quantity of viral genome sequences includes at ten thousand viral genome sequences.

In Example 13, the subject matter of Examples 1-12 includes, wherein generating the sampling date distribution includes fitting a quantity of viral genome sequences from the second set of viral genome sequences to a log curve or an exponential curve.

In Example 14, the subject matter of Example 13 includes, wherein fitting the quantity of viral genome sequences from the second set of viral genome sequences to the log curve or the exponential curve includes generating the sampling date distribution by fitting the quantity of viral genome sequences from the second set of viral genome sequences for each month to the log curve or the exponential curve.

In Example 15, the subject matter of Examples 1-14 includes, wherein selecting, for each timepoint of the plurality of timepoints, based on the sampling date distribution, viral genome sequences from the second set of viral genome sequences until each subset contains the second predetermined quantity of viral genome sequences includes: for any timepoint with more than the second predetermined quantity of viral genome sequences available, randomly selecting viral genome sequences from the second set of viral genome sequences until the subset of viral genome sequences associated with the timepoint contains the second predetermined quantity of viral genome sequences, and for any timepoint with less than the second predetermined quantity of viral genome sequences available, retaining all viral genome sequences for the subset of viral genome sequences associated with the timepoint.

In Example 16, the subject matter of Examples 1-15 includes, wherein each timepoint of the plurality of timepoints represents an end of a three-month quarter.

In Example 17, the subject matter of Examples 1-16 includes, aligning coding sequences of each full-length gene-based open reading frame includes applying a codon-aware multiple sequence aligner.

In Example 18, the subject matter of Examples 1-17 includes, wherein the subsets viral genome sequences are configured for selection analysis, wherein the selection analysis is facilitated by the computationally feasible number of viral genome sequences within each subset of viral genome sequences.

In Example 19, the subject matter of Examples 1-18 includes, developing a therapeutic based on the one or more future mutations.

Example 20 is at least one computer-readable medium including instructions that, when executed by one or more processor units, cause the one or more processor units to perform operations to implement of any of Examples 1-19.

Example 21 is an apparatus comprising means to implement of any of Examples 1-19.

Example 22 is a system to implement of any of Examples 1-19.

Example 23 is a method to implement of any of Examples 1-19.

Example 23 is a method comprising: selecting data samples from a first set of data samples, wherein each data sample of the first set of data samples is associated with a sampling date, wherein each data sample of the first set of data samples that meets at least one quality control (QC) criterion, is selected to generate a second set of data samples; and down-sampling the second set of data samples into a plurality of subsets of data samples, wherein each subset of data samples of the plurality of subsets of data samples contains a predetermined quantity of data samples, and wherein each subset of the plurality of subsets of data samples is associated with a different time period of a plurality of sequential time periods, wherein down-sampling the second set of data samples into the plurality of subsets of data samples includes: generating, based on the sampling date associated with each data sample of the second set of data samples, a sampling date distribution for the second set of data samples, and selecting, for each time period of the plurality of sequential time periods, based on the sampling date distribution, data samples from the second set of data samples until each subset of the plurality of subsets of data samples contains the predetermined quantity of data samples.

In Example 24, the subject matter of Example 23 includes, wherein the predetermined quantity of data samples in the first set of data samples includes at least two million data samples.

In Example 25, the subject matter of Examples 23-24 includes, selecting, for each time period of the plurality of sequential time periods, a representative data sample from the second set of data samples of each data subtype of a plurality of data subtype for each subset of the plurality of subsets of data samples.

In Example 26, the subject matter of Examples 23-25 includes, wherein the at least one QC criterion comprises one or more of requiring data source information, requiring the sampling date, requiring lack of data errors, requiring expected information associated with a data sample, requiring a complete data sample, requiring data samples from a particular source, or excluding data samples from a particular source.

In Example 27, the subject matter of Examples 23-26 includes, wherein the quantity of data samples in the first set of data samples comprises at least ten thousand data samples.

In Example 28, the subject matter of Examples 23-27 includes, wherein generating the sampling date distribution comprises fitting a quantity of data samples from the second set of data samples to a predetermined statistical curve or trend.

In Example 29, the subject matter of Examples 23-28 includes, wherein generating the sampling date distribution comprises fitting a quantity of data samples from the second set of data samples to an exponential curve.

In Example 30, the subject matter of Example 29 includes, wherein fitting the quantity of data samples from the second set of data samples to the exponential curve comprises generating the sampling date distribution by fitting the quantity of data samples from the second set of data samples for each of a second time period to the exponential curve.

In Example 31, the subject matter of Examples 23-30 includes, wherein selecting, for each time period of the plurality of sequential time periods, based on the sampling date distribution, data samples from the second set of data samples until each subset contains the predetermined quantity of data samples comprises: for any time period of the plurality of sequential time periods with more than the predetermined quantity of data samples available, randomly selecting data samples from the second set of data samples until the subset of the plurality of subsets of data samples associated with the time period contains the predetermined quantity of data samples, and for any time period of the plurality of sequential time periods with less than the predetermined quantity of data samples available, retaining all data samples for the subset of the plurality of subsets of data samples associated with the time period.

In Example 32, the subject matter of Examples 23-31 includes, analyzing a characteristic of the data samples within each subset of the plurality of subsets of data samples to generate analysis data; and predicting a trend or change in future data samples based on the analysis data.

In Example 33, the subject matter of Examples 23-32 includes, wherein the plurality of subsets of data samples are configured for selection analysis, wherein the selection analysis is facilitated by a computationally feasible number of data samples within each subset of the plurality of subsets of data samples.

In Example 34, the subject matter of Examples 23-33 includes, wherein the first set of data samples are associated with a common data type.

In Example 35, the subject matter of Example 34 includes, wherein the common data type is a common viral species.

In Example 36, the subject matter of Example 35 includes, wherein the common viral species is SARS-CoV-2.

In Example 37, the subject matter of Examples 34-36 includes, wherein the common data type is selected from at least one of: genomic data; data used to track genetic variations in endangered species; data used to monitor disease progression; data used to analyze drug response; data used to monitor ecosystem; data used to monitor genetic changes in plants over time; data used for data compression for storing and transmitting large datasets; data used for early diagnosis of genetic disorders; data used for constructing phylogenetic trees; or genetic data used for training or testing machine learning models.

In Example 38, the subject matter of Examples 34-37 includes, wherein the common data type is selected from stock or financial market data, data used for fraud detection, or data used for network traffic analysis.

In Example 39, the subject matter of Examples 23-38 includes, wherein down-sampling the second set of data samples into the plurality of subsets of data samples is via a machine learning model trained using labeled data to down-sample data samples according to one or more predefined criteria.

In Example 40, the subject matter of Examples 23-39 includes, wherein down-sampling the second set of data samples into the plurality of subsets of data samples is via a large language model trained using labeled data to down-sample data samples according to one or more predefined criteria.

Example 41 is a method comprising: selecting viral genome sequences from a first set of viral genome sequences, wherein each of the first set of viral genome sequences is associated with a sampling date, wherein each of the first set of viral genome sequences that meet at least one quality control (QC) criterion is selected to generate a second set of viral genome sequences; and down-sampling the second set of viral genome sequences into a plurality of subsets of viral genome sequences, wherein each subset of the plurality of subsets of viral genome sequences contains a predetermined quantity of viral genome sequences, and wherein each subset of the plurality of subsets of viral genome sequences is associated with a different timepoint of a plurality of timepoints, comprising: generating, based on the sampling date associated with each viral genome sequence of the second set of viral genome sequences, a sampling date distribution for the second set of viral genome sequences, and selecting, for each timepoint of the plurality of timepoints, based on the sampling date distribution, viral genome sequences from the second set of viral genome sequences until each subset of the plurality of subsets of viral genome sequences contains the predetermined quantity of viral genome sequences; identifying, based on full-length coding genomes for each subset of the plurality of subsets of viral genome sequences, one or more sites of the full-length coding genomes under diversifying selection; and predicting a future mutation at the one or more sites of the full-length coding genomes under diversifying selection.

In Example 42, the subject matter of Example 41 includes, wherein each of the first set of viral genome sequences is associated with a common viral species.

In Example 43, the subject matter of Example 42 includes, wherein the common viral species is SARS-CoV-2.

In Example 44, the subject matter of Examples 41-43 includes, wherein a predetermined quantity of viral genome sequences in the first set of viral genome sequences includes at least two million viral genome sequences.

In Example 45, the subject matter of Examples 41-44 includes, selecting, for each timepoint of the plurality of timepoints, a representative viral genome sequence from the second set of viral genome sequences of each lineage of a plurality of lineages for each subset of the plurality of subsets of viral genome sequences.

In Example 46, the subject matter of Example 45 includes, wherein the plurality of lineages comprise a plurality of Phylogenetic Assignment of Named Global Outbreak (Pango) viral lineages.

In Example 47, the subject matter of Examples 41-46 includes, wherein the at least one QC criterion comprises one or more of requiring complete accession metadata, allowing less than five percent ambiguous DNA nucleotides or translated amino sequences, preventing sequences with runs of six or more consecutive ambiguous nucleotides or amino sequences, requiring the presence of all canonical gene-based open reading frames with full-length BLAST hits against a reference genome, or excluding sequences with open reading frames truncated by premature stop codons, with the exception of ORF8.

In Example 48, the subject matter of Examples 41-47 includes, wherein the predetermined quantity of viral genome sequences comprises at most ten thousand viral genome sequences.

In Example 49, the subject matter of Examples 41-48 includes, wherein generating the sampling date distribution comprises fitting a quantity of viral genome sequences from the second set of viral genome sequences to a predetermined statistical curve or trend.

In Example 50, the subject matter of Examples 41-49 includes, wherein generating the sampling date distribution comprises fitting a quantity of viral genome sequences from the second set of viral genome sequences to a log curve or an exponential curve.

In Example 51, the subject matter of Example 50 includes, wherein fitting the quantity of viral genome sequences from the second set of viral genome sequences to the log curve or the exponential curve comprises generating the sampling date distribution by fitting the quantity of viral genome sequences from the second set of viral genome sequences for each month to the log curve or the exponential curve.

In Example 52, the subject matter of Examples 41-51 includes, wherein selecting, for each timepoint of the plurality of timepoints, based on the sampling date distribution, viral genome sequences from the second set of viral genome sequences until each subset of the plurality of subsets of viral genome sequences contains the predetermined quantity of viral genome sequences comprises: for any timepoint of the plurality of timepoints with more than the predetermined quantity of viral genome sequences available, randomly selecting viral genome sequences from the second set of viral genome sequences until the subset of the plurality of subsets of viral genome sequences associated with the timepoint contains the predetermined quantity of viral genome sequences, and for any timepoint with less than the predetermined quantity of viral genome sequences available, retaining all viral genome sequences for the subset of the plurality of subsets of viral genome sequences associated with the timepoint.

In Example 53, the subject matter of Examples 41-52 includes, wherein each timepoint of the plurality of timepoints represents an end of a three-month quarter.

In Example 54, the subject matter of Examples 41-53 includes, within each subset of the plurality of subsets of viral genome sequences, aligning coding sequences of each full-length gene-based open reading frame; and for each subset of the plurality of subsets of viral genome sequences, assembling the aligned open reading frame sequences into the full-length coding genome.

In Example 55, the subject matter of Example 54 includes, wherein aligning the coding sequences of each full-length gene-based open reading frame comprises applying a codon-aware multiple sequence aligner.

In Example 56, the subject matter of Examples 54-55 includes, developing a therapeutic based on the future mutation.

In Example 57, the subject matter of Examples 41-56 includes, wherein the plurality of subsets of viral genome sequences are configured for selection analysis, wherein the selection analysis is facilitated by a computationally feasible number of viral genome sequences within each subset of the plurality of subsets of viral genome sequences.

In Example 58, the subject matter of Examples 41-57 includes, wherein down-sampling the second set of viral genome sequences into the plurality of subsets of viral genome sequences is via a machine learning model.

In Example 59, the subject matter of Examples 41-58 includes, wherein down-sampling the second set of viral genome sequences into the plurality of subsets of viral genome sequences is via a large language model.

Example 60 is at least one computer-readable medium including instructions that, when executed by one or more processor units, cause the one or more processor units to perform operations to implement of any of Examples 23-40.

Example 61 is an apparatus comprising means to implement of any of Examples 23-40.

Example 62 is a system to implement of any of Examples 23-40.

Example 63 is a method to implement of any of Examples 23-40.

Example 64 is at least one computer-readable medium including instructions that, when executed by one or more processor units, cause the one or more processor units to perform operations to implement of any of Examples 41-59.

Example 65 is an apparatus comprising means to implement of any of Examples 41-59.

Example 66 is a system to implement of any of Examples 41-59.

Example 67 is a method to implement of any of Examples 41-59.

Those skilled in the art will recognize, or be able to ascertain using no more than routine experimentation, many equivalents to the specific embodiments of the method and compositions described herein. Such equivalents are intended to be encompassed by the following claims.

We claim:

1. A method comprising:
selecting data samples from a first set of data samples, wherein each data sample of the first set of data samples is associated with a sampling date, wherein each data sample of the first set of data samples that meets at least one quality control (QC) criterion, is selected to generate a second set of data samples; and
down-sampling the second set of data samples into a plurality of subsets of data samples, wherein each subset of data samples of the plurality of subsets of data samples contains a predetermined quantity of data samples, and wherein each subset of the plurality of subsets of data samples is associated with a different time period of a plurality of sequential time periods, wherein down-sampling the second set of data samples into the plurality of subsets of data samples includes:
generating, based on the sampling date associated with each data sample of the second set of data samples, a sampling date distribution for the second set of data samples, and
selecting, for each time period of the plurality of sequential time periods, based on the sampling date distribution, data samples from the second set of data samples until each subset of the plurality of subsets of data samples contains the predetermined quantity of data samples.

2. The method of claim 1, further comprising selecting, for each time period of the plurality of sequential time periods, a representative data sample from the second set of data samples of each data subtype of a plurality of data subtype for each subset of the plurality of subsets of data samples.

3. The method of claim 1, wherein the at least one QC criterion comprises one or more of: requiring data source information, requiring the sampling date, requiring lack of data errors, requiring expected information associated with a data sample, requiring a complete data sample, requiring data samples from a particular source, or excluding data samples from a particular source.

4. The method of claim 1, wherein generating the sampling date distribution comprises fitting a quantity of data samples from the second set of data samples to a predetermined statistical curve or trend.

5. The method of claim 4, wherein fitting the quantity of data samples from the second set of data samples to the exponential curve comprises generating the sampling date distribution by fitting the quantity of data samples from the second set of data samples for each of a second time period to the exponential curve.

6. The method of claim 1, wherein selecting, for each time period of the plurality of sequential time periods, based on the sampling date distribution, data samples from the second set of data samples until each subset contains the predetermined quantity of data samples comprises:
for any time period of the plurality of sequential time periods with more than the predetermined quantity of data samples available, randomly selecting data samples from the second set of data samples until the subset of the plurality of subsets of data samples associated with the time period contains the predetermined quantity of data samples, and
for any time period of the plurality of sequential time periods with less than the predetermined quantity of data samples available, retaining all data samples for the subset of the plurality of subsets of data samples associated with the time period.

7. The method of claim 1, further comprising:
analyzing a characteristic of the data samples within each subset of the plurality of subsets of data samples to generate analysis data; and
predicting a trend or change in future data samples based on the analysis data.

8. The method of claim 1, wherein the first set of data samples are associated with a common data type, wherein the common data type is selected from at least one of:
genomic data;
data used to track genetic variations in endangered species;
data used to monitor disease progression;
data used to analyze drug response;
data used to monitor ecosystem;
data used to monitor genetic changes in plants over time;
data used for data compression for storing and transmitting large datasets;
data used for early diagnosis of genetic disorders;
data used for constructing phylogenetic trees;
genetic data used for training or testing machine learning models;
stock or financial market data;
data used for fraud detection; or
data used for network traffic analysis.

9. A method comprising:
selecting viral genome sequences from a first set of viral genome sequences, wherein each of the first set of viral genome sequences is associated with a sampling date, wherein each of the first set of viral genome sequences that meet at least one quality control (QC) criterion is selected to generate a second set of viral genome sequences; and
down-sampling the second set of viral genome sequences into a plurality of subsets of viral genome sequences, wherein each subset of the plurality of subsets of viral genome sequences contains a predetermined quantity of viral genome sequences, and wherein each subset of the plurality of subsets of viral genome sequences is associated with a different timepoint of a plurality of timepoints, comprising:
generating, based on the sampling date associated with each viral genome sequence of the second set of viral genome sequences, a sampling date distribution for the second set of viral genome sequences, and
selecting, for each timepoint of the plurality of timepoints, based on the sampling date distribution, viral genome sequences from the second set of viral genome sequences until each subset of the plurality of subsets of viral genome sequences contains the predetermined quantity of viral genome sequences;
identifying, based on full-length coding genomes for each subset of the plurality of subsets of viral genome sequences, one or more sites of the full-length coding genomes under diversifying selection; and
predicting a future mutation at the one or more sites of the full-length coding genomes under diversifying selection.

10. The method of claim 9, wherein each of the first set of viral genome sequences is associated with a common viral species.

11. The method of claim 10, wherein the common viral species is SARS-CoV-2.

12. The method of claim 9, further comprising selecting, for each timepoint of the plurality of timepoints, a representative viral genome sequence from the second set of viral genome sequences of each lineage of a plurality of lineages for each subset of the plurality of subsets of viral genome sequences.

13. The method of claim 9, wherein the at least one QC criterion comprises one or more of: requiring complete accession metadata, allowing less than five percent ambiguous DNA nucleotides or translated amino sequences, preventing sequences with runs of six or more consecutive ambiguous nucleotides or amino sequences, requiring the presence of all canonical gene-based open reading frames with full-length BLAST hits against a reference genome, or excluding sequences with open reading frames truncated by premature stop codons, with the exception of ORF8.

14. The method of claim 9, wherein generating the sampling date distribution comprises fitting a quantity of viral genome sequences from the second set of viral genome sequences to a log curve or an exponential curve.

15. The method of claim 9, wherein selecting, for each timepoint of the plurality of timepoints, based on the sampling date distribution, viral genome sequences from the second set of viral genome sequences until each subset of the plurality of subsets of viral genome sequences contains the predetermined quantity of viral genome sequences comprises:
for any timepoint of the plurality of timepoints with more than the predetermined quantity of viral genome sequences available, randomly selecting viral genome sequences from the second set of viral genome sequences until the subset of the plurality of subsets of viral genome sequences associated with the timepoint contains the predetermined quantity of viral genome sequences, and
for any timepoint with less than the predetermined quantity of viral genome sequences available, retaining all viral genome sequences for the subset of the plurality of subsets of viral genome sequences associated with the timepoint.

16. The method of claim 9, further comprising developing a therapeutic based on the future mutation.

17. A system, comprising:
one or more processor units; and
one or more memories coupled to the one or more processor units, wherein the one or more memories are configured to store processor-executable instructions, which, when executed by the one or more processor units, cause the one or more processor units to:
select data samples from a first set of data samples, wherein each data sample of the first set of data samples is associated with a sampling date, wherein each data sample of the first set of data samples that meets at least one quality control (QC) criterion, is selected to generate a second set of data samples; and
down-sample the second set of data samples into a plurality of subsets of data samples, wherein each subset of data samples of the plurality of subsets of data samples contains a predetermined quantity of data samples, and wherein each subset of the plurality of subsets of data samples is associated with a different time period of a plurality of sequential time periods, wherein down-sampling the second set of data samples into the plurality of subsets of data samples includes:
generate, based on the sampling date associated with each data sample of the second set of data samples, a sampling date distribution for the second set of data samples, and
select, for each time period of the plurality of sequential time periods, based on the sampling date distribution, data samples from the second set of data samples until each subset of the plurality of subsets of data samples contains the predetermined quantity of data samples.

18. The system of claim 17, wherein the processor-executable instructions further cause the one or more processor units to select, for each time period of the plurality of sequential time periods, a representative data sample from the second set of data samples of each data subtype of a plurality of data subtype for each subset of the plurality of subsets of data samples.

19. The system of claim 17, wherein the at least one QC criterion comprises one or more of requiring data source information, requiring the sampling date, requiring lack of data errors, requiring expected information associated with a data sample, requiring a complete data sample, requiring data samples from a particular source, or excluding data samples from a particular source.

20. The system of claim 17, wherein the processor-executable instructions that cause the one or more processor units to generate the sampling date distribution further cause the one or more processor units to fit a quantity of data samples from the second set of data samples to an exponential curve.
